(19) United States
(12) Patent Application Publication
(10) Pub. No.: US 2025/0249101 A1
(43) Pub. Date: Aug. 7, 2025

(54) CELLS AND COMPOSITIONS FOR TREATING CANCER

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Michel Sadelain, New York, NY (US); Sascha P. Haubner, New York, NY (US); Jorge Mansilla-Soto, Tampa, FL (US); Sophie Alexandra Hanina, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/184,397

(22) Filed: Apr. 21, 2025

Related U.S. Application Data
(63) Continuation of application No. PCT/US2023/077500, filed on Oct. 23, 2023.
(60) Provisional application No. 63/418,302, filed on Oct. 21, 2022.

(51) Int. Cl. A61K 40/42; A61K 40/11; A61K 40/31; C07K 14/725; C07K 16/28

(52) U.S. Cl. CPC A61K 40/4232; A61K 40/11; A61K 40/31; A61K 40/4224; C07K 14/7051; C07K 16/2875; C07K 16/2896; A61K 2239/22; A61K 2239/28; C07K 2319/03; C07K 2319/33

(57) ABSTRACT

The presently disclosed subject matter provides cells, compositions and methods for enhancing immune responses toward tumor antigens. It relates to cells comprising a first antigen-recognizing receptor (e.g., a chimeric antigen receptor (CAR)) and a second antigen-recognizing receptor (e.g., a TCR-like fusion molecule). These cells have improved activity and/or efficiency.

### CROSS-REFERENCE TO RELATED APPLICATIONS

This application is a continuation of International Application No. PCT/US2023/077500, filed Oct. 23, 2023, which claims priority to U.S. Provisional Application No. 63/418,302, filed Oct. 21, 2022, the contents of both of which are incorporated by reference in their entireties, and to which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Apr. 21, 2025, is entitled “072734_1744_SL.xml”, and is 235,510 bytes in size.

### INTRODUCTION

The presently disclosed subject matter provides compositions and methods for targeting immune responses toward tumor antigen-bearing cells. It relates to compositions, e.g., modified immunoresponsive cells, comprising a first antigen-recognizing receptor (e.g., a chimeric antigen receptor (CAR)) and a second antigen-recognizing receptor (e.g., a TCR-like fusion molecule), where the compositions mediate an immune response toward cells bearing the first and/or second antigens.

### BACKGROUND OF THE INVENTION

Target heterogeneity can limit the therapeutic success of certain CAR therapies in cancers (e.g., B cell and other malignancies). Several approaches to address target heterogeneity in CAR-based therapies have been described and include the use of dual CARs (T cells co-expressing two CARs), tandem CARs (T cells expressing a single bi-specific CAR) and pooled single CAR-T cells (T cells expressing a single CAR). The clinical efficacy of such CAR therapies is still under ongoing investigation, and emerging pre-clinical data suggests that these approaches may require fine-tuning of combinatorial CAR signaling to balance effector and memory functions, avoiding premature differentiation, and eventual exhaustion. Therefore, novel concepts for addressing target heterogeneity are urgently needed.

### SUMMARY OF THE INVENTION

The presently disclosed subject matter provides compositions, e.g., modified immunoresponsive cells, comprising: (1) a CAR targeting a first antigen; and (2) a TCR-like fusion molecule targeting a second antigen. The presently disclosed subject matter further provides uses of these compositions in reducing tumor burden, treating and/or preventing a neoplasm or a tumor, preventing and/or treating a pathogen infection, preventing and/or treating an autoimmune disease, and/or preventing and/or treating an infectious disease.

In certain embodiments, the CAR comprises an extracellular antigen-binding domain that binds to the first antigen, and an intracellular signaling domain that is capable of delivering an activation signal to the cell. In certain embodiments, the intracellular signaling domain of the CAR comprises a CD3ζ polypeptide. In certain embodiments, the CD3ζ polypeptide is a native CD3ζ polypeptide or a modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the modified CD3ζ polypeptide comprises the amino acid sequence set forth in SEQ ID NO: 22. In certain embodiments, the intracellular signaling domain of the CAR further comprises at least one costimulatory signaling region. In certain embodiments, the at least one costimulatory signaling region comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D. In certain embodiments, the costimulatory molecule comprises amino acids 180 to 220 of SEQ ID NO: 7. In certain embodiments, the CAR comprises a transmembrane domain.

In certain embodiments, the TCR-like fusion molecule comprises i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the second antigen, wherein the TCR-like fusion molecule binds to the second antigen in an HLA-independent manner. In certain embodiments, at least one of the TRAC polypeptide and the TRBC polypeptide is endogenous. In certain embodiments, the first and the second antigen-binding chains bind to the second antigen with a dissociation constant (KD) of about 1×10⁻⁸ M or less. In certain embodiments, the first and the second antigen-binding chains bind to the second antigen with a dissociation constant (KD) of about 5×10⁻⁹ M or less.

In certain embodiments, the first antigen-binding chain comprises an antigen-binding fragment of a VH of an antibody and a TRBC polypeptide, and the second antigen-binding chain comprises an antigen-binding fragment of a VL of the antibody and a TRAC polypeptide. In certain embodiments, the first antigen-binding chain comprises an antigen-binding fragment of a VH of an antibody and a TRAC polypeptide, and the second antigen-binding chain comprises an antigen-binding fragment of a VL of the antibody and a TRBC polypeptide. In certain embodiments, the first and second antigen binding chains are capable of associating with a CD3ζ polypeptide. In certain embodiments, the first and second antigen binding chains, upon binding to the second antigen, are capable of activating the CD3ζ polypeptide. In certain embodiments, the activation of the CD3ζ polypeptide is capable of activating the cell.

In certain embodiments, the cell further comprises a gene disruption of a TRAC locus and/or a TRBC locus. In certain embodiments, the cell further comprises a gene disruption of a CD70 locus. In certain embodiments, the cell further comprises a gene disruption of a TRAC locus, a TRBC locus, and/or a CD70 locus.

In certain embodiments, the cell further comprises a gene modification of a TRAC gene and/or a TRBC gene. In certain embodiments, the cell further comprises a gene modification of a CD70 gene. In certain embodiments, the cell further comprises a gene modification of a TRAC gene, a TRBC gene, and/or a CD70 gene.

In certain embodiments, the immunoresponsive cell is a cell of the lymphoid lineage or a cell of the myeloid lineage. In certain embodiments, the cell of the lymphoid lineage is selected from the group consisting of a T cell, a B cell, a Natural Killer (NK) cell, and a dendritic cell. In certain embodiments, the cell is a T cell. In certain embodiments, the T cell is derived from an induced pluripotent stem cell. In certain embodiments, the T cell is a CD8⁺ T cell. In certain embodiments, the CD8⁺ T cell is CD4 independent.

In certain embodiments, the T cell is selected from the group consisting of a cytotoxic T lymphocyte (CTL), a γδ T cell, a tumor-infiltrating lymphocyte (TIL), a regulatory T cell, and a Natural Killer T (NKT) cell. In certain embodiments, the T cell is CD62L⁺. In certain embodiments, the T cell is CD45RA⁺. In certain embodiments, the T cell is CD45RA⁺ and CD62L⁺.

In certain embodiments, the CAR and/or the TCR-like fusion molecule is integrated at a locus within the genome of the immunoresponsive cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is a TRAC locus.

In certain embodiments, the first antigen and/or the second antigen is a tumor antigen or a pathogen antigen. In certain embodiments, the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, k-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the first antigen is selected from the group consisting of CD312, CD19, CD20, CD22, CD276, and CAIX. In certain embodiments, the second antigen is selected from the group consisting of CD70, CD19, CD20, and CD22.

In certain embodiments, the first antigen and the second antigen are CD312 and CD70. In certain embodiments, the extracellular antigen-binding domain of the CAR targeting CD312 comprises a VH comprising a CDR1 comprising amino acids having the sequence set forth in SEQ ID NO: 73, a CDR2 comprising the amino acid sequence set forth in SEQ ID NO: 74, and a CDR3 comprising the amino acid sequence set forth in SEQ ID NO: 75; and a VL comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78. In certain embodiments, the TCR-like fusion molecule targeting CD70 comprises a first antigen binding chain comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and a second antigen binding chain comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138.

In certain embodiments, the first antigen and the second antigen are CD276 and CD70. In certain embodiments, the extracellular antigen-binding domain of the CAR targeting CD276 comprises a VH comprising a CDR1 comprising amino acids having the sequence set forth in SEQ ID NO: 85, a CDR2 comprising the amino acid sequence set forth in SEQ ID NO: 86, and a CDR3 comprising the amino acid sequence set forth in SEQ ID NO: 87; and a VL comprising a CDR1 comprising the amino acid sequence set forth in SEQ ID NO: 88, a CDR2 comprising the amino acid sequence set forth in SEQ ID NO: 89, and a CDR3 comprising the amino acid sequence set forth in SEQ ID NO: 90. In certain embodiments, the TCR-like fusion molecule targeting CD70 comprises a first antigen binding chain comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and a second antigen binding chain comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138.

In certain embodiments, the first antigen and the second antigen are CAIX and CD70. In certain embodiments, the first antigen and the second antigen are CD19 and CD22. In certain embodiments, the first antigen and the second antigen are CD19 and CD20. In certain embodiments, the first antigen and the second antigen are CD20 and CD22. In certain embodiments, the first antigen and the second antigen are CD20 and CD19. In certain embodiments, the first antigen and the second antigen are CD22 and CD20.

In certain embodiments, the first antigen and the second antigen are CD22 and CD19. In certain embodiments, the extracellular antigen-binding domain of the CAR targeting CD22 comprises: a) a VH comprising a CDR1 comprising amino acids having the sequence set forth in SEQ ID NO: 97, a CDR2 comprising the amino acid sequence set forth in SEQ ID NO: 98, and a CDR3 comprising the amino acid sequence set forth in SEQ ID NO: 99; and a VL comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102; b) a VH comprising a CDR1 comprising amino acids having the sequence set forth in SEQ ID NO: 97, a CDR2 comprising the amino acid sequence set forth in SEQ ID NO: 153, and a CDR3 comprising the amino acid sequence set forth in SEQ ID NO: 99; and a VL comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102; c) a VH comprising a CDR1 comprising amino acids having the sequence set forth in SEQ ID NO: 119, a CDR2 comprising the amino acid sequence set forth in SEQ ID NO: 120, and a CDR3 comprising the amino acid sequence set forth in SEQ ID NO: 121; and a VL comprising a CDR1 comprising the amino acid sequence set forth in SEQ ID NO: 122, a CDR2 comprising the amino acid sequence set forth in SEQ ID NO: 123, and a CDR3 comprising the amino acid sequence set forth in SEQ ID NO: 124; or d) a V_{H} comprising a CDR1 comprising amino acids having the sequence set forth in SEQ ID NO: 97, a CDR2 comprising the amino acid sequence set forth in SEQ ID NO: 154, and a CDR3 comprising the amino acid sequence set forth in SEQ ID NO: 99; and a VL comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. In certain embodiments, the TCR-like fusion molecule targeting CD19 comprises a first antigen binding chain comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and a second antigen binding chain comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148.

In certain embodiments, the first antigen and the second antigen are selected from Table 8.

In certain embodiments, the cell further comprises a chimeric co-stimulating receptor (CCR). In certain embodiments, the CCR comprises an extracellular antigen-binding domain that binds to the third antigen and an intracellular domain that is capable of delivering a costimulatory signal to the cell but does not alone deliver an activation signal to the cell. In certain embodiments, the intracellular domain of the CCR comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the cell further comprises at least one exogenous costimulatory ligand. In certain embodiments, the at least one exogenous co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, FasL, GITRL, TNF-related apoptosis-inducing ligand (TRAIL), CD30L, LIGHT (TNFSF14), CD40L. In certain embodiments, the Ig superfamily member is selected from the group consisting of CD80, CD86, ICOSLG, and combinations thereof. In certain embodiments, the at least one exogenous costimulatory ligand comprises CD80. In certain embodiments, the at least one exogenous a costimulatory ligand comprises 4-1BBL. In certain embodiments, the cell comprises two exogenous costimulatory ligands. In certain embodiments, the at least two exogenous costimulatory ligands comprise CD80 and 4-1BBL. In certain embodiments, the at least two exogenous costimulatory ligands comprise the amino acid sequence set forth in SEQ ID NO: 67 and/or the amino acid sequence set forth in SEQ ID NO: 69.

In certain embodiments, the cell further comprises a fusion polypeptide comprising a) an extracellular domain and a transmembrane domain of a co-stimulatory ligand, and b) an intracellular domain of a first co-stimulatory molecule. In certain embodiments, the co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, GITRL, CD40L, and combinations thereof. In certain embodiments, the Ig superfamily member is selected from the group consisting of CD80, CD86, ICOSLG, and combinations thereof. In certain embodiments, the co-stimulatory ligand is CD80. In certain embodiments, the first co-stimulatory molecule is selected from the group consisting of CD28, 4-1BB, OX40, ICOS, DAP-10, CD27, CD40, NKG2D, CD2, and combinations thereof. In certain embodiments, the first co-stimulatory molecule is 4-1BB. In certain embodiments, the co-stimulatory ligand is CD80 and the first co-stimulatory molecule is 4-1BB. In certain embodiments, the fusion polypeptide comprises the amino acid sequence set forth in SEQ ID NO: 71. In certain embodiments, the fusion polypeptide further comprises an intracellular domain of a second co-stimulatory molecule. In certain embodiments, the second co-stimulatory molecule is selected from the group consisting of CD28, 4-1BB, OX40, ICOS, DAP-10, CD27, CD40, NKG2D, CD2, and combinations thereof. In certain embodiments, the second co-stimulatory molecule is CD28. In certain embodiments, the co-stimulatory ligand is CD80, the first co-stimulatory molecule is 4-1BB, and the second co-stimulatory molecule is CD28. In certain embodiments, the fusion polypeptide comprises the amino acid sequence set forth in SEQ ID NO: 72.

In certain embodiments, the cell is autologous. In certain embodiments, the cell is allogeneic.

In certain embodiments, the presently disclosed subject matter provides compositions comprising the cell disclosed herein. In certain embodiments, the composition is a pharmaceutical composition further comprising a pharmaceutically acceptable excipient.

In certain embodiments, the presently disclosed subject matter provides nucleic acid compositions, vectors, or lipid nanoparticles comprising a first polynucleotide encoding a chimeric antigen receptor (CAR) that targets a first antigen and a second polynucleotide encoding a TCR-like fusion molecule that targets a second antigen. In certain embodiments, the presently disclosed subject matter provides vectors comprising a first polynucleotide encoding a chimeric antigen receptor (CAR) that targets a first antigen and a second polynucleotide encoding a TCR-like fusion molecule that targets a second antigen. In certain embodiments, the vector is a lentiviral vector. In certain embodiments, the vector is a γ-retroviral vector.

In certain embodiments, the presently disclosed subject matter provides polynucleotides encoding a chimeric antigen receptor (CAR) that targets a first antigen and a TCR-like fusion molecule that targets a second antigen. The presently disclosed subject matter also provides vectors and lipid nanoparticles comprising the polynucleotides disclosed herein.

In certain embodiments, the presently disclosed subject matter provides compositions comprising the polynucleotides, vectors, and lipid nanoparticles disclosed herein. In certain embodiments, the composition is a pharmaceutical composition further comprising a pharmaceutically acceptable excipient.

In certain embodiments, the presently disclosed subject matter provides methods for producing an immunoresponsive cell disclosed herein. In certain embodiments, the method comprising introducing into the immunoresponsive cell the nucleic acid composition, a polynucleotide, a vector, or a lipid nanoparticle disclosed herein.

In certain embodiments, the method further comprises generating a gene disruption of a CD70 locus. In certain embodiments, the method further comprises generating a gene disruption of a TRAC locus. In certain embodiments, the gene disruption comprises a substitution, a deletion, an insertion, a mutation, or a combination thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof. In certain embodiments, the gene disruption of the CD70 locus results in a non-functional CD70 protein or in knockout of the CD70 gene expression. In certain embodiments, the gene disruption of the CD70 locus results in a non-functional TRAC protein or in knockout of the TRAC gene expression.

In certain embodiments, generating the gene disruption of comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of a CD70 locus or a TRAC locus is generated before activation of a cell. In certain embodiments, the gene disruption of a CD70 locus or a TRAC locus is generated after activation of a cell. In certain embodiments, a) the gene disruption of a CD70 locus is generated before activation of a cell, and b) the gene disruption of a TRAC locus is generated after activation of a cell. In certain embodiments, a) the gene disruption of a TRAC locus is generated before activation of a cell, and b) the gene disruption of a CD70 locus is generated after activation of a cell.

In certain embodiments, the method further comprises generating a gene modification of a CD70 gene. In certain embodiments, the method further comprises generating a gene modification of a TRAC gene and/or a TRBC gene. In certain embodiments, the method further comprises the gene modification of the CD70 gene results in a non-functional CD70 protein or in knockdown of the CD70 gene expression. In certain embodiments, the method further comprises the gene modification of the TRAC locus results in a non-functional TRAC protein or in knockdown of the TRAC gene expression. In certain embodiments, the method further comprises the gene modification of the TRBC locus results in a non-functional TRBC protein or in knockdown of the TRBC gene expression.

In certain embodiments, the method further comprises introducing a chimeric co-simulating receptor (CCR). In certain embodiments, the method further comprises introducing at least one exogenous costimulatory ligand. In certain embodiments, the method further comprises introducing a fusion polypeptide comprising a) an extracellular domain and a transmembrane domain of a co-stimulatory ligand, and b) an intracellular domain of a first co-stimulatory molecule.

In certain embodiments, the presently disclosed subject matter provides immunoresponsive cells produced by the methods disclosed herein.

In certain embodiments, the presently disclosed subject matter provides methods for reducing tumor burden, treating and/or preventing a neoplasm or a tumor, preventing and/or treating a pathogen infection, preventing and/or treating an autoimmune disease, and/or preventing and/or treating an infectious disease, in a subject, using the immunoresponsive cells or compositions disclosed herein.

In certain embodiments, the neoplasm or tumor is cancer. In certain embodiments, the neoplasm or tumor comprises antigen heterogeneity of the first antigen and the second antigen. In certain embodiments, the second antigen has a low antigen density. In certain embodiments, the second antigen is expressed on tumor cells having a low tumor cell frequency. In certain embodiments, the first antigen and the second antigen are independently selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11. In certain embodiments, the first antigen is selected from the group consisting of CD312, CD19, CD20, CD22, CD276, and CAIX. In certain embodiments, the second antigen is selected from the group consisting of CD70, CD19, CD20, and CD22. In certain embodiments, the first antigen and the second antigen are selected from a) CD312 and CD70; b) CD276 and CD70; c) CAIX and CD70; d) CD19 and CD22; e) CD19 and CD20; f) CD20 and CD22; g) CD20 and CD19; h) CD22 and CD20; or i) CD22 and CD19. In certain embodiments, the first antigen and the second antigen are selected from Table 8.

In certain embodiments, the neoplasm or tumor is a solid tumor. In certain embodiments, the solid tumor is selected from the group consisting of melanoma, renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma. In certain embodiments, the solid tumor is melanoma.

In certain embodiments, the neoplasm or tumor is a blood cancer. In certain embodiments, the neoplasm or tumor is a myeloid disorder. In certain embodiments, the myeloid disorder is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera. In certain embodiments, the myeloid disorder is acute myeloid leukemia (AML).

In certain embodiments, the neoplasm or tumor is a B-cell malignancy. In certain embodiments, the B-cell malignancy is selected from the group consisting of B cell non-Hodgkin lymphoma (NHL), B cell Hodgkin's lymphoma, B cell acute lymphocytic leukemia (ALL), B cell chronic lymphocytic leukemia (CLL), multiple myeloma (MM), CLL with Richter's transformation, and CNS lymphoma. In certain embodiments, the B-cell malignancy is B cell acute lymphocytic leukemia.

In certain embodiments, the neoplasm or tumor is a leukemia. In certain embodiments, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, B-cell precursor acute lymphoblastic leukemia, and T-cell precursor acute lymphoblastic leukemia.

In certain embodiments, the neoplasm or tumor is a lymphoma. In certain embodiments, the lymphoma is selected from the group consisting of Hodgkin's lymphoma, non-Hodgkin's lymphoma, B-cell non-Hodgkin's lymphoma, T-cell non-Hodgkin's lymphoma, and T-cell precursor acute lymphoblastic lymphoma

In certain embodiments, the subject has a relapse of the neoplasm or tumor. In certain embodiments, the subject received treatment which leads to residual tumor cells.

In certain embodiments, the presently disclosed subject matter provides kits comprising the cell or the compositions disclosed herein. In certain embodiments, the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 depicts FACS analysis of TCR-like fusion molecule targeting CD70 (“70HIT”) expressed from the TRAC locus under control of the endogenous TRAC promoter (“TRAC-70HIT”), or expressed from an SFG vector (“SFG-70HIT”).

FIGS. 2A and 2B depict effects of TRAC-70HIT and SFG-70HIT on MOLM13 AML xenograft models. FIG. 2A shows bioluminescence-based tumor quantification. FIG. 2B shows survival curves.

FIGS. 3A-3C depict the effects of SFG-70HIT in patient-derived AML xenograft model. FIG. 3A shows analysis of peripheral blood cells by FACS analysis and AML burden. FIG. 2B shows the total T cell count. FIG. 3C shows FACS analysis at day 20 post-injection.

FIG. 4 depicts FACS analysis of MOL13 wild type and edited cells to model target heterogeneity in AML.

FIGS. 5A-5C depict the effect of 70H_312C-28z1XX in AML heterogeneity model. FIG. 5A shows FACS analysis of the AML heterogeneity model. FIG. 5B shows bioluminescence-based tumor quantification. FIG. 5C shows survival curves.

FIGS. 6A-6C depict the effect of 70H_312C-28z1XX in AML heterogeneity model compared to alternative HIT+CAR designs. FIG. 6A shows FACS analysis of the AML heterogeneity model. FIG. 6B shows bioluminescence-based tumor quantification. FIG. 6C shows survival curves.

FIGS. 7A-7C depict the effect of 70H_312C-28z1XX in AML heterogeneity model compared to alternative dual CAR approaches. FIG. 7A shows FACS analysis of the AML heterogeneity model. FIG. 7B shows bioluminescence-based tumor quantification. FIG. 7C shows survival curves.

FIGS. 8A and 8B depict SFG-70HIT+312CAR-1XX is associated with less differentiated T cell phenotype compared to CAR+CAR. FIG. 8A shows CD8 differentiation. FIG. 8B shows CD4 differentiation.

FIGS. 9A-9C depict SFG-70HIT+312CAR-1XX is efficacious across various heterogenous phenotypes. FIG. 9A shows FACS analysis of CD70 and CD312 in the different tested heterologous phenotypes. FIG. 9B shows bioluminescence-based tumor quantification. FIG. 9C shows survival curves.

FIGS. 10A-10C depict editing strategies for manufacturing of 70H+312C platform. FIG. 10A shows current standard. FIG. 10B shows a first alternative strategy including electroporation at day 0. FIG. 10C shows sequential editing strategy to avoid translocation.

FIGS. 11A and 11B depict effects of different editing strategies for manufacturing of 70H+312C platform on AML tumor model. FIG. 11A shows effects on MOLM13-WT model. FIG. 11B shows effects on an AML heterogeneity model.

FIGS. 12A-12D depict effect of HIT+CAR strategy in solid tumor model. FIG. 12A shows FACS analysis of CD70 and CD276 in human melanoma cell line SK-MEL37. FIG. 12B shows effects in a lung-metastasized melanoma model. FIG. 12C shows effects in an orthotopic skin melanoma model. FIG. 12D shows FACS analysis of the AML heterogeneity model.

FIGS. 13A-13C depict effect of HIT+CAR strategy in renal cell carcinoma model. FIG. 13A shows effects in K5 orthotopic kidney tumor model. FIG. 13B shows effects in K7 orthotopic kidney tumor model. FIG. 13C shows bioluminescence-based tumor quantification of K5 and K7 tumor models.

FIGS. 14A-14D depict efficacy of the cells disclosed herein (HIT+CAR) efficacy in settings of B-ALL. FIG. 14A shows analysis of Nalm6 xenograft cells with wild-type (WT) levels of CD22 and CD19, a first Nalm6 xenograft heterogeneity cells including Nalm6 cells knockout for CD19 (Nalm6-19KO) and CD22 (Nalm6-22KO) at a 1:1 ratio (1:1), and a second Nalm6 xenograft heterogeneity cells including WT, Nalm6-19KO, and Nalm6-22KO at a 1:1:1 ratio (1:1:1). FIG. 14B shows survival curves of animal challenged with the Nalm6 xenograft cells described above and receiving 5E5 T cells including a HIT receptor targeting CD19 and a 1XX CAR targeting CD22 (19-HIT+22-CAR-1XX) or a first CAR targeting CD19 and including a 4-1BB/CD3zeta intracellular domain and a 1XX CAR targeting CD22 (19-CAR-BBz+22-CAR-1XX). FIG. 14C shows analysis of Nalm6 xenograft cells including the indicated percentage values of WT cells (WT), Nalm6-19KO cells (19-KO), and Nalm6-22KO cells (22-KO). FIG. 14D shows survival curves of animal including the Nalm6 xenograft cells described in FIG. 14C and receiving 5E5 T cells including either 19-HIT+22-CAR-1XX or 19-CAR-BBz+22-CAR-1XX.

FIG. 15 shows survival curves of animal including the Nalm6 xenograft cells described above and receiving 1E6 T cells including 19-HIT+22-CAR-1XX, 19-CAR-BBz+22-CAR-1XX, or T cells expressing a single receptor.

FIGS. 16A-16J depict phenotypical and biochemical characterization of T cells in mouse models receiving 19-HIT+22-CAR-1XX or 19-CAR-BBz+22-CAR-1XX. FIG. 16A shows analysis of tumor burden determined by bioluminescence imaging in animals challenged with a 1:1:1 Nalm6 tumor and receiving different doses (5E5 or 1E6) of T cells including 19-HIT+22-CAR-1XX or 19-CAR-BBz+22-CAR-1XX. FIG. 16B shows quantification of Nalm6-GFP⁺ expressing CD19 and/or CD22 in bone marrow 10 days after administration of 5E5 T cells including 19-HIT+22-CAR-1XX (H+C) or 19-CAR-BBz+22-CAR-1XX (C+C). FIG. 16C shows quantification of Nalm6-GFP+expressing CD19 and/or CD22 in bone marrow 16 days after administration of 5E5 T cells including 19-HIT+22-CAR-1XX (H+C) or 19-CAR-BBz+22-CAR-1XX (C+C). FIG. 16D shows cell count of CD8+ and CD4⁺ T cells in bone marrow and spleen of mice challenging with 1:1:1 Nalm6 tumors 10 days and 16 days after administration of 5E5 T cells including 19-HIT+22-CAR-1XX (H+C) or 19-CAR-BBz+22-CAR-1XX (C+C). FIG. 16E shows cell phenotyping of CD8⁺ T cells in bone marrow and spleen of mice challenged with 1:1:1 Nalm6 tumors 10 days and 16 days after administration of 5E5 T cells including 19-HIT+22-CAR-1XX (H+C) or 19-CAR-BBz+22-CAR-1XX (C+C). FIG. 16F shows cell phenotyping of CD4⁺ T cells in bone marrow and spleen of mice challenged with 1:1:1 Nalm6 tumors 10 days and 16 days after administration of 5E5 T cells including 19-HIT+22-CAR-1XX (H+C) or 19-CAR-BBz+22-CAR-1XX (C+C). FIG. 16G shows cell exhaustion phenotyping of CD8⁺ T cells in bone marrow and spleen of mice challenged with 1:1:1 Nalm6 tumors 10 days and 16 days after administration of 5E5 T cells including 19-HIT+22-CAR-1XX (H+C) or 19-CAR-BBz+22-CAR-1XX (C+C). FIG. 16H shows cell exhaustion phenotyping of CD4⁺ T cells in bone marrow and spleen of mice challenged with 1:1:1 Nalm6 tumors 10 days and 16 days after administration of 5E5 T cells including 19-HIT+22-CAR-1XX (H+C) or 19-CAR-BBz+22-CAR-1XX (C+C). FIG. 16I shows expression of cell exhaustion markers in CD8⁺ T cells in bone marrow and spleen of mice challenged with 1:1:1 Nalm6 tumors 10 days and 16 days after administration of 5E5 T cells including 19-HIT+22-CAR-1XX (H+C) or 19-CAR-BBz+22-CAR-1XX (C+C). FIG. 16J shows expression of cell exhaustion markers in CD4⁺ T cells in bone marrow and spleen of mice challenged with 1:1:1 Nalm6 tumors 10 days and 16 days after administration of 5E5 T cells including 19-HIT+22-CAR-1XX (H+C) or 19-CAR-BBz+22-CAR-1XX (C+C).

### DETAILED DESCRIPTION OF THE INVENTION

The presently disclosed subject matter provides compositions, e.g., modified immune cells, useful for immunotherapy (e.g., T cell immunotherapy). The presently disclosed compositions, e.g., modified immune cells, comprise: (1) a chimeric antigen receptor (CAR) that targets a first antigen; and (2) a TCR-like fusion molecule that targets a second antigen. The presently disclosed subject matter also provides methods for producing such compositions, and methods of using such compositions for treating and/or preventing tumors (e.g., cancer, e.g., a solid tumor or a blood cancer, e.g., a myeloid disorder, e.g., acute myeloid leukemia (AML)). The presently disclosed subject matter is based, at least in part, on the discovery that an OR-gated targeting approach including a HIT and a CAR can address antigen heterogeneity (i.e., the presence of a tumor cell population with mixed phenotype, including tumor cells with either low antigen density or low tumor cell frequency). Co-expression of a HIT and a CAR that are directed against two independent target antigens can enhance at least one activity of the cells, e.g., cytotoxicity, cell proliferation, and/or cell persistence. Further, co-expression of a HIT and a CAR reduces cumulative costimulation driving excessive T cell differentiation and limiting functional persistence.

Non-limiting embodiments of the presently disclosed subject matter are described by the present specification and Examples.

For purposes of clarity of disclosure and not by way of limitation, the detailed description is divided into the following subsections:
1. Definitions;
2. Cells;
3. Nucleic Acid Compositions and Vectors;
4. Formulations and Administration;
5. Methods of Treatment;
6. Kits; and
7. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refer to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, ICOS, DAP-10, CD27, NKG2D, CD2, CD150, CD226. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

As used herein, the term “antigen heterogeneity” refers to the differential expression of a number of antigens (e.g., tumor antigens, e.g., CD70, CD312) which results in variation in the tumor cell phenotype and distribution of tumor antigen-positive cells.

As used herein, the term “low antigen density” refers to a target molecule (e.g., an antigen) having a cell surface density of less than about 5,000 molecules per cell. In certain embodiments, the low antigen density is a cell surface density is less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, the low antigen density is a cell surface density is less than about 2,000 molecules per cell. In certain embodiments, the low antigen density is a cell surface density is less than about 1,500 molecules per cell. In certain embodiments, the low antigen density is a cell surface density is less than about 1,000 molecules per cell. In certain embodiments, the low antigen density is a cell surface density is between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

As used herein, the term “low tumor cell frequency” refers to a target cell having a target cell frequency of less than about 50% per tumor. In certain embodiments, the low tumor cell frequency is less than about 40% per tumor, less than about 30% per tumor, less than about 20% per tumor, less than about 15% per tumor, less than about 10% per tumor, less than about 5% per tumor, less than about 2% per tumor, or less than about 1% per tumor. In certain embodiments, the low tumor cell frequency is less than about 2% per tumor. In certain embodiments, the low tumor cell frequency is less than about 1.5% per tumor. In certain embodiments, the low tumor cell frequency is less than about 1% per tumor. In certain embodiments, the low tumor cell frequency is between about 40% per tumor and about 20% per tumor, between about 20% per tumor and about 10% per tumor, between about 15% per tumor and about 10% per tumor, between about 20% per tumor and about 5% per tumor, between about 10% per tumor and about 2% per tumor, or between about 10% per tumor and about 1% per tumor.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain variable fragment (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L}region. The light chain constant region is comprised of one domain, C_{L}. The V_{H} and V_{L}regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs regions are delineated using the PyIgClassify system (Adolf-Bryfogle et al., Nucleic acids research 43.D1 (2015): D432-D438).

As used herein, the term “Linker” shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker is a G4S linker. In certain embodiments, the linker comprises or consists of the amino acid sequence set forth in SEQ ID NO: 1, which is provided below:

[SEQ ID NO: 1]
GGGGSGGGGSGGGGS

In certain embodiments, the linker comprises or consists of the amino acid sequence set forth in SEQ ID NO: 2, which is provided below:

[SEQ ID NO: 2]
GGGGGGGGSGGGSGGGGS

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
SGGGGS

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol1997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed antigen recognizing receptors (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” or “malignancy” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

Other aspects of the presently disclosed subject matter are described in the following disclosure and are within the ambit of the presently disclosed subject matter.

### 2. Cells

The presently disclosed subject matter provides cells comprising a) a first antigen-recognizing receptor that targets a first antigen, and b) a second antigen-recognizing receptor that targets a second antigen.

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the T cell is a CD62L⁺ T cell. In certain embodiments, the T cell is a CD45RA⁺ T cell. In certain embodiments, the T cell is a CD62L⁺/CD45RA⁺ T cell.

In certain embodiments, the cell is a NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. In certain embodiments, the cell (e.g., T cell) is non-autologous. In certain embodiments, the cell (e.g., T cell) is allogeneic. In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

### 2.1. First Antigen-Recognizing Receptor

The first antigen-recognizing receptor targets a first antigen. The first antigen can be a tumor antigen or a pathogen antigen. In certain embodiments, the first antigen-recognizing receptor is a chimeric receptor. In certain embodiments, the chimeric receptor is a chimeric antigen receptor (CAR).

### 2.1.1. FirstAntigen

In certain embodiments, the first antigen is a tumor antigen. In certain embodiments, the tumor antigen is an antigen with low antigen density. In certain embodiments, the tumor antigen is expressed on a cell with low tumor cell frequency.

Any tumor antigen (antigenic peptide) can be used in the tumor-related embodiments described herein. Sources of antigen include, but are not limited to, cancer proteins. The first antigen can be expressed as a peptide or as an intact protein or a portion thereof. The intact protein or portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, K-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the first antigen is selected from the group consisting of CD312, CLEC12A, CD33, CD123, IL1RAP, SIGLEC-6, GRP78, TIM3, CD70, CD20, CD22, CD19, GPRC5D, SLAMF7, BCMA, CD276, and CAIX. In certain embodiments, the first antigen is CD312. In certain embodiments, the first antigen is CD276.

In certain embodiments, the first antigen is a pathogen antigen. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 2.1.2. Chimeric Antigen Receptors (CARs)

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4+ and CD8⁺ T cells through their CD3ζ chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40,NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the first antigen-recognizing receptor is a CAR comprising an extracellular antigen-binding domain that binds to the first antigen, and an intracellular signaling domain. In certain embodiments, the CAR further comprises a transmembrane domain. In certain embodiments, the CAR further comprises a hinger/spacer region.

### 2.1.2.1. Extracellular Antigen-Binding Domain

In certain embodiments, the extracellular antigen-binding domain of the CAR (for example, an scFv) binds to the first antigen with a dissociation constant (K_{D}) of about 5×10⁻⁷ M or less, about 1×10⁻⁷ M or less, about 5×10⁻⁸M or less, about 1×10⁻⁸M or less, about 5×10⁻⁹ M or less, or about 1×10⁻⁹ M or less, or about 1×10⁻¹⁰ M or less. In certain embodiments, the extracellular antigen-binding domain of the CAR (for example, an scFv) binds to the first antigen with a K_{D} of about 1×10⁻⁸M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

### 2.1.2.1.1. Exemplary Extracellular Antigen-Binding Domains

In certain embodiments, the first antigen-recognizing receptor is a CAR comprising an extracellular antigen-binding domain that binds to CD312. In certain embodiments, the extracellular antigen-binding domain of the CAR comprises or consists of the amino acid sequence set forth in SEQ ID NO: 83 and specifically binds to CD312, e.g., a human CD312 polypeptide. SEQ ID NO: 83 is provided in Table 1 below.

In certain embodiments, the extracellular antigen-binding domain of the CAR comprises a heavy chain variable region (V_{H}) comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof. In certain embodiments, the extracellular antigen-binding domain of the CAR comprises a V_{H} comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75. SEQ ID NOs: 73-75 are provided in Table 1 below.

In certain embodiments, the extracellular antigen-binding domain of the CAR comprises a light chain variable region (V_{L}) comprising a CDR1 comprising the amino acid sequence set forth in SEQ ID NO: 76 or a conservative modification thereof, a CDR2 comprising the amino acid sequence set forth in SEQ ID NO: 77 or a conservative modification thereof, and a CDR3 comprising the amino acid sequence set forth in SEQ ID NO: 78 or a conservative modification thereof. In certain embodiments, the extracellular antigen-binding domain of the CAR comprises a V_{L} comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78. SEQ ID NOs: 76-78 are provided in Table 1 below.

In certain embodiments, the extracellular antigen-binding domain of the CAR comprises a V_{H} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75, a conservative modification thereof; a V_{L} comprising a CDR1 comprising the amino acid sequence set forth in SEQ ID NO: 76 or a conservative modification thereof, a CDR2 comprising the amino acid sequence set forth in SEQ ID NO: 77 or a conservative modification thereof, and a CDR3 comprising the amino acid sequence set forth in SEQ ID NO: 78 or a conservative modification thereof. In certain embodiments, the extracellular antigen-binding domain of the CAR comprises a V_{H} comprising a CDR1 comprising amino acids having the sequence set forth in SEQ ID NO: 73, a CDR2 comprising the amino acid sequence set forth in SEQ ID NO: 74, and a CDR3 comprising the amino acid sequence set forth in SEQ ID NO: 75; and a V_{L} comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78.

In certain embodiments, extracellular antigen-binding domain of the CAR comprises a V_{H} comprising a CDR1, a CDR2, and a CDR3 of the V_{H} having the amino acid sequence set forth in SEQ ID NO: 79. In certain embodiments, extracellular antigen-binding domain of the CAR comprises a V_{L} comprising a CDR1, a CDR2, and a CDR3 of the V_{L} having the amino acid sequence set forth in SEQ ID NO: 81. In certain embodiments, extracellular antigen-binding domain of the CAR comprises a V_{H} comprising a CDR1, a CDR2, and a CDR3 of the V_{H} having the amino acid sequence set forth in SEQ ID NO: 79; and a V_{L} Comprising a CDR1, a CDR2, and a CDR3 of the V_{L} having the amino acid sequence set forth in SEQ ID NO: 81.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 79. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 79. In certain embodiments, the extracellular antigen-binding domain of the CAR comprises a V_{H} comprising the amino acid sequence set forth in SEQ ID NO: 79. SEQ ID NO: 79 is provided in Table 1 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 81. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 81. In certain embodiments, the extracellular antigen-binding domain of the CAR comprises a V_{L}comprising the amino acid sequence set forth in SEQ ID NO: 81. SEQ ID NO: 81 is provided in Table 1 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 79, and a V_{L} comprising an amino acid sequence that is at least about 80% (e.g., at least about 85%, at least about 90%, or at least about 95%) homologous or identical to the amino acid sequence set forth in SEQ ID NO: 81.

In certain embodiments, the extracellular antigen-binding domain of the CAR comprises a V_{H} comprising the amino acid sequence set forth in SEQ ID NO: 79. In certain embodiments, the extracellular antigen-binding domain of the CAR comprises a V_{L} comprising the amino acid sequence set forth in SEQ ID NO: 81. In certain embodiments, the extracellular antigen-binding domain of the CAR comprises V_{H} comprising the amino acid sequence set forth in SEQ ID NO: 79 and a V_{L} comprising the amino acid sequence set forth in SEQ ID NO: 81.

In certain embodiments, the extracellular antigen-binding domain of the CAR is an scFv that comprises or consists of the amino acid sequence set forth in SEQ ID NO: 83. SEQ ID NOs: 73-83 are provided in the following Table 1. In certain embodiments, the V_{H}- and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the CDRs regions/sequences disclosed herein are delineated using the Kabat system (Swindells et al, J Mol Biol. 2017 Feb. 3; 429(3):356-364).

TABLE 1
anti-CD312 scFv

| CDRs | 1 | 2 | 3 |
|---|---|---|---|
| V_{H} | NYWMQ | AVYPGDGDTRHTQKFKG | GFTAYGMDY |
| | [SEQ ID NO: 73] | [SEQ ID NO: 74] | [SEQ ID NO: 75] |
| V_{L} | SASSSVSYMH | DTSKLAS | QQWSSNPLT |
| | [SEQ ID NO: 76] | [SEQ ID NO: 77] | [SEQ ID NO: 78] |

| | |
|---|---|
| Full V_{H} | QVQLQQSGAELARPGASVKLSCKASGYTFTNYWMQWIKQRPGQGLEWIGAVYPGDGDT |
| | RHTQKFKGKATLTADKSSSTAYMQVSSLASEDSAVYYCARGFTAYGMDYWGQGTSVTV |
| | SS [SEQ ID NO: 79] |
| DNA-Full | CAAGTTCAGCTGCAACAGTCTGGCGCCGAGCTTGCTAGACCTGGCGCTTCTGTGAAGC |
| V_{H} | TGAGCTGTAAAGCCAGCGGCTACACCTTCACCAACTACTGGATGCAGTGGATCAAGCA |
| | GCGGCCTGGACAGGGACTCGAGTGGATTGGAGCTGTGTATCCTGGCGACGGCGACACC |
| | AGACACACCCAGAAGTTCAAGGGCAAAGCCACACTGACCGCCGACAAGAGCAGCAGCA |
| | CAGCCTACATGCAGGTTTCCAGCCTGGCCAGCGAAGATAGCGCCGTGTACTATTGCGC |
| | CAGAGGCTTTACCGCCTACGGCATGGATTATTGGGGCCAGGGCACAAGCGTGACCGTG |
| | TCTAGC [SEQ ID NO: 80] |
| Full V_{L} | QIVLTQSPAVMSASPGEKVTMTCSASSSVSYMHWYQQKSGTSPKRWIYDTSKLASGVP |
| | NRFSGSGSGTSYSFTIRSMEAEDAATYYCQQWSSNPLTFGAGTKLELKR [SEQ ID |
| | NO: 81] |
| DNA-Full | CAAATTGTGCTGACACAGAGCCCTGCCGTGATGTCTGCTAGCCCTGGCGAGAAAGTGA |
| V_{L} | CCATGACCTGTAGCGCCAGCAGCAGCGTGTCCTACATGCACTGGTATCAGCAGAAGTC |
| | CGGCACAAGCCCCAAGCGGTGGATCTACGATACAAGCAAGCTGGCCTCCGGCGTGCCC |
| | AATAGATTTTCTGGCAGCGGCTCTGGCACCAGCTACAGCTTCACCATCAGATCCATGG |
| | AAGCCGAGGATGCCGCCACCTACTACTGCCAGCAGTGGTCCAGCAATCCCCTGACATT |
| | TGGAGCCGGCACCAAGCTGGAACTGAAGAGA [SEQ ID NO: 82] |
| scFv | MALPVTALLLPLALLLHAQVQLQQSGAELARPGASVKLSCKASGYTFTNYWMQWIKQR |
| | PGQGLEWIGAVYPGDGDTRHTQKFKGKATLTADKSSSTAYMQVSSLASEDSAVYYCAR |
| | GFTAYGMDYWGQGTSVTVSSGGGGSGGGGSGGGGSQIVLTQSPAVMSASPGEKVTMTC |
| | SASSSVSYMHWYQQKSGTSPKRWIYDTSKLASGVPNRESGSGSGTSYSFTIRSMEAED |
| | AATYYCQQWSSNPLTFGAGTKLELKR [SEQ ID NO: 83] |
| scFv DNA | ATGGCTCTCCCAGTGACTGCCCTACTGCTTCCCCTAGCGCTTCTCCTGCATGCACAAG |
| | TTCAGCTGCAACAGTCTGGCGCCGAGCTTGCTAGACCTGGCGCTTCTGTGAAGCTGAG |
| | CTGTAAAGCCAGCGGCTACACCTTCACCAACTACTGGATGCAGTGGATCAAGCAGCGG |
| | CCTGGACAGGGACTCGAGTGGATTGGAGCTGTGTATCCTGGCGACGGCGACACCAGAC |
| | ACACCCAGAAGTTCAAGGGCAAAGCCACACTGACCGCCGACAAGAGCAGCAGCACAGC |
| | CTACATGCAGGTTTCCAGCCTGGCCAGCGAAGATAGCGCCGTGTACTATTGCGCCAGA |
| | GGCTTTACCGCCTACGGCATGGATTATTGGGGCCAGGGCACAAGCGTGACCGTGTCTA |
| | GCGGTGGAGGTGGATCAGGTGGAGGTGGATCTGGTGGAGGTGGATCTCAAATTGTGCT |
| | GACACAGAGCCCTGCCGTGATGTCTGCTAGCCCTGGCGAGAAAGTGACCATGACCTGT |
| | AGCGCCAGCAGCAGCGTGTCCTACATGCACTGGTATCAGCAGAAGTCCGGCACAAGCC |
| | CCAAGCGGTGGATCTACGATACAAGCAAGCTGGCCTCCGGCGTGCCCAATAGATTTTC |
| | TGGCAGCGGCTCTGGCACCAGCTACAGCTTCACCATCAGATCCATGGAAGCCGAGGAT |
| | GCCGCCACCTACTACTGCCAGCAGTGGTCCAGCAATCCCCTGACATTTGGAGCCGGCA |
| | CCAAGCTGGAACTGAAGAGA [SEQ ID NO: 84] |

In certain embodiments, the first antigen-recognizing receptor is a CAR comprising an extracellular antigen-binding domain that binds to CD276. In certain embodiments, the extracellular antigen-binding domain of the CAR comprises or consists of the amino acid sequence set forth in SEQ ID NO: 95 and specifically binds to CD276, e.g., a human CD276 polypeptide. SEQ ID NO: 95 is provided in Table 2 below.

In certain embodiments, the extracellular antigen-binding domain of the CAR comprises a heavy chain variable region (V_{H}) comprising a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87 or a conservative modification thereof. In certain embodiments, the extracellular antigen-binding domain of the CAR comprises a V_{H} comprising a CDR1 comprising the amino acid sequence set forth in SEQ ID NO: 85, a CDR2 comprising the amino acid sequence set forth in SEQ ID NO: 86, and a CDR3 comprising the amino acid sequence set forth in SEQ ID NO: 87. SEQ ID NOs: 85-87 are provided in Table 2 below.

In certain embodiments, the extracellular antigen-binding domain of the CAR comprises a light chain variable region (V_{L}) comprising a CDR1 comprising the amino acid sequence set forth in SEQ ID NO: 88 or a conservative modification thereof, a CDR2 comprising the amino acid sequence set forth in SEQ ID NO: 89 or a conservative modification thereof, and a CDR3 comprising the amino acid sequence set forth in SEQ ID NO: 90 or a conservative modification thereof. In certain embodiments, the extracellular antigen-binding domain of the CAR comprises a V_{L} comprising a CDR1 comprising the amino acid sequence set forth in SEQ ID NO: 88, a CDR2 comprising the amino acid sequence set forth in SEQ ID NO: 89, and a CDR3 comprising the amino acid sequence set forth in SEQ ID NO: 90. SEQ ID NOs: 88-90 are provided in Table 2 below.

In certain embodiments, the extracellular antigen-binding domain of the CAR comprises a V_{H} comprising a CDR1 comprising the amino acid sequence set forth in SEQ ID NO: 85 or a conservative modification thereof, a CDR2 comprising the amino acid sequence set forth in SEQ ID NO: 86 or a conservative modification thereof, and a CDR3 comprising the amino acid sequence set forth in SEQ ID NO: 87, a conservative modification thereof; a V_{L} comprising a CDR1 comprising the amino acid sequence set forth in SEQ ID NO: 88 or a conservative modification thereof, a CDR2 comprising the amino acid sequence set forth in SEQ ID NO: 89 or a conservative modification thereof, and a CDR3 comprising the amino acid sequence set forth in SEQ ID NO: 90 or a conservative modification thereof. In certain embodiments, the extracellular antigen-binding domain of the CAR comprises a V_{H} comprising a CDR1 comprising amino acids having the sequence set forth in SEQ ID NO: 85, a CDR2 comprising the amino acid sequence set forth in SEQ ID NO: 86, and a CDR3 comprising the amino acid sequence set forth in SEQ ID NO: 87; and a V_{L} comprising a CDR1 comprising the amino acid sequence set forth in SEQ ID NO: 88, a CDR2 comprising the amino acid sequence set forth in SEQ ID NO: 89, and a CDR3 comprising the amino acid sequence set forth in SEQ ID NO: 90.

In certain embodiments, extracellular antigen-binding domain of the CAR comprises a V_{H} comprising a CDR1, a CDR2, and a CDR3 of the V_{H} having the amino acid sequence set forth in SEQ ID NO: 91. In certain embodiments, extracellular antigen-binding domain of the CAR comprises a V_{L} comprising a CDR1, a CDR2, and a CDR3 of the V_{L} having the amino acid sequence set forth in SEQ ID NO: 93. In certain embodiments, extracellular antigen-binding domain of the CAR comprises a V_{H} comprising a CDR1, a CDR2, and a CDR3 of the V_{H} having the amino acid sequence set forth in SEQ ID NO: 91; and a V_{L} Comprising a CDR1, a CDR2, and a CDR3 of the V_{L} having the amino acid sequence set forth in SEQ ID NO: 93.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 91. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 91. In certain embodiments, the extracellular antigen-binding domain of the CAR comprises a V_{H} comprising the amino acid sequence set forth in SEQ ID NO: 91. SEQ ID NO: 91 is provided in Table 2 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 93. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 93. In certain embodiments, the extracellular antigen-binding domain of the CAR comprises a V_{L}comprising the amino acid sequence set forth in SEQ ID NO: 93. SEQ ID NO: 93 is provided in Table 2 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 91, and a V_{L} comprising an amino acid sequence that is at least about 80% (e.g., at least about 85%, at least about 90%, or at least about 95%) homologous or identical to the amino acid sequence set forth in SEQ ID NO: 93.

In certain embodiments, the extracellular antigen-binding domain of the CAR comprises a V_{H} comprising the amino acid sequence set forth in SEQ ID NO: 91. In certain embodiments, the extracellular antigen-binding domain of the CAR comprises a V_{L} comprising the amino acid sequence set forth in SEQ ID NO: 93. In certain embodiments, the extracellular antigen-binding domain of the CAR comprises V_{H} comprising the amino acid sequence set forth in SEQ ID NO: 91 and a V_{L} comprising the amino acid sequence set forth in SEQ ID NO: 93.

In certain embodiments, the extracellular antigen-binding domain of the CAR is an scFv that comprises or consists of the amino acid sequence set forth in SEQ ID NO: 95. SEQ ID NOs: 85-95 are provided in the following Table 2. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the CDRs regions/sequences disclosed herein are delineated using the Kabat system (Swindells et al., J Mol Biol. 2017 Feb. 3; 429(3):356-364).

TABLE 2
anti-CD276 scFv

| CDRs | 1 | 2 | 3 |
|---|---|---|---|
| V_{H} | KASQDVRTAVA [SEQ ID | SASYRYT [SEQ ID NO: | QQHYGTPPWT [SEQ |
| | NO: 85] | 86] | ID NO: 87] |
| V_{L} | SYAMS [SEQ ID NO: | AISGGGRYTYYPDSMKG | HYDGYLDY [SEQ ID |
| | 88] | [SEQ ID NO: 89] | NO: 90] |

| | |
|---|---|
| Full V_{H} | DIVMTQSHKEMSTSIGARVSITCKASQDVRTAVAWYQQKPGQSPKLLIYSASYRYTGV |
| | PDRFTGSGSGTDFTFTISSVQAEDLAVYYCQQHYGTPPWTFGGGTKLEIK [SEQ ID |
| | NO: 91] |
| DNA-Full V_{H} | GACATTGTGATGACCCAGTCTCACAAATTCATGTCCACATCAATTGGAGCCAGGGTCA |
| | GCATCACCTGCAAGGCCAGTCAGGATGTGAGAACTGCTGTAGCCTGGTATCAACAGAA |
| | ACCAGGCCAGTCTCCTAAACTACTAATTTACTCGGCATCCTACCGGTACACTGGAGTC |
| | CCTGATCGCTTCACTGGCAGTGGATCTGGGACGGATTTCACTTTCACCATCAGCAGTG |
| | TGCAGGCTGAAGACCTGGCAGTTTATTACTGTCAGCAACATTATGGTACTCCTCCGTG |
| | GACGTTCGGTGGAGGCACCAAGCTGGAAATCAAA |
| | [SEQ ID NO: 92] |
| Full V_{L} | EVQLVESGGGLVKPGGSLKLSCEASRFTESSYAMSWVRQTPEKRLEWVAAISGGGRYT |
| | YYPDSMKGRFTISRDNAKNFLYLQMSSLRSEDTAMYYCARHYDGYLDYWGQGTTLTVS |
| | S |
| | [SEQ ID NO: 93] |
| DNA-Full | GAAGTGCAGCTGGTGGAGTCTGGGGGAGGCTTAGTGAAGCCTGGAGGGTCCCTGAAAC |
| V_{L} | TCTCCTGTGAAGCCTCTAGATTCACTTTCAGTAGCTATGCCATGTCTTGGGTTCGCCA |
| | GACTCCGGAGAAGAGGCTGGAGTGGGTCGCAGCCATTAGTGGAGGTGGTAGGTACACC |
| | TACTATCCAGACAGTATGAAGGGTCGATTCACCATCTCCAGAGACAATGCCAAGAATT |
| | TCCTGTACCTGCAAATGAGCAGTCTGAGGTCTGAGGACACGGCCATGTATTACTGTGC |
| | AAGACACTATGATGGTTATCTTGACTACTGGGGCCAAGGCACCACTCTCACAGTCTCC |
| | TCA [SEQ ID NO: 94] |
| scFv | MEFGLSWLFLVAILKGVQCDIVMTQSHKFMSTSIGARVSITCKASQDVRTAVAWYQQK |
| | PGQSPKLLIYSASYRYTGVPDRFTGSGSGTDFTFTISSVQAEDLAVYYCQQHYGTPPW |
| | TFGGGTKLEIKGGGGSGGGGSGGGGSEVQLVESGGGLVKPGGSLKLSCEASRFTESSY |
| | AMSWVRQTPEKRLEWVAAISGGGRYTYYPDSMKGRFTISRDNAKNFLYLQMSSLRSED |
| | TAMYYCARHYDGYLDYWGQGTTLTVSS [SEQ ID NO: 95] |
| scFv DNA | ATGGAATTCGGCCTGAGCTGGCTGTTCCTGGTGGCCATCCTGAAGGGCGTGCAGTGCG |
| | ACATTGTGATGACCCAGTCTCACAAATTCATGTCCACATCAATTGGAGCCAGGGTCAG |
| | CATCACCTGCAAGGCCAGTCAGGATGTGAGAACTGCTGTAGCCTGGTATCAACAGAAA |
| | CCAGGCCAGTCTCCTAAACTACTAATTTACTCGGCATCCTACCGGTACACTGGAGTCC |
| | CTGATCGCTTCACTGGCAGTGGATCTGGGACGGATTTCACTTTCACCATCAGCAGTGT |
| | GCAGGCTGAAGACCTGGCAGTTTATTACTGTCAGCAACATTATGGTACTCCTCCGTGG |
| | ACGTTCGGTGGAGGCACCAAGCTGGAAATCAAAGGCGGCGGAGGATCTGGCGGAGGCG |
| | GAAGTGGCGGAGGGGGCTCTGAAGTGCAGCTGGTGGAGTCTGGGGGAGGCTTAGTGAA |
| | GCCTGGAGGGTCCCTGAAACTCTCCTGTGAAGCCTCTAGATTCACTTTCAGTAGCTAT |
| | GCCATGTCTTGGGTTCGCCAGACTCCGGAGAAGAGGCTGGAGTGGGTCGCAGCCATTA |
| | GTGGAGGTGGTAGGTACACCTACTATCCAGACAGTATGAAGGGTCGATTCACCATCTC |
| | CAGAGACAATGCCAAGAATTTCCTGTACCTGCAAATGAGCAGTCTGAGGTCTGAGGAC |
| | ACGGCCATGTATTACTGTGCAAGACACTATGATGGTTATCTTGACTACTGGGGCCAAG |
| | GCACCACTCTCACAGTCTCCTCA [SEQ ID NO: 96] |

In certain embodiments, the first antigen-recognizing receptor is a CAR comprising an extracellular antigen-binding domain that binds to CD22. In certain embodiments, the extracellular antigen-binding domain of the CAR comprises or consists of the amino acid sequence set forth in SEQ ID NO: 107 or SEQ ID NO: 109 and specifically binds to CD22, e.g., a human CD22 polypeptide. SEQ ID NO: 107 and SEQ ID NO: 109 are provided in Table 3 below.

In certain embodiments, the extracellular antigen-binding domain of the CAR comprises a heavy chain variable region (V_{H}) comprising a CDR1 comprising the amino acid sequence set forth in SEQ ID NO: 97 or a conservative modification thereof, a CDR2 comprising the amino acid sequence set forth in SEQ ID NO: 98 or a conservative modification thereof, and a CDR3 comprising the amino acid sequence set forth in SEQ ID NO: 99 or a conservative modification thereof. In certain embodiments, the extracellular antigen-binding domain of the CAR comprises a V_{H} comprising a CDR1 comprising the amino acid sequence set forth in SEQ ID NO: 97, a CDR2 comprising the amino acid sequence set forth in SEQ ID NO: 98, and a CDR3 comprising the amino acid sequence set forth in SEQ ID NO: 99. SEQ ID NOs: 97-99 are provided in Table 3 below.

In certain embodiments, the extracellular antigen-binding domain of the CAR comprises a light chain variable region (V_{L}) comprising a CDR1 comprising the amino acid sequence set forth in SEQ ID NO: 100 or a conservative modification thereof, a CDR2 comprising the amino acid sequence set forth in SEQ ID NO: 101 or a conservative modification thereof, and a CDR3 comprising the amino acid sequence set forth in SEQ ID NO: 102 or a conservative modification thereof. In certain embodiments, the extracellular antigen-binding domain of the CAR comprises a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. SEQ ID NOs: 100-102 are provided in Table 3 below.

In certain embodiments, the extracellular antigen-binding domain of the CAR comprises a V_{H} comprising a CDR1 comprising the amino acid sequence set forth in SEQ ID NO: 97 or a conservative modification thereof, a CDR2 comprising the amino acid sequence set forth in SEQ ID NO: 98 or a conservative modification thereof, and a CDR3 comprising the amino acid sequence set forth in SEQ ID NO: 99, a conservative modification thereof; a V_{L} comprising a CDR1 comprising the amino acid sequence set forth in SEQ ID NO: 100 or a conservative modification thereof, a CDR2 comprising the amino acid sequence set forth in SEQ ID NO: 101 or a conservative modification thereof, and a CDR3 comprising the amino acid sequence set forth in SEQ ID NO: 102 or a conservative modification thereof. In certain embodiments, the extracellular antigen-binding domain of the CAR comprises a V_{H} comprising a CDR1 comprising amino acids having the sequence set forth in SEQ ID NO: 97, a CDR2 comprising the amino acid sequence set forth in SEQ ID NO: 98, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102.

In certain embodiments, extracellular antigen-binding domain of the CAR comprises a V_{H} comprising a CDR1, a CDR2, and a CDR3 of the V_{H} having the amino acid sequence set forth in SEQ ID NO: 103. In certain embodiments, extracellular antigen-binding domain of the CAR comprises a V_{L} comprising a CDR1, a CDR2, and a CDR3 of the V_{L} having the amino acid sequence set forth in SEQ ID NO: 105. In certain embodiments, extracellular antigen-binding domain of the CAR comprises a V_{H} comprising a CDR1, a CDR2, and a CDR3 of the V_{H} having the amino acid sequence set forth in SEQ ID NO: 103; and a V_{L} Comprising a CDR1, a CDR2, and a CDR3 of the V_{L} having the amino acid sequence set forth in SEQ ID NO: 105.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 103. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 103. In certain embodiments, the extracellular antigen-binding domain of the CAR comprises a V_{H} comprising the amino acid sequence set forth in SEQ ID NO: 103. SEQ ID NO: 103 is provided in Table 3 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 105. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 105. In certain embodiments, the extracellular antigen-binding domain of the CAR comprises a V_{L}comprising the amino acid sequence set forth in SEQ ID NO: 105. SEQ ID NO: 105 is provided in Table 3 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 103, and a V_{L} comprising an amino acid sequence that is at least about 80% (e.g., at least about 85%, at least about 90%, or at least about 95%) homologous or identical to the amino acid sequence set forth in SEQ ID NO: 105.

In certain embodiments, the extracellular antigen-binding domain of the CAR comprises a V_{H} comprising the amino acid sequence set forth in SEQ ID NO: 103. In certain embodiments, the extracellular antigen-binding domain of the CAR comprises a V_{L} comprising the amino acid sequence set forth in SEQ ID NO: 105. In certain embodiments, the extracellular antigen-binding domain of the CAR comprises V_{H} comprising the amino acid sequence set forth in SEQ ID NO: 103 and a V_{L} comprising the amino acid sequence set forth in SEQ ID NO: 105.

In certain embodiments, the extracellular antigen-binding domain of the CAR is an scFv that comprises or consists of the amino acid sequence set forth in SEQ ID NO: 107 or SEQ ID NO: 109. SEQ ID NOs: 97-109 are provided in the following Table 3. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the CDRs regions/sequences disclosed herein are delineated using the Kabat system (Swindells et al, J Mol Biol. 2017 Feb. 3; 429(3):356-364).

TABLE 3
anti-CD22 scFv

| CDRs | 1 | 2 | 3 |
|---|---|---|---|
| V_{H} | SNSAAWN [SEQ ID NO: | RTYYRSKWYNDYAVSVKS | EVTGDLEDAFDI |
| | 97] | [SEQ ID NO: 98] | [SEQ ID NO: 99] |
| V_{L} | RASQTIWSYLN [SEQ ID | AASSLQS [SEQ ID NO: | QQSYSIPQT [SEQ |
| | NO: 100] | 101] | ID NO: 102] |

| | |
|---|---|
| Full V_{H} | QVQLQQSGPGLVKPSQTLSLTCAISGDSVSSNSAAWNWIRQSPSRGLEWLGRTYYRSK |
| | WYNDYAVSVKSRITINPDTSKNQFSLQLNSVTPEDTAVYYCAREVTGDLEDAFDIWGQ |
| | GTMVTVSS [SEQ ID NO: 103] |
| DNA-Full | CAGTGCCAAGTACAGCTTCAACAGTCAGGTCCGGGGCTGGTAAAGCCTTCTCAGACAC |
| V_{H} | TCAGCCTGACTTGTGCTATAAGTGGGGATAGTGTTTCATCCAACTCAGCGGCGTGGAA |
| | CTGGATCCGGCAAAGTCCATCTCGGGGCCTGGAGTGGTTGGGGCGGACCTATTATAGG |
| | TCTAAGTGGTACAATGATTACGCCGTCTCAGTGAAGTCACGGATCACAATCAATCCCG |
| | ATACGAGTAAGAATCAGTTCTCACTTCAGCTTAACAGTGTGACACCTGAAGATACGGC |
| | AGTATATTATTGCGCGAGAGAGGTTACTGGGGACCTCGAAGATGCCTTCGATATCTGG |
| | GGTCAAGGCACAATGGTTACAGTCAGCTCC [SEQ ID NO: 104] |
| Full V_{L} | DIQMTQSPSSLSASVGDRVTITCRASQTIWSYLNWYQQRPGKAPNLLIYAASSLQSGV |
| | PSRFSGRGSGTDFTLTISSLQAEDFATYYCQQSYSIPQTFGQGTKLEIK [SEQ ID |
| | NO: 105] |
| DNA-Full | GACATACAGATGACACAATCTCCGAGTAGCCTTTCCGCATCCGTAGGTGATAGGGTTA |
| V_{L} | CCATAACTTGCCGCGCATCTCAAACGATCTGGTCCTATCTGAACTGGTACCAGCAGAG |
| | ACCAGGAAAAGCTCCTAATCTGCTTATCTACGCCGCAAGCTCACTGCAGTCTGGGGTT |
| | CCGAGTAGATTTTCTGGGCGAGGCAGCGGAACGGATTTTACTCTGACCATAAGCTCTC |
| | TGCAAGCAGAAGATTTTGCCACGTACTACTGCCAGCAATCTTACAGCATCCCACAAAC |
| | ATTTGGACAAGGCACAAAGTTGGAGATCAAA [SEQ ID NO: 106] |
| scFv | MELGLSWIFLLAILKGVQCQVQLQQSGPGLVKPSQTLSLTCAISGDSVSSNSAAWNWI |
| | RQSPSRGLEWLGRTYYRSKWYNDYAVSVKSRITINPDTSKNQFSLQLNSVTPEDTAVY |
| | YCAREVTGDLEDAFDIWGQGTMVTVSSGGGGSGGGGSGGGGSDIQMTQSPSSLSASVG |
| | DRVTITCRASQTIWSYLNWYQQRPGKAPNLLIYAASSLQSGVPSRESGRGSGTDFTLT |
| | ISSLQAEDFATYYCQQSYSIPQTFGQGTKLEIK [SEQ ID NO: 107] |
| scFv DNA | ATGGAACTCGGTCTGTCCTGGATTTTTCTGCTTGCGATCTTGAAAGGAGTGCAGTGCC |
| | AAGTACAGCTTCAACAGTCAGGTCCGGGGCTGGTAAAGCCTTCTCAGACACTCAGCCT |
| | GACTTGTGCTATAAGTGGGGATAGTGTTTCATCCAACTCAGCGGCGTGGAACTGGATC |
| | CGGCAAAGTCCATCTCGGGGCCTGGAGTGGTTGGGGCGGACCTATTATAGGTCTAAGT |
| | GGTACAATGATTACGCCGTCTCAGTGAAGTCACGGATCACAATCAATCCCGATACGAG |
| | TAAGAATCAGTTCTCACTTCAGCTTAACAGTGTGACACCTGAAGATACGGCAGTATAT |
| | TATTGCGCGAGAGAGGTTACTGGGGACCTCGAAGATGCCTTCGATATCTGGGGTCAAG |
| | GCACAATGGTTACAGTCAGCTCCGGTGGAGGTGGATCAGGTGGAGGTGGATCTGGTGG |
| | AGGTGGATCTGACATACAGATGACACAATCTCCGAGTAGCCTTTCCGCATCCGTAGGT |
| | GATAGGGTTACCATAACTTGCCGCGCATCTCAAACGATCTGGTCCTATCTGAACTGGT |
| | ACCAGCAGAGACCAGGAAAAGCTCCTAATCTGCTTATCTACGCCGCAAGCTCACTGCA |
| | GTCTGGGGTTCCGAGTAGATTTTCTGGGCGAGGCAGCGGAACGGATTTTACTCTGACC |
| | ATAAGCTCTCTGCAAGCAGAAGATTTTGCCACGTACTACTGCCAGCAATCTTACAGCA |
| | TCCCACAAACATTTGGACAAGGCACAAAGTTGGAGATCAAA [SEQ ID NO: 108] |
| scFv v2 | MELGLSWIFLLAILKGVQCQVQLQQSGPGLVKPSQTLSLTCAISGDSVSSNSAAWNWI |
| | RQSPSRGLEWLGRTYYRSKWYNDYAVSVKSRITINPDTSKNQFSLQLNSVTPEDTAVY |
| | YCAREVTGDLEDAFDIWGQGTMVTVSSGGGGSDIQMTQSPSSLSASVGDRVTITCRAS |
| | QTIWSYLNWYQQRPGKAPNLLIYAASSLQSGVPSRESGRGSGTDFTLTISSLQAEDFA |
| | TYYCQQSYSIPQTFGQGTKLEIK [SEQ ID NO: 109] |
| scFv DNA | ATGGAACTCGGTCTGTCCTGGATTTTTCTGCTTGCGATCTTGAAAGGAGTGCAGTGCC |
| v2 | AAGTACAGCTTCAACAGTCAGGTCCGGGGCTGGTAAAGCCTTCTCAGACACTCAGCCT |
| | GACTTGTGCTATAAGTGGGGATAGTGTTTCATCCAACTCAGCGGCGTGGAACTGGATC |
| | CGGCAAAGTCCATCTCGGGGCCTGGAGTGGTTGGGGCGGACCTATTATAGGTCTAAGT |
| | GGTACAATGATTACGCCGTCTCAGTGAAGTCACGGATCACAATCAATCCCGATACGAG |
| | TAAGAATCAGTTCTCACTTCAGCTTAACAGTGTGACACCTGAAGATACGGCAGTATAT |
| | TATTGCGCGAGAGAGGTTACTGGGGACCTCGAAGATGCCTTCGATATCTGGGGTCAAG |
| | GCACAATGGTTACAGTCAGCTCCGGAGGAGGAGGCAGCGACATACAGATGACACAATC |
| | TCCGAGTAGCCTTTCCGCATCCGTAGGTGATAGGGTTACCATAACTTGCCGCGCATCT |
| | CAAACGATCTGGTCCTATCTGAACTGGTACCAGCAGAGACCAGGAAAAGCTCCTAATC |
| | TGCTTATCTACGCCGCAAGCTCACTGCAGTCTGGGGTTCCGAGTAGATTTTCTGGGCG |
| | AGGCAGCGGAACGGATTTTACTCTGACCATAAGCTCTCTGCAAGCAGAAGATTTTGCC |
| | ACGTACTACTGCCAGCAATCTTACAGCATCCCACAAACATTTGGACAAGGCACAAAGT |
| | TGGAGATCAAA [SEQ ID NO: 110] |

In certain embodiments, the first antigen-recognizing receptor is a CAR comprising an extracellular antigen-binding domain that binds to CD22. In certain embodiments, the extracellular antigen-binding domain of the CAR comprises or consists of the amino acid sequence set forth in SEQ ID NO: 115 or SEQ ID NO: 117 and specifically binds to CD22, e.g., a human CD22 polypeptide. SEQ ID NO: 115 and SEQ ID NO: 117 are provided in Table 4 below.

In certain embodiments, the extracellular antigen-binding domain of the CAR comprises a heavy chain variable region (V_{H}) comprising a CDR1 comprising the amino acid sequence set forth in SEQ ID NO: 97 or a conservative modification thereof, a CDR2 comprising the amino acid sequence set forth in SEQ ID NO: 153 or a conservative modification thereof, and a CDR3 comprising the amino acid sequence set forth in SEQ ID NO: 99 or a conservative modification thereof. In certain embodiments, the extracellular antigen-binding domain of the CAR comprises a V_{H} comprising a CDR1 comprising the amino acid sequence set forth in SEQ ID NO: 97, a CDR2 comprising the amino acid sequence set forth in SEQ ID NO: 153, and a CDR3 comprising the amino acid sequence set forth in SEQ ID NO: 99. SEQ ID NOs: 97, 99, and 153 are provided in Table 4 below.

In certain embodiments, the extracellular antigen-binding domain of the CAR comprises a light chain variable region (V_{L}) comprising a CDR1 comprising the amino acid sequence set forth in SEQ ID NO: 100 or a conservative modification thereof, a CDR2 comprising the amino acid sequence set forth in SEQ ID NO: 101 or a conservative modification thereof, and a CDR3 comprising the amino acid sequence set forth in SEQ ID NO: 102 or a conservative modification thereof. In certain embodiments, the extracellular antigen-binding domain of the CAR comprises a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. SEQ ID NOs: 100-102 are provided in Table 4 below.

In certain embodiments, the extracellular antigen-binding domain of the CAR comprises a V_{H} comprising a CDR1 comprising the amino acid sequence set forth in SEQ ID NO: 97 or a conservative modification thereof, a CDR2 comprising the amino acid sequence set forth in SEQ ID NO: 153 or a conservative modification thereof, and a CDR3 comprising the amino acid sequence set forth in SEQ ID NO: 99, a conservative modification thereof; a V_{L} comprising a CDR1 comprising the amino acid sequence set forth in SEQ ID NO: 100 or a conservative modification thereof, a CDR2 comprising the amino acid sequence set forth in SEQ ID NO: 101 or a conservative modification thereof, and a CDR3 comprising the amino acid sequence set forth in SEQ ID NO: 102 or a conservative modification thereof. In certain embodiments, the extracellular antigen-binding domain of the CAR comprises a V_{H} comprising a CDR1 comprising amino acids having the sequence set forth in SEQ ID NO: 97, a CDR2 comprising the amino acid sequence set forth in SEQ ID NO: 153, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102.

In certain embodiments, extracellular antigen-binding domain of the CAR comprises a V_{H} comprising a CDR1, a CDR2, and a CDR3 of the V_{H} having the amino acid sequence set forth in SEQ ID NO: 111. In certain embodiments, extracellular antigen-binding domain of the CAR comprises a V_{L} comprising a CDR1, a CDR2, and a CDR3 of the V_{L} having the amino acid sequence set forth in SEQ ID NO: 113. In certain embodiments, extracellular antigen-binding domain of the CAR comprises a V_{H} comprising a CDR1, a CDR2, and a CDR3 of the V_{H} having the amino acid sequence set forth in SEQ ID NO: 111; and a V_{L} Comprising a CDR1, a CDR2, and a CDR3 of the V_{L} having the amino acid sequence set forth in SEQ ID NO: 113.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 111. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 111. In certain embodiments, the extracellular antigen-binding domain of the CAR comprises a V_{H} comprising the amino acid sequence set forth in SEQ ID NO: 111. SEQ ID NO: 111 is provided in Table 4 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 113. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 113. In certain embodiments, the extracellular antigen-binding domain of the CAR comprises a V_{L}comprising the amino acid sequence set forth in SEQ ID NO: 113. SEQ ID NO: 113 is provided in Table 4 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 111, and a V_{L} comprising an amino acid sequence that is at least about 80% (e.g., at least about 85%, at least about 90%, or at least about 95%) homologous or identical to the amino acid sequence set forth in SEQ ID NO: 113.

In certain embodiments, the extracellular antigen-binding domain of the CAR comprises a V_{H} comprising the amino acid sequence set forth in SEQ ID NO: 111. In certain embodiments, the extracellular antigen-binding domain of the CAR comprises a V_{L} comprising the amino acid sequence set forth in SEQ ID NO: 113. In certain embodiments, the extracellular antigen-binding domain of the CAR comprises V_{H} comprising the amino acid sequence set forth in SEQ ID NO: 111 and a V_{L} comprising the amino acid sequence set forth in SEQ ID NO: 113.

In certain embodiments, the extracellular antigen-binding domain of the CAR is an scFv that comprises or consists of the amino acid sequence set forth in SEQ ID NO: 115 or SEQ ID NO: 117. SEQ ID NOs: 99-102 and 111-117 are provided in the following Table 4. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the CDRs regions/sequences disclosed herein are delineated using the Kabat system (Swindells et al., J Mol Biol. 2017 Feb. 3; 429(3):356-364).

TABLE 4
anti-CD22 scFv

| CDRs | 1 | 2 | 3 |
|---|---|---|---|
| V_{H} | SNSAAWN [SEQ ID NO: | RTYYRSKWYNDYAVSVKS | EVTGDLEDAFDI |
| | 97] | [SEQ ID NO: 153] | [SEQ ID NO: 99] |
| V_{L} | RASQTIWSYLN [SEQ ID | AASSLQS [SEQ ID NO: | QQSYSIPQT [SEQ |
| | NO: 100] | 101] | ID NO: 102] |

| | |
|---|---|
| Full V_{H} | QVQLQQSGPGLVKPSQTLSLTCAISGDSVSSNSAAWNWIRQSPSRGLEWLGRTYYRSK |
| | WYNDYAVSVKSRITINPDTSKNQFSLQLNSVTPEDTAVYYCAREVTGDLEDAFDIWGQ |
| | GTMVTVSS [SEQ ID NO: 111] |
| DNA-Full | CAAGTACAGCTTCAACAGTCAGGTCCGGGGCTGGTAAAGCCTTCTCAGACACTCAGCC |
| V_{H} | TGACTTGTGCTATAAGTGGGGATAGTGTTTCATCCAACTCAGCGGCGTGGAACTGGAT |
| | CCGGCAAAGTCCATCTCGGGGCCTGGAGTGGTTGGGGCGGACCTATTATAGGTCTAAG |
| | TGGTACAATGATTACGCCGTCTCAGTGAAGTCACGGATCACAATCAATCCCGATACGA |
| | GTAAGAATCAGTTCTCACTTCAGCTTAACAGTGTGACACCTGAAGATACGGCAGTATA |
| | TTATTGCGCGAGAGAGGTTACTGGGGACCTCGAAGATGCCTTCGATATCTGGGGTCAA |
| | GGCACAATGGTTACAGTCAGCTCC [SEQ ID NO: 112] |
| Full V_{L} | DIQMTQSPSSLSASVGDRVTITCRASQTIWSYLNWYQQRPGKAPNLLIYAASSLQSGV |
| | PSRFSGRGSGTDFTLTISSLQAEDFATYYCQQSYSI PQTFGQGTKLEIK [SEQ ID |
| | NO: 113] |
| DNA-Full | GACATACAGATGACACAATCTCCGAGTAGCCTTTCCGCATCCGTAGGTGATAGGGTTA |
| V_{L} | CCATAACTTGCCGCGCATCTCAAACGATCTGGTCCTATCTGAACTGGTACCAGCAGAG |
| | ACCAGGAAAAGCTCCTAATCTGCTTATCTACGCCGCAAGCTCACTGCAGTCTGGGGTT |
| | CCGAGTAGATTTTCTGGGCGAGGCAGCGGAACGGATTTTACTCTGACCATAAGCTCTC |
| | TGCAAGCAGAAGATTTTGCCACGTACTACTGCCAGCAATCTTACAGCATCCCACAAAC |
| | ATTTGGACAAGGCACAAAGTTGGAGATCAAA [SEQ ID NO: 114] |
| scFv | MELGLSWIFLLAILKGVQCQVQLQQSGPGLVKPSQTLSLTCAISGDSVSSNSAAWNWI |
| | RQSPSRGLEWLGRTYYRSKWYNDYAVSVKSRITINPDTSKNQFSLQLNSVTPEDTAVY |
| | YCAREVTGDLEDAFDIWGQGTMVTVSSGGGGSGGGGSGGGGSDIQMTQSPSSLSASVG |
| | DRVTITCRASQTIWSYLNWYQQRPGKAPNLLIYAASSLQSGVPSRESGRGSGTDETLT |
| | ISSLQAED [SEQ ID NO: 115] |
| scFv DNA | ATGGAACTCGGTCTGTCCTGGATTTTTCTGCTTGCGATCTTGAAAGGAGTGCAGTGCC |
| | AAGTACAGCTTCAACAGTCAGGTCCGGGGCTGGTAAAGCCTTCTCAGACACTCAGCCT |
| | GACTTGTGCTATAAGTGGGGATAGTGTTTCATCCAACTCAGCGGCGTGGAACTGGATC |
| | CGGCAAAGTCCATCTCGGGGCCTGGAGTGGTTGGGGCGGACCTATTATAGGTCTAAGT |
| | GGTACAATGATTACGCCGTCTCAGTGAAGTCACGGATCACAATCAATCCCGATACGAG |
| | TAAGAATCAGTTCTCACTTCAGCTTAACAGTGTGACACCTGAAGATACGGCAGTATAT |
| | TATTGCGCGAGAGAGGTTACTGGGGACCTCGAAGATGCCTTCGATATCTGGGGTCAAG |
| | GCACAATGGTTACAGTCAGCTCCGGTGGAGGTGGATCAGGTGGAGGTGGATCTGGTGG |
| | AGGTGGATCTGACATACAGATGACACAATCTCCGAGTAGCCTTTCCGCATCCGTAGGT |
| | GATAGGGTTACCATAACTTGCCGCGCATCTCAAACGATCTGGTCCTATCTGAACTGGT |
| | ACCAGCAGAGACCAGGAAAAGCTCCTAATCTGCTTATCTACGCCGCAAGCTCACTGCA |
| | GTCTGGGGTTCCGAGTAGATTTTCTGGGCGAGGCAGCGGAACGGATTTTACTCTGACC |
| | ATAAGCTCTCTGCAAGCAGAAGAT [SEQ ID NO: 116] |
| scFv v2 | MELGLSWIFLLAILKGVQCQVQLQQSGPGLVKPSQTLSLTCAISGDSVSSNSAAWNWI |
| | RQSPSRGLEWLGRTYYRSKWYNDYAVSVKSRITINPDTSKNQFSLQLNSVTPEDTAVY |
| | YCAREVTGDLEDAFDIWGQGTMVTVSSGGGGSDIQMTQSPSSLSASVGDRVTITCRAS |
| | QTIWSYLNWYQQRPGKAPNLLIYAASSLQSGVPSRESGRGSGTDFTLTISSLQAEDFA |
| | TYYCQQSYSIPQTFGQGTKLEIK [SEQ ID NO: 117] |
| scFv DNA | ATGGAACTCGGTCTGTCCTGGATTTTTCTGCTTGCGATCTTGAAAGGAGTGCAGTGCC |
| v2 | AAGTACAGCTTCAACAGTCAGGTCCGGGGCTGGTAAAGCCTTCTCAGACACTCAGCCT |
| | GACTTGTGCTATAAGTGGGGATAGTGTTTCATCCAACTCAGCGGCGTGGAACTGGATC |
| | CGGCAAAGTCCATCTCGGGGCCTGGAGTGGTTGGGGCGGACCTATTATAGGTCTAAGT |
| | GGTACAATGATTACGCCGTCTCAGTGAAGTCACGGATCACAATCAATCCCGATACGAG |
| | TAAGAATCAGTTCTCACTTCAGCTTAACAGTGTGACACCTGAAGATACGGCAGTATAT |
| | TATTGCGCGAGAGAGGTTACTGGGGACCTCGAAGATGCCTTCGATATCTGGGGTCAAG |
| | GCACAATGGTTACAGTCAGCTCCGGAGGAGGAGGCAGCGACATACAGATGACACAATC |
| | TCCGAGTAGCCTTTCCGCATCCGTAGGTGATAGGGTTACCATAACTTGCCGCGCATCT |
| | CAAACGATCTGGTCCTATCTGAACTGGTACCAGCAGAGACCAGGAAAAGCTCCTAATC |
| | TGCTTATCTACGCCGCAAGCTCACTGCAGTCTGGGGTTCCGAGTAGATTTTCTGGGCG |
| | AGGCAGCGGAACGGATTTTACTCTGACCATAAGCTCTCTGCAAGCAGAAGATTTTGCC |
| | ACGTACTACTGCCAGCAATCTTACAGCATCCCACAAACATTTGGACAAGGCACAAAGT |
| | TGGAGATCAAA [SEQ ID NO: 118] |

In certain embodiments, the first antigen-recognizing receptor is a CAR comprising an extracellular antigen-binding domain that binds to CD22. In certain embodiments, the extracellular antigen-binding domain of the CAR comprises or consists of the amino acid sequence set forth in SEQ ID NO: 129 or SEQ ID NO: 131 and specifically binds to CD22, e.g., a human CD22 polypeptide. SEQ ID NO: 129 and SEQ ID NO: 131 are provided in Table 5 below.

In certain embodiments, the extracellular antigen-binding domain of the CAR comprises a heavy chain variable region (V_{H}) comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121 or a conservative modification thereof. In certain embodiments, the extracellular antigen-binding domain of the CAR comprises a V_{H} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. SEQ ID NOs: 119-121 are provided in Table 5 below.

In certain embodiments, the extracellular antigen-binding domain of the CAR comprises a light chain variable region (V_{L}) comprising a CDR1 comprising the amino acid sequence set forth in SEQ ID NO: 122 or a conservative modification thereof, a CDR2 comprising the amino acid sequence set forth in SEQ ID NO: 123 or a conservative modification thereof, and a CDR3 comprising the amino acid sequence set forth in SEQ ID NO: 124 or a conservative modification thereof. In certain embodiments, the extracellular antigen-binding domain of the CAR comprises a V_{L} comprising a CDR1 comprising the amino acid sequence set forth in SEQ ID NO: 122, a CDR2 comprising the amino acid sequence set forth in SEQ ID NO: 123, and a CDR3 comprising the amino acid sequence set forth in SEQ ID NO: 124. SEQ ID NOs: 122-124 are provided in Table 5 below.

In certain embodiments, the extracellular antigen-binding domain of the CAR comprises a V_{H} comprising a CDR1 comprising the amino acid sequence set forth in SEQ ID NO: 119 or a conservative modification thereof, a CDR2 comprising the amino acid sequence set forth in SEQ ID NO: 120 or a conservative modification thereof, and a CDR3 comprising the amino acid sequence set forth in SEQ ID NO: 121, a conservative modification thereof; a V_{L} Comprising a CDR1 comprising the amino acid sequence set forth in SEQ ID NO: 122 or a conservative modification thereof, a CDR2 comprising the amino acid sequence set forth in SEQ ID NO: 123 or a conservative modification thereof, and a CDR3 comprising the amino acid sequence set forth in SEQ ID NO: 124 or a conservative modification thereof. In certain embodiments, the extracellular antigen-binding domain of the CAR comprises a V_{H} comprising a CDR1 comprising amino acids having the sequence set forth in SEQ ID NO: 119, a CDR2 comprising the amino acid sequence set forth in SEQ ID NO: 120, and a CDR3 comprising the amino acid sequence set forth in SEQ ID NO: 121; and a V_{L} comprising a CDR1 comprising the amino acid sequence set forth in SEQ ID NO: 122, a CDR2 comprising the amino acid sequence set forth in SEQ ID NO: 123, and a CDR3 comprising the amino acid sequence set forth in SEQ ID NO: 124.

In certain embodiments, extracellular antigen-binding domain of the CAR comprises a V_{H} comprising a CDR1, a CDR2, and a CDR3 of the V_{H} having the amino acid sequence set forth in SEQ ID NO: 125. In certain embodiments, extracellular antigen-binding domain of the CAR comprises a V_{L} comprising a CDR1, a CDR2, and a CDR3 of the V_{L} having the amino acid sequence set forth in SEQ ID NO: 127. In certain embodiments, extracellular antigen-binding domain of the CAR comprises a V_{H} comprising a CDR1, a CDR2, and a CDR3 of the V_{H} having the amino acid sequence set forth in SEQ ID NO: 125; and a V_{L} Comprising a CDR1, a CDR2, and a CDR3 of the V_{L} having the amino acid sequence set forth in SEQ ID NO: 127.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 125. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 125. In certain embodiments, the extracellular antigen-binding domain of the CAR comprises a V_{H} comprising the amino acid sequence set forth in SEQ ID NO: 125. SEQ ID NO: 125 is provided in Table 5 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 127. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 127. In certain embodiments, the extracellular antigen-binding domain of the CAR comprises a V_{L}comprising the amino acid sequence set forth in SEQ ID NO: 127. SEQ ID NO: 127 is provided in Table 5 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 125, and a V_{L} comprising an amino acid sequence that is at least about 80% (e.g., at least about 85%, at least about 90%, or at least about 95%) homologous or identical to the amino acid sequence set forth in SEQ ID NO: 127.

In certain embodiments, the extracellular antigen-binding domain of the CAR comprises a V_{H} comprising the amino acid sequence set forth in SEQ ID NO: 125. In certain embodiments, the extracellular antigen-binding domain of the CAR comprises a V_{L} comprising the amino acid sequence set forth in SEQ ID NO: 127. In certain embodiments, the extracellular antigen-binding domain of the CAR comprises V_{H} comprising the amino acid sequence set forth in SEQ ID NO: 125 and a V_{L} comprising the amino acid sequence set forth in SEQ ID NO: 127.

In certain embodiments, the extracellular antigen-binding domain of the CAR is an scFv that comprises or consists of the amino acid sequence set forth in SEQ ID NO: 129 or SEQ ID NO: 131. SEQ ID NOs: 119-132 are provided in the following Table 5. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the CDRs regions/sequences disclosed herein are delineated using the Kabat system (Swindells et al., J Mol Biol. 2017 Feb. 3; 429(3):356-364).

TABLE 5
anti-CD22 scFv

| | CDRs | | |
|---|---|---|---|
| | 1 | 2 | 3 |
| V_{H} | IYDMS [SEQ ID NO: | YISSGGGTTYYPDTVKG | HSGYGTHWGVLFAY |
| | 119] | [SEQ ID NO: 120] | [SEQ ID NO: 121] |
| V_{L} | RASQDIGRYLN [SEQ | YTSILHS [SEQ ID | QQGNTLPWT [SEQ |
| | ID NO: 122] | NO: 123] | ID NO: 124] |

| | |
|---|---|
| Full V_{H} | EVQLVESGGGLVKPGGSLKLSCAASGFAFSIYDMSWVRQTPEKRLEWVAYISSGGGTT |
| | YYPDTVKGRFTISRDNAKNTLYLQMSSLKSEDTAMYYCARHSGYGTHWGVLFAYWGQG |
| | TLVTVSA [SEQ ID NO: 125] |
| DNA - | GAAGTGCAGCTGGTGGAGTCTGGGGGAGGCTTAGTGAAGCCTGGAGGGTCCCTGAAAC |
| Full V_{H} | TCTCCTGTGCAGCCTCTGGATTCGCTTTCAGTATCTATGACATGTCTTGGGTTCGCCA |
| | GACTCCGGAGAAGAGGCTGGAGTGGGTCGCATACATTAGTAGTGGTGGTGGTACCACC |
| | TACTATCCAGACACTGTGAAGGGCCGATTCACCATCTCCAGAGACAATGCCAAGAACA |
| | CCCTGTACCTGCAAATGAGCAGTCTGAAGTCTGAGGACACAGCCATGTATTACTGTGC |
| | AAGACATAGTGGCTACGGTACCCACTGGGGGGTTTTGTTTGCTTACTGGGGCCAAGGG |
| | ACTCTGGTCACTGTCTCTGCA [SEQ ID NO: 126] |
| Full V_{L} | DIQMTQTTSSLSASLGDRVTISCRASQDIGRYLNWYQQKPDGTVKLLIYYTSILHSGV |
| | PSRESGSGSGTDYSLTISNLEQEDFATYFCQQGNTLPWTFGGGTKLEIK [SEQ ID |
| | NO: 127] |
| DNA - | GATATCCAGATGACCCAGACTACATCCTCCCTGTCTGCCTCTCTGGGAGACAGAGTCA |
| Full V_{L} | CCATTAGTTGCAGGGCAAGTCAGGACATTGGCCGCTATTTAAACTGGTATCAGCAGAA |
| | ACCAGATGGAACTGTTAAACTCCTGATCTACTACACATCAATATTACACTCAGGAGTC |
| | CCATCAAGGTTCAGTGGCAGTGGGTCTGGAACAGATTATTCTCTCACCATTAGCAACC |
| | TGGAGCAAGAAGATTTTGCCACTTACTTTTGCCAACAGGGTAATACGCTTCCGTGGAC |
| | GTTCGGTGGAGGCACCAAGCTGGAAATCAAA [SEQ ID NO: 128] |
| scFv | MEFGLSWIFLAAILKGVQCEVQLVESGGGLVKPGGSLKLSCAASGFAFSIYDMSWVRQ |
| | TPEKRLEWVAYISSGGGTTYYPDTVKGRFTISRDNAKNTLYLQMSSLKSEDTAMYYCA |
| | RHSGYGTHWGVLFAYWGQGTLVTVSAGGGGSGGGGSGGGGSDIQMTQTTSSLSASLGD |
| | RVTISCRASQDIGRYLNWYQQKPDGTVKLLIYYTSILHSGVPSRESGSGSGTDYSLTI |
| | SNLEQEDFATYFCQQGNTLPWTFGGGTKLEIK [SEQ ID NO: 129] |
| scFv DNA | ATGGAGTTTGGGCTGAGCTGGATTTTCCTTGCTGCTATTTTAAAAGGTGTCCAGTGTG |
| | AAGTGCAGCTGGTGGAGTCTGGGGGAGGCTTAGTGAAGCCTGGAGGGTCCCTGAAACT |
| | CTCCTGTGCAGCCTCTGGATTCGCTTTCAGTATCTATGACATGTCTTGGGTTCGCCAG |
| | ACTCCGGAGAAGAGGCTGGAGTGGGTCGCATACATTAGTAGTGGTGGTGGTACCACCT |
| | ACTATCCAGACACTGTGAAGGGCCGATTCACCATCTCCAGAGACAATGCCAAGAACAC |
| | CCTGTACCTGCAAATGAGCAGTCTGAAGTCTGAGGACACAGCCATGTATTACTGTGCA |
| | AGACATAGTGGCTACGGTACCCACTGGGGGGTTTTGTTTGCTTACTGGGGCCAAGGGA |
| | CTCTGGTCACTGTCTCTGCAGGTGGAGGTGGATCAGGTGGAGGTGGATCTGGTGGAGG |
| | TGGATCTGATATCCAGATGACCCAGACTACATCCTCCCTGTCTGCCTCTCTGGGAGAC |
| | AGAGTCACCATTAGTTGCAGGGCAAGTCAGGACATTGGCCGCTATTTAAACTGGTATC |
| | AGCAGAAACCAGATGGAACTGTTAAACTCCTGATCTACTACACATCAATATTACACTC |
| | AGGAGTCCCATCAAGGTTCAGTGGCAGTGGGTCTGGAACAGATTATTCTCTCACCATT |
| | AGCAACCTGGAGCAAGAAGATTTTGCCACTTACTTTTGCCAACAGGGTAATACGCTTC |
| | CGTGGACGTTCGGTGGAGGCACCAAGCTGGAAATCAAA [SEQ ID NO: 130] |
| scFv v2 | MEFGLSWIFLAAILKGVQCEVQLVESGGGLVKPGGSLKLSCAASGFAFSIYDMSWVRQ |
| | TPEKRLEWVAYISSGGGTTYYPDTVKGRFTISRDNAKNTLYLQMSSLKSEDTAMYYCA |
| | RHSGYGTHWGVLFAYWGQGTLVTVSAGGGGSDIQMTQTTSSLSASLGDRVTISCRASQ |
| | DIGRYLNWYQQKPDGTVKLLIYYTSILHSGVPSRESGSGSGTDYSLTISNLEQEDFAT |
| | YFCQQGNTLPWTFGGGTKLEIK [SEQ ID NO: 131] |
| scFv DNA | ATGGAGTTTGGGCTGAGCTGGATTTTCCTTGCTGCTATTTTAAAAGGTGTCCAGTGTG |
| v2 | AAGTGCAGCTGGTGGAGTCTGGGGGAGGCTTAGTGAAGCCTGGAGGGTCCCTGAAACT |
| | CTCCTGTGCAGCCTCTGGATTCGCTTTCAGTATCTATGACATGTCTTGGGTTCGCCAG |
| | ACTCCGGAGAAGAGGCTGGAGTGGGTCGCATACATTAGTAGTGGTGGTGGTACCACCT |
| | ACTATCCAGACACTGTGAAGGGCCGATTCACCATCTCCAGAGACAATGCCAAGAACAC |
| | CCTGTACCTGCAAATGAGCAGTCTGAAGTCTGAGGACACAGCCATGTATTACTGTGCA |
| | AGACATAGTGGCTACGGTACCCACTGGGGGGTTTTGTTTGCTTACTGGGGCCAAGGGA |
| | CTCTGGTCACTGTCTCTGCAGGAGGAGGAGGCAGCGATATCCAGATGACCCAGACTAC |
| | ATCCTCCCTGTCTGCCTCTCTGGGAGACAGAGTCACCATTAGTTGCAGGGCAAGTCAG |
| | GACATTGGCCGCTATTTAAACTGGTATCAGCAGAAACCAGATGGAACTGTTAAACTCC |
| | TGATCTACTACACATCAATATTACACTCAGGAGTCCCATCAAGGTTCAGTGGCAGTGG |
| | GTCTGGAACAGATTATTCTCTCACCATTAGCAACCTGGAGCAAGAAGATTTTGCCACT |
| | TACTTTTGCCAACAGGGTAATACGCTTCCGTGGACGTTCGGTGGAGGCACCAAGCTGG |
| | AAATCAAA [SEQ ID NO: 132] |

In certain embodiments, the first antigen-recognizing receptor is a CAR comprising an extracellular antigen-binding domain that binds to CD22. In certain embodiments, the extracellular antigen-binding domain of the CAR comprises or consists of the amino acid sequence set forth in SEQ ID NO: 157 or SEQ ID NO: 158 and specifically binds to CD22, e.g., a human CD22 polypeptide. SEQ ID NO: 157 and SEQ ID NO: 158 are provided in Table 6 below.

In certain embodiments, the extracellular antigen-binding domain of the CAR comprises a heavy chain variable region (V_{H}) comprising a CDR1 comprising the amino acid sequence set forth in SEQ ID NO: 97 or a conservative modification thereof, a CDR2 comprising the amino acid sequence set forth in SEQ ID NO: 154 or a conservative modification thereof, and a CDR3 comprising the amino acid sequence set forth in SEQ ID NO: 99 or a conservative modification thereof. In certain embodiments, the extracellular antigen-binding domain of the CAR comprises a V_{H} comprising a CDR1 comprising the amino acid sequence set forth in SEQ ID NO: 97, a CDR2 comprising the amino acid sequence set forth in SEQ ID NO: 154, and a CDR3 comprising the amino acid sequence set forth in SEQ ID NO: 99. SEQ ID NOs: 97, 99, and 154 are provided in Table 6 below.

In certain embodiments, the extracellular antigen-binding domain of the CAR comprises a light chain variable region (V_{L}) comprising a CDR1 comprising the amino acid sequence set forth in SEQ ID NO: 100 or a conservative modification thereof, a CDR2 comprising the amino acid sequence set forth in SEQ ID NO: 101 or a conservative modification thereof, and a CDR3 comprising the amino acid sequence set forth in SEQ ID NO: 102 or a conservative modification thereof. In certain embodiments, the extracellular antigen-binding domain of the CAR comprises a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. SEQ ID NOs: 100-102 are provided in Table 6 below.

In certain embodiments, the extracellular antigen-binding domain of the CAR comprises a V_{H} comprising a CDR1 comprising the amino acid sequence set forth in SEQ ID NO: 97 or a conservative modification thereof, a CDR2 comprising the amino acid sequence set forth in SEQ ID NO: 154 or a conservative modification thereof, and a CDR3 comprising the amino acid sequence set forth in SEQ ID NO: 99, a conservative modification thereof; a V_{L} comprising a CDR1 comprising the amino acid sequence set forth in SEQ ID NO: 100 or a conservative modification thereof, a CDR2 comprising the amino acid sequence set forth in SEQ ID NO: 101 or a conservative modification thereof, and a CDR3 comprising the amino acid sequence set forth in SEQ ID NO: 102 or a conservative modification thereof. In certain embodiments, the extracellular antigen-binding domain of the CAR comprises a V_{H} comprising a CDR1 comprising amino acids having the sequence set forth in SEQ ID NO: 97, a CDR2 comprising the amino acid sequence set forth in SEQ ID NO: 154, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102.

In certain embodiments, extracellular antigen-binding domain of the CAR comprises a V_{H} comprising a CDR1, a CDR2, and a CDR3 of the V_{H} having the amino acid sequence set forth in SEQ ID NO: 155. In certain embodiments, extracellular antigen-binding domain of the CAR comprises a V_{L} comprising a CDR1, a CDR2, and a CDR3 of the V_{L} having the amino acid sequence set forth in SEQ ID NO: 105. In certain embodiments, extracellular antigen-binding domain of the CAR comprises a V_{H} comprising a CDR1, a CDR2, and a CDR3 of the V_{H} having the amino acid sequence set forth in SEQ ID NO: 155; and a V_{L} Comprising a CDR1, a CDR2, and a CDR3 of the V_{L} having the amino acid sequence set forth in SEQ ID NO: 105.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 155. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 155. In certain embodiments, the extracellular antigen-binding domain of the CAR comprises a V_{H} comprising the amino acid sequence set forth in SEQ ID NO: 155. SEQ ID NO: 155 is provided in Table 6 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 105. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 113. In certain embodiments, the extracellular antigen-binding domain of the CAR comprises a V_{L}comprising the amino acid sequence set forth in SEQ ID NO: 105. SEQ ID NO: 105 is provided in Table 6 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 155, and a V_{L} comprising an amino acid sequence that is at least about 80% (e.g., at least about 85%, at least about 90%, or at least about 95%) homologous or identical to the amino acid sequence set forth in SEQ ID NO: 105.

In certain embodiments, the extracellular antigen-binding domain of the CAR comprises a V_{H} comprising the amino acid sequence set forth in SEQ ID NO: 155. In certain embodiments, the extracellular antigen-binding domain of the CAR comprises a V_{L} comprising the amino acid sequence set forth in SEQ ID NO: 105. In certain embodiments, the extracellular antigen-binding domain of the CAR comprises V_{H} comprising the amino acid sequence set forth in SEQ ID NO: 155 and a V_{L} comprising the amino acid sequence set forth in SEQ ID NO: 105.

In certain embodiments, the extracellular antigen-binding domain of the CAR is an scFv that comprises or consists of the amino acid sequence set forth in SEQ ID NO: 157 or SEQ ID NO: 158. SEQ ID NOs: 154-160 are provided in the following Table 6. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the CDRs regions/sequences disclosed herein are delineated using the Kabat system (Swindells et al., J Mol Biol. 2017 Feb. 3; 429(3):356-364).

TABLE 6

| | CDRs | | |
|---|---|---|---|
| | 1 | 2 | 3 |
| V_{H} | SNSAAWN [SEQ | RTYRRSKWYNDYAVSVKS | EVTGDLEDAFDI [SEQ |
| | ID NO: 97] | [SEQ ID NO: 154] | ID NO: 99] |
| V_{L} | RASQTIWSYLN | AASSLQS [SEQ ID | QQSYSIPQT [SEQ ID |
| | [SEQ ID NO: | NO: 101] | NO: 102] |
| | 100] | | |

| | |
|---|---|
| V_{H} | QVQLQQSGPGLVKPSQTLSLTCAISGDSVSSNSAAW |
| | NWIRQSPSRGLEWLGRTYRRSKWYNDYAVSVKSRIT |
| | INPDTSKNQFSLQLNSVTPEDTAVYYCAREVTGDLE |
| | DAFDIWGQGTMVTVSS [SEQ ID NO: 155] |

| | |
|---|---|
| V_{L} | DIQMTQSPSSLSASVGDRVTITCRASQTIWSYLNWYQQRPGKA |
| | PNLLIYAASSLQSGVPSRFSGRGSGTDFTLTISSLQAEDFATY |
| | YCQQSYSIPQTFGQGTKLEIK [SEQ ID NO: 105] |
| DNA - Full | CAAGTACAGCTTCAACAGTCAGGTCCGGGGCTGGTAAAGCCTT |
| V_{H} | CTCAGACACTCAGCCTGACTTGTGCTATAAGTGGGGATAGTGT |
| | TTCATCCAACTCAGCGGCGTGGAACTGGATCCGGCAAAGTCCA |
| | TCTCGGGGCCTGGAGTGGTTGGGGCGGACCTATCGCAGGTCTA |
| | AGTGGTACAATGATTACGCCGTCTCAGTGAAGTCACGGATCAC |
| | AATCAATCCCGATACGAGTAAGAATCAGTTCTCACTTCAGCTT |
| | AACAGTGTGACACCTGAAGATACGGCAGTATATTATTGCGCGA |
| | GAGAGGTTACTGGGGACCTCGAAGATGCCTTCGATATCTGGGG |
| | TCAAGGCACAATGGTTACAGTCAGCTCC [SEQ ID NO: |
| | 156] |
| DNA - Full | GACATACAGATGACACAATCTCCGAGTAGCCTTTCCGCATCCG |
| V_{L} | TAGGTGATAGGGTTACCATAACTTGCCGCGCATCTCAAACGAT |
| | CTGGTCCTATCTGAACTGGTACCAGCAGAGACCAGGAAAAGCT |
| | CCTAATCTGCTTATCTACGCCGCAAGCTCACTGCAGTCTGGGG |
| | TTCCGAGTAGATTTTCTGGGCGAGGCAGCGGAACGGATTTTAC |
| | TCTGACCATAAGCTCTCTGCAAGCAGAAGATTTTGCCACGTAC |
| | TACTGCCAGCAATCTTACAGCATCCCACAAACATTTGGACAAG |
| | GCACAAAGTTGGAGATCAAA [SEQ ID NO: 106] |
| SCFV v1 | QVQLQQSGPGLVKPSQTLSLTCAISGDSVSSNSAAWNWIRQSP |
| | SRGLEWLGRTYRRSKWYNDYAVSVKSRITINPDTSKNQFSLOL |
| | NSVTPEDTAVYYCAREVTGDLEDAFDIWGQGTMVTVSSGGGGS |
| | GGGGSGGGGSDIQMTQSPSSLSASVGDRVTITCRASQTIWSYL |
| | NWYQQRPGKAPNLLIYAASSLQSGVPSRESGRGSGTDFTLTIS |
| | SLQAEDFATYYCQQSYSIPQTFGQGTKLEIK [SEQ ID NO: |
| | 157] |
| SCFV v2 | MELGLSWIFLLAILKGVQCQVQLQQSGPGLVKPSQTLSLTCAI |
| | SGDSVSSNSAAWNWIRQSPSRGLEWLGRTYRRSKWYNDYAVSV |
| | KSRITINPDTSKNQFSLQLNSVTPEDTAVYYCAREVTGDLEDA |
| | FDIWGQGTMVTVSSGGGGSGGGGSGGGGSDIQMTQSPSSLSAS |
| | VGDRVTITCRASQTIWSYLNWYQQRPGKAPNLLIYAASSLQSG |
| | VPSRESGRGSGTDFTLTISSLQAEDFATYYCQQSYSIPQTFGQ |
| | GTKLEIK [SEQ ID NO: 158] |
| DNA - ScFv | CAAGTACAGCTTCAACAGTCAGGTCCGGGGCTGGTAAAGCCTT |
| v1 | CTCAGACACTCAGCCTGACTTGTGCTATAAGTGGGGATAGTGT |
| | TTCATCCAACTCAGCGGCGTGGAACTGGATCCGGCAAAGTCCA |
| | TCTCGGGGCCTGGAGTGGTTGGGGCGGACCTATCGCAGGTCTA |
| | AGTGGTACAATGATTACGCCGTCTCAGTGAAGTCACGGATCAC |
| | AATCAATCCCGATACGAGTAAGAATCAGTTCTCACTTCAGCTT |
| | AACAGTGTGACACCTGAAGATACGGCAGTATATTATTGCGCGA |
| | GAGAGGTTACTGGGGACCTCGAAGATGCCTTCGATATCTGGGG |
| | TCAAGGCACAATGGTTACAGTCAGCTCCGGTGGAGGTGGATCA |
| | GGTGGAGGTGGATCTGGTGGAGGTGGATCTGACATACAGATGA |
| | CACAATCTCCGAGTAGCCTTTCCGCATCCGTAGGTGATAGGGT |
| | TACCATAACTTGCCGCGCATCTCAAACGATCTGGTCCTATCTG |
| | AACTGGTACCAGCAGAGACCAGGAAAAGCTCCTAATCTGCTTA |
| | TCTACGCCGCAAGCTCACTGCAGTCTGGGGTTCCGAGTAGATT |
| | TTCTGGGCGAGGCAGCGGAACGGATTTTACTCTGACCATAAGC |
| | TCTCTGCAAGCAGAAGATTTTGCCACGTACTACTGCCAGCAAT |
| | CTTACAGCATCCCACAAACATTTGGACAAGGCACAAAGTTGGA |
| | GATCAAA [SEQ ID NO: 159] |
| DNA ScFV | ATGGAACTCGGTCTGTCCTGGATTTTTCTGCTTGCGATCTTGA |
| v2 | AAGGAGTGCAGTGCCAAGTACAGCTTCAACAGTCAGGTCCGGG |
| | GCTGGTAAAGCCTTCTCAGACACTCAGCCTGACTTGTGCTATA |
| | AGTGGGGATAGTGTTTCATCCAACTCAGCGGCGTGGAACTGGA |
| | TCCGGCAAAGTCCATCTCGGGGCCTGGAGTGGTTGGGGCGGAC |
| | CTATCGCAGGTCTAAGTGGTACAATGATTACGCCGTCTCAGTG |
| | AAGTCACGGATCACAATCAATCCCGATACGAGTAAGAATCAGT |
| | TCTCACTTCAGCTTAACAGTGTGACACCTGAAGATACGGCAGT |
| | ATATTATTGCGCGAGAGAGGTTACTGGGGACCTCGAAGATGCC |
| | TTCGATATCTGGGGTCAAGGCACAATGGTTACAGTCAGCTCCG |
| | GTGGAGGTGGATCAGGTGGAGGTGGATCTGGTGGAGGTGGATC |
| | TGACATACAGATGACACAATCTCCGAGTAGCCTTTCCGCATCC |
| | GTAGGTGATAGGGTTACCATAACTTGCCGCGCATCTCAAACGA |
| | TCTGGTCCTATCTGAACTGGTACCAGCAGAGACCAGGAAAAGC |
| | TCCTAATCTGCTTATCTACGCCGCAAGCTCACTGCAGTCTGGG |
| | GTTCCGAGTAGATTTTCTGGGCGAGGCAGCGGAACGGATTTTA |
| | CTCTGACCATAAGCTCTCTGCAAGCAGAAGATTTTGCCACGTA |
| | CTACTGCCAGCAATCTTACAGCATCCCACAAACATTTGGACAA |
| | GGCACAAAGTTGGAGATCAAA [SEQ ID NO: 160] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 79, 81, 91, 93, 103, 105, 111, 113, 125, 127, and 155) may contain substitutions (e.g., conservative substitutions), insertions, or deletions relative to the specified sequence(s), but retain the ability to bind to a target antigen (e.g., CD312, CD276, CD22). In certain embodiments, a total of 1 to 10 amino acids are substituted, inserted and/or deleted in a specific sequence (e.g., SEQ ID NOs: 79, 81, 91, 93, 103, 105, 111, 113, 125, 127, and 155). In certain embodiments, substitutions, insertions, or deletions occur in regions outside the CDRs (e.g., in the FRs) of the extracellular antigen-binding domain. In certain embodiments, the extracellular antigen-binding domain of the CAR comprises V_{H} and/or V_{L} sequence selected from SEQ ID NOs: 79, 81, 91, 93, 103, 105, 111, 113, 125, 127, and 155, including post-translational modifications of that sequence (SEQ ID NO: 79, 81, 91, 93, 103, 105, 111, 113, 125, 127, and 155).

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., a human IL-2 signal sequence comprising or consisting of the amino acid sequence set forth in SEQ ID NO: 32), a mouse IL-2 signal sequence (e.g., a mouse IL-2 signal sequence comprising or consisting of the amino acid sequence set forth in SEQ ID NO: 33); a human kappa leader sequence (e.g., a human kappa leader sequence comprising or consisting of the amino acid sequence set forth in SEQ ID NO: 34), a mouse kappa leader sequence (e.g., a mouse kappa leader sequence comprising or consisting of the amino acid sequence set forth in SEQ ID NO: 35); a human CD8 leader sequence (e.g., a human CD8 leader sequence comprising or consisting of the amino acid sequence set forth in SEQ ID NO: 36); a truncated human CD8 signal peptide (e.g., a truncated human CD8 signal peptide comprising or consisting of the amino acid sequence set forth in SEQ ID NO: 37); a human albumin signal sequence (e.g., a human albumin signal sequence comprising or consisting of the amino acid sequence set forth in SEQ ID NO: 38); and a human prolactin signal sequence (e.g., a human prolactin signal sequence comprising or consisting of the amino acid sequence set forth in SEQ ID NO: 39). SEQ ID NO: 32-39 are provided below.

| [SEQ ID NO: 32] | |
|---|---|
| | MYRMQLLSCIALSLALVINS |
| [SEQ ID NO: 33] | |
| | MYSMQLASCVTLTLVLLVNS |
| [SEQ ID NO: 34] | |
| | METPAQLLFLLLLWLPDTTG |
| [SEQ ID NO: 35] | |
| | METDTLLLWVLLLWVPGSTG |
| [SEQ ID NO: 36] | |
| | MALPVTALLLPLALLLHAARP |
| [SEQ ID NO: 37] | |
| | MALPVTALLLPLALLLHA |
| [SEQ ID NO: 38] | |
| | MKWVTFISLLESSAYS |
| [SEQ ID NO: 39] | |
| | MDSKGSSQKGSRLLLLLVVSNLLLCQGVVS |

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 37.

### 2.1.2.2. Transmembrane Domain and Hinge/Spacer Region

In certain embodiments, the first antigen-recognizing receptor is a CAR that comprises a transmembrane domain. Different transmembrane domains result in different receptor stability. After antigen recognition, receptors cluster and a signal are transmitted to the cell. In accordance with the presently disclosed subject matter, the transmembrane domain of the first antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a transmembrane domain of human CD28 or a portion thereof. In certain embodiments, the CD28 polypeptide comprises or consists of an amino acid sequence that is a consecutive portion of the amino acid sequence having a NCBI Reference No: NP_006130 (SEQ ID NO: 7), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 7. In certain embodiments, the transmembrane domain of the CAR comprises a CD28 polypeptide that comprises or consists of amino acids 153 to 179 of SEQ ID NO: 7. SEQ ID NO: 7 is provided below.

[SEQ ID NO: 7]
MLRLLLALNLFPSIQVTGNKILVKQSPMLVAYDNAVNLSCKYSYNLESRE
FRASLHKGLDSAVEVCVVYGNYSQQLQVYSKTGENCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In certain embodiments, the first antigen-recognizing receptor is a CAR that further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 7), a portion of a CD8 polypeptide, or a synthetic spacer sequence.

In certain embodiments, the first antigen-recognizing receptor is a CAR that further comprises a hinge/spacer region comprising a native or modified hinge region of a CD28 polypeptide. In certain embodiments, the hinge/spacer region of the first antigen-recognizing receptor (e.g., a CAR) comprises a CD28 polypeptide comprising or consisting of amino acids 114 to 152 of SEQ ID NO: 7.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

### 2.1.2.3. Intracellular Signaling Domain

In certain embodiments, the first antigen-recognizing receptor is a CAR that comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 8) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 12, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 8. In certain embodiments, the intracellular signaling domain of the CAR comprises a native CD3ζ comprising or consisting of the amino acid sequence of amino acids 52 to 164 of SEQ ID NO: 8. SEQ ID NO: 8 is provided below:

[SEQ ID NO: 8]
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the native CD3ζ comprises or consists of an amino acid sequence that is at least about 85%, about 90%, about 95%, about 96%, about 97%, about 98%, about 99% or about 100% identical or homologous to the amino acid sequence set forth in SEQ ID NO: 9. SEQ ID NO: 9 is provided below:

[SEQ ID NO: 9]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 10.

| [SEQ ID NO: 10] | |
|---|---|
| | QNQLYNELNLGRREEYDVLDKR |

An exemplary nucleic acid sequence encoding the amino acid sequence of SEQ ID NO: 10 is set forth in SEQ ID NO: 11, which is provided below.

[SEQ ID NO: 11]
CAGAACCAGCTCTATAACGAGCTCAATCTAGGACGAAGAGAGGAGTACGA
TGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations.

In certain embodiments, the ITAM1 variant comprises or consists of the amino acid sequence set forth in SEQ ID NO: 12, which is provided below.

| [SEQ ID NO 12] | |
|---|---|
| | QNQLENELNLGRREEFDVLDKR |

An exemplary nucleic acid sequence encoding the amino acid sequence of SEQ ID NO: 12 is set forth in SEQ ID NO: 13, which is provided below.

[SEQ ID NO: 13]
CAGAACCAGCTCTTTAACGAGCTCAATCTAGGACGAAGAGAGGAGTTCGA
TGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 14, which is provided below.

| [SEQ ID NO: 14] | |
|---|---|
| | QEGLYNELQKDKMAEAYSEIGMK |

An exemplary nucleic acid sequence encoding the amino acid sequence of SEQ ID NO: 14 is set forth in SEQ ID NO: 15, which is provided below.

[SEQ ID NO: 15]
CAGGAAGGCCTGTACAATGAACTGCAGAAAGATAAGATGGCGGAGGCCTA
CAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 16, which is provided below.

| [SEQ ID NO: 16] | |
|---|---|
| | QEGLENELQKDKMAEAFSEIGMK |

An exemplary nucleic acid sequence encoding the amino acid sequence of SEQ ID NO: 16 is set forth in SEQ ID NO: 17, which is provided below.

[SEQ ID NO: 17]
CAGGAAGGCCTGTTCAATGAACTGCAGAAAGATAAGATGGCGGAGGCCTT
CAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 18, which is provided below.

| [SEQ ID NO: 18] | |
|---|---|
| | HDGLYQGLSTATKDTYDALHMQ |

An exemplary nucleic acid sequence encoding the amino acid sequence of SEQ ID NO: 18 is set forth in SEQ ID NO: 19, which is provided below.

[SEQ ID NO: 19]
CACGATGGCCTTTACCAGGGTCTCAGTACAGCCACCAAGGACACCTACGA
CGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 20, which is provided below.

[SEQ ID NO: 20]
HDGLFQGLSTATKDTFDALHMQ

An exemplary nucleic acid sequence encoding the amino acid sequence of SEQ ID NO: 20 is set forth in SEQ ID NO: 21, which is provided below.

[SEQ ID NO: 21]
CACGATGGCCTTTTCCAGGGGCTCAGTACAGCCACCAAGGACACCTTCGA
CGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 10, an ITAM2 variant consisting of the amino acid sequence set forth in SEQ ID NO: 16, and an ITAM3 variant consisting of the amino acid sequence set forth in SEQ ID NO: 20. In certain embodiments, the CAR is designated as “1XX”. In certain embodiments, the modified CD3ζ polypeptide comprises or consists of the amino acid sequence set forth in SEQ ID NO: 22. SEQ ID NO: 22 is provided below:

[SEQ ID NO: 22]
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
FDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 22 or a fragment thereof, and/or may optionally comprise up to one or up to two or up to three conservative amino acid substitutions.

An exemplary nucleic acid sequence encoding the amino acid sequence of SEQ ID NO: 22 is set forth in SEQ ID NO: 23, which is provided below.

[SEQ ID NO: 23]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTTCAATGAACTGCAGAAAGATAAGAT
GGCGGAGGCCTTCAGTGAGATTGGGATGAAAGGCGAGCGCCGGAGGGGCA
AGGGGCACGATGGCCTTTTCCAGGGGCTCAGTACAGCCACCAAGGACACC
TTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, CD150, CD226, and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the first antigen-recognizing receptor comprise or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 7 or a fragment thereof, and/or may optionally comprise up to one or up to two or up to three conservative amino acid substitutions. In certain embodiments, the CD28 polypeptide comprised in the co-stimulatory signaling region of the CAR comprises or consist of an amino acid sequence that is a consecutive portion of SEQ ID NO: 7, which is at least about 20, or at least about 30, or at least about 40, or at least about 50, and up to about 220 amino acids in length. Alternatively or additionally, in certain embodiments, the CD28 polypeptide comprised in the co-stimulatory signaling region of the CAR comprises or consists of amino acids 1 to 220, 1 to 50, 50 to 100, 100 to 150, 114 to 220, 150 to 200, 180 to 220, or 200 to 220 of SEQ ID NO: 7. In certain embodiments, the intracellular signaling domain of the CAR comprises a co-stimulatory signaling region that comprises a CD28 polypeptide comprising or consisting of amino acids 180 to 220 of SEQ ID NO: 7.

An exemplary nucleic acid sequence encoding the amino acid sequence of amino acids 180 to 220 of SEQ ID NO: 7 is set forth in SEQ ID NO: 24, which is provided below.

[SEQ ID NO: 24]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the first antigen-recognizine receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 25) or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 25, which is at least about 20, or at least about 30, or at least about 40, or at least about 50, and up to 218 amino acids in length. In certain embodiments, the CD28 polypeptide comprised in the co-stimulatory signaling region of the CAR comprises or consists of the amino acid sequence of amino acids 1 to 218, 1 to 50, 50 to 100, 100 to 150, 150 to 218, 178 to 218, or 200 to 218 of SEQ ID NO: 25. In certain embodiments, the co-stimulatory signaling region of the CAR comprises a CD28 polypeptide that comprises or consists of amino acids 178 to 218 of SEQ ID NO: 25. SEQ ID NO: 25 is provided below.

[SEQ ID NO: 25]
MTLRLLFLALNFFSVQVTENKILVKQSPLLVVDSNEVSLSCRYSYNLLAK
EFRASLYKGVNSDVEVCVGNGNFTYQPQFRSNAEFNCDGDFDNETVTFRL
WNLHVNHTDIYFCKIEFMYPPPYLDNERSNGTIIHIKEKHLCHTQSSPKL
FWALVVVAGVLFCYGLLVTVALCVIWTNSRRNRLLQSDYMNMTPRRPGLT
RKPYQPYAPARDFAAYRP

In certain embodiments, the intracellular signaling domain of the CAR comprises a co stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region of the CAR comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_001552 (SEQ ID NO: 26) or a fragment thereof, and/or may optionally comprise up to one or up to two or up to three conservative amino acid substitutions. In certain embodiments, the 4-1BB comprised in the co-stimulatory signaling region of the CAR comprises or consists of an amino acid sequence that is a consecutive portion of SEQ ID NO: 26, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the co-stimulatory signaling region of the CAR comprises a 4-1BB polypeptide that comprises or consists of the amino acid sequence of amino acids 1 to 255, 1 to 50, 50 to 100, 100 to 150, 150 to 200, or 200 to 255 of SEQ ID NO: 26. In certain embodiments, the co-stimulatory signaling region of the CAR comprises a 4-1BB polypeptide comprising or consisting of the amino acid sequence of amino acids 214 to 255 of SEQ ID NO: 26. SEQ ID NO: 26 is provided below.

[SEQ ID NO: 26]
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In certain embodiments, the intracellular signaling domain of the CAR comprises two co stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, CD150, CD226, and NKG2D. In certain embodiments, the intracellular signaling domain of the CAR comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

In certain embodiments, the first antigen-recognizing receptor is a CAR that comprises i) an extracellular antigen-binding domain, ii) a transmembrane domain comprising a CD28 polypeptide (e.g., human CD28 polypeptide, e.g., a transmembrane domain of CD28 (e.g., human CD28) or a portion thereof), iii) a hinge/spacer region derived from a CD28 polypeptide (e.g., a human CD28 polypeptide), iv) an intracellular signaling domain comprising a) a native CD3ζ polypeptide, and b) a co-stimulatory signaling region comprising a CD28 polypeptide (e.g., a human CD28 polypeptide, e.g., an intracellular domain of CD28 (e.g., human CD28) of a portion thereof). In certain embodiments, the transmembrane domain comprises a CD28 polypeptide comprising or consisting of amino acids 153 to 179 of SEQ ID NO: 7. In certain embodiments, the hinge/spacer region comprises a CD28 polypeptide comprising or consisting of amino acids 114 to 152 of SEQ ID NO: 7. In certain embodiments, the intracellular signaling domain comprises a native CD3ζ polypeptide comprising or consisting of the amino acid sequence set forth in SEQ ID NO: 9, and a co-stimulatory signaling region comprising a CD28 polypeptide that comprises or consists of 180 to 220 of SEQ ID NO: 7. In certain embodiments, the CAR is designated as “28z”. In certain embodiments, the CAR (e.g., 28z) is encoded by a nucleotide sequence that is at least about 80%, at least about 85%, at least about 90%, at least about 95%, at least about 96%, at least about 97%, at least about 98%, or at least about 99%, at least about 100% identical to the nucleotide sequence set forth in SEQ ID NO: 27. In certain embodiments, the CAR (e.g., 28z) comprises the nucleotide sequence set forth in SEQ ID NO: 27. SEQ ID NO: 27 is provided below.

[SEQ ID NO: 27]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCCAGAGTGAAGTTCAGCAGGAGCGCAGACGC
CCCCGCGTACCAGCAGGGCCAGAACCAGCTCTATAACGAGCTCAATCTAG
GACGAAGAGAGGAGTACGATGTTTTGGACAAGAGACGTGGCCGGGACCCT
GAGATGGGGGGAAAGCCGAGAAGGAAGAACCCTCAGGAAGGCCTGTACAA
TGAACTGCAGAAAGATAAGATGGCGGAGGCCTACAGTGAGATTGGGATGA
AAGGCGAGCGCCGGAGGGGCAAGGGGCACGATGGCCTTTACCAGGGTCTC
AGTACAGCCACCAAGGACACCTACGACGCCCTTCACATGCAGGCCCTGCC
CCCTCGC

In certain embodiments, the first antigen-recognizing receptor is a CAR that comprises i) an extracellular antigen-binding domain, ii) a transmembrane domain comprising a CD28 polypeptide (e.g., human CD28 polypeptide, e.g., a transmembrane domain of CD28 (e.g., human CD28) or a portion thereof), iii) a hinge/spacer region derived from a CD28 polypeptide (e.g., a human CD28 polypeptide), iv) an intracellular signaling domain comprising a) a modified CD3ζ polypeptide (e.g., a modified human CD3ζ polypeptide) comprising a native ITAM1, an ITAM2 variant consisting of two loss-of-function mutations, and an ITAM3 variant consisting of two loss-of-function mutations, and b) a co-stimulatory signaling region comprising a CD28 polypeptide (e.g., a human CD28 polypeptide, e.g., an intracellular domain of CD28 (e.g., human CD28) of a portion thereof). In certain embodiments, the transmembrane domain comprises a CD28 polypeptide comprising or consisting of amino acids 153 to 179 of SEQ ID NO: 7. In certain embodiments, the hinge/spacer region comprises a CD28 polypeptide comprising or consisting of amino acids 114 to 152 of SEQ ID NO: 7. In certain embodiments, the intracellular signaling domain comprises a modified CD3ζ polypeptide comprising or consisting of the amino acid sequence set forth in SEQ ID NO: 22, and a co-stimulatory signaling region comprising a CD28 polypeptide that comprises or consists of 180 to 220 of SEQ ID NO: 7. In certain embodiments, the CAR is designated as “28z1xx”. In certain embodiments, the CAR (e.g., 28z1xx) is encoded by a nucleotide sequence that is at least about 80%, at least about 85%, at least about 90%, at least about 95%, at least about 96%, at least about 97%, at least about 98%, or at least about 99%, at least about 100% identical to the nucleotide sequence set forth in SEQ ID NO: 28 or SEQ ID NO: 29. In certain embodiments, the CAR (e.g., 28z1xx) comprises the nucleotide sequence set forth in SEQ ID NO: 28 or SEQ ID NO: 29. SEQ ID NO: 28 and SEQ ID NO: 29 are provided below.

[SEQ ID NO: 28]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCCAGAGTGAAGTTCAGCAGGAGCGCAGACGC
CCCCGCGTACCAGCAGGGCCAGAACCAGCTCTATAACGAGCTCAATCTAG
GACGAAGAGAGGAGTACGATGTTTTGGACAAGAGACGTGGCCGGGACCCT
GAGATGGGGGGAAAGCCGAGAAGGAAGAACCCTCAGGAAGGCCTGTTCAA
TGAACTGCAGAAAGATAAGATGGCGGAGGCCTTCAGTGAGATTGGGATGA
AAGGCGAGCGCCGGAGGGGCAAGGGGCACGATGGCCTTTTCCAGGGTCTC
AGTACAGCCACCAAGGACACCTTCGACGCCCTTCACATGCAGGCCCTGCC
CCCTCGC
[SEQ ID NO: 29]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCCAGAGTGAAGTTCAGCAGGAGCGCAGACGC
CCCCGCGTACCAGCAGGGCCAGAACCAGCTCTATAACGAGCTCAATCTAG
GACGAAGAGAGGAGTACGATGTTTTGGACAAGAGACGTGGCCGGGACCCT
GAGATGGGGGGAAAGCCGAGAAGGAAGAACCCTCAGGAAGGCCTGTTCAA
TGAACTGCAGAAAGATAAGATGGCGGAGGCCTTCAGTGAGATTGGGATGA
AAGGCGAGCGCCGGAGGGGCAAGGGGCACGATGGCCTTTTCCAGGGTCTC
AGTACAGCCACCAAGGACACCTTCGACGCCCTTCACATGCAGGCCCTGCC
CCCTCGC

In certain embodiments, the first antigen-recognizing receptor is a CAR that comprises i) an extracellular antigen-binding domain, ii) a transmembrane domain, iii) a hinge/spacer region, iv) an intracellular signaling domain comprising a) a native CD3ζ polypeptide, and b) a co-stimulatory signaling region comprising a 4-1BB polypeptide (e.g., a human 4-1BB polypeptide, e.g., an intracellular domain of 4-1BB (e.g., human 4-1BB) of a portion thereof). In certain embodiments, the intracellular signaling domain comprises a native CD3ζ polypeptide comprising or consisting of the amino acid sequence set forth in SEQ ID NO: 9, and a co-stimulatory signaling region comprising a 4-1BB polypeptide that comprises or consists of 214 to 255 of SEQ ID NO: 26. In certain embodiments, the CAR is designated as “BBz”. In certain embodiments, the CAR (e.g., BBz) is encoded by a nucleotide sequence that is at least about 80%, at least about 85%, at least about 90%, at least about 95%, at least about 96%, at least about 97%, at least about 98%, or at least about 99%, at least about 100% identical to the nucleotide sequence set forth in SEQ ID NO: 30 or SEQ ID NO: 31. In certain embodiments, the CAR (e.g., BBz) comprises the nucleotide sequence set forth in SEQ ID NO: 30 or SEQ ID NO: 31. SEQ ID NO: 30 and SEQ ID NO: 31 are provided below.

[SEQ ID NO: 30]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAACAAACGGGGCAGAAAGAAGCTCCTGTATATATTCAAAC
AACCATTTATGAGACCAGTACAAACTACTCAAGAGGAAGATGGCTGTAGC
TGCCGATTTCCAGAAGAAGAAGAAGGAGGATGTGAACTGAGAGTGAAGTT
CAGCAGGAGCGCAGACGCCCCCGCGTACCAGCAGGGCCAGAACCAGCTCT
ATAACGAGCTCAATCTAGGACGAAGAGAGGAGTACGATGTTTTGGACAAG
AGACGTGGCCGGGACCCTGAGATGGGGGGAAAGCCGAGAAGGAAGAACCC
TCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGAT
GGCCTTTACCAGGGTCTCAGTACAGCCACCAAGGACACCTACGACGCCCT
TCACATGCAGGCCCTGCCCCCTCGC
[SEQ ID NO: 31]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAACAAACGGGGCAGAAAGAAGCTCCTGTATATATTCAAAC
AACCATTTATGAGACCAGTACAAACTACTCAAGAGGAAGATGGCTGTAGC
TGCCGATTTCCAGAAGAAGAAGAAGGAGGATGTGAACTGAGAGTGAAGTT
TAGCCGGAGTGCCGATGCTCCAGCCTATCAGCAAGGACAAAACCAACTTT
ACAACGAACTTAATCTCGGTAGGCGCGAGGAATACGATGTGCTTGATAAA
CGCCGGGGTCGCGATCCCGAAATGGGCGGGAAACCTCGGCGCAAGAATCC
TCAAGAAGGACTCTATAATGAGTTGCAGAAGGACAAGATGGCTGAGGCAT
ATAGTGAGATCGGTATGAAGGGAGAACGGAGAAGGGGGAAAGGGCATGAT
GGATTGTACCAAGGACTTAGCACAGCTACTAAAGATACATATGACGCCCT
GCACATGCAAGCATTGCCTCCACGC

### 2.1.3. Chimeric Ligand Receptors

In certain embodiments, the first antigen-recognizing receptor is a chimeric ligand receptor that comprises a ligand or a portion thereof that binds to the first antigen. In certain embodiments, the chimeric ligand receptor further comprises a transmembrane domain and an intracellular signaling domain.

In certain embodiments, the transmembrane domain is fused to the ligand or portion thereof. In certain embodiments, the transmembrane domain is fused to the intracellular signaling domain. In certain embodiments, the transmembrane domain is positioned between the ligand or portion thereof and the intracellular signaling domain. In certain embodiments the transmembrane domain of the chimeric ligand receptor is a transmembrane domain disclosed in Section 2.1.2.2. In certain embodiments, the intracellular signaling domain of the chimeric ligand receptor comprises a CD3ζ polypeptide (e.g., as disclosed in Section 2.1.2.3). Additional information on the presently disclosed chimeric ligand receptor can be found in Sauer et al., Blood (2021) 138 (4): 318-330, the content of which is incorporated by reference in its entirety.

### 2.1.4. Delivery of the First Antigen-Recognizing Receptor

In certain embodiments, the first antigen-recognizing receptor is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the first antigen-recognizing receptor is delivered to the cell by a non-viral method. Any targeted genome editing methods can also be used to deliver the first antigen-recognizing receptor to the cell. In certain embodiments, the first antigen-recognizing receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the first antigen-recognizing receptor to the cell.

In certain embodiments, the cell is a T cell, and the first antigen-recognizing receptor is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the first antigen-recognizing receptor is integrated at a TRAC locus. Methods of targeting a CAR to a site within the genome of T cell are disclosed in WO2017180989 and Eyquem et al., Nature. (2017 Mar. 2); 543(7643): 113-117, both of which are incorporated by reference in their entireties. In certain embodiments, the cell is a T cell, the first antigen-recognizing receptor is a CAR, and the first antigen-recognizing receptor is integrated at a TRAC locus. In certain embodiments, the cell further comprises a gene disruption of a TRBC locus. In certain embodiments, the gene disruption of a TRBC locus results in knockout of TRBC locus.

### 2.2. Second Antigen-Recognizing Receptor

The second antigen-recognizing receptor targets a second antigen. The second antigen can be a tumor antigen or a pathogen antigen. In certain embodiments, the second antigen-recognizing receptor is a TCR-like fusion molecule.

### 2.2.1. Second Antigen

In certain embodiments, the second antigen is a tumor antigen, e.g., one disclosed in Section 2.1.1. In certain embodiments, the tumor antigen is an antigen with low antigen density. In certain embodiments, the tumor antigen is expressed on a cell with low tumor cell frequency.

In certain embodiments, the second antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the second antigen is selected from the group consisting of CD70, SIGLEC-6, IL1RAP, CLEC12A, GRP78, TIM3, CD19, CD20, CD22, BCMA, GPRC5D, SLAMF7, CD276, and CAIX. In certain embodiments, the second antigen is CD70.

In certain embodiments, the first antigen and the second antigen are different. In certain embodiments, the second antigen is CD70. In certain embodiments, the first antigen is CD312 and the second antigen is CD70. In certain embodiments, the first antigen is CD276 and the second antigen is CD70.

In certain embodiments, the second antigen is CD19. In certain embodiments, the first antigen is CD22 and the second antigen is CD19.

In certain embodiments, the second antigen is a pathogen antigen, e.g., one disclosed in Section 2.1.1.

### 2.2.2. TCR-Like Fusion Molecules

In certain embodiments, the second antigen-recognizing receptor is a TCR-like fusion molecule. Non-limiting examples of TCR fusion molecules include HLA-Independent TCR-based Chimeric Antigen Receptor (also known as “HIT”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic 20 TRuC receptors engaging the complete T cell receptor for potent anti-tumor response,” Nature Communications volume 10, Article number: 2087 (2019), which is incorporated by reference in its entirety).

In certain embodiments, the TCR-like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprises a constant domain. In certain embodiments, the recombinant TCR binds to an antigen (e.g., a first antigen or a second antigen) in an HLA-independent manner. Thus, in certain embodiments, the recombinant TCR is an HLA-independent (or non-HLA restricted) TCR (referred to as “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR-like fusion molecule is capable of integrating with a CD3 complex and providing HLA-independent antigen recognition. In certain embodiments, the TCR-like fusion molecule replaces an endogenous TCR in a CD3/TCR complex.

In certain embodiments, the first and second antigen binding chains bind to an antigen with a dissociation constant (K_{D}) of about 2×10⁻⁷ M or less. In certain embodiments, the first and second antigen binding chains bind to an antigen with a high binding affinity. In certain embodiments, the K_{D} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain embodiments, the K_{D} is about 1×10⁻⁸ M or less. In certain embodiments, the K_{D} is about 3×10⁻⁹ M or less. In certain embodiments, the K_{D} is about 5×10⁻⁹ M or less. In certain embodiments, the K_{D} is from about 1×10⁻⁹ M to about 1×10⁻⁸ M. In certain embodiments, the K_{D} is from about 1.5×10⁻⁹ M to about 1×10⁻⁸ M. In certain embodiments, the K_{D} is from about 5×10⁻⁹ M to about 1×10⁻⁸M.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the first antigen binding chain or the second antigen binding chain comprises a constant domain that comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 40 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 40. SEQ ID NO: 40 is provided below.

[SEQ ID NO: 40]
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGFNLLMTLRLWSS

An exemplary nucleotide sequence encoding the amino acid sequence of SEQ ID NO: 40 is set forth in SEQ ID NO: 41, which is provided below.

[SEQ ID NO: 41]
ATCCAGAACCCTGACCCTGCCGTGTACCAGCTGAGAGACTCTAAATCCAG
TGACAAGTCTGTCTGCCTATTCACCGATTTTGATTCTCAAACAAATGTGT
CACAAAGTAAGGATTCTGATGTGTATATCACAGACAAAACTGTGCTAGAC
ATGAGGTCTATGGACTTCAAGAGCAACAGTGCTGTGGCCTGGAGCAACAA
ATCTGACTTTGCATGTGCAAACGCCTTCAACAACAGCATTATTCCAGAAG
ACACCTTCTTCCCCAGCCCAGAAAGTTCCTGTGATGTCAAGCTGGTCGAG
AAAAGCTTTGAAACAGATACGAACCTAAACTTTCAAAACCTGTCAGTGAT
TGGGTTCCGAATCCTCCTCCTGAAAGTGGCCGGGTTTAATCTGCTCATGA
CGCTGCGGCTGTGGTCCAGC

In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 42 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 42. SEQ ID NO: 42 is provided below.

[SEQ ID NO: 42]
IQNPDPAVYQLRDSKSSDKSVCLFTDFDSQTNVSQSKDSDVYITDKTVLD
MRSMDFKSNSAVAWSNKSDFACACAFNNSIIPEDTFFPSPESSCDVKLVE
KSFETDTNLNFQNLSVIGFRILLLKVAGFNLLMTLRLWSS

An exemplary nucleotide sequence encoding the amino acid sequence of SEQ ID NO: 42 is set forth in SEQ ID NO: 43, which is provided below.

[SEQ ID NO: 43]
ATTCCCAATATCCAGAACCCTGACCCTGCCGTGTACCAGCTGAGAGACTC
TAAATCCAGTGACAAGTCTGTCTGCCTATTCACCGATTTTGATTCTCAAA
CAAATGTGTCACAAAGTAAGGATTCTGATGTGTATATCACAGACAAAACT
GTGCTAGACATGAGGTCTATGGACTTCAAGAGCAACAGTGCTGTGGCCTG
GAGCAACAAATCTGACTTTGCATGTGCAAACGCCTTCAACAACAGCATTA
TTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCT
GTCAGTGATTGGGTTCCGAATCCTCCTCCTGAAAGTGGCCGGGTTTAATC
TGCTCATGACGCTGCGGCTGTGGTCCAGC

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 44) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 44. SEQ ID NO: 44 is provided below.

[SEQ ID NO: 44]
ATATCCAGAACCCTGACCCTGCCGTGTACCAGCTGAGAGACTCTAAATCCAGTGACAAGTCTGTCTGCCT
ATTCACCGATTTTGATTCTCAAACAAATGTGTCACAAAGTAAGGATTCTGATGTGTATATCACAGACAAA
ACTGTGCTAGACATGAGGTCTATGGACTTCAAGAGCAACAGTGCTGTGGCCTGGAGCAACAAATCTGACT
TTGCATGTGCAAACGCCTTCAACAACAGCATTATTCCAGAAGACACCTTCTTCCCCAGCCCAGGTAAGGG
CAGCTTTGGTGCCTTCGCAGGCTGTTTCCTTGCTTCAGGAATGGCCAGGTTCTGCCCAGAGCTCTGGTCA
ATGATGTCTAAAACTCCTCTGATTGGTGGTCTCGGCCTTATCCATTGCCACCAAAACCCTCTTTTTACTA
AGAAACAGTGAGCCTTGTTCTGGCAGTCCAGAGAATGACACGGGAAAAAAGCAGATGAAGAGAAGGTGGC
AGGAGAGGGCACGTGGCCCAGCCTCAGTCTCTCCAACTGAGTTCCTGCCTGCCTGCCTTTGCTCAGACTG
TTTGCCCCTTACTGCTCTTCTAGGCCTCATTCTAAGCCCCTTCTCCAAGTTGCCTCTCCTTATTTCTCCC
TGTCTGCCAAAAAATCTTTCCCAGCTCACTAAGTCAGTCTCACGCAGTCACTCATTAACCCACCAATCAC
TGATTGTGCCGGCACATGAATGCACCAGGTGTTGAAGTGGAGGAATTAAAAAGTCAGATGAGGGGTGTGC
CCAGAGGAAGCACCATTCTAGTTGGGGGAGCCCATCTGTCAGCTGGGAAAAGTCCAAATAACTTCAGATT
GGAATGTGTTTTAACTCAGGGTTGAGAAAACAGCTACCTTCAGGACAAAAGTCAGGGAAGGGCTCTCTGA
AGAAATGCTACTTGAAGATACCAGCCCTACCAAGGGCAGGGAGAGGACCCTATAGAGGCCTGGGACAGGA
GCTCAATGAGAAAGGAGAAGAGCAGCAGGCATGAGTTGAATGAAGGAGGCAGGGCCGGGTCACAGGGCCT
TCTAGGCCATGAGAGGGTAGACAGTATTCTAAGGACGCCAGAAAGCTGTTGATCGGCTTCAAGCAGGGGA
GGGACACCTAATTTGCTTTTCTTTTTTTTTTTTTTTTTTTTTTTTTTTTTTGAGATGGAGTTTTGCTCTT
GTTGCCCAGGCTGGAGTGCAATGGTGCATCTTGGCTCACTGCAACCTCCGCCTCCCAGGTTCAAGTGATT
CTCCTGCCTCAGCCTCCCGAGTAGCTGAGATTACAGGCACCCGCCACCATGCCTGGCTAATTTTTTGTAT
TTTTAGTAGAGACAGGGTTTCACTATGTTGGCCAGGCTGGTCTCGAACTCCTGACCTCAGGTGATCCACC
CGCTTCAGCCTCCCAAAGTGCTGGGATTACAGGCGTGAGCCACCACACCCGGCCTGCTTTTCTTAAAGAT
CAATCTGAGTGCTGTACGGAGAGTGGGTTGTAAGCCAAGAGTAGAAGCAGAAAGGGAGCAGTTGCAGCAG
AGAGATGATGGAGGCCTGGGCAGGGTGGTGGCAGGGAGGTAACCAACACCATTCAGGTTTCAAAGGTAGA
ACCATGCAGGGATGAGAAAGCAAAGAGGGGATCAAGGAAGGCAGCTGGATTTTGGCCTGAGCAGCTGAGT
CAATGATAGTGCCGTTTACTAAGAAGAAACCAAGGAAAAAATTTGGGGTGCAGGGATCAAAACTTTTTGG
AACATATGAAAGTACGTGTTTATACTCTTTATGGCCCTTGTCACTATGTATGCCTCGCTGCCTCCATTGG
ACTCTAGAATGAAGCCAGGCAAGAGCAGGGTCTATGTGTGATGGCACATGTGGCCAGGGTCATGCAACAT
GTACTTTGTACAAACAGTGTATATTGAGTAAATAGAAATGGTGTCCAGGAGCCGAGGTATCGGTCCTGCC
AGGGCCAGGGGCTCTCCCTAGCAGGTGCTCATATGCTGTAAGTTCCCTCCAGATCTCTCCACAAGGAGGC
ATGGAAAGGCTGTAGTTGTTCACCTGCCCAAGAACTAGGAGGTCTGGGGTGGGAGAGTCAGCCTGCTCTG
GATGCTGAAAGAATGTCTGTTTTTCCTTTTAGAAAGTTCCTGTGATGTCAAGCTGGTCGAGAAAAGCTTT
GAAACAGGTAAGACAGGGGTCTAGCCTGGGTTTGCACAGGATTGCGGAAGTGATGAACCCGCAATAACCC
TGCCTGGATGAGGGAGTGGGAAGAAATTAGTAGATGTGGGAATGAATGATGAGGAATGGAAACAGCGGTT
CAAGACCTGCCCAGAGCTGGGTGGGGTCTCTCCTGAATCCCTCTCACCATCTCTGACTTTCCATTCTAAG
CACTTTGAGGATGAGTTTCTAGCTTCAATAGACCAAGGACTCTCTCCTAGGCCTCTGTATTCCTTTCAAC
AGCTCCACTGTCAAGAGAGCCAGAGAGAGCTTCTGGGTGGCCCAGCTGTGAAATTTCTGAGTCCCTTAGG
GATAGCCCTAAACGAACCAGATCATCCTGAGGACAGCCAAGAGGTTTTGCCTTCTTTCAAGACAAGCAAC
AGTACTCACATAGGCTGTGGGCAATGGTCCTGTCTCTCAAGAATCCCCTGCCACTCCTCACACCCACCCT
GGGCCCATATTCATTTCCATTTGAGTTGTTCTTATTGAGTCATCCTTCCTGTGGTAGCGGAACTCACTAA
GGGGCCCATCTGGACCCGAGGTATTGTGATGATAAATTCTGAGCACCTACCCCATCCCCAGAAGGGCTCA
GAAATAAAATAAGAGCCAAGTCTAGTCGGTGTTTCCTGTCTTGAAACACAATACTGTTGGCCCTGGAAGA
ATGCACAGAATCTGTTTGTAAGGGGATATGCACAGAAGCTGCAAGGGACAGGAGGTGCAGGAGCTGCAGG
CCTCCCCCACCCAGCCTGCTCTGCCTTGGGGAAAACCGTGGGTGTGTCCTGCAGGCCATGCAGGCCTGGG
ACATGCAAGCCCATAACCGCTGTGGCCTCTTGGTTTTACAGATACGAACCTAAACTTTCAAAACCTGTCA
GTGATTGGGTTCCGAATCCTCCTCCTGAAAGTGGCCGGGTTTAATCTGCTCATGACGCTGCGGCTGTGGT
CCAGCTGAGGTGAGGGGCCTTGAAGCTGGGAGTGGGGTTTAGGGACGCGGGTCTCTGGGTGCATCCTAAG
CTCTGAGAGCAAACCTCCCTGCAGGGTCTTGCTTTTAAGTCCAAAGCCTGAGCCCACCAAACTCTCCTAC
TTCTTCCTGTTACAAATTCCTCTTGTGCAATAATAATGGCCTGAAACGCTGTAAAATATCCTCATTTCAG
CCGCCTCAGTTGCACTTCTCCCCTATGAGGTAGGAAGAACAGTTGTTTAGAAACGAAGAAACTGAGGCCC
CACAGCTAATGAGTGGAGGAAGAGAGACACTTGTGTACACCACATGCCTTGTGTTGTACTTCTCTCACCG
TGTAACCTCCTCATGTCCTCTCTCCCCAGTACGGCTCTCTTAGCTCAGTAGAAAGAAGACATTACACTCA
TATTACACCCCAATCCTGGCTAGAGTCTCCGCACCCTCCTCCCCCAGGGTCCCCAGTCGTCTTGCTGACA
ACTGCATCCTGTTCCATCACCATCAAAAAAAAACTCCAGGCTGGGTGCGGGGGCTCACACCTGTAATCCC
AGCACTTTGGGAGGCAGAGGCAGGAGGAGCACAGGAGCTGGAGACCAGCCTGGGCAACACAGGGAGACCC
CGCCTCTACAAAAAGTGAAAAAATTAACCAGGTGTGGTGCTGCACACCTGTAGTCCCAGCTACTTAAGAG
GCTGAGATGGGAGGATCGCTTGAGCCCTGGAATGTTGAGGCTACAATGAGCTGTGATTGCGTCACTGCAC
TCCAGCCTGGAAGACAAAGCAAGATCCTGTCTCAAATAATAAAAAAAATAAGAACTCCAGGGTACATTTG
CTCCTAGAACTCTACCACATAGCCCCAAACAGAGCCATCACCATCACATCCCTAACAGTCCTGGGTCTTC
CTCAGTGTCCAGCCTGACTTCTGTTCTTCCTCATTCCAGATCTGCAAGATTGTAAGACAGCCTGTGCTCC
CTCGCTCCTTCCTCTGCATTGCCCCTCTTCTCCCTCTCCAAACAGAGGGAACTCTCCTACCCCCAAGGAG
GTGAAAGCTGCTACCACCTCTGTGCCCCCCCGGCAATGCCACCAACTGGATCCTACCCGAATTTATGATT
AAGATTGCTGAAGAGCTGCCAAACACTGCTGCCACCCCCTCTGTTCCCTTATTGCTGCTTGTCACTGCCT
GACATTCACGGCAGAGGCAAGGCTGCTGCAGCCTCCCCTGGCTGTGCACATTCCCTCCTGCTCCCCAGAG
ACTGCCTCCGCCATCCCACAGATGATGGATCTTCAGTGGGTTCTCTTGGGCTCTAGGTCCTGCAGAATGT
TGTGAGGGGTTTATTTTTTTTTAATAGTGTTCATAAAGAAATACATAGTATTCTTCTTCTCAAGACGTGG
GGGGAAATTATCTCATTATCGAGGCCCTGCTATGCTGTGTATCTGGGCGTGTTGTATGTCCTGCTGCCGA
TGCCTTC

In certain embodiments, the first antigen binding chain or the second antigen binding chain comprises a constant domain comprising a native or modified TRBC polypeptide. In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 45 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 45. SEQ ID NO: 45 is provided below.

[SEQ ID NO: 45]
DLKNVFPPEVAVFEPSEAEISHTQKATLVCLATGFYPDHVELSWWVNGKE
VHSGVSTDPQPLKEQPALNDSRYCLSSRLRVSATFWQNPRNHERCQVQFY
GLSENDEWTQDRAKPVTQIVSAEAWGRADCGFTSESYQQGVLSATILYEI
LLGKATLYAVLVSALVLMAMVKRKDSRG

An exemplary nucleotide sequence encoding the amino acid sequence of SEQ ID NO: 45 is set forth in SEQ ID NO: 46, which is provided below.

[SEQ ID NO: 46]
GATCTGAAAAACGTGTTCCCTCCTGAAGTGGCTGTCTTTGAACCATCCGA
GGCCGAGATTTCCCATACCCAGAAAGCAACTCTGGTCTGTCTGGCCACTG
GATTCTACCCCGATCACGTGGAACTGTCTTGGTGGGTGAACGGCAAGGAA
GTCCATTCCGGAGTCTCTACCGACCCTCAGCCCCTCAAGGAGCAGCCTGC
TCTCAACGATTCTCGGTACTGCCTGTCATCTCGACTGAGAGTGTCTGCCA
CCTTCTGGCAGAACCCTAGAAACCACTTTCGGTGTCAGGTCCAGTTTTAC
GGCCTGAGCGAGAACGATGAGTGGACACAGGATAGAGCCAAACCTGTGAC
ACAGATTGTGAGCGCCGAGGCTTGGGGACGAGCCGATTGTGGCTTCACAT
CCGAGTCTTACCAGCAGGGAGTGCTGTCTGCTACAATCCTCTACGAAATT
CTCCTGGGGAAGGCCACCCTGTACGCTGTCCTCGTGTCTGCTCTGGTGCT
CATGGCTATGGTCAAACGAAAGGACTCTAGAGGC

In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 47 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 47. SEQ ID NO: 47 is provided below.

[SEQ ID NO: 47]
LEDLKNVFPPEVAVFEPSEAEISHTQKATLVCLATGFYPDHVELSWWVNG
KEVHSGVSTDPQPLKEQPALNDSRYCLSSRLRVSATFWQNPRNHFRCQVQ
FYGLSENDEWTQDRAKPVTQIVSAEAWGRADCGFTSESYQQGVLSATILY
EILLGKATLYAVLVSALVLMAMVKRKDSRG

An exemplary nucleotide sequence encoding the amino acid sequence of SEQ ID NO: 47 is set forth in SEQ ID NO: 48, which is provided below.

[SEQ ID NO: 48]
CTGGAGGATCTGAAAAACGTGTTCCCTCCTGAAGTGGCTGTCTTTGAACC
ATCCGAGGCCGAGATTTCCCATACCCAGAAAGCAACTCTGGTCTGTCTGG
CCACTGGATTCTACCCCGATCACGTGGAACTGTCTTGGTGGGTGAACGGC
AAGGAAGTCCATTCCGGAGTCTCTACCGACCCTCAGCCCCTCAAGGAGCA
GCCTGCTCTCAACGATTCTCGGTACTGCCTGTCATCTCGACTGAGAGTGT
CTGCCACCTTCTGGCAGAACCCTAGAAACCACTTTCGGTGTCAGGTCCAG
TTTTACGGCCTGAGCGAGAACGATGAGTGGACACAGGATAGAGCCAAACC
TGTGACACAGATTGTGAGCGCCGAGGCTTGGGGACGAGCCGATTGTGGCT
TCACATCCGAGTCTTACCAGCAGGGAGTGCTGTCTGCTACAATCCTCTAC
GAAATTCTCCTGGGGAAGGCCACCCTGTACGCTGTCCTCGTGTCTGCTCT
GGTGCTCATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 49 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 49. SEQ ID NO: 49 is provided below.

[SEQ ID NO: 49]
LNKVFPPEVAVFEPSEAEISHTQKATLVCLATGFFPDHVELSWWVNGKEV
HSGVSTDPQPLKEQPALNDSRYCLSSRLRVSATFWQNPRNHFRCQVQFYG
LSENDEWTQDRAKPVTQIVSAEAWGRADCGFTSVSYQQGVLSATILYEIL
LGKATLYAVLVSALVLMAMVKRKDF

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 50 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 50. SEQ ID NO: 50 is provided below.

[SEQ ID NO: 50]
DLNKVFPPEVAVFEPSEAEISHTQKATLVCLATGFFPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSVSYQQGVLSATILYEI
LLGKATLYAVLVSALVLMAMVKRKDF

An exemplary nucleotide sequence encoding the amino acid sequence of SEQ ID NO: 50 is set forth in SEQ ID NO: 51, which is provided below.

[SEQ ID NO: 51]
GACCTGAACAAGGTGTTCCCACCCGAGGTCGCTGTGTTTGAGCCATCAGA
AGCAGAGATCTCCCACACCCAAAAGGCCACACTGGTGTGCCTGGCCACAG
GCTTCTTCCCCGACCACGTGGAGCTGAGCTGGTGGGTGAATGGGAAGGAG
GTGCACAGTGGGGTCAGCACAGACCCGCAGCCCCTCAAGGAGCAGCCCGC
CCTCAATGACTCCAGATACTGCCTGAGCAGCCGCCTGAGGGTCTCGGCCA
CCTTCTGGCAGAACCCCCGCAACCACTTCCGCTGTCAAGTCCAGTTCTAC
GGGCTCTCGGAGAATGACGAGTGGACCCAGGATAGGGCCAAACCCGTCAC
CCAGATCGTCAGCGCCGAGGCCTGGGGTAGAGCAGACTGTGGCTTTACCT
CGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTT
GATGGCCATGGTCAAGAGAAAGGATTTC

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI Genbank ID: 28639, NG_001333.2, range 645749 to 647196 (TRBC1, SEQ ID NO: 52), NCBI Genbank ID: 28638, NG_001333.2 range 655095 to 656583 (TRBC2, SEQ ID NO: 53) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 52.

In certain embodiments, the TRBC polypeptide comprises or consists of the amino acid sequence encoded by the nucleotide sequence of SEQ ID NO: 53. SEQ ID NO: 52 and 53 are provided below.

[SEQ ID NO: 52]
AGGACCTGAACAAGGTGTTCCCACCCGAGGTCGCTGTGTTTGAGCCATCA
GAAGCAGAGATCTCCCACACCCAAAAGGCCACACTGGTGTGCCTGGCCAC
AGGCTTCTTCCCCGACCACGTGGAGCTGAGCTGGTGGGTGAATGGGAAGG
AGGTGCACAGTGGGGTCAGCACAGACCCGCAGCCCCTCAAGGAGCAGCCC
GCCCTCAATGACTCCAGATACTGCCTGAGCAGCCGCCTGAGGGTCTCGGC
CACCTTCTGGCAGAACCCCCGCAACCACTTCCGCTGTCAAGTCCAGTTCT
ACGGGCTCTCGGAGAATGACGAGTGGACCCAGGATAGGGCCAAACCCGTC
ACCCAGATCGTCAGCGCCGAGGCCTGGGGTAGAGCAGGTGAGTGGGGCCT
GGGGAGATGCCTGGAGGAGATTAGGTGAGACCAGCTACCAGGGAAAATGG
AAAGATCCAGGTAGCAGACAAGACTAGATCCAAAAAGAAAGGAACCAGCG
CACACCATGAAGGAGAATTGGGCACCTGTGGTTCATTCTTCTCCCAGATT
CTCAGCCCAACAGAGCCAAGCAGCTGGGTCCCCTTTCTATGTGGCCTGTG
TAACTCTCATCTGGGTGGTGCCCCCCATCCCCCTCAGTGCTGCCACATGC
CATGGATTGCAAGGACAATGTGGCTGACATCTGCATGGCAGAAGAAAGGA
GGTGCTGGGCTGTCAGAGGAAGCTGGTCTGGGCCTGGGAGTCTGTGCCAA
CTGCAAATCTGACTTTACTTTTAATTGCCTATGAAAATAAGGTCTCTCAT
TTATTTTCCTCTCCCTGCTTTCTTTCAGACTGTGGCTTTACCTCGGGTAA
GTAAGCCCTTCCTTTTCCTCTCCCTCTCTCATGGTTCTTGACCTAGAACC
AAGGCATGAAGAACTCACAGACACTGGAGGGTGGAGGGTGGGAGAGACCA
GAGCTACCTGTGCACAGGTACCCACCTGTCCTTCCTCCGTGCCAACAGTG
TCCTACCAGCAAGGGGTCCTGTCTGCCACCATCCTCTATGAGATCCTGCT
AGGGAAGGCCACCCTGTATGCTGTGCTGGTCAGCGCCCTTGTGTTGATGG
CCATGGTAAGCAGGAGGGCAGGATGGGGCCAGCAGGCTGGAGGTGACACA
CTGACACCAAGCACCCAGAAGTATAGAGTCCCTGCCAGGATTGGAGCTGG
GCAGTAGGGAGGGAAGAGATTTCATTCAGGTGCCTCAGAAGATAACTTGC
ACCTCTGTAGGATCACAGTGGAAGGGTCATGCTGGGAAGGAGAAGCTGGA
GTCACCAGAAAACCCAATGGATGTTGTGATGAGCCTTACTATTTGTGTGG
TCAATGGGCCCTACTACTTTCTCTCAATCCTCACAACTCCTGGCTCTTAA
TAACCCCCAAAACTTTCTCTTCTGCAGGTCAAGAGAAAGGATTTCTGA
[SEQ ID NO: 53]
AGGACCTGAAAAACGTGTTCCCACCCGAGGTCGCTGTGTTTGAGCCATCA
GAAGCAGAGATCTCCCACACCCAAAAGGCCACACTGGTATGCCTGGCCAC
AGGCTTCTACCCCGACCACGTGGAGCTGAGCTGGTGGGTGAATGGGAAGG
AGGTGCACAGTGGGGTCAGCACAGACCCGCAGCCCCTCAAGGAGCAGCCC
GCCCTCAATGACTCCAGATACTGCCTGAGCAGCCGCCTGAGGGTCTCGGC
CACCTTCTGGCAGAACCCCCGCAACCACTTCCGCTGTCAAGTCCAGTTCT
ACGGGCTCTCGGAGAATGACGAGTGGACCCAGGATAGGGCCAAACCCGTC
ACCCAGATCGTCAGCGCCGAGGCCTGGGGTAGAGCAGGTGAGTGGGGCCT
GGGGAGATGCCTGGAGGAGATTAGGTGAGACCAGCTACCAGGGAAAATGG
AAAGATCCAGGTAGCGGACAAGACTAGATCCAGAAGAAAGCCAGAGTGGA
CAAGGTGGGATGATCAAGGTTCACAGGGTCAGCAAAGCACGGTGTGCACT
TCCCCCACCAAGAAGCATAGAGGCTGAATGGAGCACCTCAAGCTCATTCT
TCCTTCAGATCCTGACACCTTAGAGCTAAGCTTTCAAGTCTCCCTGAGGA
CCAGCCATACAGCTCAGCATCTGAGTGGTGTGCATCCCATTCTCTTCTGG
GGTCCTGGTTTCCTAAGATCATAGTGACCACTTCGCTGGCACTGGAGCAG
CATGAGGGAGACAGAACCAGGGCTATCAAAGGAGGCTGACTTTGTACTAT
CTGATATGCATGTGTTTGTGGCCTGTGAGTCTGTGATGTAAGGCTCAATG
TCCTTACAAAGCAGCATTCTCTCATCCATTTTTCTTCCCCTGTTTTCTTT
CAGACTGTGGCTTCACCTCCGGTAAGTGAGTCTCTCCTTTTTCTCTCTAT
CTTTCGCCGTCTCTGCTCTCGAACCAGGGCATGGAGAATCCACGGACACA
GGGGCGTGAGGGAGGCCAGAGCCACCTGTGCACAGGTGCCTACATGCTCT
GTTCTTGTCAACAGAGTCTTACCAGCAAGGGGTCCTGTCTGCCACCATCC
TCTATGAGATCTTGCTAGGGAAGGCCACCTTGTATGCCGTGCTGGTCAGT
GCCCTCGTGCTGATGGCCATGGTAAGGAGGAGGGTGGGATAGGGCAGATG
ATGGGGGCAGGGGATGGAACATCACACATGGGCATAAAGGAATCTCAGAG
CCAGAGCACAGCCTAATATATCCTATCACCTCAATGAAACCATAATGAAG
CCAGACTGGGGAGAAAATGCAGGGAATATCACAGAATGCATCATGGGAGG
ATGGAGACAACCAGCGAGCCCTACTCAAATTAGGCCTCAGAGCCCGCCTC
CCCTGCCCTACTCCTGCTGTGCCATAGCCCCTGAAACCCTGAAAATGTTC
TCTCTTCCACAGGTCAAGAGAAAGGATTCCAGAGGCTAG

In certain embodiments, the first antigen binding chain or the second antigen binding chain comprises a constant domain comprising a native or modified TRGC polypeptide. In certain embodiments, the TRGC polypeptide is a native or modified TRGC1 polypeptide. In certain embodiments, the TRGC1 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 54, which is provided below. In certain embodiments, the TRGC1 polypeptide comprises or consists of the amino acid sequence set forth in SEQ ID NO: 54.

[SEQ ID NO: 54]
DKQLDADVSPKPTIFLPSIAETKLQKAGTYLCLLEKFFPDVIKIHWQEKK
SNTILGSQEGNTMKINDTYMKFSWLTVPEKSLDKEHRCIVRHENNKNGVD
QEIIFPPIKTDVITMDPKDNCSKDANDTLLLQLTNTSAYYMYLLLLLKSV
VYFAIITCCLLRRTAFCCNGEKS

In certain embodiments, the TRGC polypeptide is a native or modified TRGC2 polypeptide. In certain embodiments, the TRGC2 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 55, which is provided below. In certain embodiments, the TRGC2 polypeptide comprises or consists of the amino acid sequence set forth in SEQ ID NO: 55.

[SEQ ID NO: 55]
DKQLDADVSPKPTIFLPSIAETKLQKAGTYLCLLEKFFPDIIKIHWQEKK
SNTILGSQEGNTMKTNDTYMKFSWLTVPEESLDKEHRCIVRHENNKNGID
QEIIFPPIKTDVTTVDPKYNYSKDANDVITMDPKDNWSKDANDTLLLQLT
NTSAYYTYLLLLLKSVVYFAIITCCLLRRTAFCCNGEKS

In certain embodiments, the TRGC polypeptide comprises or consists of an amino acid sequence that is at least about 85%, about 90%, about 95%, about 96%, about 97%, about 98%, about 99% or 100% homologous or identical to the amino acid sequence encoded by a transcript expressed by a gene of NCBI Genbank ID: 6966, NG_001336.2, range 108270 to 113860 (TRGC1, SEQ ID NO: 56), NCBI Genbank ID: 6967, NG_001336.2, range 124376 to 133924 (TRGC2, SEQ ID NO: 57) or a fragment thereof, and/or may optionally comprise up to one or up to two or up to three conservative amino acid substitutions. In certain embodiments, the TRGC polypeptide comprises or consists of the amino acid sequence encoded by the nucleotide sequence of SEQ ID NO: 56. In certain embodiments, the TRGC polypeptide comprises or consists of the amino acid sequence encoded by the nucleotide sequence of SEQ ID NO: 57. SEQ ID NO: 56 and 57 are provided below.

[SEQ ID NO: 56]
ATAAACAACTTGATGCAGATGTTTCCCCCAAGCCCACTATTTTTCTTCCTTCAATTGCTGAAACAAAGCT
CCAGAAGGCTGGAACATACCTTTGTCTTCTTGAGAAATTTTTCCCTGATGTTATTAAGATACATTGGCAA
GAAAAGAAGAGCAACACGATTCTGGGATCCCAGGAGGGGAACACCATGAAGACTAACGACACATACATGA
AATTTAGCTGGTTAACGGTGCCAGAAAAGTCACTGGACAAAGAACACAGATGTATCGTCAGACATGAGAA
TAATAAAAACGGAGTTGATCAAGAAATTATCTTTCCTCCAATAAAGACAGGTATGTGTTTACGCATATCA
TCTGTCAGAACACTTCTTTGAAAGTGAATGCTGCATTTTTTCCTTTCAGTATTAATGAAAAACAAACATA
AATCTTTCTTAAATATTGTTACATTTAATGGTAGCATAAATGCCCTGCTACTTTTCTATAGAATTAAAAT
GGTATAGGTTTTGGAGAAAACAAAATTGAAAAAGTTACTGAAGGTTTGTCAGCCTCAGCTCCATTATCCA
AAATAAGAAAGTCACGTGCTGGTTTTTAGGGTTGTTAGATGGATTAAAGAAACAACATACACAGAAGCAT
CTAGCAACGTGACACGTGGTAAACGCTCAAAAAGTGTTCTCCCTTCTTTTGATGACTTTACTTGATCAGG
AAATAACATATATATGTCTTTCAGGAATGTTCTGCCCAAGCAGGAGAGTCACTCACCTCAATCTTGCTAC
CCACAAAGTTTAACCTAAAAACAACGGGTTCATTGTTGACAAAATGATGTTTATCTGTTGTTGACAGAAT
GATGTTTATCTAAAAACAGTTCCAATTTTCTATTTCCTTTGCTGAGACACAAAGGGGAGGCAAATGTGCA
AAGCTTGAGGGTAGTCTTACCACTGTGCTTAAGTGTTCTGATTTTTCTAGTGATCAGGGCAAAATAAAAA
GTATAGTAAGTTCCAAGGCAGTGAATATTATACAGGAGAGAAGTTACAGTTTTATAATGTGTTTTCCTTT
ACACTAAATTCTAAAAGTAAAAAGTCTTTTTTTTTTTTTGACAGAGTTTCACTCTTGTTGCCCAAGCAGG
TGTGCTATGGTATGATCTCAGCTCACTGCAACCTCCACCTCCCGGGTTCAAGTGATTCTCTTACTTCAGC
CTCCCGACAGGCTGGGATTGCAGGCGCCTGCCACCACACCTGGCTAATTTTTGTGTTTTTAGTAGAGATG
GGGTTTCACCATGTTGGCCAGGCTGGTCTCAAATTCCTGACCTCAAGTGATCCATCCACCTCGGCCTCCA
AGTGCTGGGATTATGGGCGTCAGCCACTGTGCCCAGCCTAAAAGTAAAATGTCTTTCATGAGCTTCCCAA
GGCAGCTACGTTAAGGAGGACACTTCTCTTAATGTCATTCTACAGTAGATTTCTAATGCTCTTTCTTGGA
AGTTTGTTTTTCTGAGAAAAGCTAAAAATATAACATGGAAGTGATCATATTATATAATCAATGAAGTGCT
TTTCAAGGAGATAAAACTAATCTGGTCCACACTTGCAACCAACCTTGATTGAGAGAGAGAGAGAACTCAG
GATACACTTGAAGATTTTATTATGGGGAACAGTTACTTTATTCTTTTTACCTCAATCAATGCATGGAAAT
AAGTGATAGTCATTTTCATTTATCTTTTAATAAATGAAGTCACCATGAGGAAAATAAAAAGACATTGAAA
ACCCATTAAAGTCAGCCCTTAAAGATATTTGGACATGCAGACTTGATAACTAACGTTTGCATTCTTGAGA
CTTACCCAAAACCCATACCTCAAGTCCAAGTTTTTAGAATTCATGAAATAAAGATCTCAGTGAGTGCATA
AAATTGCGCACCAGAATCATATCCGTATAGACAAGAACACATCTACTAGAAAAATAATAAACCAACACAC
CAATGCAACTGTGTTTTCTTCTGTTTTAAAGTATGTTGTCTTTGTATGCATGTTTGCTTCTTCCTTTTTT
TTTTTAACATCACAGATAAATTCAACTCTCACCTCAGGTTTTATTGAGAGAACTGTCAATGTGACTTGGC
CTCTGTCTTTCTAGTCCCAGAAAGAATTGCACTGAAATCTGAGCTCCTGTAATAAAAACAACCATTTGCT
GAGAGTAATTAACATACTGAAAGAGATTTTCTTAGAGTACACAATGGTGACATTATATTGCCTCTTTATA
AATAACTTTCTATCTATTTCTGTGGATTATTCCTACAAAGTACTTTTCATATGTCCAATTTCTTTTCTTC
CCCTACAACTACTGTCTGAATACTGGCTCTGCTATTTGCTGATATGATTCTCGGCAAGTTGCCTGCACTT
TTTAAACTTTATTTCCTCATTCAGAACATGGGGCCATACATAATACAACTCACTTCAGTGTTATTGGGGA
ATTAAACAAAAAATGCATGGGAAGCATTTAACATAGTGCCTGACACAATAATGAGTACTCAGTAGATGTT
AGCTTTTATTAATATTGTTGTTGTTATGTCCAGAAACACTATACCTCCAGAAAATCATGGGTACTTGCTG
GGGACATTGGGGATATGCATGATTTGGAAAAGAATGACTGCTTTTTTTGCTTAGATGAGAAATTTTTCTA
AGCCAGACTCCTTCAAATATGTAAGATTCTGTTGTGGATTCAAGGACTGAAAGAATTCTTGGCCGAGTGT
GGTGGCTTATCCCTGTAATCCCAGCATTTTGTGAGGACAAGGCAGGAAGATTGCTTGAGTCCAGGAGTTT
GAAACCAGCCTGCGCAACATGGCGAAACCCTGTCTCTACAAAAAATACAAACATTAGCTCGGAGTGAGTG
CTGACATGTGCCTGTACTCCCAGCTACTCAGAAGGCTGAGATGGGAGGATCTCATGAGCCTGGGGAGTTT
GAGGCTTCAGTGAGCCGTGATGACACCGTACTATACTCCACTCCAGCCTGGGTGACAGTGAGACCCTGCC
TCAAAAAACAAACAAACAAACAAACAAAACAAAATTAATCTTTTTGCTGATGTCATGTCAGCAGTGTGTG
TTGAAGGCTGTAAAGCAGCCATTTGTTCAGTTTATTTTTCCATTGAACAAGTATTTATCAAAAACATACT
TTGTGGCAGTCACTATGCTAGGAGCTATGAATACAGAAGGAAAAGTAAATGCTCTTGGATACTACACTCC
AGTTGTGATAAAAAAGAAAAAATGTATTCTTCACCAACTTCAACATCTTGATGTGCAAAAACATAATACA
TGAATTAGATCTACCTAATTACACAGAATTAGACCAATTGTTTCTGGAATTGTGGGCTCATATTTTTAAT
AACTGTCCTCCTGCCTCTCTGTCGACAGGTTTTATAAATATTCATTTAATTACACACACACACACGAACA
ATTGACTAGTACTTGCTCTCATTCTTCTAGATGTCATCACAATGGATCCCAAAGACAATTGTTCAAAAGA
TGCAAATGGTAAGCTTTTGTGTTTTTCCCTTCCTCCTGATCATTTTGTTTTGAACTTCTCTGGCTTGAAA
AATCAGGGAATGGATTTTGCTAGGTTGGATGCTGCAGAATGGACCTAGTGATATTTTAAATTAGTCCCTC
ATTTTCTAGGAGTTGTATTAACAAACCTAACTACTGCTTTGGGGTATGAGATGACTGTAAATTAGAGAGG
GTACAGTGGTATAGTGATATGCTTTTAATTATTTCAAAAAAAAGATTTTATTCATTCATGTGTCTTTTTT
CTTTTTCTTTTCTTTTTTTTTTTTTTTTGGACAGAGTCTTGCTCTGTCACCCAGGCTGGAGTGCGGTGGC
AGTATCTCAGCTCACCACAACCTCCGCCTCCCGGCTTCAAGTGATTCTCCTGCCTCAGCTTCTCGAGTAG
CTGGGACTACAGGCGCGTGCCACCATGCCCGGCTAATTTTTGTATTTTTAGTAGAGTTGGGGTTTCACCA
TGTTGGCCAGGATGGCCTCGAATTTGTGACCTCGTGATCTGCCCCCTCGCCCTCCCGAACTGTTGGGATT
ACAGGCGTGAGTCACTGTGCCCGGCCTCCTGTCCTGTCTTTTGTTTAATGACTGGGAAAAACATGATACC
ATGTTGCTTCTCGAGTTGTTTTGTTTTAGTCTTTGGTCTTTGCTAGTAGCTAATAACACGAACTAGTGTT
TATCAAGTGCTTTTTACACAGAAGGGCTTGGGCTGTGTTCTGCATTTTCTTGTTTAACCCTCTTAAAACT
CCTATAAAATGGTACATATTTTTCTCCCAATTTACAGTCCCTTTAAAGCAAATAATTATAAAAATCCCTA
TACATGTCACACAGCTAGATCTGGGATTTCAAATCAGGCC
ATCAAACAAAGAGTTTATGTACTTAGTAAGTTTTCTGTTCTTTTTCTACAATAGAGTCAGATAGCAAGAA
ATTACCAAGCCAGGAACCTGAAACAAAACGGACATCATGTGGGGCTGGGTGGGTGCATGGGCTTTGCAGA
CTGGACTTTCACTCCAGCTCTTTTAATGATTAGGTGTAAGTGACCTACATTTTGTGAGCAACAGTTTTCT
CATCAGCCAACAAAGAATAATTACACCAGATTCACAGTTATTGAAGAGATAAAGGCATGAATGTGAGATG
TCTGGCATAGGGCATCTCATTTAGCAGACACAGAATGAGTACTTGTTTCTGGCTTTTTCTCTCTACATAT
GCACAAAGAATGCGACTAGAAGCATGGGCTCTAGCCCTGCTCAACTTTCCTCTATTTCCAATACCAAGGG
GCTCTGACTTAGGCTGCCACACCAGGCAAGGAGGGCAGTACCACCTCACTTGACCAAGGGCAGGGAGTCA
CGGACACATCACTTCTTGAGATCCTTTTCCACACCAAGGACTGATGTTTCTGGAATTCTCACTTTATGAA
GACAAAACATATAAATGGAAATTTTCTCAGGTAGAGACTCACTCTTGTAGCTCATTGAGTAGGCACTAGT
GGTCCACCCCCACTGTCTTTACTTATTCCTTGACATCACATATCTCTTGCAAAACCTCAAATAATATTAA
ATGCAATCACCCAATAATAGCATAGCCATAATTAGAGGCATTTAGGAAAGACAGGTGAGTGTGCCACAAC
TACCTAACACATCAGCAAATCTGGATTAACCACTTTCTTTGATTTTCCACAATGCAACCTTACTTTTTAA
TAGTTGGGAATGTTCTAAGTGAATTTAGCAGAGGTTGTTAATCAACTTGAAAGCTGAATTCTGACTTGTC
TGACTCTTGGTGGTGCTGGTAGCAGTAGATGTTTACTTTTAGGTTTTGGTGGTGGTGGAATATCACTTCA
ACGTAAATCATCAGAAATAAGTATTTGTGAACCCCTCTCGCATTAATGTATCTTATTCTGTAAAAAGAAC
ATGTGCAATTTCTCTTAGATACACTACTGCTGCAGCTCACAAACACCTCTGCATATTACATGTACCTCCT
CCTGCTCCTCAAGAGTGTGGTCTATTTTGCCATCATCACCTGCTGTCTGCTTAGAAGAACGGCTTTCTGC
TGCAATGGAGAGAAATCATAA
[SEQ ID NO: 57]
ATAAACAACTTGATGCAGATGTTTCCCCCAAGCCCACTATTTTTCTTCCTTCGATTGCTGAAACAAAACT
CCAGAAGGCTGGAACATACCTTTGTCTTCTTGAGAAATTTTTCCCAGATATTATTAAGATACATTGGCAA
GAAAAGAAGAGCAACACGATTCTGGGATCCCAGGAGGGGAACACCATGAAGACTAACGACACATACATGA
AATTTAGCTGGTTAACGGTGCCAGAAGAGTCACTGGACAAAGAACACAGATGTATCGTCAGACATGAGAA
TAATAAAAACGGAATTGATCAAGAAATTATCTTTCCTCCAATAAAGACAGGTATGTGTTTACACATATCA
TCTGTCAGAACACTTCTTTGAAAGTGAATGCTGCATTTTTTCCTTTCAGTATTAATGAAAAACATAAATC
TTTCTTAAAAATTGTTACATTTAATGGTAGCGTAAATGCCCTGCTACTTTTCTATAGAATTAAAATGGTA
TAGGTTTTGGAGAAAACAAAATTGAAAAAGTTGCTGAAGGTTTGTCAGCCTCAGCTCCATTATCCAAAAT
AAGAAAGTCACGTGCTGGTTTTTAGGGTTGTTAGATGGATTAAAGAAACAACATACACAGAAGCATCTAG
CAACGTGACACGTGGTAAACGCTCAAAAAGTGTTCTCCCTTCTTTTGATGACTTTACTTGATCAGGAAAT
AACATATATATGTCTTTCAGGAATGTTCTGCCCAAGCAGGAGAGTCACTCACCTCAATCTTGCTACCCAC
AAAGTTTAACCTAAAAACAACGGGTTCATTGTTGACAAAATAATGTTTATCTGAAGATAACTGTAGATCA
TATTTATCTGTAGATAATGTTTATCTGTGGAGTGTGGCTCTACAAAACATAGAATAGTCTTGGTCACTGC
AGTTTTATAGAGGCCTTGGGTTTTTCAGAGTTTCATTTTATATATCACCATAAAGTAACATTTCATAATT
ACAGGTTGGTAAGGCTTACATGTACAAACATTCTTCCATTTTCCATAATAAATGCATTTCCTGCCATTGG
TGAATGCAGCTCAATAAACATTTATTGTACAATTATGACACGCCAGGCTTAGTGGAAATGTGGATGAACA
GACAAGGATGAGTTACTGTCCTAAGGATGATGCATGACAGTGCAGAGAATATACTCTCTTCCTGATCACT
CAGGGTCACTCATGATTCATGCGCGAGGTCCCAAAACAGTGCCTTTGATGCAGATTCTGTACATCTCTAG
ACGATTGGTCCAAGGGCTGAATGTGCTCTGGCCCAGTGGTCCAGTCTGTCACTATATGTCAACATCCTGA
ATATGAACATAACAGTCCAACATCTCAAGAGTGGGCATGAAAAGGACTCATTTTGTGCTTTTTCCTGTGG
TTAACAAGTCCTTTTTAGCCTGGGGGAACAAGCATTAACAAAATGTTTGAAGATCTTTGCCACGTACCAT
TCCAAATTTCTAGGGTAAGTCTTTAGCTTTTCAGATCCTGAGTTTCTGCAATGATCAAATGTGATTTGGA
CAGTTGCGTTGACTTTCTCCTGGGGCTATAATGGAGTGCAAAGGAAACAATGGCAGGGAAAATGCTTGCT
TTCAAAATGGTAGCATGGATGTGTTCATTCGTGTAGTTACTGTATTAGGTATAGCCTTTCCTGAAACTAA
CTGAAGTGGGGTTATAAAAACAGTCCCAATTTTCTATTTCCTTTGCTGAGACACAAAGAGGAGACAAAAG
AGCAAAGCTTGAGGGTAGTTTTACCACTGTGCTTAAGTGTTCTGATTTTTCCAGTGATCAGGGTGAAATA
AAAAGCATAGTAAGTTCCAGGGCAGTGAATACCATACAGGAGACAAGTTACAGTTTTATAATGTGTTTTA
CTTTACACTAAATTCTAAAAGTAAAATGTCTTTTTTTTTTTCCGAGACAGAGTTTCACTCTTGTAGCCCA
GGCAGGAGTGCTATGGTGTGATCTCGGCTCACAGCAACCTCCACCTCCCAGTTTCAAGCGATTCTTCTGC
CTCAGCCTCCCGAGAAGTTGAAATTACAGGTGCCTGGCACCATATCTCGCTAATTATTCTATTTTTAGTA
GAGATCGGGTTTTACCATGTTGGCCAGGCTGGTCTCGAACTCCTGACTTCAAGTGATCCACCCGCCTCAG
CCTCCCAAAGTGCTGGGATTACAGGTGTGAGTCACTGTGCCGGACCTAACAGTAAAATGTCTTTCATGTG
CTTCTCAAGGCAACTACATTAAGGAGGACACATCTCTTAATGTCATTCTACAGTAGATTTCTAATGCTCT
TTCTTGGAAGTTTGTTTTTCTGAGAAGAGCTAAAAATATAATAACATGGAAGTGATCATATTATATAATC
AATGAAGTGCTTTCAAAGGAGATAAAACTAACCTGGTCTGCATTTGCAACCAGCCTTGATTGAGAGAGAG
AGAACTCAGGATACACTTAGAGATTTTATTATGGGGAATAGTTACTTTATTCATTTTACCTCAATCAATG
CATGGAAATAAGTGACAGTCATTTTCATTTATCTTTTAATAAATAAAGTCACCATGAGGAAAATGAAAAC
CCATTAAAGTCAGTCCTTAAAGATATTTGGACATGCAGACATGATAACTAACATTTCCATTCGTGAGACT
TACCCAAAACCTATACCTCAAGTCCATTTCTTAGAATACATGAAATAAAGATCTCAGTGAGTGTATAAAA
CTGCACACCAGAATCATATCCGTATAGACAAGAATACATCTACTAGAAAAATATAAACCAAAACACCAAG
GTGACTCTGTTTTTTTCTGTTTTAAAATATGTTGTCTTTGTATGCATGTTTGCTTCTTCCTTTTTTTTTT
TAAACATCGCAGATAAATTCAACTCTCACCTCAGTTGAGAGAGAACTGTCAATGTGACTTGGCCTCTCTC
TTTCTAGTCCCAGAAAGAATTGCACTGAAATGCTGAGCTCCTGTAATAAAAATGACCATTTGCTGAGAGT
AATTAACATACTGAAAGAGATTTTCTTAGAATAGTGCACAATGGCCCAATGGTGACATTATATTGTCTCT
TTATAAATTATTTTCTATCTATTTCTGTGGATTATTTCTACAAAGCACTTTTCATATGTCCAATTCCTTT
TATTCCCCTACAAGTACTGACTGACTACTGGCTCTGCTGTTCACTGATATGACTTTCGGCAAGTTGCCTG
CACTTTTTAAACGTTATTTCCTCATTCAGAACATGGGGCCATACAAAATACAACTCACTTCAGTGTTATT
GGGGAATTAAACAAATAAATGCATGGGAAGCATTTAACATAGTGCCTGACACAATAATGAGCACTCAGTA
GATGTTAGCTTTTATTAATATTGTTGTTGCTATGTCCAGAAACACTATACCTCCAGAAAATCATGGGTAC
TTGCTGGGGACGTTGGGGATATGCATGATTTTGAAAGGAGTGACTGCTCTTTACTGCTCAGATGAGAAAT
TTTTCTAAGCCAGACTCCTTCAAACATGTAAGATTCTGTTGTGGATTCTAGGACTGAAAGAATTCTTGGC
CGAGTGTGGTGGCTTATCCTGGTAATCTCATCATTTGGGAGGACAAGGCAGGAAGATTGCTTGAGCCCAG
GAGTTGGAAACAAGCCTGGACAACATGGCGAAACCCTGTCTCTACAAAAAATACAAACATTAGCTGGTCA
TGGGAGTGAGTGCCTGTACTCCCAGCTACTCAGGAGGCTAAGATAGGAGGATCACCTGAGCCTGGGCAGT
TTGAGGTTTCAGTGAGCCGTGATGACACCATACTATACTCCACTCCAGCCTGGGTGACAGTGACATCCTG
CCTCAAAAAAACCCCCAAAATTATTCTTTTTGCTGATTTCATGTCAGCAGTGTGTGCTGAAGGCTGTAAA
GTAGCCACTTGTTCTGTTTATTTTTCCATTGAACAAGTATTTATCAAAAACGTACTTTGTGGAAGGCACT
GTGCTAGGAACTATGCATACAGAAGGAAAACCAAATGTTCTTGGATACTACACTCCAGTTGTGATAAAAA
AGAAAAAAGTATTCTTCACAAACTTCAACATTTTGATGTGCAAAAACATAATATATGAATTAGATCTACC
TAACTACACAGAATTAGACCAATTATTTCTGGGATTATGGGCTCATATTTTTAATAACTGTCCTCCTACC
TCTCTGTTGACAGGTTTTATAAATATTCATTTAATTACACACAGTCACAGACACACTCAGACACACACAC
ATACACACACACACACACCTTGACAAATAATGGGCATGAACAATTGACTGGTACTTGCTCTCATTCTTCT
AGATGTCACCACAGTGGATCCCAAATACAATTATTCAAAG
GATGCAAATGGTAAGTTTTTGTGTTTTTTATTTCCTCCTGATCATTTTAAGTTTTGAACTTCTCTGGCTT
GAAAAATCAGGGAATGGATTTTGCTAGGTTGGATGCTGCAGAATGGACCTAATCATATTTTAAATTAGTC
CCTCTTTTTCTAGGAGTTGTATTAACAAACCTAACTACTGCTTCATGTAAGAGATGACTGTAAATTGAAG
GGTACAGTGATATGCTTTCAGTTATTTCAAAAAACAGACTTTACTCATCCATGTGTCTTTTTTCTTTTCT
TTTTTTTCTTTTTTGAGACGGAGTCTCGCTCTGTTGAACAGGCTGGATTGCAGTGACGCGATCTCACCTC
ACTACAACCTCCGCCTCTGGAGTTCAAGCGATTCTCCAGCCTCAGCTTCTCAAGTAGCTGGGACTACAGG
CACATGCCACCATGTCCGGGTCATCTTTGTATTTTTAGCAGAGACCGGGTTTCACTATGTTGGCCAGGCT
GGTCTAGAATTCCTGACTTCGTGATCTGCCCCCTCAGCCCTCCGAAGTGCTGGGATTACAGACGTGAGTC
ACTGTGCCCGGCCTAACAGTAAAATGTCTTTCATGCGCTTCTCAAGGCAACTACGTTAAGGAGGACACTT
CTCTTAATGTCATTCTACAGTAGATTTCTAATGCTCTTTCTTGGAAGTTTGTTTTTCTGAGAAAAGCTAA
AAATATAACATGGAAGTGATCATATTGTATAATCAATGAAGTGCTTTTCAAGGAGATAAAACTAATCTGG
TCCACGTTTGCAACCAACCTTGATTGAGAGAGAGAGAGAACTCAGGATACACTTGGAGATTTTATTATGG
GGAATAGTTACTTTATTCTTTTTTCCTCAATCAATTCATGGAAATAAGTGATAGTCATATTCATTTATCT
TTTAATAAATGAAGTCACCATGAGGAAAATAAAAAGACATTGAAAACCCATTAAAGTTAGCCCTTAAAGA
TATTTGGACATGCAGACTTGATAACTAACGTTTGCATTCTTGAGACTTACCCAAAACCCATACCTCAAGT
CCATGTTTTTAGAATTCATGAAATAAAGATCTCAGTGAGTGCATAAAATTGCGCACCAGAATCATATCCG
TATAGACAAGAACACATCTACTAGAAAAATAATAAACCAACACACCAATGCAACTGTGTTTTCTTCTGTT
TTAAAATATGTTGTCTTTGTATGCATGTTTGCTTCTTCCTTTTTTTTTTTTAACATCACAGATAAATTCA
ACTCTCACCTCAGGTTTTATTGAGAGAACTGTCAATGTGACTTGGCCTCTGTCTTTCTAGTCCCAGAAAG
AATCGCACTGAAATGCTGAGCTCCTGTAATAAAAATGACCATTTGCTGAGAGTAATTAACATACTGAAAG
AGATTTTCTTAGAGTACACAATGGTGACATTATATTGTCTCTTTATAAATAACTTTCTATCTATTTCTGT
GGATTATTCCTACAAAGTACTTTTCATATGTCCAGTTTCTTTTCTTCCCCTACAACTACCGTCTGAATAC
TGGCTCTGCTATTTGCTGATATGATTCTCGGCAAGTTGCCTGCACTTTTTAAACTTTATTTCCTCATTCA
GAACATGGGGCCATGTAATACTCATGTACGTGAGTATTACGTAATAATGCTCACTTAAGTGTTACTGGGG
AATTAAACAAAAAAATGCATGGCAAGCATTTAACATAGTGCCTGACACAATAATGAGCACTCAGTAGATG
TTAGATTTTATTAATATTGTTGTTGTTATGTCCGGAAACACTATACCTCCAGAAAATCATGGGTACTTGC
TTGGGATGTTGGGGATATGCATGATTTGGAAAGGTATGACTGCTTTTTTCTGCTTAGATGAGAAATTTTT
CTAAGCCAGACTCCTTCAAATATGTAAGATTCTGTTGTGGATTCTAGGACGGAAAGAATTCTTGGTCAGG
TGTGGTTTCTTATCCCTGTAATCCCAGAATTTTGGGAGGACAAGGCAGGAAGATTGCTTGAGCCCAGGAG
TTTGAAACCAGCCTGGGCAACAAGACGAAACCCTGTCTCTACAAAAGTACATAAATTAGCTTGGCTTGGT
GGTGTGTGCCTGTATTACCAGCTATTCGGGAGACTGAGATGGGAGGATCTCCTGAACCTGTGAAGTTTGA
GGCTTCAGTGAGCCGTGATGACACCATACTATACTCGACTCCAGCCTGTGCGACAGTGAGACTCTGCGTC
AAAAAAAAAACCCCAAAATTATTGTTTTTGCTGATTTCAGGTCAGCAGTGTGTGCTGAAGGGTGTAAAGT
AGCCACTTGATCAGTTTATTTTTCCACTGAACAAGTATTTATCAAAAACATACTTTGTGGTCTGTTTTTG
ATAAATAAAAAGGCACTGTGCTAGGAGCCATGAATACAGAAGGAAAACCAAATGTTCTTGGATACTACAC
TCCAGTTGTGATAAAAAAGAAAAATGTATTCTTCACGAACTTCAACATTTTGATATGCAAAAACATAGTA
TATAAATTAGATCTACCTGATTACGTAGAATCAGACCAATTATTTCTGGAATTGAGGGCTCATATTTTTA
ATAACTGTCCTCCTGCCTCTCTGTTGACAGGTTTTATAAATATTCATTTAATTACACACACACACACACA
CACCTTGACAAATAATGGACATGAACAATTGACTAGTACTTGCTCTCATTCTTCTAGATGTCATCACAAT
GGATCCCAAAGACAATTGGTCAAAAGATGCAAATGGTAAGCTTTTGTGTTTTTCCTTTCCTCCTGATCAT
TTTAAGTTTTGAACTTCTCTGGCTTGAAAAATCAGGGAATGGGCCGGGTGCGGTGGCTCACGCCTGTAAT
CCCAGCACTTTGGGAGGCCGAGGCGGGCGGATCACGAGGTCAGGAGATCGAGACCATCCCGGCTAAAACG
GTGAAACCCCGTCTCTACTAAAAATACAAAAAATTAGCCGGGCTTAGTGGCGGGCGCCTGTAGTCCCAGC
TACTTGGGAGGCTGAGGCAGGAGAATGGCGTGAACCCGGGAGGCGGAGCTTGCAGTGAGCCGAGATTGCG
CCACTGCACTCCACTCCAGCCTGGGCGACAGAGCGAGACTCCGTCTCAAAAAAAAAAAAAAAAAAAAAAA
AAGAAAAATCAGGGAATGGATTTTGCTAGGTTGGATGCTGCAGAATGGACCTAGTGATATTTTAAATTAG
TCCCTCTTTTTCTAGGAGTTGTATTAACAAACCTAACTACTGCTTCGGGTATGAGATGACTGTAAATTAG
AGGGTACAGTGATATGCTTTCAGTTATTTCAAAAAACAGACTTTATTCATCCGTCTGTCTTTTTTTTTTT
TTTTTTTTTTTTTTTTTGAGACGGAGGAGTCTCACTCTATCACCCAGGCTGGAGTGCAGTGGCGCGATCT
CGGCTCACCATAACCTCCGCCTTACTGGTTCAAGCGATTCTCCAGCCTCAGCTTCTCAAGTAGCTGGGAC
TACAGGTGCACACCACCATACCTGGCTAATTTTTGTATTTTTAATAGAGATGGGGTTTCACCACGCTGGC
CAGGATGGTCTTGAATTCTTGACCTCGTGATCTGCCCCCTCGGGCTCCCAAACTTCTGGGATTATAGGCG
TGAGCCACTGTGCCCGGCCTTCTGTCTTTTGTTATAATGACTGGGGAAAACATGATACCATGTTGCTTCT
TGAGTTGTTTTGTTTTAGTCTTTGGTCTTTGCTAGTAGCTAATAACACGAACTAGTGTTTATCAAGTGCT
TTTTACACAGAAGGGCTTGTTCTGCATTTTCTAGTTTAATCATCTTAATACTCCTATAAAGTAGTACAAT
ATATTTTCTCCCATTTTACAGTCCCTTTAAAGTAAATAACTATAAAAATCCCTTATACATGTCACACAGC
TAGGTCTGGCATTTCAAATCAGGACATCAAACAAAGAATTCGTGCAGTTACTAAGTCCTCTATTTTTTCT
ACAATAGAAAAAATAGCAAGAATTACAGATAGCAAGACATTACAAGGCAGGAATCTGAAACGAAAGGGAC
ATAATGTGGGGCTGGGTGGGTGCATGAGCTTTGCAGACTAGACTTTCATTCCAGCTCTTTTAATGATTAG
GTGTAAGTGACCTACATTTTGTGAGTAACAGTTTTCTCATCAGCCAACTAAGAATAATTACACCAGATTC
ACAGTTATTGAAGAGATAAGGGCATGAATGTGAGATGTCTGGCGTAGGGTATCTCATTTAGCAGACACAG
AATGAATACTTGTTTCTGGCTTTTTCTCTCTACATATGCACAAAGAATGTGACTAGAAGCATTGGCTCTA
GCCCTGCTCAACTTTCCTCTATTTCCAATACCAAGGGGCTCTGACTTAGGCTGCCACACCAGGCAAGGAG
GGGCAGTACCACCTCACTTGACCAAGGGCAGGGAGTCACGGACACATCACTTCCTGAGATCCTTTTCCAC
ACCAAGGACTGATGTTTCTGGAATTCTCACTTTATGAAGACAAAACATATAAATGGAAATTTCTGCAGGA
AGAGACTCACTCTTGTAGCTCATTGAGTAGGCACTAGTGGTCCACCCCCACTGTCTTTACTTATTCCTTG
ACATCACATATCTCTTGTAAAACCTCAAATAATGTTAAATGCAATCACCCAATAATAGCATAGCCATAAT
TAGAGGCATTTAGGAAAGACAGGTGAGTGTGCCACAACTACCTAACACATCAGCAAATCTGGATTAACCA
CTTTCTTTGATTTTCCACAATGCAACCTTACTTTTTAATAGTTGGGAATGTTCTAAGTGAATTTAGCAGA
GGTTGTTAATCAACTTGAAAGCTGAATTCTGACTTGTCTGACTCTTGGTGGTGCTGGTAGCAGTAGATGT
TTACTTTTAGGTTTTGGTGGTGGTGGAATATCACTTCAACGTAAATCATCAGAAATAAGTATTTGTGAAC
CCCTCTCGCATTAATATATCTTATTCTGTAAAAAGAACATGTGCAATTTCTCTTAGATACACTACTGCTG
CAGCTCACAAACACCTCTGCATATTACACGTACCTCCTCCTGCTCCTCAAGAGTGTGGTCTATTTTGCCA
TCATCACCTGCTGTCTGCTTAGAAGAACGGCTTTCTGCTGCAATGGAGAGAAATCATAA

In certain embodiments, the first antigen binding chain or the second antigen binding chain comprises a constant domain comprising a native or modified TRDC polypeptide. In certain embodiments, the TRDC polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 58, which is provided below. In certain embodiments, the TRDC polypeptide comprises or consists of the amino acid sequence set forth in SEQ ID NO: 58.

[SEQ ID NO: 58]
SQPHTKPSVFVMKNGTNVACLVKEFYPKDIRINLVSSKKITEFDPAIVIS
PSGKYNAVKLGKYEDSNSVTCSVQHDNKTVHSTDFEVKTDSTDHVKPKET
ENTKQPSKSCHKPKAIVHTEKVNMMSLTVLGLRMLFAKTVAVNFLLTAKL
FFL

In certain embodiments, the TCR-like fusion molecule comprises a hinge/spacer region that links the first antigen binding chain to the constant domain. In certain embodiments, the TCR-like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH₂CH₃ region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 59. In certain embodiments, the hinge/spacer region comprises or consists of amino acids 1 to 3 of the sequence set forth in SEQ ID NO: 59. An exemplary nucleic acid sequence encoding the amino acid sequence of SEQ ID NO: 59 is set forth in SEQ ID NO: 60. SEQ ID NO: 59 and 60 are provided below.

[SEQ ID NO: 59]
IPNIQNPDPA
[SEQ ID NO: 60]
ATTCCCAATATCCAGAACCCTGACCCTGCC

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 61. In certain embodiments, the hinge/spacer region comprises or consists of amino acid 1 to 2 of the sequence set forth in SEQ ID NO: 61. An exemplary nucleic acid sequence encoding the amino acid sequence of SEQ ID NO: 61 is set forth in SEQ ID NO: 62. SEQ ID NO: 61 and 62 are provided below.

[SEQ ID NO: 61]
LEDLKNVFPPE
[SEQ ID NO: 62]
CTGGAGGATCTGAAAAACGTGTTCCCTCCTGAA

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR-like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 12 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 8, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 8. In certain embodiments, the CD3ζ polypeptide comprises or consists of amino acids 52 to 164 of SEQ ID NO: 8.

In certain embodiments, the CD3ζ polypeptide comprises or consists of an amino acid sequence that is at least about 85%, about 90%, about 95%, about 96%, about 97%, about 98%, about 99% or about 100% homologous or identical to SEQ ID NO: 9 or a fragment thereof, and/or may optionally comprise up to one or up to two or up to three conservative amino acid substitutions. In certain embodiments, the CD3ζ polypeptide comprises or consists of the amino acid sequence set forth in SEQ ID NO: 9.

In certain embodiments, the TCR-like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR-like fusion molecule is capable of associating with a CD3 complex (also known as “T-cell co-receptor”). In certain embodiments, the TCR-like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR-like fusion molecule replaces a native and/or an endogenous TCR in the CD3/TCR complex. In certain embodiments, the CD3 complex comprises a CD37 chain, a CD36 chain, and two CD3F chains.

In certain embodiments, the CD37 chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 63) or a fragment thereof, and/or may optionally comprise up to one or up to two or up to three conservative amino acid substitutions. SEQ ID NO: 63 is provided below.

[SEQ ID NO: 63]
MEQGKGLAVLILAIILLQGTLAQSIKGNHLVKVYDYQEDGSVLLTCDAEA
KNITWFKDGKMIGELTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN

In certain embodiments, the CD36 chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 64) or a fragment thereof, or the amino acid sequence having a NCBI reference numbers: NP_001035741.1 (SEQ ID NO: 65) or a fragment thereof, and/or may optionally comprise up to one or up to two or up to three conservative amino acid substitutions. SEQ ID NO: 64 and 65 are provided below.

[SEQ ID NO: 64]
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK
[SEQ ID NO: 65]
MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQVYQPLRDRDDAQYSHLGGNWARNK

In certain embodiments, the CD3F chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 66) or a fragment thereof, and/or may optionally comprise up to one or up to two or up to three conservative amino acid substitutions. SEQ ID NO: 66 is provided below.

[SEQ ID NO: 66]
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

In certain embodiments, the TCR-like fusion molecule exhibits a greater antigen sensitivity than a CAR targeting the same antigen. In certain embodiments, the TCR-like fusion molecule is capable of inducing an immune response when binding to an antigen that has a low antigen density on the surface of a tumor cell. In certain embodiments, cells comprising the TCR-like fusion molecule can be used to treat a subject having tumor cells with a low expression level of a surface antigen, e.g., from a relapse of a disease, wherein the subject received treatment which leads to residual tumor cells. In certain embodiments, the tumor cells have a low antigen density of a target molecule on the surface of the tumor cells. In certain embodiments, a target molecule having a low antigen density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low antigen density on the cell surface has a density of less than about 2,000 molecules per cell. In certain embodiments, a target molecule having a low antigen density on the cell surface has a density of less than about 1,500 molecules per cell. In certain embodiments, a target molecule having a low antigen density on the cell surface has a density of less than about 1,000 molecules per cell. In certain embodiments, a target molecule having a low antigen density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

In certain embodiments, the TCR-like fusion molecule is capable of inducing an immune response when binding to an antigen that is expressed on the surface of a tumor cell having a low tumor cell frequency. In certain embodiments, cells comprising the TCR-like fusion molecule can be used to treat a subject having tumor cells with a low tumor cell frequency, e.g., from a relapse of a disease, wherein the subject received treatment which leads to residual tumor cells. In certain embodiments, the tumor having a low tumor cell frequency has a frequency that is less than about 40% per tumor, less than about 30% per tumor, less than about 20% per tumor, less than about 15% per tumor, less than about 10% per tumor, less than about 5% per tumor, less than about 2% per tumor, or less than about 1% per tumor. In certain embodiments, the low tumor cell frequency is less than about 2% per tumor. In certain embodiments, the low tumor cell frequency is less than about 1.5% per tumor. In certain embodiments, the low tumor cell frequency is less than about 1% per tumor. In certain embodiments, the low tumor cell frequency is between about 40% per tumor and about 20% per tumor, between about 20% per tumor and about 10% per tumor, between about 15% per tumor and about 10% per tumor, between about 20% per tumor and about 5% per tumor, between about 10% per tumor and about 2% per tumor, or between about 10% per tumor and about 1% per tumor.

In certain embodiments, the second antigen-recognizing receptor is a TCR-like fusion molecule that comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRBC polypeptide. In certain embodiments, the hinge region comprises or consists of the amino acid sequence set forth in SEQ ID NO: 59 or SEQ ID NO: 61. In certain embodiments, the second antigen binding chain comprises a hinge region between the V_{L} and the TRAC polypeptide. In certain embodiments, the hinge region comprises or consists of the amino acid sequence set forth in SEQ ID NO: 59 or SEQ ID NO: 61.

In certain embodiments, the second antigen-recognizing receptor is a TCR-like fusion molecule that comprises a first antigen binding chain comprising a V_{H} of an antibody and a constant domain comprising a TRAC polypeptide; and a second antigen binding chain comprising a V_{L} of an antibody and a constant domain comprising a TRBC polypeptide. In certain embodiments, the first antigen binding chain is designated as “V_{H}-TRAC chain”. In certain embodiments, the second antigen binding chain is designated as “V_{L}-TRBC chain”. In certain embodiments, the first antigen binding chain comprises a hinge region between the V_{H} and the TRAC polypeptide. In certain embodiments, the second antigen binding chain comprises a hinge region between the V_{L} and the TRBC polypeptide. In certain embodiments, the first antigen binding chain and the second antigen binding chain bind to a second antigen (e.g., human CD70).

In certain embodiments, the second antigen-recognizing receptor is a TCR-like fusion molecule that comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRAC polypeptide. In certain embodiments, the second antigen binding chain comprises a hinge region between the V_{L} and the TRBC polypeptide. In certain embodiments, the first antigen binding chain and the second antigen binding chain bind to a second antigen (e.g., human CD70).

### 2.2.2.1. Exemplary TCR-Like Fusion Molecules

In certain embodiments, the second antigen-recognizing receptor is a TCR-like fusion molecule that binds to CD70 (e.g., human CD70) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the V_{H} comprises a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135. In certain embodiments, the V_{H} comprises the amino acid sequence set forth in SEQ ID NO: 139. In certain embodiments, the V_{L} comprises a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138. In certain embodiments, the V_{L} Comprises the amino acid sequence set forth in SEQ ID NO: 141. In certain embodiments, the TRAC polypeptide comprises the amino acid sequence set forth in SEQ ID NO: 42. In certain embodiments, the TRBC polypeptide is a TRBC2 polypeptide. In certain embodiments, the TRBC2 polypeptide comprises the amino acid sequence set forth in SEQ ID NO: 47. In certain embodiments, the TCR-like fusion molecule is designated as “70-HIT” or “70H”. SEQ ID NO: 133-142 are provided in Table 7 below.

In certain embodiments, the CDRs regions/sequences disclosed herein are delineated using the Kabat system (Swindells et al., J Mol Biol. 2017 Feb. 3; 429(3):356-364).

TABLE 7

| CDRs | 1 | 2 | 3 |
|---|---|---|---|
| V_{H} | SYIMH [SEQ ID NO: | VISYDGRNKYYADSVKG | DTDGYDFDY [SEQ |
| | 133] | [SEQ ID NO: 134] | ID NO: 135] |
| V_{L} | RASQSVSSYLA [SEQ ID | DASNRAT [SEQ ID NO: | QQRTNWPLT [SEQ |
| | NO: 136] | 137] | ID NO: 138] |

| | |
|---|---|
| Full V_{H} | MELGLSWIFLLAILKGVQCQVQLVESGGGVVQPGRSLRLSCAASGFTFSSYIMHWVRQ |
| | APGKGLEWVAVISYDGRNKYYADSVKGRFTISRDNSKNTLYLQMNSLRAEDTAVYYCA |
| | RDTDGYDFDYWGQGTLVTV [SEQ ID NO: 139] |
| Full V_{H} - | ATGGAACTCGGTCTGTCCTGGATTTTTCTGCTTGCGATCTTGAAAGGAGTGCAGTGCC |
| DNA | AGGTGCAGCTGGTGGAGTCTGGGGGAGGCGTGGTCCAGCCTGGGAGGTCCCTGAGACT |
| | CTCCTGTGCAGCCTCTGGATTTACCTTCAGTAGCTATATTATGCACTGGGTCCGCCAG |
| | GCTCCAGGCAAGGGGCTGGAGTGGGTGGCAGTTATATCATATGATGGAAGAAACAAAT |
| | ACTACGCAGACTCCGTGAAGGGCCGATTCACCATCTCCAGAGACAATTCCAAGAACAC |
| | GCTGTATCTGCAAATGAACAGCCTGAGAGCTGAGGACACGGCTGTGTATTACTGTGCG |
| | AGAGATACGGATGGCTACGATTTTGACTACTGGGGCCAGGGAACCCTGGTCACCGTC |
| | [SEQ ID NO: 140] |
| Full V_{L} | EIVLTQSPATLSLSPGERATLSCRASQSVSSYLAWYQQKPGQAPRLLIYDASNRATGI |
| | PARFSGSGSGTDFTLTISSLEPEDFAVYYCQQRTNWPLTFGGGTKVEIK [SEQ ID |
| | NO: 141] |
| Full V_{L} - | ATGGAAGCCCCAGCTCAGCTTCTCTTCCTCCTGCTACTCTGGCTCCCAGATACCACCG |
| DNA | GAGAAATTGTGTTGACACAGTCTCCAGCCACCCTGTCTTTGTCTCCAGGGGAAAGAGC |
| | CACCCTCTCCTGCAGGGCCAGTCAGAGTGTTAGCAGCTACTTAGCCTGGTACCAACAG |
| | AAACCTGGCCAGGCTCCCAGGCTCCTCATCTATGATGCATCCAACAGGGCCACTGGCA |
| | TCCCAGCCAGGTTCAGTGGCAGTGGGTCTGGGACAGACTTCACTCTCACCATCAGCAG |
| | CCTAGAGCCTGAAGATTTTGCAGTTTATTACTGTCAGCAGCGTACCAACTGGCCGCTC |
| | ACTTTCGGCGGAGGGACCAAGGTGGAGATC [SEQ ID NO: 142] |

In certain embodiments, the second antigen-recognizing receptor is a TCR-like fusion molecule that binds to CD70 (e.g., human CD70) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the V_{H}comprises a CDR1, a CDR2, and a CDR3 of a V_{H} sequence of an anti-CD70 antibody disclosed in International Patent Publication No. WO 2007/038637, which is incorporated by reference in its entirety. In certain embodiments, the V_{H} comprises a CDR1, a CDR2, and a CDR3 of a V_{H} sequence of an anti-CD70 antibody 2H5 disclosed in International Patent Publication No. WO 2007/038637. In certain embodiments, the V_{L} comprises a CDR1, a CDR2, and a CDR3 of a V_{H} sequence of an anti-CD70 antibody disclosed in International Patent Publication No. WO 2007/038637. In certain embodiments, the V_{L} comprises a CDR1, a CDR2, and a CDR3 of a V_{H} sequence of an anti-CD70 antibody 2H5 disclosed in International Patent Publication No. WO 2007/038637.

In certain embodiments, the second antigen-recognizing receptor is a TCR-like fusion molecule that binds to CD19 (e.g., human CD19) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD19. In certain embodiments, the V_{H} comprises a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145. In certain embodiments, the V_{H} comprises the amino acid sequence set forth in SEQ ID NO: 149. In certain embodiments, the V_{L} comprises a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148. In certain embodiments, the V_{L} Comprises the amino acid sequence set forth in SEQ ID NO: 151. In certain embodiments, the TRAC polypeptide comprises the amino acid sequence set forth in SEQ ID NO: 42. In certain embodiments, the TRBC polypeptide is a TRBC2 polypeptide. In certain embodiments, the TRBC2 polypeptide comprises the amino acid sequence set forth in SEQ ID NO: 47. In certain embodiments, the TCR-like fusion molecule is designated as “19-HIT” or “19H”. SEQ ID NO: 143-152 are provided in Table 8 below.

In certain embodiments, the CDRs regions/sequences disclosed herein are delineated using the Kabat system (Swindells et al., J Mol Biol. 2017 Feb. 3; 429(3):356-364).

TABLE 8

| CDRs | 1 | 2 | 3 |
|---|---|---|---|
| V_{H} | SYWMN [SEQ ID NO: | QIYPGDGDTNYNGKFKG | KTISSVVDFYFDY |
| | 143] | [SEQ ID NO: 144] | [SEQ ID NO: 145] |
| V_{L} | KASQNVGTNVA [SEQ ID | SATYRNS [SEQ ID NO: | QQYNRYPYT [SEQ |
| | NO: 146] | 147] | ID NO: 148] |

| | |
|---|---|
| Full V_{H} | MALPVTALLLPLALLLHAEVKLQQSGAELVRPGSSVKISCKASGYAFSSYWMNWVKQR |
| | PGQGLEWIGQIYPGDGDTNYNGKFKGQATLTADKSSSTAYMQLSGLTSEDSAVYFCAR |
| | KTISSVVDFYFDYWGQGTTVTV [SEQ ID NO: 149] |
| Full V_{H}- | ATGGCTCTCCCAGTGACTGCCCTACTGCTTCCCCTAGCGCTTCTCCTGCATGCAGAGG |
| DNA | TGAAGCTGCAGCAGTCTGGGGCTGAGCTGGTGAGGCCTGGGTCCTCAGTGAAGATTTC |
| | CTGCAAGGCTTCTGGCTATGCATTCAGTAGCTACTGGATGAACTGGGTGAAGCAGAGG |
| | CCTGGACAGGGTCTTGAGTGGATTGGACAGATTTATCCTGGAGATGGTGATACTAACT |
| | ACAATGGAAAGTTCAAGGGTCAAGCCACACTGACTGCAGACAAATCCTCCAGCACAGC |
| | CTACATGCAGCTCAGCGGCCTAACATCTGAGGACTCTGCGGTCTATTTCTGTGCAAGA |
| | AAGACCATTAGTTCGGTAGTAGATTTCTACTTTGACTACTGGGGCCAAGGGACCACGG |
| | TCACCGTC [SEQ ID NO: 150] |
| Full V_{L} | MALPVTALLLPLALLLHADIELTQSPKFMSTSVGDRVSVTCKASQNVGTNVAWYQQKP |
| | GQSPKPLIYSATYRNSGVPDRFTGSGSGTDFTLTITNVQSKDLADYFCQQYNRYPYTS |
| | GGGTKLEI [SEQ ID NO: 151] |
| Full V_{L} - | ATGGCACTGCCCGTGACCGCTCTGCTTCTCCCACTAGCTCTGCTTCTCCACGCAGACA |
| DNA | TTGAGCTCACCCAGTCTCCAAAATTCATGTCCACATCAGTAGGAGACAGGGTCAGCGT |
| | CACCTGCAAGGCCAGTCAGAATGTGGGTACTAATGTAGCCTGGTATCAACAGAAACCA |
| | GGACAATCTCCTAAACCACTGATTTACTCGGCAACCTACCGGAACAGTGGAGTCCCTG |
| | ATCGCTTCACAGGCAGTGGATCTGGGACAGATTTCACTCTCACCATCACTAACGTGCA |
| | GTCTAAAGACTTGGCAGACTATTTCTGTCAACAATATAACAGGTATCCGTACACGTCC |
| | GGAGGGGGGACCAAGCTGGAGATC [SEQ ID NO: 152] |

Various TCR-like fusion molecules are disclosed in International Patent Application Publication No. WO2019/133969, which is incorporated by reference hereby in its entirety.

### 2.2.3. Delivery of the Second Antigen-Recognizing Receptor

In certain embodiments, the second antigen-recognizing receptor is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the second antigen-recognizing receptor is delivered to the cell by a non-viral method. Any targeted genome editing methods can also be used to deliver the second antigen-recognizing receptor to the cell. In certain embodiments, the second antigen-recognizing receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the second antigen-recognizing receptor to the cell.

In certain embodiments, the cell is a T cell, and the second antigen-recognizing receptor is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the second antigen-recognizing receptor is integrated at a TRAC locus.

### 2.3. CCRs

In certain embodiments, a presently disclosed cell comprising a first antigen-recognizing receptor and a second antigen-recognizing receptor further comprises a CCR. The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in US20020018783 the contents of which are incorporated by reference in their entireties. CCRs mimic co-stimulatory signals, but unlike, CARs, do not provide a T-cell activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a third antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the third antigen is selected so that expression of both of the first/second antigen and the third antigen is restricted to the targeted cells (e.g., cancerous tissue or cancerous cells, or LSCs, or AML HSPCs). Similar to a CAR, the extracellular antigen-binding domain can be an scFv, a Fab, a F(ab)₂, or a fusion protein with a heterologous sequence to form the extracellular antigen-binding domain.

In certain embodiments, the cell comprising the first antigen-recognizing receptor, the second antigen-recognizing receptor, and the CCR exhibits a greater degree of cytolytic activity against cells that are positive for both the first/second antigen and the third antigen as compared to against cells that are singly positive for the first/second antigen. In certain embodiments, the cell comprising the first antigen-recognizing receptor, the second antigen-recognizing receptor, and the CCR exhibits substantially no or negligible cytolytic activity against cells that are singly positive for the first/second antigen.

In certain embodiments, the first antigen recognizing receptor and/or the second antigen recognizing receptor binds to the first antigen and the second antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the first antigen recognizing receptor (e.g., a CAR, a TCR, or a TCR-like fusion molecule) binds to the first antigen with a low binding avidity. In certain embodiments, the first antigen recognizing receptor (e.g., a CAR, a TCR, or a TCR-like fusion molecule) binds to the first antigen at an epitope of low accessibility. In certain embodiments, the first antigen recognizing receptor (e.g., a CAR, a TCR, or a TCR-like fusion molecule) binds to the first antigen with a binding affinity that is lower compared to the binding affinity with which the second antigen-recognizing receptor (e.g., a CCR) binds to the second antigen. In certain embodiments, the CCR binds to the third antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 2.4. T Cell Receptors (TCRs)

In certain embodiments, a presently disclosed cell comprising a first antigen-recognizing receptor and a second antigen-recognizing receptor further comprises a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the TCR is naturally occurring TCR.

In certain embodiments, the TCR is an exogenous TCR. In certain embodiments, the TCR is a recombinant TCR. In certain embodiments, the TCR is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 2.5. Co-stimulatory Ligands

In certain embodiments, a presently disclosed cell comprising a first antigen-recognizing receptor and a second antigen-recognizing receptor further comprises at least one recombinant or exogenous co-stimulatory ligand. For example, a presently disclosed cell can be further transduced with at least one co-stimulatory ligand, such that the cell expresses or is induced to express the first antigen-recognizing receptor, the second antigen-recognizing receptor, and the at least one co-stimulatory ligand. The at least one co-stimulatory ligand provides a co-stimulation signal to the cell.

Non-limiting examples of co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Non-limiting examples of TNF superfamily members include nerve growth factor (NGF), CD40L (also known as “CD154”), 4-1BBL, TNF-α, OX40L, CD70, Fas ligand (FasL), CD30L, tumor necrosis factor beta (TNFβ)/lymphotoxin-alpha (LTα), lymphotoxin-beta (LTO), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Non-limiting examples of immunoglobulin superfamily ligands include CD80, CD86, and ICOSLG. In certain embodiments, the at least one co-stimulatory ligand is selected from the group consisting of 4-1BBL, CD80, CD86, CD70, GITRL, CD40L, OX40L, CD30L, TNFRSF14, ICOSLG, TRAIL, and combinations thereof.

In certain embodiments, the cell further comprises one exogenous co-stimulatory ligand that is 4-1BBL. In certain embodiments, the co-stimulatory ligand is human 4-1BBL. In certain embodiments, the 4-1BBL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a Uniprot Reference No: P41273-1 (SEQ ID NO: 67) or a fragment thereof, and/or may optionally comprise up to one or up to two or up to three conservative amino acid substitutions. In certain embodiments, the 4-1BBL comprises or consists of an amino acid sequence that is a consecutive portion of the amino acid sequence of SEQ ID NO: 67. SEQ ID NO: 67 is provided below.

[SEQ ID NO: 67]
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

An exemplary nucleotide sequence encoding the amino acid sequence of SEQ ID NO: 62 is set forth in SEQ ID NO: 68.

[SEQ ID NO: 68]
ATGGAATACGCCTCTGACGCTTCACTGGACCCCGAAGCCCCGTGGCCTCC
CGCGCCCCGCGCTCGCGCCTGCCGCGTACTGCCTTGGGCCCTGGTCGCGG
GGCTGCTGCTGCTGCTGCTGCTCGCTGCCGCCTGCGCCGTCTTCCTCGCC
TGCCCCTGGGCCGTGTCCGGGGCTCGCGCCTCGCCCGGCTCCGCGGCCAG
CCCGAGACTCCGCGAGGGTCCCGAGCTTTCGCCCGACGATCCCGCCGGCC
TCTTGGACCTGCGGCAGGGCATGTTTGCGCAGCTGGTGGCCCAAAATGTT
CTGCTGATCGATGGGCCCCTGAGCTGGTACAGTGACCCAGGCCTGGCAGG
CGTGTCCCTGACGGGGGGCCTGAGCTACAAAGAGGACACGAAGGAGCTGG
TGGTGGCCAAGGCTGGAGTCTACTATGTCTTCTTTCAACTAGAGCTGCGG
CGCGTGGTGGCCGGCGAGGGCTCAGGCTCCGTTTCACTTGCGCTGCACCT
GCAGCCACTGCGCTCTGCTGCTGGGGCCGCCGCCCTGGCTTTGACCGTGG
ACCTGCCACCCGCCTCCTCCGAGGCTCGGAACTCGGCCTTCGGTTTCCAG
GGCCGCTTGCTGCACCTGAGTGCCGGCCAGCGCCTGGGCGTCCATCTTCA
CACTGAGGCCAGGGCACGCCATGCCTGGCAGCTTACCCAGGGCGCCACAG
TCTTGGGACTCTTCCGGGTGACCCCCGAAATCCCAGCCGGACTCCCTTCA
CCGAGGTCGGAA

In certain embodiments, the cell further comprises one exogenous co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005182 (SEQ ID NO: 69) or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 69. SEQ ID NO: 69 is provided below.

[SEQ ID NO: 69]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFAPRCRERRRNERLRRESVRPV

An exemplary nucleotide sequence encoding the amino acid sequence of SEQ ID NO: 69 is set forth in SEQ ID NO: 70. SEQ ID NO: 70 is provided below.

[SEQ ID NO: 70]
ATGGGCCACACACGGAGGCAGGGAACATCACCATCCAAGTGTCCATACCT
CAATTTCTTTCAGCTCTTGGTGCTGGCTGGTCTTTCTCACTTCTGTTCAG
GTGTTATCCACGTGACCAAGGAAGTGAAAGAAGTGGCAACGCTGTCCTGT
GGTCACAATGTTTCTGTTGAAGAGCTGGCACAAACTCGCATCTACTGGCA
AAAGGAGAAGAAAATGGTGCTGACTATGATGTCTGGGGACATGAATATAT
GGCCCGAGTACAAGAACCGGACCATCTTTGATATCACTAATAACCTCTCC
ATTGTGATCCTGGCTCTGCGCCCATCTGACGAGGGCACATACGAGTGTGT
TGTTCTGAAGTATGAAAAAGACGCTTTCAAGCGGGAACACCTGGCTGAAG
TGACGTTATCAGTCAAAGCTGACTTCCCTACACCTAGTATATCTGACTTT
GAAATTCCAACTTCTAATATTAGAAGGATAATTTGCTCAACCTCTGGAGG
TTTTCCAGAGCCTCACCTCTCCTGGTTGGAAAATGGAGAAGAATTAAATG
CCATCAACACAACAGTTTCCCAAGATCCTGAAACTGAGCTCTATGCTGTT
AGCAGCAAACTGGATTTCAATATGACAACCAACCACAGCTTCATGTGTCT
CATCAAGTATGGACATTTAAGAGTGAATCAGACCTTCAACTGGAATACAA
CCAAGCAAGAGCATTTTCCTGATAACCTGCTCCCATCCTGGGCCATTACC
TTAATCTCAGTAAATGGAATTTTTGTGATATGCTGCCTGACCTACTGCTT
TGCCCCAAGATGCAGAGAGAGAAGGAGGAATGAGAGATTGAGAAGGGAAA
GTGTACGCCCTGTA

In certain embodiments, the cell further comprises two exogenous co-stimulatory ligands that are 4-1BBL and CD80. In certain embodiments, the cell further comprises two exogenous co-stimulatory ligands that are 4-1BBL and CD80, wherein the 4-1BBL comprises or consists of the amino acid sequence set forth in SEQ ID NO: 67, and the CD80 comprises or consists of the amino acid sequence set forth in SEQ ID NO: 69.

Receptor-comprising cells comprising at least one exogenous co-stimulatory ligand are described in U.S. Pat. No. 8,389,282, which is incorporated by reference in its entirety.

### 2.6. Fusion Polypeptides

In certain embodiments, a presently disclosed cell comprising a first antigen-recognizing receptor and a second antigen-recognizing receptor further comprises a fusion polypeptide. For example, a presently disclosed cell can be further transduced with the fusion polypeptide, such that the cell expresses or is induced to express the first antigen-recognizing receptor, the second antigen-recognizing receptor, and the fusion polypeptide. The fusion polypeptide provides a co-stimulation signal to the cell. The fusion polypeptides are capable of enhancing the activity and/or efficacy of a cell comprising the first antigen-recognizing receptor (e.g., a CAR or a TCR-like fusion molecule). In certain embodiments, the fusion polypeptide comprises a) an extracellular domain and a transmembrane domain of a co-stimulatory ligand, and b) an intracellular domain of a first co-stimulatory molecule.

Non-limiting examples of the co-stimulatory ligand include tumor necrosis factor (TNF) family members, immunoglobulin (Ig) superfamily members, and combinations thereof. The TNF family member can be selected from the group consisting of 4-1BBL, OX40L, CD70, GITRL, CD40L, and combinations thereof. The Ig superfamily member can be selected from the group consisting of CD80, CD86, ICOS ligand (ICOSLG (also known as “CD275”), and combinations thereof. In certain embodiments, the co-stimulatory ligand is selected from the group consisting of 4-1BBL, OX40L, CD70, GITRL, CD40L, CD80, CD86, ICOSLG, and combinations thereof.

In certain embodiments, the fusion polypeptide comprises an extracellular domain and a transmembrane domain of a co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 69 or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 69.

In certain embodiments, the extracellular domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 1-242 of SEQ ID NO: 69. In certain embodiments, the extracellular domain of CD80 comprises or consists of amino acids 1-242 of SEQ ID NO: 69 or a functional fragment thereof. A functional fragment can be a consecutive portion of amino acids 1-242 of SEQ ID NO: 69, which is at least about 50, at least about 75, at least about 100, at least about 125, at least about 150, at least about 175, or at least about 200, or at least about 220 amino acids in length. In certain embodiments, the functional fragment retains at least about 80%, at least about 85%, at least about 90%, at least about 95%, or at least about 100% of the primary function of the extracellular domain of CD80. Non-limiting examples of the primary functions of the extracellular domain of CD80 include binding to/interacting with CD28, binding to/interacting with CTLA-4, binding to/interacting with PD-L1, and contributing to CD80 homodimerization. In certain embodiments, an extracellular domain of CD80 comprises or consists of amino acids 1-242 of SEQ ID NO: 69.

In certain embodiments, the transmembrane domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 243-263 of SEQ ID NO: 69. In certain embodiments, the transmembrane domain of CD80 comprises or consists of amino acids 243-263 of SEQ ID NO: 69 or a fragment thereof. Such fragment can be at least about 5, at least about 10, at least about 15, or at least about 20 amino acids in length. In certain embodiments, the transmembrane domain of CD80 comprises or consists of amino acids 243-263 of SEQ ID NO: 69.

Non-limiting examples of co-stimulatory molecules include CD28, 4-1BB, OX40, ICOS, DAP-10, CD27, CD40, NKG2D, CD2, and combinations thereof.

In certain embodiments, the fusion polypeptide comprises an extracellular domain and a transmembrane domain of a co-stimulatory molecule that is 4-1BB. In certain embodiments, the co-stimulatory molecule is human 4-1BB. In certain embodiments, the 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 26 or a fragment thereof, and/or may optionally comprise up to one or up to two or up to three conservative amino acid substitutions. In certain embodiments, the 4-1BB comprises or consists of an amino acid sequence that is a consecutive portion of the amino acid sequence of SEQ ID NO: 26. In certain embodiments, the intracellular domain of 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 214-255 of SEQ ID NO: 26 or a fragment thereof, and/or may optionally comprise up to one or up to two or up to three conservative amino acid substitutions. In certain embodiments, the intracellular domain of 4-1BB comprises or consists of amino acids 214-255 of SEQ ID NO: 26 or a functional fragment thereof. Such functional fragment can be a consecutive portion of amino acids 214-255 of SEQ ID NO: 26, which is at least about 20, at least about 25, at least about 30, at least about 35, or at least about 40 amino acids in length. In certain embodiments, the functional fragment of amino acids 214-255 of SEQ ID NO: 26 retains at least about 80%, at least about 85%, at least about 90%, at least about 95%, or at least about 100% of the primary functions of the intracellular domain of 4-1BB. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs). In certain embodiments, the intracellular domain of 4-1BB comprises or consists of amino acids 214-255 of SEQ ID NO: 26.

In certain embodiments, the co-stimulatory molecule is CD28. In certain embodiments, the co-stimulatory molecule is human CD28. In certain embodiments, the CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 7 or a fragment thereof, and/or may optionally comprise up to one or up to two or up to three conservative amino acid substitutions. In certain embodiments, the CD28 comprises or consists of an amino acid sequence that is a consecutive portion of the amino acid sequence of SEQ ID NO: 7. In certain embodiments, the intracellular domain of CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 180 to 219 of SEQ ID NO: 7 or a fragment thereof, and/or may optionally comprise up to one or up to two or up to three conservative amino acid substitutions. In certain embodiments, the intracellular domain of CD28 comprises or consists of amino acids 180 to 219 of SEQ ID NO: 7 or a functional fragment thereof. A functional fragment of amino acids 180 to 219 of SEQ ID NO: 7 can be a consecutive portion of amino acids 180 to 219 of SEQ ID NO: 7, which is at least about 20, at least about 25, at least about 30, or at least about 35 amino acids in length. In certain embodiments, such functional fragment retains at least about 80%, at least about 85%, at least about 90%, at least about 95%, or at least about 100% of the primary function of the intracellular domain of CD28. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK). In certain embodiments, the intracellular domain of CD28 comprises or consists of amino acids 180 to 219 of SEQ ID NO: 7.

In certain embodiments, the fusion polypeptide comprises an intracellular domain of a second co-stimulatory molecule. In certain embodiments, the fusion polypeptide comprises an intracellular domain of a third co-stimulatory molecule. In certain embodiments, the fusion polypeptide comprises an intracellular domain of a fourth co-stimulatory molecule. In certain embodiments, the fusion polypeptide comprises an intracellular domain of a fifth co-stimulatory molecule. In certain embodiments, the first, second, third, fourth, and fifth co-stimulatory molecule can be the same or different among each other.

In certain embodiments, the fusion polypeptide comprises an extracellular domain and a transmembrane domain of a co-stimulatory ligand that is CD80, and an intracellular domain of a co-stimulatory molecule that is 4-1BB. In certain embodiments, the fusion polypeptide comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 71. In certain embodiments, the fusion polypeptide comprises or consists of the amino acid sequence set forth in SEQ ID NO: 71. SEQ ID NO: 71 is provided below.

[SEQ ID NO: 71]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFKRGRKKLLYIFKQPFMRPVQTTQEEDGCSCRFP
EEEEGGCEL

In certain embodiments, the fusion polypeptide comprises an extracellular domain and a transmembrane domain of a co-stimulatory ligand that is CD80, an intracellular domain of a first co-stimulatory molecule that is 4-1BB, and an intracellular domain of a second co-stimulatory molecule that is CD28.

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 72. In certain embodiments, the fusion polypeptide comprises or consists of the amino acid sequence set forth in SEQ ID NO: 72. SEQ ID NO: 72 is provided below.

[SEQ ID NO: 72]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFRSKRSRLLHSDYMNMTPRRPGPTRKHYQPYAPP
RDFAAYRKRGRKKLLYIFKQPFMRPVQTTQEEDGCSCRFPEEEEGGCEL

Various modified fusion polypeptides are disclosed in International Patent Application No. PCT/US20/42753, which is incorporated by reference hereby in its entirety.

### 2.7. Gene Disruptions and Gene Modifications

In certain embodiments, a presently disclosed cell comprising a first antigen-recognizing receptor and a second antigen-recognizing receptor further comprises a gene disruption of a CD70 locus. The gene disruption of the CD70 locus can result in a non-functional CD70 protein or a knockout of the CD70 gene expression. In certain embodiments, the gene disruption of the CD70 locus results in knockout of the CD70 gene expression.

Non-limiting examples of gene disruptions include substitutions, deletions, insertions, or combinations thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof.

In certain embodiments, the CD70 locus is a human CD70 locus. The gene disruption of the CD70 locus can be generated by any suitable gene editing methods. In certain embodiments, the gene disruption of the CD70 locus (e.g., knockout of the CD70 locus) is generated using a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adena-associated viral vectors, vaccinia viruses, bovine papilloma viruses, and herpes viruses (e.g., such as Epstein-Barr Virus).

In certain embodiments, the gene disruption of the CD70 locus (e.g., knockout of the CD70 locus) is generated using a non-viral method. Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to generate the gene disruption of the CD70 locus. In certain embodiments, the gene disruption of the CD70 locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a CRISPR system is used to generate the gene disruption of the CD70 locus.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises is a CRISPRoff system. Additional details on the CRISPR systems of the presently disclosed subject matter can be found in Anzalone et al., Nature biotechnology 38.7 (2020): 824-844 and in Nuňez et al., Cell 184.9 (2021): 2503-2519, the contents of each of which are incorporated by reference in their entireties.

In certain embodiments, the CD70 locus is disrupted using a gRNA molecule to knockout expression of CD70. The gRNA molecule can target a coding sequence of a CD70 gene (e.g., a human CD70 gene) or a non-coding sequence of a CD70 gene (e.g., a human CD70 gene). In certain embodiments, the gRNA molecule targets a coding sequence of a CD70 gene (e.g., a human CD70 gene). In certain embodiments, the gRNA molecule targets a target sequence within a human CD70 gene.

In certain embodiments, zinc-finger nucleases are used to generate the gene disruption of the CD70 locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to generate the gene disruption of the CD70 locus. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the gene disruption of the CD70 locus can be a disruption of the coding region of the CD70 locus and/or a disruption of the non-coding region of the CD70 locus. In certain embodiments, the gene disruption of the CD70 locus comprises a disruption of the coding region of the CD70 locus. In certain embodiments, the gene disruption of the CD70 locus comprises an insertion at the coding region of the CD70 locus. Human CD70 protein comprises three exons: exon 1, exon 2, and exon 3. In certain embodiments, the gene disruption of the CD70 locus comprises a disruption at one or more of exon 1, exon 2, and exon 3 of the CD70 locus. In certain embodiments, the gene disruption of the CD70 locus comprises a disruption at exon 1 of the CD70 locus. In certain embodiments, the gene disruption of the CD70 locus comprises an insertion at exon 1 of the CD70 locus.

In certain embodiments, a presently disclosed cell comprising a first antigen-recognizing receptor and a second antigen-recognizing receptor further comprises a gene modification of a CD70 gene. The gene modification of the CD70 gene can result in a non-functional CD70 protein or a knockdown of the CD70 gene expression. In certain embodiments, the gene modification of the CD70 gene results in knockout of the CD70 gene expression.

In certain embodiments, the modification of the CD70 gene comprises use of an RNAi agent, including, but not limited to, shRNA, siRNA, LNA, dsRNA, and miRNA. In certain embodiments, the RNAi agent comprises a shRNA. In certain embodiments, the RNAi agent (e.g., shRNA) targets one or more isoforms of the CD70 gene and thereby reduces or eliminates the expression of the CD70 gene or CD70 protein. In certain embodiments, the RNAi agent (e.g., shRNA) is expressed from the same construct that expresses the first antigen-recognizing receptor and/or the second antigen-recognizing receptor disclosed herein. In certain embodiments, the expressions of the RNAi agent (e.g., shRNA), the first antigen-recognizing receptor, and the second antigen-recognizing receptor are driven by identical promoters (e.g., a same promoter). In certain embodiments, the expressions of the shRNA, the first antigen-recognizing receptor, and the second antigen-recognizing receptor disclosed herein are driven by different promoters.

In certain embodiments, the RNAi agent (e.g., shRNA) comprises a nucleotide sequence that is at least about 75%, at least about 80%, at least about 85%, at least about 90%, at least about 95%, at least about 96%, at least about 97%, at least about 98%, at least about 99%, or about 100% homologous or identical to at least a portion of a CD70 nucleic acid sequence. In certain embodiments, the RNAi agent (e.g., shRNA) comprises a nucleotide sequence complementary to the CD70 gene that is at least about 10 nucleotides, at least about 15 nucleotides, at least about 20 nucleotides, at least about 25 nucleotides, at least about 30 nucleotides. In certain embodiments, the RNAi agent (e.g., shRNA) comprises a nucleotide sequence that is up to 15 nucleotides, up to 20 nucleotides, up to 25 nucleotides, up to 30 nucleotides, up to 35 nucleotides, up to 40 nucleotides, up to 55 nucleotides, up to 60 nucleotides, up to 65 nucleotides, up to 70 nucleotides, up to 75 nucleotides, up to 80 nucleotides, up to 85 nucleotides, up to 90 nucleotides, up to 95 nucleotides, or up to 100 nucleotides in length. In certain embodiments, the RNAi agent comprises DNA or atypical or non-naturally occurring residues, for example, but not limited to, phosphorothioate residues.

In certain embodiments, the RNAi agent reduces the expression (e.g., endogenous expression) of CD70 by about 5%, about 10%, about 20%, about 30%, about 40%, about 50%, about 60%, about 70%, about 80%, about 90%, about 95%, about 100% or any intermediate value or range thereof. In certain embodiments, the RNAi agent reduces the expression (e.g., endogenous expression) of CD70 by about 70%.

In certain embodiments, the RNAi agent targeting the CD70 gene comprises or consists of the nucleotide sequence set forth in SEQ ID NOs: 161-175. In certain embodiments, the RNAi agent targeting the CD70 gene comprises or consists of the nucleotide sequence set forth in SEQ ID NO: 162. In certain embodiments, the RNAi agent targeting the CD70 gene comprises or consists of the nucleotide sequence set forth in SEQ ID NOs: 167. In certain embodiments, the RNAi agent targeting the CD70 gene comprises or consists of the nucleotide sequence set forth in SEQ ID NOs: 172. SEQ ID Nos: 161-175 are provided below.

[SEQ ID NO: 161]
AUGACAGGUUGAAGCAAGUAGAUA
[SEQ ID NO: 162]
CAAGUUCAAGAGAAAAAGUGUAUA
[SEQ ID NO: 163]
CGCUGCUGAUUAGGGUUUUUUAUA
[SEQ ID NO: 164]
CCGUGAUGGCAUCUACAUGGUAUA
[SEQ ID NO: 165]
AAAGAGAAAAAGUGUACACACAUA
[SEQ ID NO: 166]
UCUACUUGCUUCAACCUGUCAGUG
[SEQ ID NO: 167]
UACACUUUUUCUCUUGAACUUAUG
[SEQ ID NO: 168]
UAAAAAACCCUAAUCAGCAGCAUG
[SEQ ID NO: 169]
UACCAUGUAGAUGCCAUCACGAUG
[SEQ ID NO: 170]
UGUGUGUACACUUUUUCUCUUGUG
[SEQ ID NO: 171]
TGCTGTTGACAGTGAGCGATGACAGGTTGAAGCAAGTAGATAGTGAAGCC
ACAGATGTATCTACTTGCTTCAACCTGTCAGTGCCTACTGCCTCGGA
[SEQ ID NO: 172]
TGCTGTTGACAGTGAGCGCAAGTTCAAGAGAAAAAGTGTATAGTGAAGCC
ACAGATGTATACACTTTTTCTCTTGAACTTATGCCTACTGCCTCGGA
[SEQ ID NO: 173]
TGCTGTTGACAGTGAGCGCGCTGCTGATTAGGGTTTTTTATAGTGAAGCC
ACAGATGTATAAAAAACCCTAATCAGCAGCATGCCTACTGCCTCGGA
[SEQ ID NO: 174]
TGCTGTTGACAGTGAGCGCCGTGATGGCATCTACATGGTATAGTGAAGCC
ACAGATGTATACCATGTAGATGCCATCACGATGCCTACTGCCTCGGA
[SEQ ID NO: 175]
TGCTGTTGACAGTGAGCGAAAGAGAAAAAGTGTACACACATAGTGAAGCC
ACAGATGTATGTGTGTACACTTTTTCTCTTGTGCCTACTGCCTCGGA

In certain embodiments, a presently disclosed cell comprising a first antigen-recognizing receptor and a second antigen-recognizing receptor further comprises a gene disruption of a TRAC locus. In certain embodiments, the gene disruption of the TRAC locus results in a non-functional TCR. In certain embodiments, the gene disruption of the TRAC locus results in knockout of the TCR gene expression.

Any methods to generate the gene disruption of the CD70 locus as disclosed above can be used to generate the gene disruption of the TRAC locus. In certain embodiments, the gene disruption of the TRAC locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the TRAC locus can be a disruption of the coding region of the TRAC locus and/or a disruption of the non-coding region of the TRAC locus. In certain embodiments, the gene disruption of the TRAC locus comprises a disruption of the coding region of the TRAC locus. In certain embodiments, the gene disruption of the TRAC locus comprises an insertion at the coding region of the TRAC locus. Human TRAC protein comprises 4 exons: exon 1, exon 2, exon 3, and exon 4. In certain embodiments, the coding region of the TRAC locus comprises exon 1, exon 2, exon 3, and exon 4. In certain embodiments, the gene disruption of the TRAC locus comprises a disruption at one or more of exon 1 through exon 4 of the TRAC locus. In certain embodiments, the gene disruption of the TRAC locus comprises a disruption at exon 1 of the TRAC locus. In certain embodiments, the gene disruption of the TRAC locus comprises an insertion at exon 1 of the TRAC locus.

In certain embodiments, a presently disclosed cell comprising a first antigen-recognizing receptor and a second antigen-recognizing receptor further comprises a gene modification of a TRAC gene. The gene modification of the TRAC gene can result in a non-functional TCR protein or a knockdown of the TCR gene expression. In certain embodiments, the gene modification of the TRAC gene results in knockout of the TCR gene expression.

In certain embodiments, the modification of the TRAC gene comprises use of an RNAi agent, including, but not limited to, shRNA, siRNA, LNA, dsRNA, and miRNA. In certain embodiments, the RNAi agent comprises a shRNA. In certain embodiments, the RNAi agent (e.g., shRNA) targets one or more isoforms of the TRAC gene and thereby reduces or eliminates the expression of the TRAC gene or TCR protein. In certain embodiments, the RNAi agent (e.g., shRNA) is expressed from the same construct that expresses the first antigen-recognizing receptor and/or the second antigen-recognizing receptor disclosed herein. In certain embodiments, the expressions of the RNAi agent (e.g., shRNA), the first antigen-recognizing receptor, and the second antigen-recognizing receptor are driven by identical promoters (e.g., a same promoter). In certain embodiments, the expressions of the shRNA, the first antigen-recognizing receptor, and the second antigen-recognizing receptor disclosed herein are driven by different promoters.

In certain embodiments, the RNAi agent (e.g., shRNA) comprises a nucleotide sequence that is at least about 75%, at least about 80%, at least about 85%, at least about 90%, at least about 95%, at least about 96%, at least about 97%, at least about 98%, at least about 99%, or about 100% homologous or identical to at least a portion of a TRAC nucleic acid sequence. In certain embodiments, the RNAi agent (e.g., shRNA) comprises a nucleotide sequence complementary to the TRAC gene that is at least about 10 nucleotides, at least about 15 nucleotides, at least about 20 nucleotides, at least about 25 nucleotides, at least about 30 nucleotides. In certain embodiments, the RNAi agent (e.g., shRNA) comprises a nucleotide sequence that is up to 15 nucleotides, up to 20 nucleotides, up to 25 nucleotides, up to 30 nucleotides, up to 35 nucleotides, up to 40 nucleotides, up to 55 nucleotides, up to 60 nucleotides, up to 65 nucleotides, up to 70 nucleotides, up to 75 nucleotides, up to 80 nucleotides, up to 85 nucleotides, up to 90 nucleotides, up to 95 nucleotides, or up to 100 nucleotides in length. In certain embodiments, the RNAi agent comprises DNA or atypical or non-naturally occurring residues, for example, but not limited to, phosphorothioate residues.

In certain embodiments, the RNAi agent reduces the expression (e.g., endogenous expression) of TCR by about 5%, about 10%, about 20%, about 30%, about 40%, about 50%, about 60%, about 70%, about 80%, about 90%, about 95%, about 100% or any intermediate value or range thereof. In certain embodiments, the RNAi agent reduces the expression (e.g., endogenous expression) of TCR by about 20%. In certain embodiments, the RNAi agent reduces the expression (e.g., endogenous expression) of TCR by about 30%.

In certain embodiments, the RNAi agent targeting the TRAC gene comprises or consists of the nucleotide sequence set forth in SEQ ID NOs: 176-190. In certain embodiments, the RNAi agent targeting the TRAC gene comprises or consists of the nucleotide sequence set forth in SEQ ID NO: 180. In certain embodiments, the RNAi agent targeting the TRAC gene comprises or consists of the nucleotide sequence set forth in SEQ ID NOs: 185. In certain embodiments, the RNAi agent targeting the TRAC gene comprises or consists of the nucleotide sequence set forth in SEQ ID NOs: 190. SEQ ID Nos: 176-190 are provided below.

[SEQ ID NO: 176]
CUCACCGAUUUUGAUUCUCAAAUA
[SEQ ID NO: 177]
AUGGUCGAGAAAAGCUUUGAAAUA
[SEQ ID NO: 178]
ACGAUUUUGAUUCUCAAACAAAUA
[SEQ ID NO: 179]
CUUCACCGAUUUUGAUUCUCAAUA
[SEQ ID NO: 180]
AAGAUACGAACCUAAACUUUCAUA
[SEQ ID NO: 181]
UUUGAGAAUCAAAAUCGGUGAAUG
[SEQ ID NO: 182]
UUUCAAAGCUUUUCUCGACCAGUG
[SEQ ID NO: 183]
UUUGUUUGAGAAUCAAAAUCGGUG
[SEQ ID NO: 184]
UUGAGAAUCAAAAUCGGUGAAUUG
[SEQ ID NO: 185]
UGAAAGUUUAGGUUCGUAUCUGUG
[SEQ ID NO: 186]
TGCTGTTGACAGTGAGCGCTCACCGATTTTGATTCTCAAATAGTGAAGCC
ACAGATGTATTTGAGAATCAAAATCGGTGAATGCCTACTGCCTCGGA
[SEQ ID NO: 187]
TGCTGTTGACAGTGAGCGATGGTCGAGAAAAGCTTTGAAATAGTGAAGCC
ACAGATGTATTTCAAAGCTTTTCTCGACCAGTGCCTACTGCCTCGGA
[SEQ ID NO: 188]
TGCTGTTGACAGTGAGCGACGATTTTGATTCTCAAACAAATAGTGAAGCC
ACAGATGTATTTGTTTGAGAATCAAAATCGGTGCCTACTGCCTCGGA
[SEQ ID NO: 189]
TGCTGTTGACAGTGAGCGCTTCACCGATTTTGATTCTCAATAGTGAAGCC
ACAGATGTATTGAGAATCAAAATCGGTGAATTGCCTACTGCCTCGGA
[SEQ ID NO: 190]
TGCTGTTGACAGTGAGCGAAGATACGAACCTAAACTTTCATAGTGAAGCC
ACAGATGTATGAAAGTTTAGGTTCGTATCTGTGCCTACTGCCTCGGA

In certain embodiments, a presently disclosed cell comprising a first antigen-recognizing receptor and a second antigen-recognizing receptor further comprises a gene disruption of a TRBC locus (e.g., a TRBC1 locus, a TRBC2 locus). In certain embodiments, the gene disruption of the TRBC locus (e.g., a TRBC1 locus, a TRBC2 locus) results in a non-functional TCR. In certain embodiments, the gene disruption of the TRBC locus (e.g., a TRBC1 locus, a TRBC2 locus) results in knockout of the TCR gene expression.

Any methods to generate the gene disruption of the CD70 locus as disclosed above can be used to generate the gene disruption of the TRBC locus (e.g., a TRBC1 locus, a TRBC2 locus). In certain embodiments, the gene disruption of the TRBC locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the TRBC locus can be a disruption of the coding region of the TRBC1 locus. In certain embodiments, the gene disruption of the TRBC locus comprises a disruption of the coding region of the TRBC1 locus. In certain embodiments, the gene disruption of the TRBC locus comprises an insertion at the coding region of the TRBC1 locus. Human TRBC1 protein comprises 4 exons: exon 1, exon 2, exon 3, and exon 4. In certain embodiments, the coding region of the TRBC1 locus comprises exon 1, exon 2, exon 3, and exon 4. In certain embodiments, the gene disruption of the TRBC locus comprises a disruption at one or more of exon 1 through exon 4 of the TRBC1 locus. In certain embodiments, the gene disruption of the TRBC locus comprises a disruption at exon 1 of the TRBC1 locus. In certain embodiments, the gene disruption of the TRBC locus comprises an insertion at exon 1 of the TRBC1 locus.

In certain embodiments, the gene disruption of the TRBC locus can be a disruption of the coding region of the TRBC2 locus. In certain embodiments, the gene disruption of the TRBC locus comprises a disruption of the coding region of the TRBC2 locus. In certain embodiments, the gene disruption of the TRBC locus comprises an insertion at the coding region of the TRBC2 locus. Human TRBC2 protein comprises 4 exons: exon 1, exon 2, exon 3, and exon 4. In certain embodiments, the coding region of the TRBC2 locus comprises exon 1, exon 2, exon 3, and exon 4. In certain embodiments, the gene disruption of the TRBC locus comprises a disruption at one or more of exon 1 through exon 4 of the TRBC2 locus. In certain embodiments, the gene disruption of the TRBC locus comprises a disruption at exon 1 of the TRBC2 locus. In certain embodiments, the gene disruption of the TRBC locus comprises an insertion at exon 1 of the TRBC2 locus.

In certain embodiments, a presently disclosed cell comprising a first antigen-recognizing receptor and a second antigen-recognizing receptor further comprises a gene modification of a TRBC gene. The gene modification of the TRBC gene can result in a non-functional TCR protein or a knockdown of the TCR gene expression. In certain embodiments, the gene modification of the TRBC gene results in knockout of the TCR gene expression.

In certain embodiments, the modification of the TRBC gene comprises use of an RNAi agent, including, but not limited to, shRNA, siRNA, LNA, dsRNA, and miRNA. In certain embodiments, the RNAi agent comprises a shRNA. In certain embodiments, the RNAi agent (e.g., shRNA) targets one or more isoforms of the TRBC gene and thereby reduces or eliminates the expression of the TRBC gene or TCR protein. In certain embodiments, the RNAi agent (e.g., shRNA) is expressed from the same construct that expresses the first antigen-recognizing receptor and/or the second antigen-recognizing receptor disclosed herein. In certain embodiments, the expressions of the RNAi agent (e.g., shRNA), the first antigen-recognizing receptor, and the second antigen-recognizing receptor are driven by identical promoters (e.g., a same promoter). In certain embodiments, the expressions of the shRNA, the first antigen-recognizing receptor, and the second antigen-recognizing receptor disclosed herein are driven by different promoters.

In certain embodiments, the RNAi agent (e.g., shRNA) comprises a nucleotide sequence that is at least about 75%, at least about 80%, at least about 85%, at least about 90%, at least about 95%, at least about 96%, at least about 97%, at least about 98%, at least about 99%, or about 100% homologous or identical to at least a portion of a TRBC nucleic acid sequence. In certain embodiments, the RNAi agent (e.g., shRNA) comprises a nucleotide sequence complementary to the TRBC gene that is at least about 10 nucleotides, at least about 15 nucleotides, at least about nucleotides, at least about 25 nucleotides, at least about 30 nucleotides. In certain embodiments, the RNAi agent (e.g., shRNA) comprises a nucleotide sequence that is up to 15 nucleotides, up to 20 nucleotides, up to 25 nucleotides, up to 30 nucleotides, up to 35 nucleotides, up to 40 nucleotides, up to 55 nucleotides, up to 60 nucleotides, up to 65 nucleotides, up to 70 nucleotides, up to 75 nucleotides, up to 80 nucleotides, up to 85 nucleotides, up to 90 nucleotides, up to 95 nucleotides, or up to 100 nucleotides in length. In certain embodiments, the RNAi agent comprises DNA or atypical or non-naturally occurring residues, for example, but not limited to, phosphorothioate residues.

In certain embodiments, the RNAi agent reduces the expression (e.g., endogenous expression) of TCR by about 5%, about 10%, about 20%, about 30%, about 40%, about 50%, about 60%, about 70%, about 80%, about 90%, about 95%, about 100% or any intermediate value or range thereof. In certain embodiments, the RNAi agent reduces the expression (e.g., endogenous expression) of TCR by about 60%.

In certain embodiments, the RNAi agent targeting the TRBC gene comprises or consists of the nucleotide sequence set forth in SEQ ID NOs: 191-208. In certain embodiments, the RNAi agent targeting the TRBC gene comprises or consists of the nucleotide sequence set forth in SEQ ID NO: 191. In certain embodiments, the RNAi agent targeting the TRBC gene comprises or consists of the nucleotide sequence set forth in SEQ ID NOs: 197. In certain embodiments, the RNAi agent targeting the TRBC gene comprises or consists of the nucleotide sequence set forth in SEQ ID NOs: 203. SEQ ID Nos: 191-208 are provided below.

[SEQ ID NO: 191]
CCCGCUGUCAAGUCCAGUUCUAUA
[SEQ ID NO: 192]
ACGCCCUCAAUGACUCCAGAUAUA
[SEQ ID NO: 193]
AACCCGAGGUCGCUGUGUUUGAUA
[SEQ ID NO: 194]
ACGCAACCACUUCCGCUGUCAAUA
[SEQ ID NO: 195]
CGCAGAGAUCUCCCACACCCAAUA
[SEQ ID NO: 196]
CCGCUGUGUUUGAGCCAUCAGAUA
[SEQ ID NO: 197]
UAGAACUGGACUUGACAGCGGAUG
[SEQ ID NO: 198]
UAUCUGGAGUCAUUGAGGGCGGUG
[SEQ ID NO: 199]
UCAAACACAGCGACCUCGGGUGUG
[SEQ ID NO: 200]
UUGACAGCGGAAGUGGUUGCGGUG
[SEQ ID NO: 201]
UUGGGUGUGGGAGAUCUCUGCUUG
[SEQ ID NO: 202]
UCUGAUGGCUCAAACACAGCGAUG
[SEQ ID NO: 203]
TGCTGTTGACAGTGAGCGCCCGCTGTCAAGTCCAGTTCTATAGTGAAGCC
ACAGATGTATAGAACTGGACTTGACAGCGGATGCCTACTGCCTCGGA
[SEQ ID NO: 204]
TGCTGTTGACAGTGAGCGACGCCCTCAATGACTCCAGATATAGTGAAGCC
ACAGATGTATATCTGGAGTCATTGAGGGCGGTGCCTACTGCCTCGGA
[SEQ ID NO: 205]
TGCTGTTGACAGTGAGCGAACCCGAGGTCGCTGTGTTTGATAGTGAAGCC
ACAGATGTATCAAACACAGCGACCTCGGGTGTGCCTACTGCCTCGGA
[SEQ ID NO: 206]
TGCTGTTGACAGTGAGCGACGCAACCACTTCCGCTGTCAATAGTGAAGCC
ACAGATGTATTGACAGCGGAAGTGGTTGCGGTGCCTACTGCCTCGGA
[SEQ ID NO: 207]
TGCTGTTGACAGTGAGCGCGCAGAGATCTCCCACACCCAATAGTGAAGCC
ACAGATGTATTGGGTGTGGGAGATCTCTGCTTGCCTACTGCCTCGGA
[SEQ ID NO: 208]
TGCTGTTGACAGTGAGCGCCGCTGTGTTTGAGCCATCAGATAGTGAAGCC
ACAGATGTATCTGATGGCTCAAACACAGCGATGCCTACTGCCTCGGA

The presently disclosed cells can be isolated and activated by using CD3/CD28 antibodies before generation of a gene disruption. In certain embodiments, the presently disclosed cell comprising a first antigen-recognizing receptor and a second antigen-recognizing receptor further comprises a gene disruption of a TRAC locus, a TRBC locus, and/or of a CD70 locus. In certain embodiments, the presently disclosed cell comprising a first antigen-recognizing receptor and a second antigen-recognizing receptor further comprises a gene modification of a TRAC locus, a TRBC locus, and/or of a CD70 locus.

In certain embodiments, the gene disruption of the TRAC locus, the gene disruption of the TRBC locus, and/or the gene disruption of the CD70 locus are generated after isolation and activation of the cells (e.g., T cells). In certain embodiments, the gene disruption of the TRAC locus, the gene disruption of the TRBC locus, and/or the gene disruption of the CD70 locus are generated before isolation and activation of the cells (e.g., T cells).

In certain embodiments, the gene disruption of the TRAC locus and/or TRBC locus is generated before isolation and activation of the cells (e.g., T cells) and the gene disruption of the CD70 locus is generated after isolation and activation of the cells (e.g., T cells). In certain embodiments, the gene disruption of the CD70 locus is generated before isolation and activation of the cells (e.g., T cells) and the gene disruption of the TRAC locus and/or TRBC locus is generated after isolation and activation of the cells (e.g., T cells).

In certain embodiments, the gene modification of the TRAC locus, the gene modification of the TRBC locus, and/or the gene modification of the CD70 locus are generated after isolation and activation of the cells (e.g., T cells). In certain embodiments, the gene modification of the TRAC locus, the gene modification of the TRBC locus, and/or the gene modification of the CD70 locus are generated before isolation and activation of the cells (e.g., T cells).

In certain embodiments, the gene modification of the TRAC locus and/or TRBC locus is generated before isolation and activation of the cells (e.g., T cells) and the gene modification of the CD70 locus is generated after isolation and activation of the cells (e.g., T cells). In certain embodiments, the gene modification of the CD70 locus is generated before isolation and activation of the cells (e.g., T cells) and the gene modification of the TRAC locus and/or TRBC locus is generated after isolation and activation of the cells (e.g., T cells).

### 2.7. Exemplary Cells

In certain embodiments, the presently disclosed cell is a T cell comprising a) a chimeric antigen receptor (CAR) comprising an extracellular antigen-binding domain that targets CD312, a transmembrane domain, and an intracellular domain; and b) a TCR-like fusion protein (HIT) that targets CD70 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 73, a CDR2 comprising the amino acid sequence set forth in SEQ ID NO: 74, and a CDR3 comprising the amino acid sequence set forth in SEQ ID NO: 75; and a V_{L} comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (V_{H}) of an antibody comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138, and b) a TRBC polypeptide. In certain embodiments, the CAR and HIT are encoded by an exogenous nucleic acid integrated in the TRAC locus. In certain embodiments, the presently disclosed T cell comprises a gene disruption of a CD70 locus.

In certain embodiments, the presently disclosed cell is a T cell comprising a) a chimeric antigen receptor (CAR) comprising an extracellular antigen-binding domain that targets CD312, a transmembrane domain, and an intracellular domain; and b) a TCR-like fusion protein (HIT) that targets CD70 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 73, a CDR2 comprising the amino acid sequence set forth in SEQ ID NO: 74, and a CDR3 comprising the amino acid sequence set forth in SEQ ID NO: 75; and a V_{L} comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (V_{H}) of an antibody comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138, and b) a TRBC polypeptide. In certain embodiments, the CAR and HIT are encoded by an exogenous nucleic acid integrated in the TRAC locus. In certain embodiments, the presently disclosed T cell comprises a gene modification of a CD70 locus.

In certain embodiments, the presently disclosed cell is a T cell comprising a) a chimeric antigen receptor (CAR) comprising an extracellular antigen-binding domain that targets CD276, a transmembrane domain, and an intracellular domain; and b) a TCR-like fusion protein (HIT) that targets CD70 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 85, a CDR2 comprising the amino acid sequence set forth in SEQ ID NO: 86, and a CDR3 comprising the amino acid sequence set forth in SEQ ID NO: 87; and a V_{L} comprising a CDR1 comprising the amino acid sequence set forth in SEQ ID NO: 88, a CDR2 comprising the amino acid sequence set forth in SEQ ID NO: 89, and a CDR3 comprising the amino acid sequence set forth in SEQ ID NO: 90. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (V_{H}) of an antibody comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138, and b) a TRBC polypeptide. In certain embodiments, the CAR and HIT are encoded by an exogenous nucleic acid integrated in the TRAC locus. In certain embodiments, the presently disclosed T cell comprises a gene disruption of a CD70 locus.

In certain embodiments, the presently disclosed cell is a T cell comprising a) a chimeric antigen receptor (CAR) comprising an extracellular antigen-binding domain that targets CD276, a transmembrane domain, and an intracellular domain; and b) a TCR-like fusion protein (HIT) that targets CD70 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 85, a CDR2 comprising the amino acid sequence set forth in SEQ ID NO: 86, and a CDR3 comprising the amino acid sequence set forth in SEQ ID NO: 87; and a V_{L} comprising a CDR1 comprising the amino acid sequence set forth in SEQ ID NO: 88, a CDR2 comprising the amino acid sequence set forth in SEQ ID NO: 89, and a CDR3 comprising the amino acid sequence set forth in SEQ ID NO: 90. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (V_{H}) of an antibody comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138, and b) a TRBC polypeptide. In certain embodiments, the CAR and HIT are encoded by an exogenous nucleic acid integrated in the TRAC locus. In certain embodiments, the presently disclosed T cell comprises a gene modification of a CD70 locus.

In certain embodiments, the presently disclosed cell is a T cell comprising a) a chimeric antigen receptor (CAR) comprising an extracellular antigen-binding domain that targets CD22, a transmembrane domain, and an intracellular domain; and b) a TCR-like fusion protein (HIT) that targets CD19 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 97, a CDR2 comprising the amino acid sequence set forth in SEQ ID NO: 98, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (V_{H}) of an antibody comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148, and b) a TRBC polypeptide. In certain embodiments, the CAR and HIT are encoded by an exogenous nucleic acid integrated in the TRAC locus.

In certain embodiments, the presently disclosed cell is a T cell comprising a) a chimeric antigen receptor (CAR) comprising an extracellular antigen-binding domain that targets CD22, a transmembrane domain, and an intracellular domain; and b) a TCR-like fusion protein (HIT) that targets CD19 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 97, a CDR2 comprising the amino acid sequence set forth in SEQ ID NO: 98, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (VH) of an antibody comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148, and b) a TRBC polypeptide. In certain embodiments, the CAR and HIT are encoded by an exogenous nucleic acid integrated in the TRAC locus.

In certain embodiments, the presently disclosed cell is a T cell comprising a) a chimeric antigen receptor (CAR) comprising an extracellular antigen-binding domain that targets CD22, a transmembrane domain, and an intracellular domain; and b) a TCR-like fusion protein (HIT) that targets CD19 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 97, a CDR2 comprising the amino acid sequence set forth in SEQ ID NO: 153, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (V_{H}) of an antibody comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148, and b) a TRBC polypeptide. In certain embodiments, the CAR and HIT are encoded by an exogenous nucleic acid integrated in the TRAC locus.

In certain embodiments, the presently disclosed cell is a T cell comprising a) a chimeric antigen receptor (CAR) comprising an extracellular antigen-binding domain that targets CD22, a transmembrane domain, and an intracellular domain; and b) a TCR-like fusion protein (HIT) that targets CD19 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 119, a CDR2 comprising the amino acid sequence set forth in SEQ ID NO: 120, and a CDR3 comprising the amino acid sequence set forth in SEQ ID NO: 121; and a V_{L} comprising a CDR1 comprising the amino acid sequence set forth in SEQ ID NO: 122, a CDR2 comprising the amino acid sequence set forth in SEQ ID NO: 123, and a CDR3 comprising the amino acid sequence set forth in SEQ ID NO: 124. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (VH) of an antibody comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and b) a TRAC polypeptide. In certain embodiments, the second antigen binding chain of the HIT comprises a) an antigen-binding fragment of a light chain variable region (VL) of an antibody comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148, and b) a TRBC polypeptide. In certain embodiments, the CAR and HIT are encoded by an exogenous nucleic acid integrated in the TRAC locus.

In certain embodiments, the presently disclosed cell is a T cell comprising a) a chimeric antigen receptor (CAR) comprising an extracellular antigen-binding domain that targets CD22, a transmembrane domain, and an intracellular domain; and b) a TCR-like fusion protein (HIT) that targets CD19 comprising a first antigen binding chain and a second antigen binding chain. In certain embodiments, the extracellular antigen-binding domain of the CAR comprises a V_{H} comprising a CDR1 comprising amino acids having the sequence set forth in SEQ ID NO: 97, a CDR2 comprising the amino acid sequence set forth in SEQ ID NO: 154, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102. In certain embodiments, the transmembrane domain of the CAR comprises a CD28 polypeptide that comprises or consists of amino acids 153 to 179 of SEQ ID NO: 7. In certain embodiments, the intracellular domain of the CAR comprises a CD28 polypeptide and a modified CD3ζ polypeptide. In certain embodiments, the CD28 polypeptide comprises amino acids 180 to 220 of SEQ ID NO: 7, and the modified CD3ζ polypeptide comprises the amino acid sequence set forth in SEQ ID NO: 22. In certain embodiments, the first antigen binding chain of the HIT comprises a) an antigen-binding fragment of a heavy chain variable region (V_{H}) of an antibody comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and b) a TRAC polypeptide. In certain embodiments, the second antigen binding chain of the HIT comprises a) an antigen-binding fragment of a light chain variable region (V_{L}) of an antibody comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148, and b) a TRBC polypeptide. In certain embodiments, the CAR and HIT are encoded by an exogenous nucleic acid integrated in the TRAC locus.

### 3. Nucleic Acid Compositions and Vectors

The presently disclosed subject matter provides nucleic acid compositions comprising a first polynucleotide encoding a first antigen-recognizing receptor disclosed herein (e.g., disclosed in Section 2.1) and a second polynucleotide encoding a second antigen-recognizing receptor disclosed herein (e.g., disclosed in Section 2.2). Also provided are cells comprising such nucleic acid compositions. In certain embodiments, the nucleic acid composition further comprises a first promoter that is operably linked to the first antigen-recognizing receptor. In certain embodiments, the nucleic acid composition further comprises a second promoter that is operably linked to the second antigen-recognizing receptor.

In addition, the presently disclosed subject matter provides nucleic acid compositions comprising a first polynucleotide encoding a first antigen-recognizing receptor disclosed herein (e.g., disclosed in Section 2.1) and a second polynucleotide encoding a fusion polypeptide disclosed herein (e.g., disclosed in Section 2.2). Also provided are cells comprising such nucleic acid compositions. In certain embodiments, the nucleic acid composition further comprises a first promoter that is operably linked to the fusion polypeptide. In certain embodiments, the nucleic acid composition further comprises a second promoter that is operably linked to the first antigen-recognizing receptor.

In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, the first and/or the second antigen-recognizing receptors are integrated at a locus within the genome of the T cell, e.g., a TRAC locus, a TRBC locus, a TRDC locus, or a TRGC locus. In certain embodiments, the locus is a TRAC locus. In certain embodiments, the expression of the first and/or second antigen-recognizing receptors are under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

In certain embodiments, the nucleic acid composition is a vector. In certain embodiments, the vector is a retroviral vector (e.g., a gamma-retroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter specific for a target cell type of interest. Non-viral vectors may be used as well.

The first polynucleotide and the second polynucleotide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation. Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 3.1. Methods of Delivering

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10- to about 10-6 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3″′-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12,3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, 0-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 4. Formulations and Administration

The presently disclosed subject matter provides compositions comprising presently disclosed cells (e.g., disclosed in Section 2). In certain embodiments, the compositions are pharmaceutical compositions that further comprise a pharmaceutically acceptable excipient.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells) or NK cells) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 5. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor (or neoplasm) in a subject. The presently disclosed cells and compositions comprising thereof can be used for prolonging the survival of a subject suffering from a tumor. In certain embodiments, the tumor is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. In certain embodiments, each of the above-noted method comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non limiting examples of solid tumor include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, B-cell precursor acute lymphoblastic leukemia, and T-cell precursor acute lymphoblastic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma. In certain embodiments, the lymphoma is T-cell precursor acute lymphoblastic lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenstrom macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

In certain embodiments, the myeloid disorder is acute myeloid leukemia (AML). In certain embodiments, the first and/or second antigens are independently selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the tumor and/or neoplasm is acute myeloid leukemia (AML), the first antigen is CD312, and the second antigen is CD70. In certain embodiments, the AML comprises tumor cells having a CD70 low antigen density. In certain embodiments, a cell having a CD70 low antigen density comprises a cell surface density of CD70 that is less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, the AML comprises CD70⁺ tumor cells having low tumor cell frequency. In certain embodiments, CD70⁺ tumor cells have a frequency of less than about 50% per tumor, less than about 40% per tumor, less than about 30% per tumor, less than about 20% per tumor, less than about 15% per tumor, less than about 10% per tumor, less than about 5% per tumor, less than about 2% per tumor, or less than about 1% per tumor.

In certain embodiments, the tumor and/or neoplasm is melanoma, the first antigen is CD276, and the second antigen is CD70. In certain embodiments, the melanoma comprises tumor cells having a CD70 low antigen density. In certain embodiments, a cell having a CD70 low antigen density comprises a cell surface density of CD70 that is less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, the melanoma comprises CD70⁺ tumor cells having low tumor cell frequency. In certain embodiments, CD70⁺ tumor cells have a frequency of less than about 50% per tumor, less than about 40% per tumor, less than about 30% per tumor, less than about 20% per tumor, less than about 15% per tumor, less than about 10% per tumor, less than about 5% per tumor, less than about 2% per tumor, or less than about 1% per tumor.

In certain embodiments, the tumor and/or neoplasm is B-cell acute lymphoblastic leukemia (B-ALL), the first antigen is CD22, and the second antigen is CD19. In certain embodiments, the B-ALL comprises tumor cells having a CD19 low antigen density. In certain embodiments, a cell having a CD19 low antigen density comprises a cell surface density of CD19 that is less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, the B-ALL comprises CD19⁺ tumor cells having low tumor cell frequency. In certain embodiments, CD19⁺ tumor cells have a frequency of less than about 50% per tumor, less than about 40% per tumor, less than about 30% per tumor, less than about 20% per tumor, less than about 15% per tumor, less than about 10% per tumor, less than about 5% per tumor, less than about 2% per tumor, or less than about 1% per tumor.

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an infectious disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into a presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 6. Kits

The presently disclosed subject matter provides kits for inducing and/or enhancing an immune response and/or treating and/or preventing a neoplasm or a pathogen infection (e.g., an autoimmune disease or an infectious disease) in a subject. In certain embodiments, the kit comprises an effective amount of presently disclosed cells, a presently disclosed composition, or a presently disclosed nucleic acid composition. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR, a TCR, or a TCR-like fusion molecule) directed toward an antigen of interest in expressible form, which may optionally be comprised in the same or different vectors.

If desired, the cells, composition, or nucleic acid composition are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acid composition for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 7. Exemplary Embodiments

Embodiment 1. A cell comprising: (a) a chimeric antigen receptor (CAR) that targets a first antigen; and (b) a TCR-like fusion molecule that targets a second antigen.

Embodiment 2. The cell of embodiment 1, wherein the CAR comprises an extracellular antigen-binding domain that binds to the first antigen, and an intracellular signaling domain that is capable of delivering an activation signal to the cell.

Embodiment 3. The cell of embodiment 2, wherein the intracellular signaling domain of the CAR comprises a CD3ζ polypeptide.

Embodiment 4. The cell of embodiment 3, wherein the CD3ζ polypeptide is a native CD3ζ polypeptide or a modified CD3ζ polypeptide.

Embodiment 5. The cell of embodiment 4, wherein the modified CD3ζ polypeptide comprises a native ITAM1, an ITAM2 variant consisting of two loss-of-function mutations, and an ITAM3 variant consisting of two loss-of-function mutations.

Embodiment 6. The cell of embodiment 4 or 5, wherein the modified CD3ζ polypeptide comprises the amino acid sequence set forth in SEQ ID NO: 22.

Embodiment 7. The cell of any one of embodiments 2-6, wherein the intracellular signaling domain of the CAR further comprises at least one costimulatory signaling region.

Embodiment 8. The cell of embodiment 7, wherein the at least one costimulatory signaling region comprises at least an intracellular domain of a co-stimulatory molecule or a portion thereof.

Embodiment 9. The cell of embodiment 8, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

Embodiment 10. The cell of embodiment 9, wherein the costimulatory molecule comprises amino acids 180 to 220 of SEQ ID NO: 7.

Embodiment 11. The cell of any one of embodiments 2-10, wherein the CAR comprises a transmembrane domain.

Embodiment 12. The cell of any one of embodiments 1-11, wherein the TCR-like fusion molecule comprises i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the second antigen, wherein the TCR-like fusion molecule binds to the second antigen in an HLA-independent manner.

Embodiment 13. The cell of embodiment 12, wherein at least one of the TRAC polypeptide and the TRBC polypeptide is endogenous.

Embodiment 14. The cell of embodiment 12 or 13, wherein the first and the second antigen-binding chains bind to the second antigen with a dissociation constant (KD) of about 1×10-8 M or less.

Embodiment 15. The cell of any one of embodiments 12-14, wherein the first and the second antigen-binding chains bind to the second antigen with a dissociation constant (KD) of about 5×10−9 M or less.

Embodiment 16. The cell of any one of embodiments 12-15, wherein the first antigen-binding chain comprises an antigen-binding fragment of a VH of an antibody and a TRBC polypeptide, and the second antigen-binding chain comprises an antigen-binding fragment of a VL of the antibody and a TRAC polypeptide.

Embodiment 17. The cell of any one of embodiments 12-15, wherein the first antigen-binding chain comprises an antigen-binding fragment of a VH of an antibody and a TRAC polypeptide, and the second antigen-binding chain comprises an antigen-binding fragment of a VL of the antibody and a TRBC polypeptide.

Embodiment 18. The cell of any one of embodiments 12-17, wherein the first and second antigen binding chains are capable of associating with a CD3ζ polypeptide.

Embodiment 19. The cell of embodiment 18, wherein the first and second antigen binding chains, upon binding to the second antigen, are capable of activating the CD3ζ polypeptide.

Embodiment 20. The cell of embodiment 19, wherein the activation of the CD3ζ polypeptide is capable of activating the cell.

Embodiment 21. The cell of any one of embodiments 12-20, wherein the cell further comprises a gene disruption of a TRAC locus and/or a TRBC locus.

Embodiment 22. The cell of any one of embodiments 12-20, wherein the cell further comprises a gene disruption of a CD70 locus.

Embodiment 23. The cell of any one of embodiments 12-20, wherein the cell further comprises a gene disruption of a TRAC locus, a TRBC locus, and/or a CD70 locus.

Embodiment 24. The cell Embodiment, wherein the cell further comprises a gene modification of a TRAC gene and/or a TRBC gene.

Embodiment 25. The cell Embodiment, wherein the cell further comprises a gene modification of a CD70 gene.

Embodiment 26. The cell Embodiment, wherein the cell further comprises a gene modification of a TRAC gene, a TRBC gene, and/or a CD70 gene.

Embodiment 27. The cell of any one of embodiments 1-26, wherein the cell is a cell of the lymphoid lineage or a cell of the myeloid lineage.

Embodiment 28. The cell of embodiment 27, wherein the cell of the lymphoid lineage is selected from the group consisting of a T cell, a B cell, a Natural Killer (NK) cell, and a dendritic cell.

Embodiment 29. The cell of any one of embodiments 1-28, wherein the cell is a T cell.

Embodiment 30. The cell of embodiment 29, wherein the T cell is derived from an induced pluripotent stem cell.

Embodiment 31. The cell of embodiment 29 or 30, wherein the T cell is a CD8+ T cell.

Embodiment 32. The cell of embodiment 31, wherein the CD8+ T cell is CD4 independent.

Embodiment 33. The cell of any one of embodiments 29-32, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

Embodiment 34. The cell of any one of embodiments 32-33, wherein the T cell is CD62L+.

Embodiment 35. The cell of any one of embodiments 32-33, wherein the T cell is CD45RA+.

Embodiment 36. The cell of any one of embodiments 32-33, wherein the T cell is CD45RA+ and CD62L+.

Embodiment 37. The cell of any one of embodiments 1-36, wherein the CAR and/or the TCR-like fusion molecule is integrated at a locus within the genome of the T cell.

Embodiment 38. The cell of embodiment 37, wherein the locus is selected from the group consisting of a TRAC locus, a TRBC locus, a TRDC locus, and a TRGC locus.

Embodiment 39. The cell of embodiment 37 or38, wherein the locus is a TRAC locus or a TRBC locus.

Embodiment 40. The cell of embodiment 39, wherein the locus is a TRAC locus.

Embodiment 41. The cell of any one of embodiments 1-40, wherein the first antigen and/or the second antigen is a tumor antigen or a pathogen antigen.

Embodiment 42. The cell of embodiment 41, wherein the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

Embodiment 43. The cell of any one of embodiments 1-42, wherein the first antigen is selected from the group consisting of CD312, CD19, CD20, CD22, CD276, and CAIX.

Embodiment 44. The cell of any one of embodiments 1-43, wherein the second antigen is selected from the group consisting of CD70, CD19, CD20, and CD22.

Embodiment 45. The cell of any one of embodiments 42-44, wherein the first antigen and the second antigen are CD312 and CD70.

Embodiment 46. The cell of embodiment 45, wherein the extracellular antigen-binding domain of the CAR targeting CD312 comprises a VH comprising a CDR1 comprising amino acids having the sequence set forth in SEQ ID NO: 73, a CDR2 comprising the amino acid sequence set forth in SEQ ID NO: 74, and a CDR3 comprising the amino acid sequence set forth in SEQ ID NO: 75; and a VL comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78.

Embodiment 47. The cell of embodiment 45 or 46, wherein the TCR-like fusion molecule targeting CD70 comprises a first antigen binding chain comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and a second antigen binding chain comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138.

Embodiment 48. The cell of any one of embodiments 42-44, wherein the first antigen and the second antigen are CD276 and CD70.

Embodiment 49. The cell of embodiment 48, wherein the extracellular antigen-binding domain of the CAR targeting CD276 comprises a VH comprising a CDR1 comprising amino acids having the sequence set forth in SEQ ID NO: 85, a CDR2 comprising the amino acid sequence set forth in SEQ ID NO: 86, and a CDR3 comprising the amino acid sequence set forth in SEQ ID NO: 87; and a VL comprising a CDR1 comprising the amino acid sequence set forth in SEQ ID NO: 88, a CDR2 comprising the amino acid sequence set forth in SEQ ID NO: 89, and a CDR3 comprising the amino acid sequence set forth in SEQ ID NO: 90.

Embodiment 50. The cell of embodiment 48 or 49, wherein the TCR-like fusion molecule targeting CD70 comprises a first antigen binding chain comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and a second antigen binding chain comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138.

Embodiment 51. The cell of any one of embodiments 42-44, wherein the first antigen and the second antigen are CAIX and CD70.

Embodiment 52. The cell of any one of embodiments 42-44, wherein the first antigen and the second antigen are CD19 and CD22.

Embodiment 53. The cell of any one of embodiments 42-44, wherein the first antigen and the second antigen are CD19 and CD20.

Embodiment 54. The cell of any one of embodiments 42-44, wherein the first antigen and the second antigen are CD20 and CD22.

Embodiment 55. The cell of any one of embodiments 42-44, wherein the first antigen and the second antigen are CD20 and CD19.

Embodiment 56. The cell of any one of embodiments 42-44, wherein the first antigen and the second antigen are CD22 and CD20.

Embodiment 57. The cell of any one of embodiments 42-44, wherein the first antigen and the second antigen are CD22 and CD19.

Embodiment 58. The cell of embodiment 57, wherein the extracellular antigen-binding domain of the CAR targeting CD22 comprises: a) a VH comprising a CDR1 comprising amino acids having the sequence set forth in SEQ ID NO: 97, a CDR2 comprising the amino acid sequence set forth in SEQ ID NO: 98, and a CDR3 comprising the amino acid sequence set forth in SEQ ID NO: 99; and a VL comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102; b) a VH comprising a CDR1 comprising amino acids having the sequence set forth in SEQ ID NO: 97, a CDR2 comprising the amino acid sequence set forth in SEQ ID NO: 153, and a CDR3 comprising the amino acid sequence set forth in SEQ ID NO: 99; and a VL comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102; c) a VH comprising a CDR1 comprising amino acids having the sequence set forth in SEQ ID NO: 119, a CDR2 comprising the amino acid sequence set forth in SEQ ID NO: 120, and a CDR3 comprising the amino acid sequence set forth in SEQ ID NO: 121; and a VL comprising a CDR1 comprising the amino acid sequence set forth in SEQ ID NO: 122, a CDR2 comprising the amino acid sequence set forth in SEQ ID NO: 123, and a CDR3 comprising the amino acid sequence set forth in SEQ ID NO: 124; or d) a V_{H} comprising a CDR1 comprising amino acids having the sequence set forth in SEQ ID NO: 97, a CDR2 comprising the amino acid sequence set forth in SEQ ID NO: 154, and a CDR3 comprising the amino acid sequence set forth in SEQ ID NO: 99; and a V_{L} comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102.

Embodiment 59. The cell of embodiment 57 or 58, wherein the TCR-like fusion molecule targeting CD19 comprises a first antigen binding chain comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and a second antigen binding chain comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148.

Embodiment 60. The cell of any one of embodiments 42-44, wherein the first antigen and the second antigen are selected from Table 8.

Embodiment 61. The cell of any one of embodiments 1-60, further comprising a chimeric co-stimulating receptor (CCR).

Embodiment 62. The cell of embodiment 61, wherein the CCR comprises an extracellular antigen-binding domain that binds to the third antigen and an intracellular domain that is capable of delivering a costimulatory signal to the cell but does not alone deliver an activation signal to the cell.

Embodiment 63. The cell of embodiment 62, wherein the intracellular domain of the CCR comprises at least an intracellular domain of a co-stimulatory molecule or a portion thereof.

Embodiment 64. The cell of embodiment 63, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

Embodiment 65. The cell of any one of embodiments 1-64, wherein the cell further comprises at least one exogenous costimulatory ligand.

Embodiment 66. The cell of embodiment 65, wherein the at least one exogenous co-stimulatory ligand is selected from the group consisting of a tumor necrosis factor (TNF) family member, an immunoglobulin (Ig) superfamily member, and combinations thereof.

Embodiment 67. The cell of embodiment 66, wherein the TNF family member is selected from the group consisting of 4-1BBL, OX40L, CD70, FasL, GITRL, TNF-related apoptosis-inducing ligand (TRAIL), CD30L, LIGHT (TNFSF14), CD40L.

Embodiment 68. The cell of embodiment 66 or 67, wherein the Ig superfamily member is selected from the group consisting of CD80, CD86, ICOSLG, and combinations thereof.

Embodiment 69. The cell of any one of embodiments 65-68, wherein the at least one exogenous costimulatory ligand comprises CD80.

Embodiment 70. The cell of any one of embodiments 65-68, wherein the at least one exogenous a costimulatory ligand comprises 4-1BBL.

Embodiment 71. The cell of any one of embodiments 65-68, wherein the cell comprises two exogenous costimulatory ligands.

Embodiment 72. The cell of embodiment 71, wherein the at least two exogenous costimulatory ligands comprise CD80 and 4-1BBL.

Embodiment 73. The cell of any one of embodiments 65-72, wherein the at least two exogenous costimulatory ligands comprise the amino acid sequence set forth in SEQ ID NO: 67 and/or the amino acid sequence set forth in SEQ ID NO: 69.

Embodiment 74. The cell of any one of embodiments 1-73, wherein the cell further comprises a fusion polypeptide comprising: a) an extracellular domain and a transmembrane domain of a co-stimulatory ligand, and b) an intracellular domain of a first co-stimulatory molecule.

Embodiment 75. The cell of embodiment 74, wherein the co-stimulatory ligand is selected from the group consisting of a tumor necrosis factor (TNF) family member, an immunoglobulin (Ig) superfamily member, and combinations thereof.

Embodiment 76. The cell of embodiment 75, wherein the TNF family member is selected from the group consisting of 4-1BBL, OX40L, CD70, GITRL, CD40L, and combinations thereof.

Embodiment 77. The cell of embodiment 75 or 76, wherein the Ig superfamily member is selected from the group consisting of CD80, CD86, ICOSLG, and combinations thereof.

Embodiment 78. The cell of any one of embodiments 74-77, wherein the co-stimulatory ligand is CD80.

Embodiment 79. The cell of any one of embodiments 74-78, wherein the first co-stimulatory molecule is selected from the group consisting of CD28, 4-1BB, OX40, ICOS, DAP-10, CD27, CD40, NKG2D, CD2, and combinations thereof.

Embodiment 80. The cell of embodiment 79, wherein the first co-stimulatory molecule is 4-1BB.

Embodiment 81. The cell of any one of embodiments 74-80, wherein the co-stimulatory ligand is CD80 and the first co-stimulatory molecule is 4-1BB.

Embodiment 82. The cell of embodiment 781, wherein the fusion polypeptide comprises the amino acid sequence set forth in SEQ ID NO: 71.

Embodiment 83. The cell of any one of embodiments 74-82, wherein the fusion polypeptide further comprises an intracellular domain of a second co-stimulatory molecule.

Embodiment 84. The cell of embodiment 83, wherein the second co-stimulatory molecule is selected from the group consisting of CD28, 4-1BB, OX40, ICOS, DAP-10, CD27, CD40, NKG2D, CD2, and combinations thereof.

Embodiment 85. The cell of embodiment 83 or 84, wherein the second co-stimulatory molecule is CD28.

Embodiment 86. The cell of any one of embodiments 74-85, wherein the co-stimulatory ligand is CD80, the first co-stimulatory molecule is 4-1BB, and the second co-stimulatory molecule is CD28.

Embodiment 87. The cell of embodiment 86, wherein the fusion polypeptide comprises the amino acid sequence set forth in SEQ ID NO: 72.

Embodiment 88. The cell of any one of embodiments 1-87, wherein the cell is autologous.

Embodiment 89. The cell of any one of embodiments 1-87, wherein the cell is allogeneic.

Embodiment 90. A composition comprising the cell of any one of embodiments 1-89.

Embodiment 91. The composition of embodiment 90, which is a pharmaceutical composition further comprising a pharmaceutically acceptable excipient.

Embodiment 92. A nucleic acid composition comprising a first polynucleotide encoding a chimeric antigen receptor (CAR) that targets a first antigen and a second polynucleotide encoding a TCR-like fusion molecule that targets a second antigen.

Embodiment 93. A vector comprising a first polynucleotide encoding a chimeric antigen receptor (CAR) that targets a first antigen and a second polynucleotide encoding a TCR-like fusion molecule that targets a second antigen.

Embodiment 94. The vector of embodiment 93, wherein the vector is a lentiviral vector.

Embodiment 95. The vector of embodiment 93 or 94, wherein the vector is a γ-retroviral vector.

Embodiment 96. A lipid nanoparticle comprising a first polynucleotide encoding a chimeric antigen receptor (CAR) that targets a first antigen and a second polynucleotide encoding a TCR-like fusion molecule that targets a second antigen.

Embodiment 97. A polynucleotide encoding a chimeric antigen receptor (CAR) that targets a first antigen and a TCR-like fusion molecule that targets a second antigen.

Embodiment 98. A vector comprising the polynucleotide of embodiment 97.

Embodiment 99. The vector of embodiment 98, wherein the vector is a lentiviral vector.

Embodiment 100. The vector of embodiment 98 or 97, wherein the vector is a γ-retroviral vector.

Embodiment 101. A lipid nanoparticle comprising the polynucleotide of embodiment 97.

Embodiment 102. A composition comprising the polynucleotide of embodiment 94, the vector of any one of embodiments 93-95 or 98-100, or the lipid nanoparticle of embodiment 96 or 101.

Embodiment 103. The composition of embodiment 102, which is a pharmaceutical composition further comprising a pharmaceutically acceptable excipient.

Embodiment 104. A method for producing a cell of any one of embodiments 1-89, the method comprising introducing into the cell the nucleic acid composition of embodiment 92, a vector of any one of embodiments 93-95 or 98-100, a polynucleotide of embodiment 97, a lipid nanoparticle of embodiment 96 or 101, or a composition of embodiment 102 or 103.

Embodiment 105. The method of embodiment 104, further comprising generating a gene disruption of a CD70 locus.

Embodiment 106. The method of embodiment 104 or 105, further comprising generating a gene disruption of a TRAC locus.

Embodiment 107. The method of embodiment 105 or 106, wherein the gene disruption comprises a substitution, a deletion, an insertion, a mutation, or a combination thereof.

Embodiment 108. The method of embodiment 107, wherein the mutation comprises a missense mutation, a nonsense mutation, or a combination thereof.

Embodiment 109. The method of embodiment 107, wherein the deletion comprises a non-frameshift deletion, a frameshift deletion, or a combination thereof.

Embodiment 110. The method of embodiment 107, wherein the insertion comprises a non-frameshift insertion, a frameshift insertion, or a combination thereof.

Embodiment 111. The method of any one of embodiments 104-110, wherein the gene disruption of the CD70 locus results in a non-functional CD70 protein or in knockout of the CD70 gene expression.

Embodiment 112. The method of any one of embodiments 104-111, wherein the gene disruption of the TRAC locus results in a non-functional TRAC protein or in knockout of the TRAC gene expression.

Embodiment 113. The method of any one of embodiments 104-112, wherein generating the gene disruption of comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

Embodiment 114. The method any one of embodiments 104-113, wherein the gene disruption of a CD70 locus or a TRAC locus is generated before activation of a cell.

Embodiment 115. The method any one of embodiments 104-114, wherein the gene disruption of a CD70 locus or a TRAC locus is generated after activation of a cell.

Embodiment 116. The method any one of embodiments 104-114, wherein a) the gene disruption of a CD70 locus is generated before activation of a cell, and b) the gene disruption of a TRAC locus is generated after activation of a cell.

Embodiment 117. The method any one of embodiments 104-114, wherein a) the gene disruption of a TRAC locus is generated before activation of a cell, and b) the gene disruption of a CD70 locus is generated after activation of a cell.

Embodiment 118. The method of embodiment 104, further comprising generating a gene modification of a CD70 gene.

Embodiment 119. The method of embodiment 104 or 118, further comprising generating a gene modification of a TRAC gene and/or a TRBC gene.

Embodiment 120. The method of embodiment 118 or 119, wherein the gene modification of the CD70 gene results in a non-functional CD70 protein or in knockdown of the CD70 gene expression.

Embodiment 121. The method of any one of embodiments 118-120, wherein the gene modification of the TRAC locus results in a non-functional TRAC protein or in knockdown of the TRAC gene expression.

Embodiment 122. The method of any one of embodiments 118-121, wherein the gene modification of the TRBC locus results in a non-functional TRBC protein or in knockdown of the TRBC gene expression.

Embodiment 123. The method of any one of embodiments 104-122, further comprising introducing a chimeric co-simulating receptor (CCR).

Embodiment 124. The method of any one of embodiments 104-123, further comprising introducing at least one exogenous costimulatory ligand.

Embodiment 125. The method of any one of embodiments 104-124, further comprising introducing a fusion polypeptide comprising: a) an extracellular domain and a transmembrane domain of a co-stimulatory ligand, and b) an intracellular domain of a first co-stimulatory molecule.

Embodiment 126. A cell produced by the method of any one of embodiments 104-125.

Embodiment 127. A method of reducing tumor burden in a subject, the method comprising administering to the subject an effective amount of the cells of any one of embodiments 1-89 or 126, or the composition of embodiment 90 or 91.

Embodiment 128. The method of embodiment 127, wherein the method reduces the number of tumor cells, reduces tumor size, and/or eradicates the tumor in the subject.

Embodiment 129. A method of preventing and/or treating a neoplasm or a tumor in the subject, administering to the subject an effective amount of the cells of any one of embodiments 1-89 or 126, or the composition of embodiment 90 or 91.

Embodiment 130. The method of any one of embodiments 127-129, wherein the neoplasm or tumor is cancer.

Embodiment 131. The method of any one of embodiments 127-130, wherein the neoplasm or tumor comprises antigen heterogeneity of the first antigen and the second antigen.

Embodiment 132. The method of embodiment 131, wherein the second antigen has a low antigen density.

Embodiment 133. The method of embodiment 131 or 132, wherein the second antigen is expressed on tumor cells having a low tumor cell frequency.

Embodiment 134. The method of any one of embodiments 131-133, wherein the first antigen and the second antigen are independently selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

Embodiment 135. The method of any one of embodiments 131-134, wherein the first antigen is selected from the group consisting of CD312, CD19, CD20, CD22, CD276, and CAIX.

Embodiment 136. The method of any one of embodiments 131-134, wherein the second antigen is selected from the group consisting of CD70, CD19, CD20, and CD22.

Embodiment 137. The method of any one of embodiments 131-134, wherein the first antigen and the second antigen are selected from a) CD312 and CD70; b) CD276 and CD70; c) CAIX and CD70; d) CD19 and CD22; e) CD19 and CD20; f) CD20 and CD22; g) CD20 and CD19; h) CD22 and CD20; or i) CD22 and CD19.

Embodiment 138. The method of any one of embodiments 131-134, wherein the first antigen and the second antigen are selected from Table 8.

Embodiment 139. The method of any one of embodiments 127-138, wherein the neoplasm or tumor is a solid tumor.

Embodiment 140. The method of embodiment 139, wherein the solid tumor is selected from the group consisting of melanoma, renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

Embodiment 141. The method of embodiment 140, wherein the solid tumor is melanoma.

Embodiment 142. The method of any one of embodiments 127-138, wherein the neoplasm or tumor is a blood cancer.

Embodiment 143. The method of any one of embodiments 127-138 or 142, wherein the neoplasm or tumor is a myeloid disorder.

Embodiment 144. The method of embodiment 143, wherein the myeloid disorder is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

Embodiment 145. The method of embodiment 144, wherein the myeloid disorder is acute myeloid leukemia (AML).

Embodiment 146. The method of any one of embodiments 127-138 or 142, wherein the neoplasm or tumor is a B-cell malignancy.

Embodiment 147. The method of embodiment 146, wherein the B-cell malignancy is selected from the group consisting of B cell non-Hodgkin lymphoma (NHL), B cell Hodgkin's lymphoma, B cell acute lymphocytic leukemia (ALL), B cell chronic lymphocytic leukemia (CLL), multiple myeloma (MM), CLL with Richter's transformation, and CNS lymphoma.

Embodiment 148. The method of embodiment 147, wherein the B-cell malignancy is B cell acute lymphocytic leukemia.

Embodiment 149. The method of any one of embodiments 127-138 or 142, wherein the neoplasm or tumor is a leukemia.

Embodiment 150. The method of embodiment 149, wherein the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, B-cell precursor acute lymphoblastic leukemia, and T-cell precursor acute lymphoblastic leukemia.

Embodiment 151. The method of any one of embodiments 127-138 or 142, wherein the neoplasm or tumor is a lymphoma.

Embodiment 152. The method of embodiment 151, wherein the lymphoma is selected from the group consisting of Hodgkin's lymphoma, non-Hodgkin's lymphoma, B-cell non-Hodgkin's lymphoma, T-cell non-Hodgkin's lymphoma, and T-cell precursor acute lymphoblastic lymphoma.

Embodiment 153. The method of any one of embodiments 127-152, wherein the subject has a relapse of the neoplasm or tumor.

Embodiment 154. The method of any one of embodiments 127-153, wherein the subject received treatment which leads to residual tumor cells.

Embodiment 155. A method of preventing and/or treating a pathogen infection in a subject, the method comprising administering to the subject an effective amount of the cells of any one of embodiments 1-89 or 126, or the composition of embodiment 90 or 91.

Embodiment 156. A method of preventing and/or treating an autoimmune disease in a subject, the method comprising administering to the subject an effective amount of the cells of any one of embodiments 1-89 or 126, or the composition of embodiment 90 or 91.

Embodiment 157. A method of preventing and/or treating an infectious disease in a subject, the method comprising administering to the subject an effective amount of the cells of any one of embodiments 1-89 or 126, or the composition of embodiment 90 or 91.

Embodiment 158. The cells of any one of embodiments 1-89 or 126, or the composition of embodiment 90 or 91 for use in reducing tumor burden, treating and/or preventing a neoplasm or a tumor, preventing and/or treating a pathogen infection, preventing and/or treating an autoimmune disease, and/or preventing and/or treating an infectious disease, in a subject.

Embodiment 159. A kit comprising the cell of any one of embodiments 1-89 or 126, or the composition of embodiment 90 or 91.

Embodiment 160. The kit of embodiment 159, wherein the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

HIT T cells have been demonstrated to be superior to conventional CAR T cells in settings of tumors with low target antigen density where the high sensitivity of the HIT receptor enables efficient tumor eradication whereas the lower sensitivity of conventional CAR T cells leads to antigen-low tumor escape. Anti-tumor HIT T cell efficacy was demonstrated in the setting of hematological and solid tumors (Mansilla-Soto et al., Nature Medicine 2022). It was also demonstrated that HIT T cell efficacy can be enhanced by providing additional co-stimulation (e.g., via co-expression of the costimulatory ligands CD80 and 4-1BBL). Specific to the reported CD70-HIT, it was shown that CD70 gene-editing is required to enable full CD70-HIT efficacy, due to CD70 expression in activated T cells resulting in CD70-mediated fratricide.

What was not yet addressed was the challenge of tumor target heterogeneity, i.e., the presence of a tumor cell population with mixed phenotype, including tumor cells with either extremely low target levels or with complete loss of the target. To overcome tumor resistance due to such target heterogeneity, novel concepts for efficient combinatorial targeting of two or more independent antigens are needed. These approaches are generally referred to as OR-gated targeting approaches, following Boolean-logic, where the presence of one of the targeted antigens is sufficient to trigger target cell killing. Several OR-gated targeting approaches based on CARs have been described and include dual CARs (T cells co-expressing two CARs), tandem CARs (T cells expressing a single bi-specific CAR) and pooled single CAR T cells (mixture of T cells expressing a single CAR). Clinical efficacy of such OR-gated CAR therapies is under ongoing investigation, and the ideal CAR design for OR-gated targeting is still unknown.

In the context of HIT, novel approaches for multi-antigen-targeting that include the HIT receptor can allow to broaden the range of targetable antigens and to further limit the risk of antigen-low or antigen-negative escape in a target-heterogenous setting with potential HIT-target-negative or HIT-target-very-low tumor cells that would escape in a single-targeted HIT setting. Therefore, the inventors of the presently disclosed subject matter hypothesized that co-expressing a HIT and a CAR that are directed against two independent target antigens can be superior to HIT alone, and also to alternative OR-gated approaches, due to the increased sensitivity of the HIT and its better compatibility to be co-expressed with a CAR, as compared to approaches co-expressing two CAR molecules with increased cumulative co-stimulation driving excessive T cell differentiation, thus eventually limiting functional persistence.

The presently disclosed subject matter presents a novel OR-gated platform based on co-expression of a HIT and a CAR, here termed as “HIT+CAR”.

Importantly, combining a HIT and a CAR directed against two independent target antigens enabled OR-gated eradication of target-heterogenous tumors, and thereby prevented antigen-low or antigen-negative tumor escape as reported clinically with conventional single-targeted CAR approaches.

Further, combining a HIT and a CAR containing a costimulatory domain provided sufficient costimulation for enhanced HIT+CAR T cell persistence, whereas T cells expressing only the HIT receptor rapidly lost their anti-tumor functional capacity.

The presently disclosed HIT+CAR approach takes advantage of specific CAR signaling architectures, with an ITAM-reduced CAR variant (28z1XX) outperforming conventional second-generation CAR T cells that are currently used clinically (28z or BBz). As outline in the Examples below, HIT+CAR-1XX outperformed HIT+CAR-28z and HIT+CAR-BBz.

The presently disclosed HIT+CAR approach led to a less differentiated T cell phenotype that is associated with enhanced functional persistence, thus outperforming alternative OR-gated dual CAR designs that lead to more differentiated T cell phenotypes.

Notably, HIT efficacy can be enhanced via additional costimulation which can be provided via co-expression of HIT together with a CAR, CCR, costimulatory ligand (e.g., CD80 and 4-1BBL) or synthetic costimulatory fusion molecules disclosed herein. HIT+CAR efficacy can benefit from adding a CCR, costimulatory ligand, or synthetic costimulatory fusion molecules.

In the context of AML, CD70 was selected as HIT target because of its low antigen density, and CD312 was selected as CAR target. As detailed below, HIT+CAR-28z1XX (CD70-HIT+CD312-28z1XX) was demonstrated to fully eradicate a heterogenous AML tumor, and to outperform either CAR-BBz+CAR-28z1XX (CD70-BBz+CD312-28z1XX) or a CAR-28z1XX+CAR-BBz (CD70-28z1XX+CD312-BBz). Further, CD70-HIT alone efficiently reduced AML burden in an AML PDX model.

As outlined in Example 3, delivery of the HIT or HIT+CAR transgene can be performed either via targeted integration into the TRAC locus (using CRISPR/Cas9 and AAV to deliver transgene DNA), or it can be delivered semi-randomly via SFG-gammaretroviral vector. In the setting of CD70-HIT, the presently disclosed subject matter demonstrated superior antitumor efficacy of SFG-CD70-HIT compared to TRAC-CD70-HIT. It was also demonstrated good antitumor efficacy based on gammaretroviral HIT+CAR delivery (SFG-CD70-HIT+312-28z1XX).

The CD70-HIT+312-CAR platform requires multiplex editing to disrupt both endogenous TCR expression (TRAC-KO) to avoid interference between full TCR and HIT assembly, and CD70 expression (CD70-KO) to prevent CD70 fratricide. As shown in Example 3, it was found that multiplex CD70/TRAC editing on unstimulated T cells on d0 (day of T cell isolation) enhances efficacy as compared to d2 multiplex editing on CD3/CD28-activated T cells. Additionally, it was found that sequential electroporation on d0+d2 allows for isolated CD70 and TRAC editing and can thereby reduce risk of translocations.

Finally, when using melanoma as solid tumor model, CD70 was selected as HIT target because of its low antigen density, and CD276 was selected as CAR target. A HIT+CAR−28z1XX (CD70-HIT+CD276−28z1XX) was demonstrated to fully eradicate a heterogenous melanoma tumor, both in a lung-metastasized melanoma model as well as in an orthotopic skin melanoma model.

Overall, the presently disclosed subject matter can be used in different context (e.g., different malignancies) by targeting different tumor antigens. The combinations that were and are investigated are shown in Table 8 below.

TABLE 8

| Malignancy | HIT target | CAR target |
|---|---|---|
| AML | CD70 | CD312 |
| AML | CD70 | CLEC12A |
| AML | CD70 | CD33 |
| AML | CD70 | CD123 |
| AML | CD70 | IL1RAP |
| AML | CD70 | SIGLEC-6 |
| AML | CD70 | GRP78 |
| AML | CD70 | TIM3 |
| AML | SIGLEC-6 | CD312 |
| AML | SIGLEC-6 | CLEC12A |
| AML | SIGLEC-6 | CD33 |
| AML | SIGLEC-6 | CD123 |
| AML | SIGLEC-6 | IL1RAP |
| AML | SIGLEC-6 | CD70 |
| AML | SIGLEC-6 | GRP78 |
| AML | SIGLEC-6 | TIM3 |
| AML | IL1RAP | CD312 |
| AML | IL1RAP | CLEC12A |
| AML | IL1RAP | CD33 |
| AML | IL1RAP | CD123 |
| AML | IL1RAP | SIGLEC-6 |
| AML | IL1RAP | CD70 |
| AML | IL1RAP | GRP78 |
| AML | IL1RAP | TIM3 |
| AML | CLEC12A | CD312 |
| AML | CLEC12A | IL1RAP |
| AML | CLEC12A | CD33 |
| AML | CLEC12A | CD123 |
| AML | CLEC12A | SIGLEC-6 |
| AML | CLEC12A | CD70 |
| AML | CLEC12A | GRP78 |
| AML | CLEC12A | TIM3 |
| AML | GRP78 | CD312 |
| AML | GRP78 | CLEC12A |
| AML | GRP78 | CD33 |
| AML | GRP78 | CD123 |
| AML | GRP78 | IL1RAP |
| AML | GRP78 | CD70 |
| AML | GRP78 | SIGLEC-6 |
| AML | GRP78 | TIM3 |
| AML | TIM3 | CD312 |
| AML | TIM3 | IL1RAP |
| AML | TIM3 | CD33 |
| AML | TIM3 | CD123 |
| AML | TIM3 | SIGLEC-6 |
| AML | TIM3 | CD70 |
| AML | TIM3 | GRP78 |
| AML | TIM3 | CLEC12A |
| B-ALL/B-NHL | CD19 | CD20 |
| B-ALL/B-NHL | CD19 | CD22 |
| B-ALL/B-NHL | CD20 | CD19 |
| B-ALL/B-NHL | CD20 | CD22 |
| B-ALL/B-NHL | CD22 | CD19 |
| B-ALL/B-NHL | CD22 | CD20 |
| Multiple myeloma | BCMA | GPRC5D |
| Multiple myeloma | BCMA | SLAMF7 |
| Multiple myeloma | GPRC5D | BCMA |
| Multiple myeloma | GPRC5D | SLAMF7 |
| Multiple myeloma | SLAMF7 | BCMA |
| Multiple myeloma | SLAMF7 | GPRC5D |
| Multiple myeloma | CD19 | BCMA |
| Multiple myeloma | CD19 | GPRC5D |
| Multiple myeloma | CD19 | SLAMF7 |
| Melanoma | CD70 | CD276 |
| Melanoma | CD276 | CD70 |
| Renal cell carcinoma | CD70 | CAIX |
| Renal cell carcinoma | CAIX | CD70 |
| Glioblastoma | CD70 | CD276 |
| Glioblastoma | CD276 | CD70 |

### Example 2

### Summary

CAR T cells have demonstrated remarkable success in the treatment of B cell malignancies. However, relapse due to antigen-low/negative malignant cells still limits therapeutic success, and target heterogeneity is a key challenge for successful CAR therapy in many other malignancies. There is a high need for effective strategies to address upfront target heterogeneity and CAR target downregulation.

Here, the presently disclosed subject matter investigated a novel OR-gate platform based on co-expression of CAR and HIT. Since HIT depends on endogenous TCR/CD3 components T cells co-expressing CAR and HIT display limited differentiation compared to dual CAR T cells, resulting in improved long-term anti-tumor activity.

The presently disclosed subject matter utilized an NSG xenograft model for target heterogeneity, based on co-engraftment of engineered MOLM13 AML cell line populations expressing either one of two, or both target antigens. To evaluate CAR+HIT designs, target-heterogeneous MOLM13 AML-bearing mice were treated with CAR+HIT T cells containing a 28z, BBz, or 28z1XX CAR. It was found that only the 28z1XX-CAR+HIT design allowed for complete AML remission and long-term survival, whereas 28z-CAR+HIT and BBz-CAR+HIT designs failed to control AML long-term, highlighting the importance for specific CAR signaling in combination with HIT. Next, the 28z1XX-CAR+HIT design was selected and compared to different alternative dual CAR designs based on co-expression of a 28z1XX-CAR and a BBz-CAR. In the presently disclosed NSG AML xenograft heterogeneity model, only 28z1XX-CAR+HIT induced complete AML remission while alternative CAR+CAR versions failed to control AML. A less differentiated T cell phenotype was also observed in 28z1XX-CAR+HIT as compared to the alternative CAR+CAR design.

Overall, the presently disclosed subject matter establishes a novel OR-gated CAR+HIT T cell platform with specific CAR design, leading to superior anti-tumor efficacy in target-heterogeneity models, and with application in various malignancies.

### Results

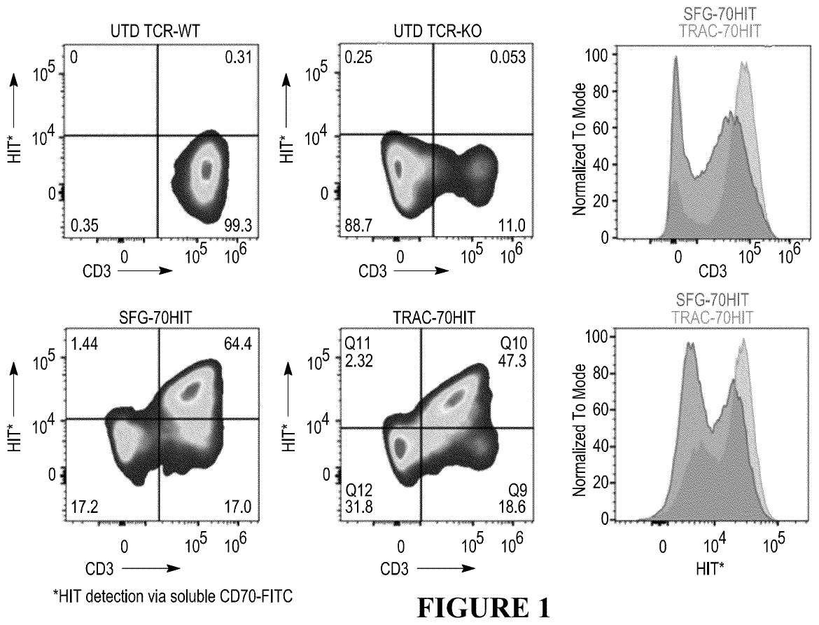

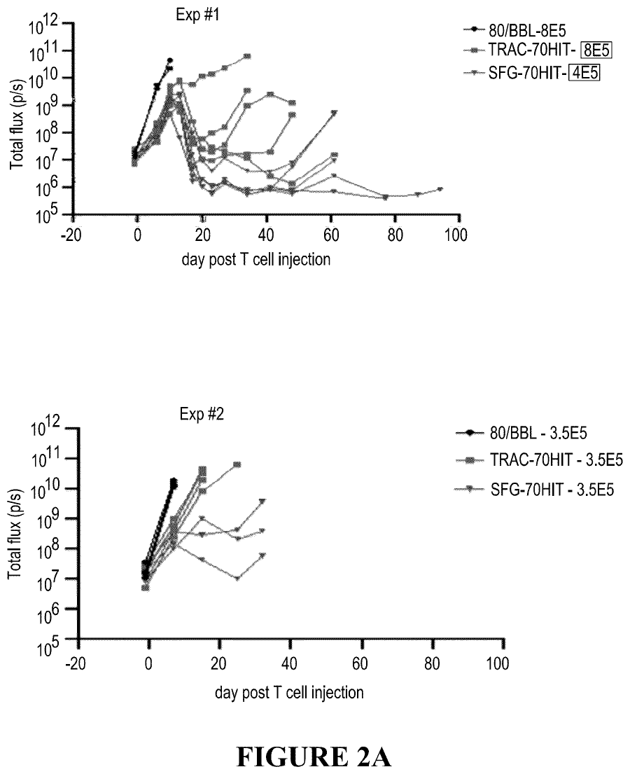

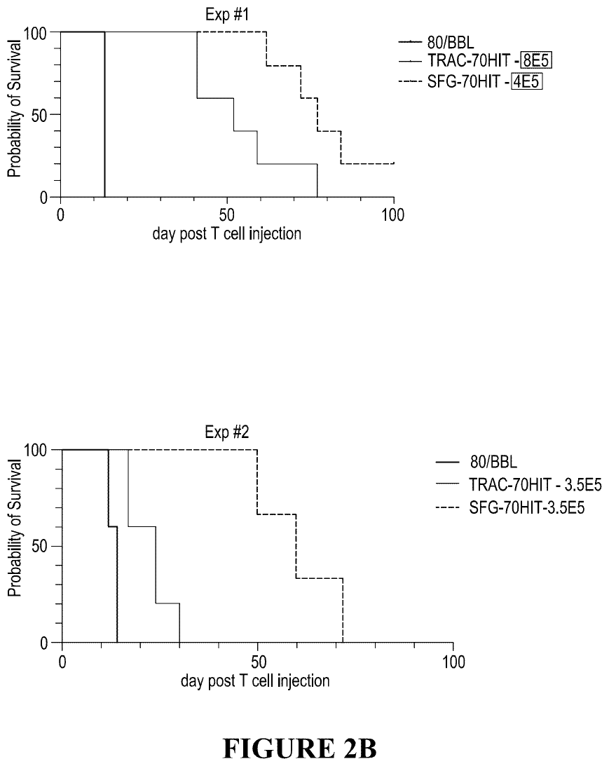

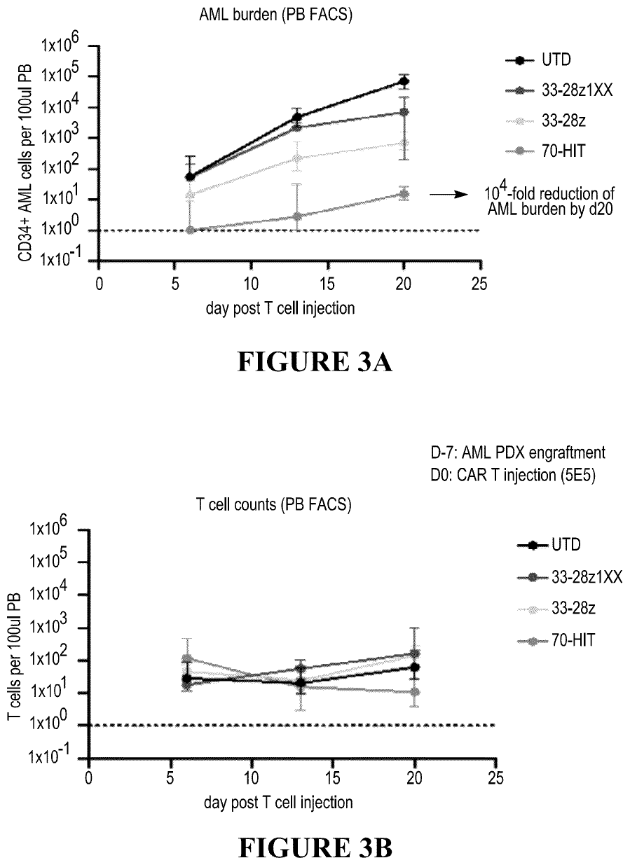

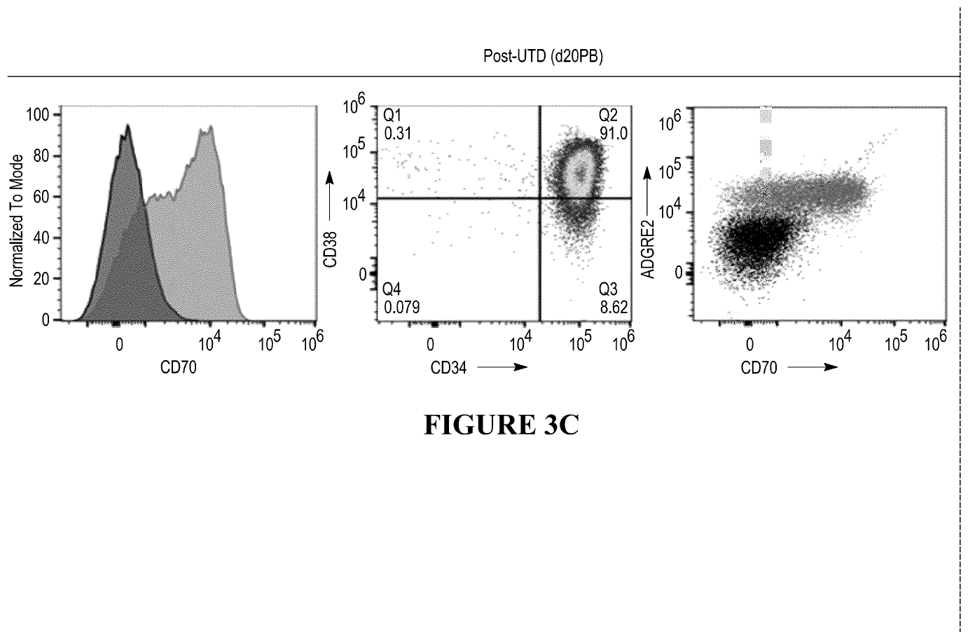

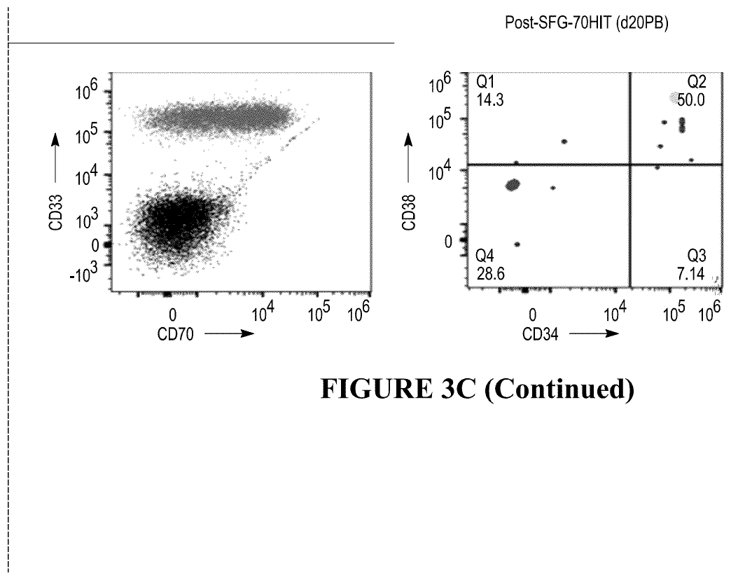

FIG. 1 shows that a TCR-like fusion molecule targeting CD70 (designated as “70HIT”) was highly expressed in T cells if its expression was driven by an endogenous TRAC locus (“TRAC-70HIT”) or an SFG vector (“SFG-70HIT”). T cells including these receptors were administered into MOLM13-WT AML xenograft models. As shown in FIGS. 2A and 2B, SFG-70HIT outperformed TRAC-70HIT in reducing the MOLM13-WT AML xenograft model. Next, it was determined whether T cells expressing the SFG-70HIT were effective in reducing tumor burden in patient-derived AML xenograft model. As illustrated in FIGS. 3A-3C, SFG-70HIT T cells reduced the AML burden of approximately 10⁴-fold as compared to cells expressing an anti-CD33 including a CD28 costimulatory domain and a native or a modified CD3ζ polypeptide (“33-28z” and “33-28z1XX”, respectively). Analysis of peripheral blood cells 20 days after SFG-70HIT T cells injection demonstrated a significant reduction of CD38+/CD34+cells compared to the control. In addition, control peripheral blood cells showed expression of CD312 (also known as ADGRE2) and CD70. In light of these data, CD70 and CD312 were identified as targets due to their expression features (e.g., low antigen density of CD70 and high antigen density of CD312) in Acute Myeloid Leukemia (AML).

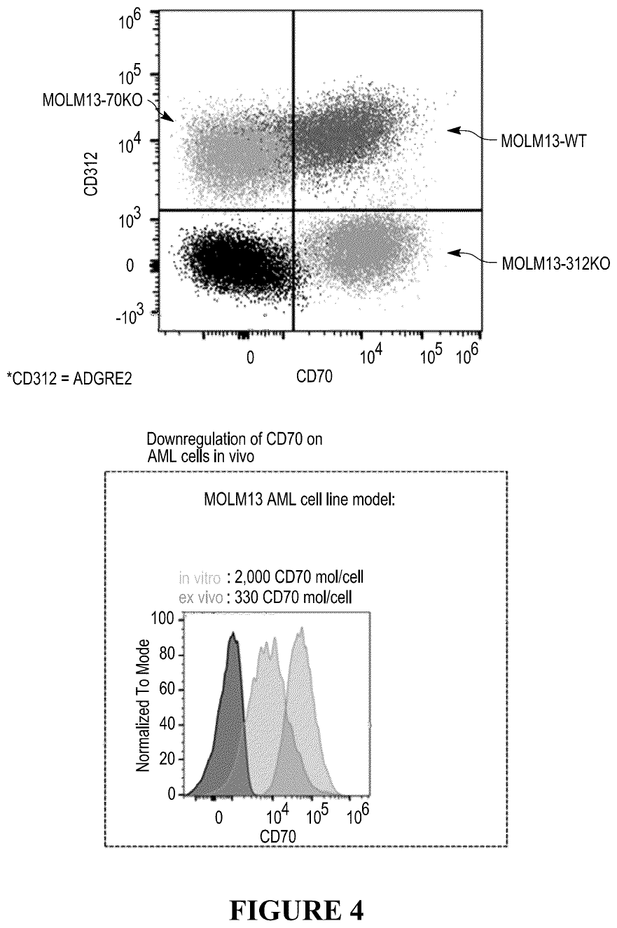

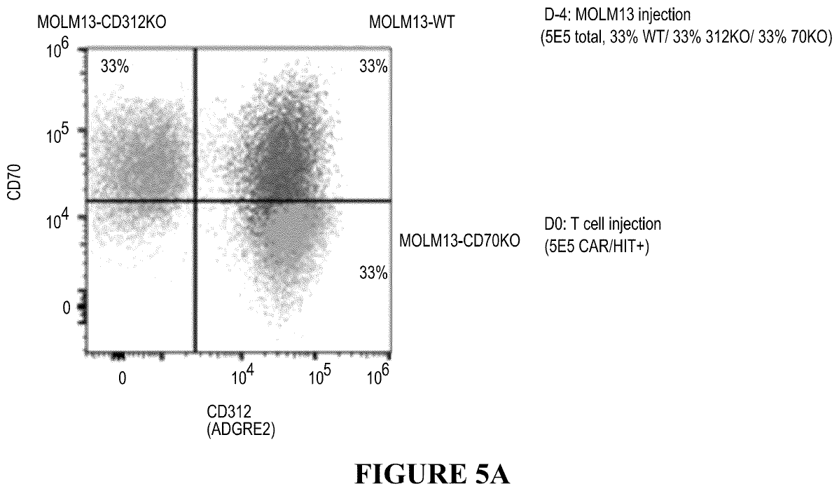

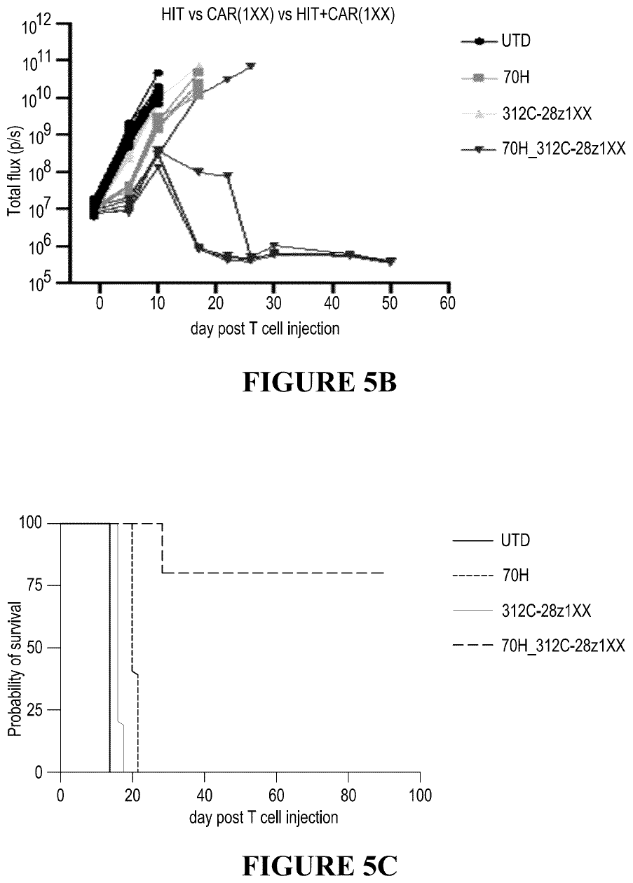

In order to mirror target heterogeneity observed in AML, MOLM13 wild type cells (“MOLM13-WT”) were edited to include downregulation of CD70 (“MOLM13-70KO”) or CD312 (“MOLM13-312KO”). See FIG. 4. The MOLM13-WT, MOLM13-70KO, and MOLM13-312KO were combined in 1:1:1 ratio and injected in order to establish AML xenograft models. See FIGS. 5A-7C. These AML xenograft models were then treated with 5×10⁵ cells including the 70HIT and an anti-CD312 CAR having a 1XX intracellular domain (“70H_312C-28z1XX”). As illustrated in FIGS. 5A-5C, 70H_312C-28z1XX cells induced long-term remission in AML heterogeneity model compared to untransduced cells (“UTD”), cells expressing only the 70HIT or an anti-CD312 CAR having a 1XX intracellular domain (“70H” and “321C-28z1XX”, respectively).

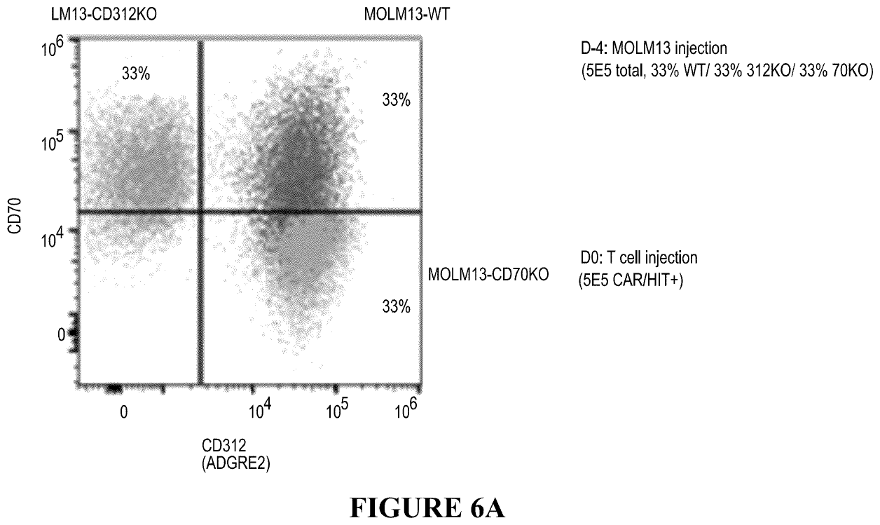

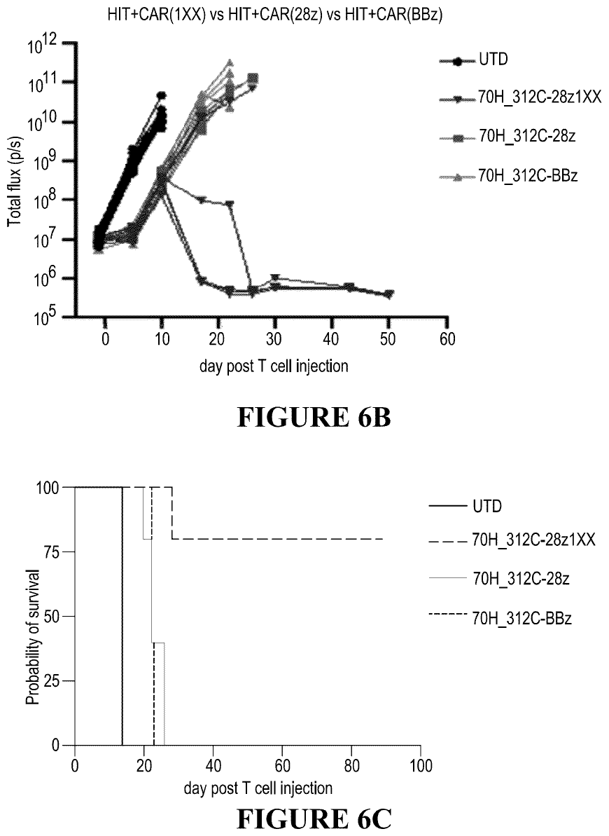

Next, it was determined whether the CAR design could modulate the ability of eradicating tumors in the AML heterogeneity model. AML xenograft models were treated with cells including the 70HIT and an anti-CD312 CAR having i) a CD28 costimulatory domain and a native CD3ζ polypeptide (“70H_312C-28z”), ii) a 4-1BB costimulatory domain and a native CD3ζ polypeptide (“70H_312C-BBz”), or iii) a CD28 costimulatory domain and a 1XX intracellular domain (“70H_312C-28z1XX”). As shown in FIGS. 6A-6C, 70H_312C-28z1XX had significantly improved tumor control and survival compared to 70H_312C-28z and 70H_312C-BBz cells. Thus, 28z1XX-CAR was identified to be superior to 28z-CAR and BBz-CAR when co-expressed with HIT.

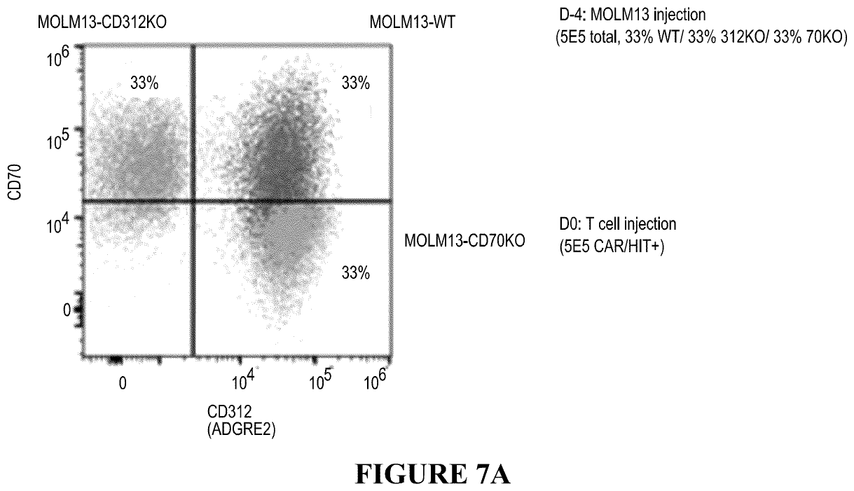

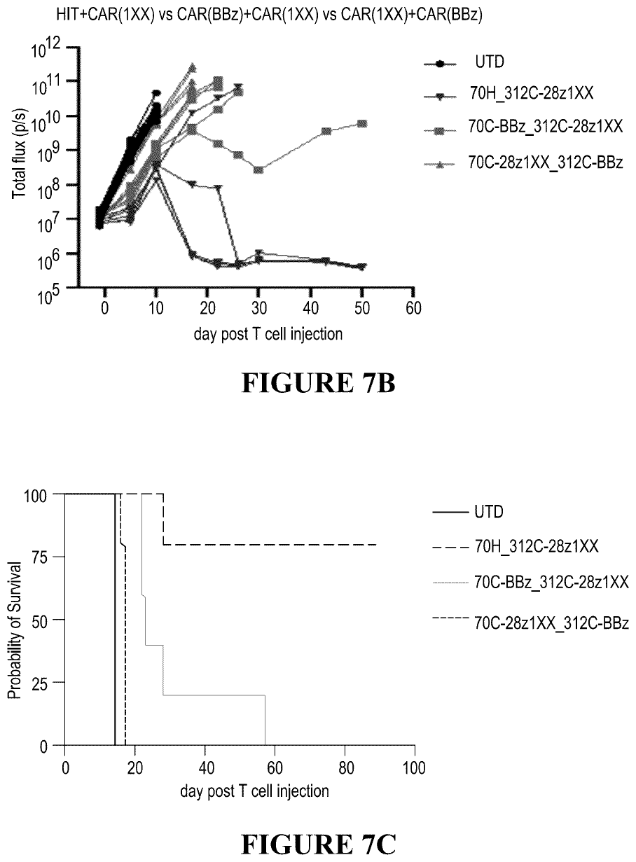

The 70H_312C-28z1XX was also compared to dual CAR strategies. Briefly, 70H_312C-28z1XX cells were compared to cells expressing i) an anti-CD70 CAR having a 4-1BB costimulatory domain and a native CD3ζ polypeptide and an anti-CD312 CAR having a CD28 costimulatory domain and a 1XX intracellular domain (“70C-BBz_312C-28z1XX”), or ii) an anti-CD70 CAR having a CD28 costimulatory domain and a 1XX intracellular domain and an anti-CD312 CAR having a 4-1BB costimulatory domain and a native CD3ζ polypeptide and (“70C-28z1XX_312C-BBz”). FIGS. 7A-7C show that 70H_312C-28z1XX outperformed alternative dual CAR strategies.

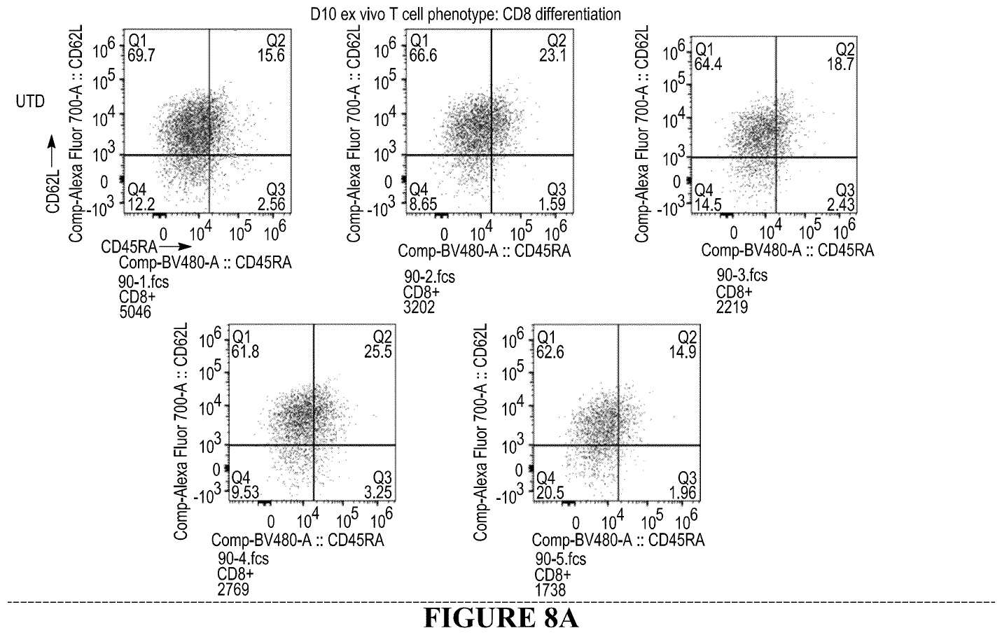

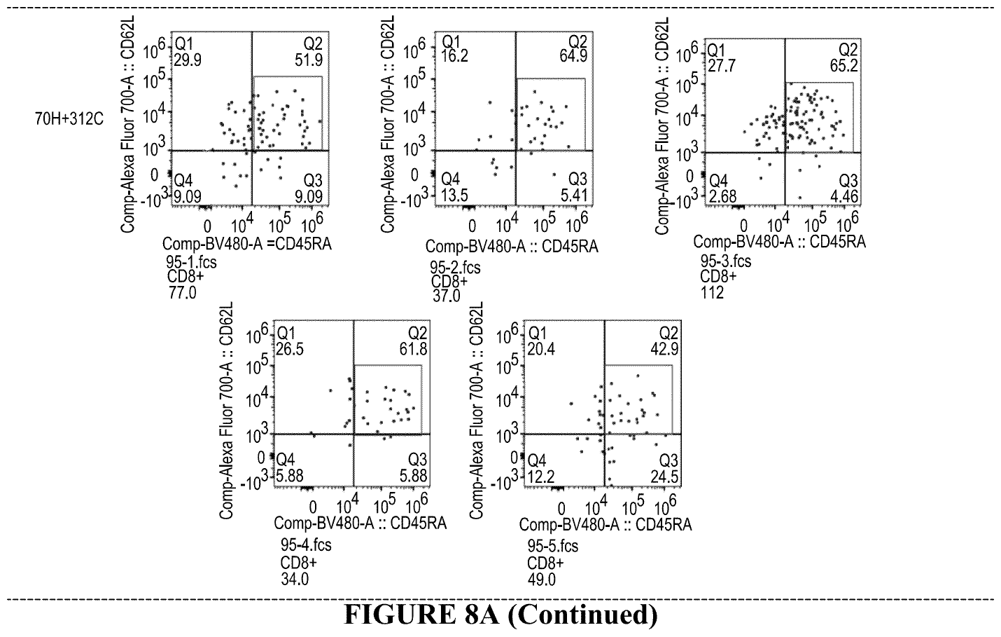

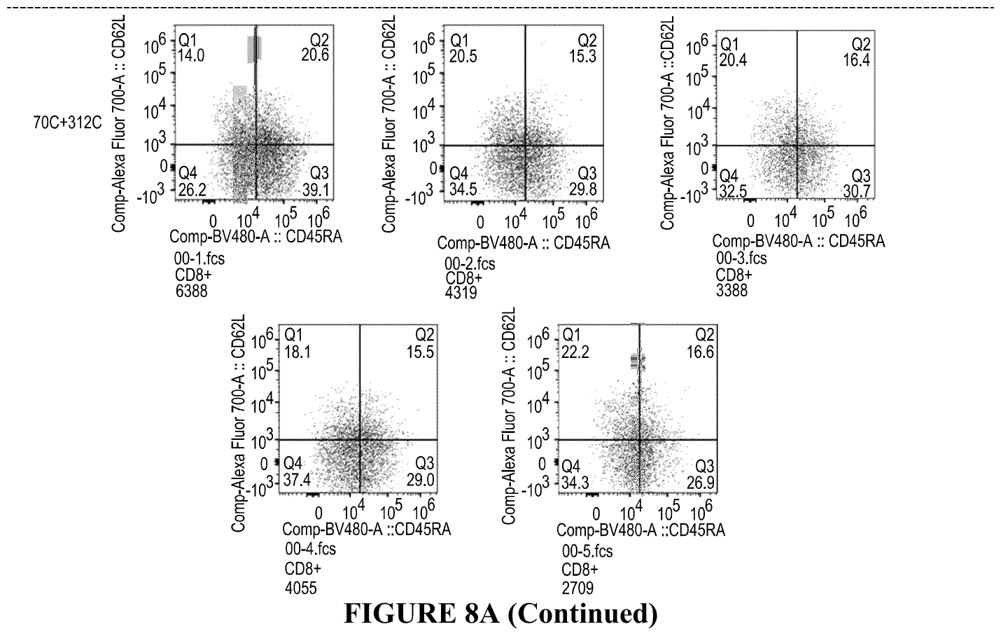

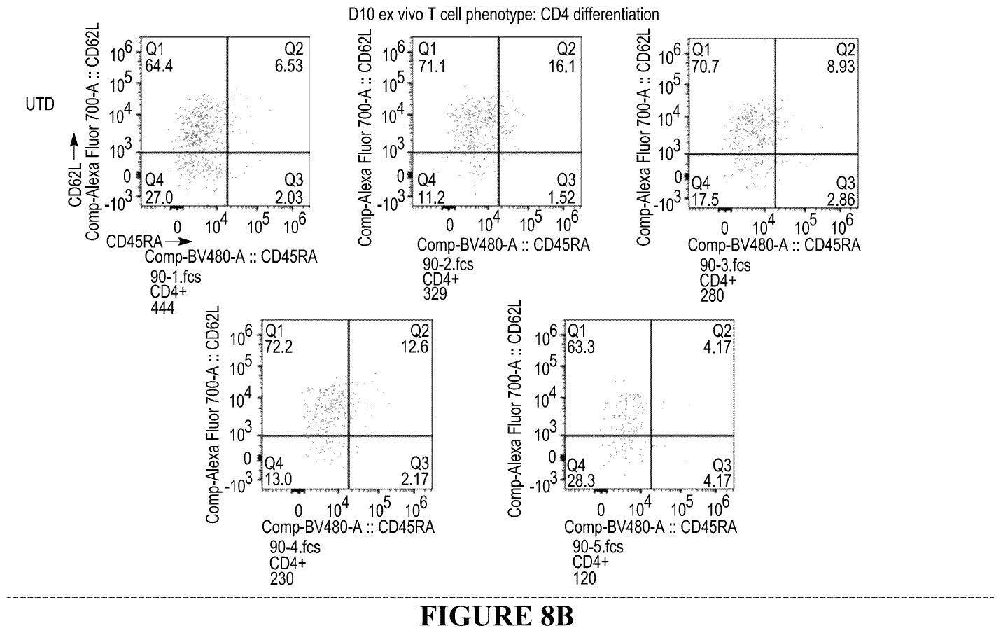

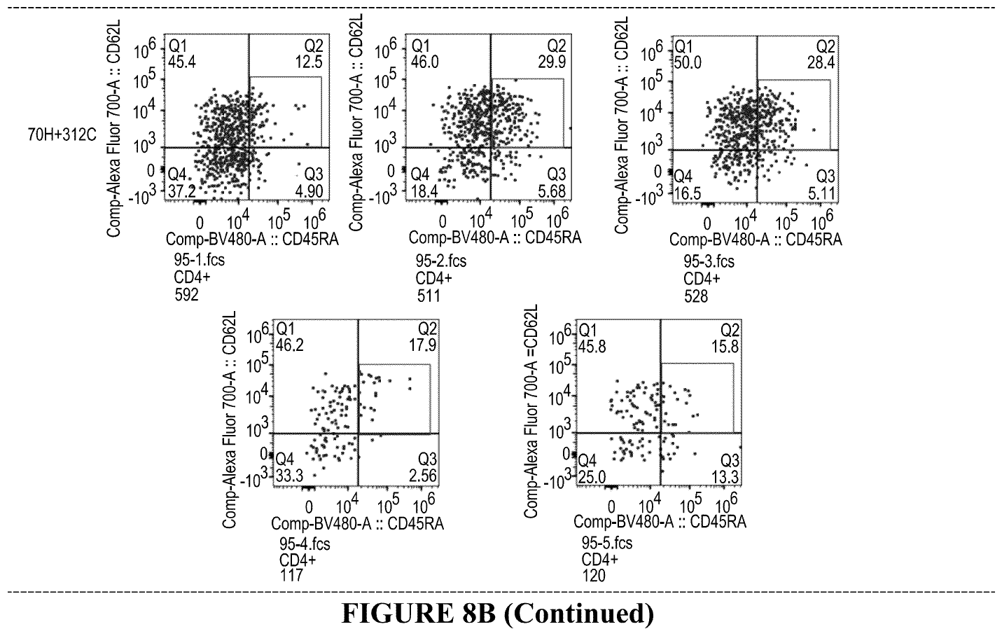

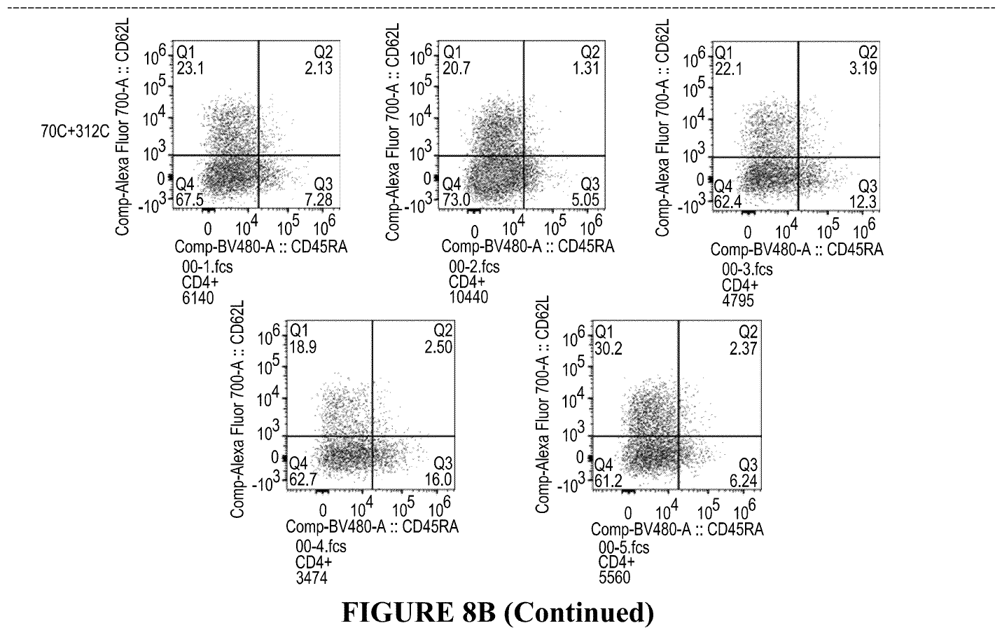

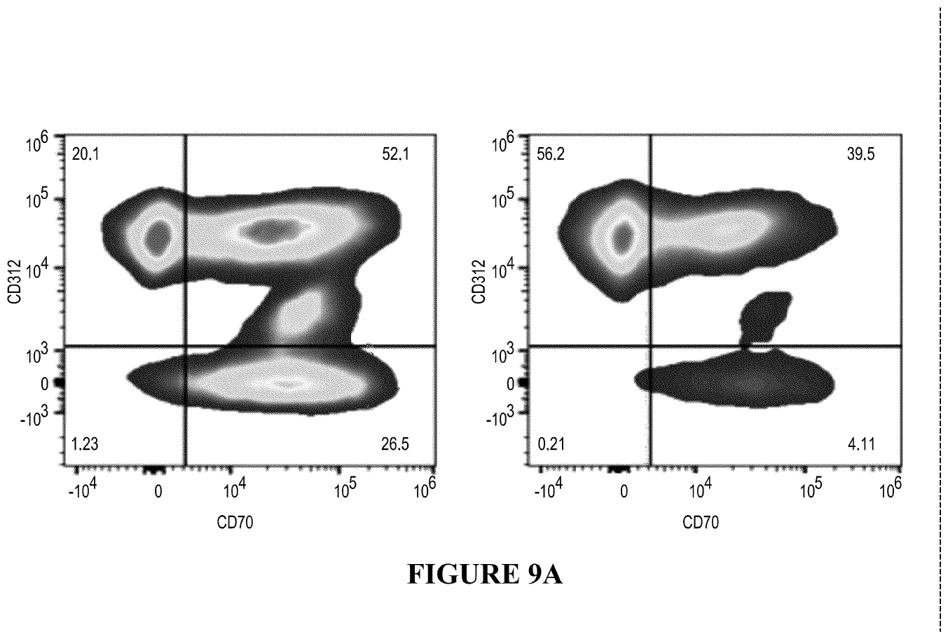

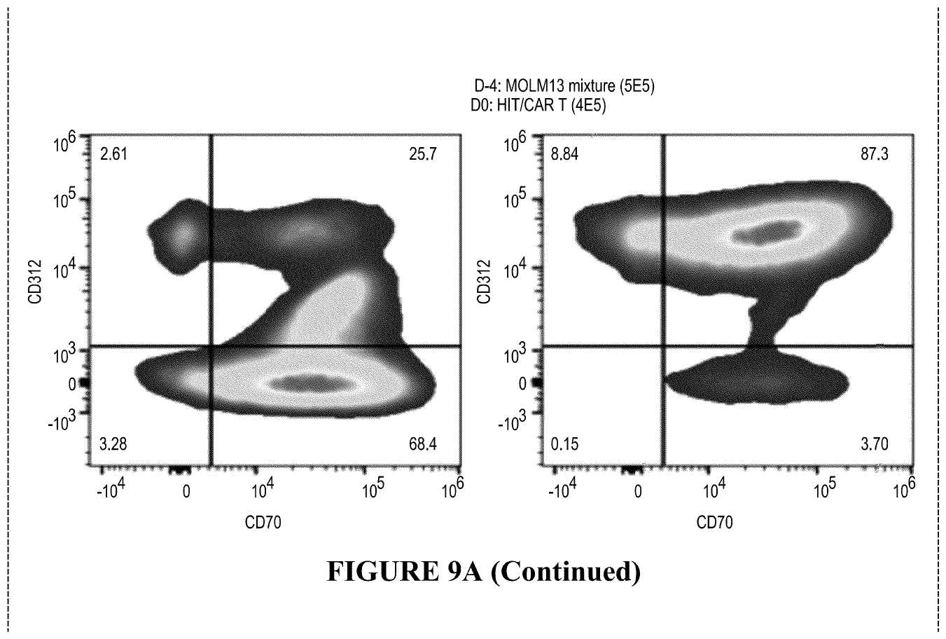

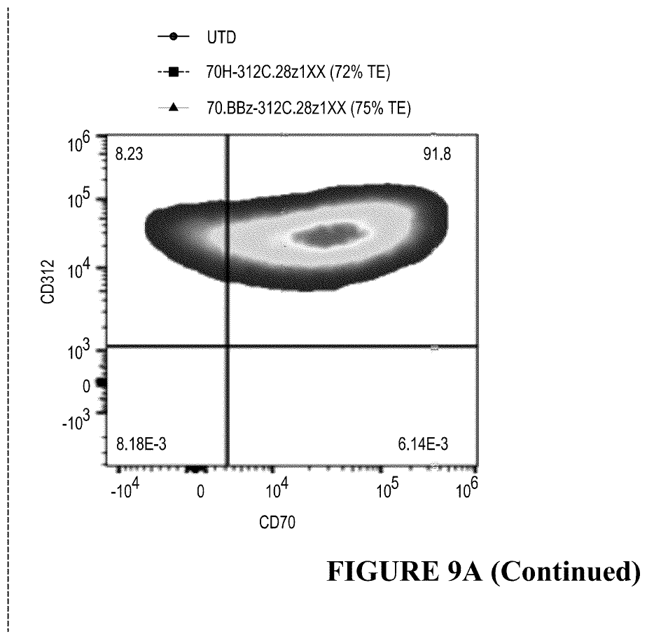

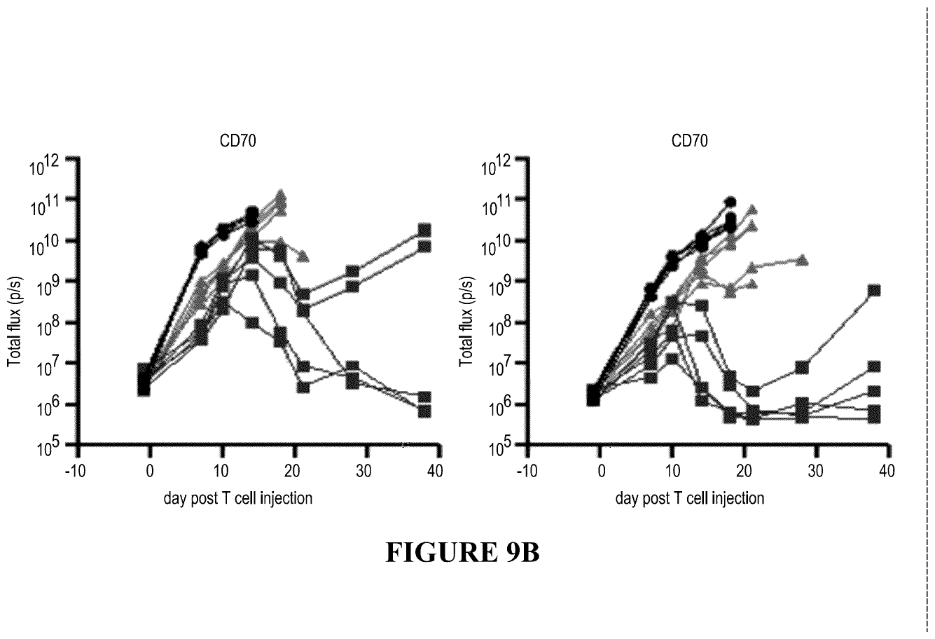

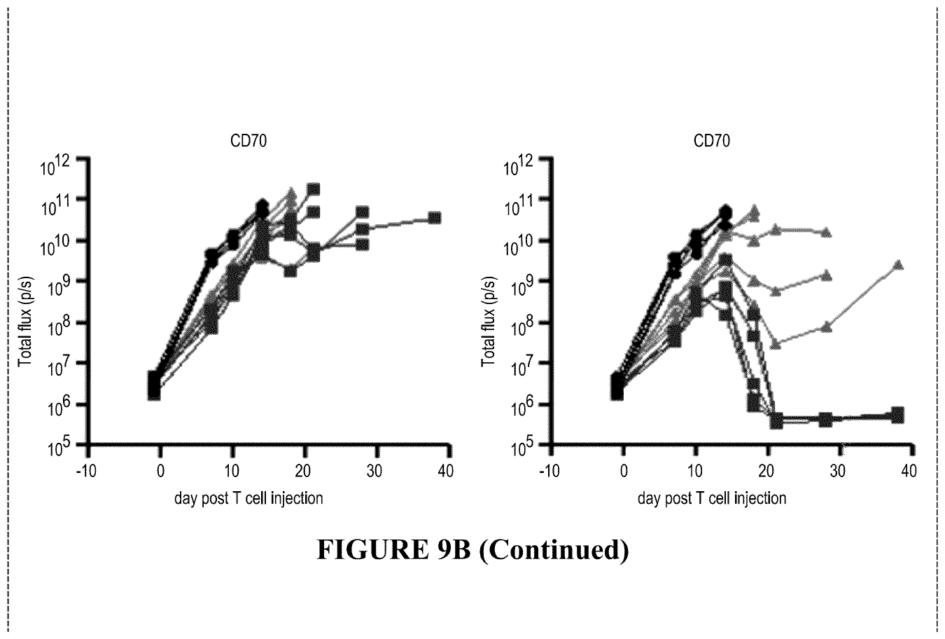

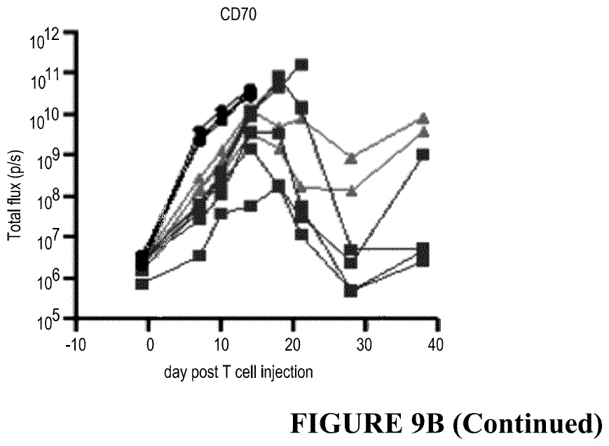

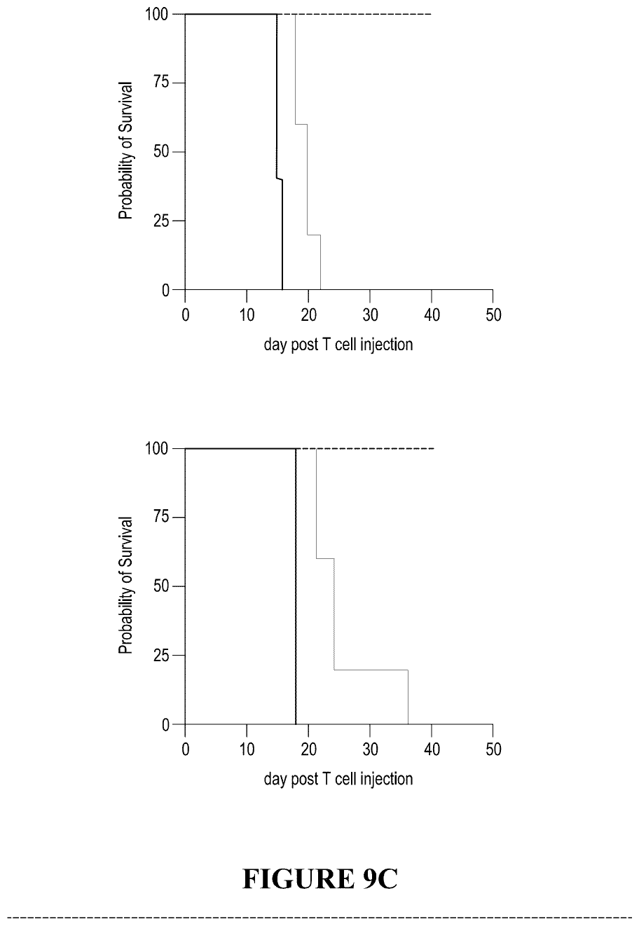

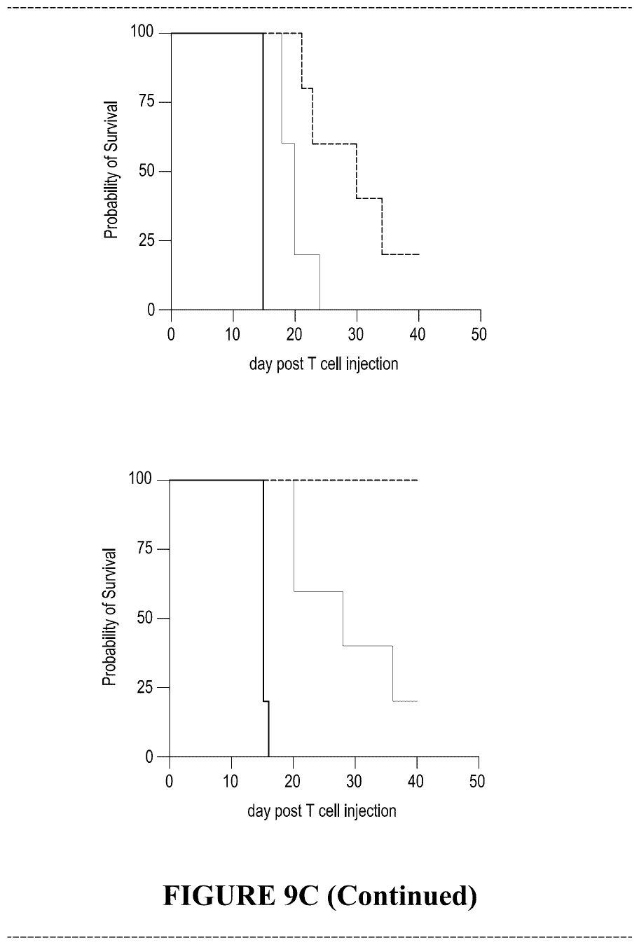

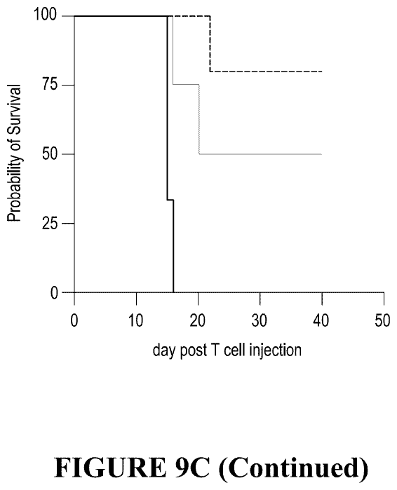

FACS analysis of 70H_312C-28z1XX cells highlighted that these cells had a less differentiated T cell phenotype and higher expression levels of CD62L and CD45RA as compared to dual CAR strategies. See FIGS. 8A and 8B. Further, 70H_312C-28z1XX cells efficiently eradicated MOLM13 AML xenograft in tumor heterogeneity model. See FIGS. 9A-9C.

### Discussion

The present example presents a novel OR-gated platform based on co-expression of a CAR and a HIT (HLA-Independent TCR), the latter being previously described to afford T cells with increased antigen sensitivity (Mansilla-Soto et al., Nature Medicine 2022). Since HIT receptors do not incorporate engineered co-stimulatory domains like conventional CARs, T cells co-expressing CAR and HIT display limited differentiation in comparison to dual CAR T cells, thus resulting in improved long-term anti-tumor activity.

This novel OR-gated therapy (designated as “HIT+CAR”) was evaluated it in the setting of AML which stands out with a high degree of inter- and intra-individual phenotypic heterogeneity. Two target antigens which we have a favorable and non-overlapping expression profile in normal hematopoietic cells and other tissues were selected (Perna et al., Cancer Cell 2017). Specifically, it was selected a lower-density target for the HIT, using its increased sensitivity to avoid antigen-low escape, and selected a higher-density target for the CAR. An NSG xenograft model for target heterogeneity, based on co-engraftment of engineered MOLM13 AML cell line populations endogenously expressing either HIT target alone, CAR target alone, or both.

To identify an optimal HIT+CAR design, target-heterogeneous MOLM13 AML-bearing mice were treated with HIT+CAR T cells containing different CAR architectures and found that only the 28z1XX CAR allowed for complete AML remission and long-term survival in conjunction with the HIT. All other CAR architectures failed to control AML long-term, highlighting the importance of specific CAR signaling in combination with HIT. See FIGS. 5A-5C and 6A-6C.

The superior HIT+CAR-1XX was then selected and compared to dual CARs containing BBz and 28z1XX architectures. In NSG AML xenograft heterogeneity model, the HIT+CAR-1XX induced complete AML remission while the alternative dual CARs failed to control AML. See FIGS. 7A-7C. T cell phenotyping demonstrated a less differentiated T cell phenotype over time with HIT+CAR-1XX as compared to the alternative dual CARs. See FIGS. 8A and 8B.

Overall, the presently disclosed subject matter established a novel OR-gated HIT+CAR T cell platform based on combinatorial expression of HIT and CAR which can be applied to overcome target heterogeneity in various malignancies. The superiority of HIT+CAR-1XX over alternatives, e.g., other CAR architectures and dual CAR approaches, was demonstrated, highlighting its suitability for clinical translation.

### Example 3

It was recently found that CD70 is highly expressed on a substantial fraction of T cells, which may lead to T cell fratricide/suicide and impair efficacy of cells expressing an antigen recognizing receptor targeting CD70. Thus, it is desirable to reduce the expression of CD70 in T cells. In addition, in order to avoid unspecific coupling of between endogenous TCR chain with a recombinant TCR chain (e.g., a first or a second chain of a TCR-like fusion molecule), knockout (KO) of a TRAC locus is also desired.

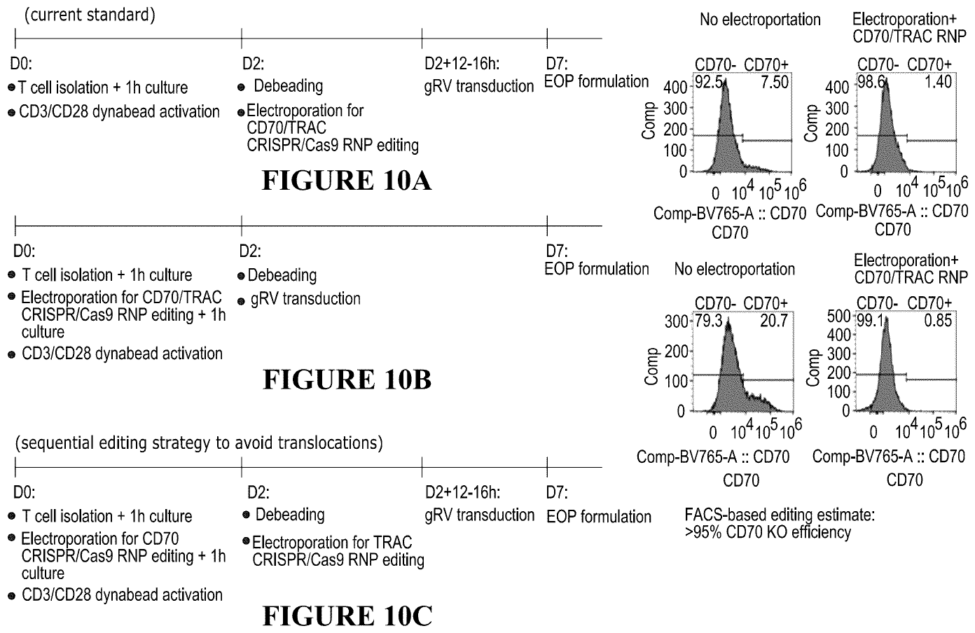

The present example describes novel strategies for achieving a highly efficient CRISPR/Cas9-based knockout (KO) of CD70 and TRAC in T cells. As shown in FIGS. 10A-10C, multiplex CD70/TRAC editing on unstimulated T cells on day 0 had enhanced efficacy as compared to day 2 multiplex editing on CD3/CD28-activated T cells. Further, sequential electroporation on day 0 and day 2 allowed for isolated CD70 and TRAC editing and could reduce risk of translocations.

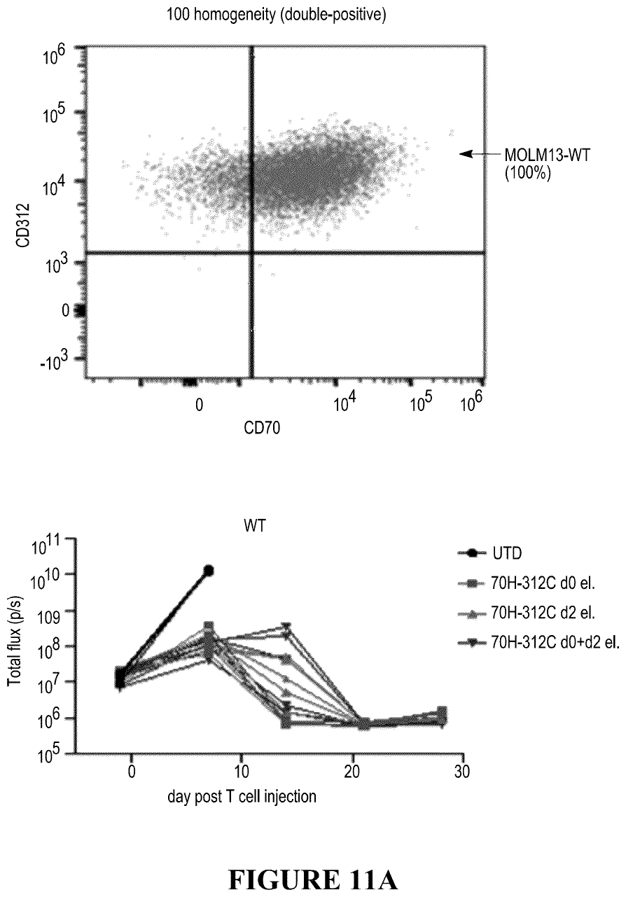

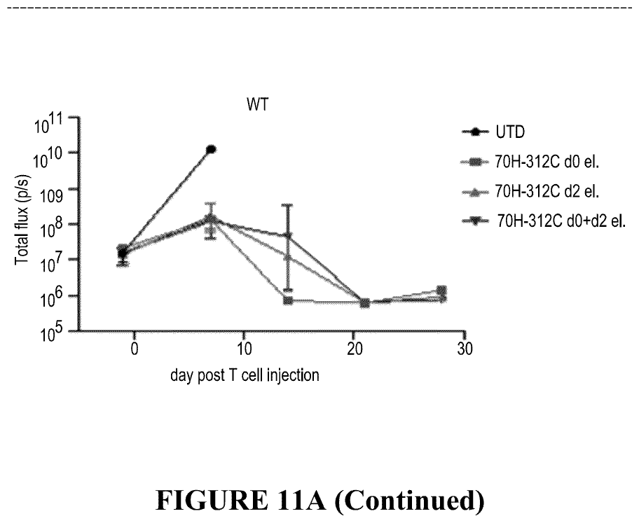

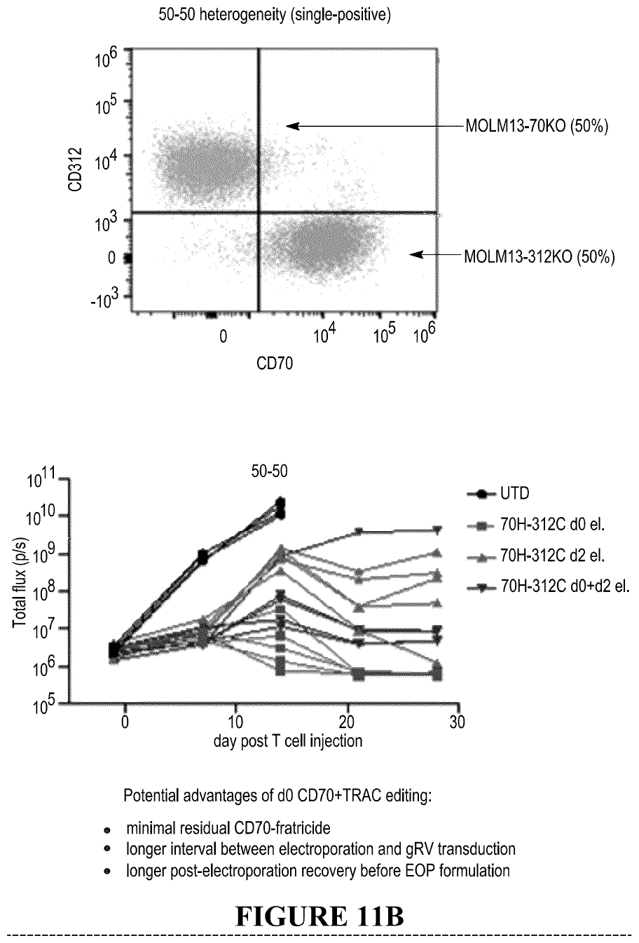

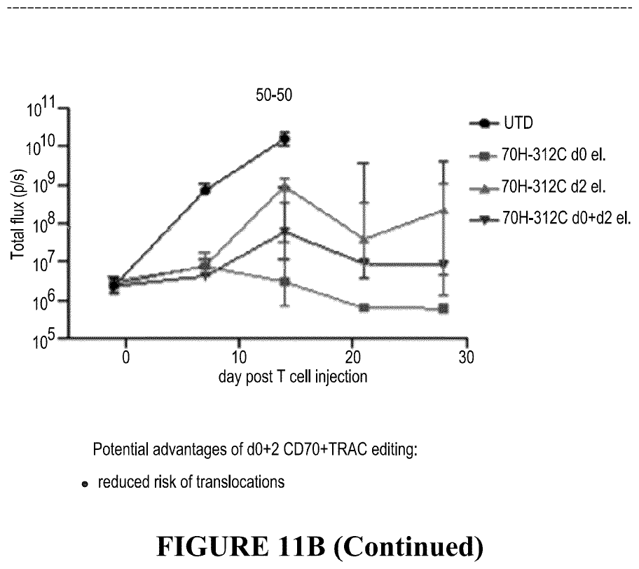

Importantly, as shown in FIGS. 11A and 11B, sequential multiplex editing efficiently eradicated MOLM13 AML xenograft in tumor heterogeneity model compared to multiple editing performed at day 2 (see 70H-312C d0+d2 el. vs. 70H-312C d2 el.). Thus, the delay between editing steps positively impact the HIT+CAR activity.

Overall, the present example shows that multiplex editing at day 0 (e.g., before activation of the T cells) allows i) minimal residual CD70-fratricide killing, ii) longer interval between electroporation and gRV transduction, and iii) longer post-electroporation recovery before EOP formulation. Further, sequential multiplex editing results in reduced risk of unwanted translocations.

### Example 4

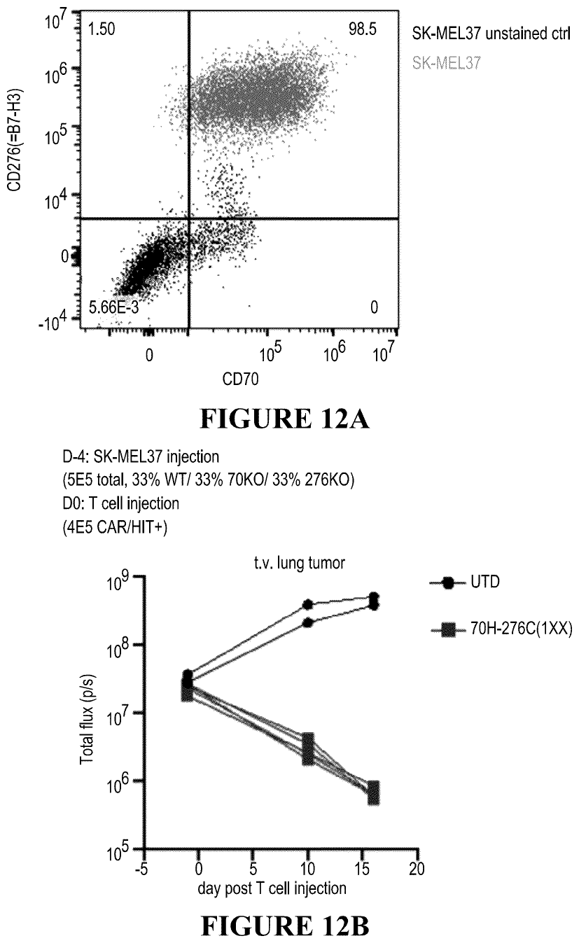

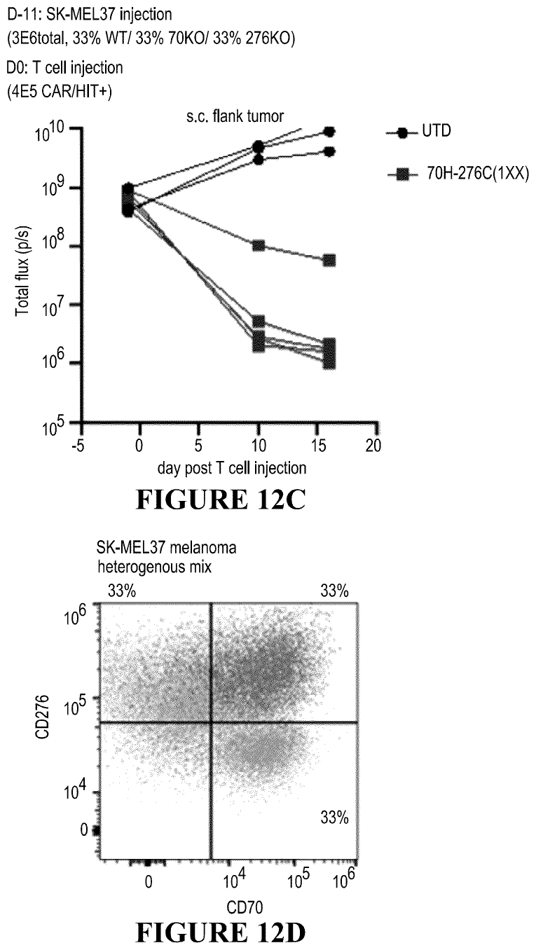

The OR-gated strategy disclosed herein was tested in a melanoma solid tumor model. As shown in FIG. 12A, CD70 and CD276 are both co-expressed in human melanoma cell line SK-MEL37. CD70 was selected as the HIT target because of its low antigen density. CD276 was selected as the CAR target. Notably, a HIT+CAR-28z1XX (CD70-HIT+CD276-28z1XX) was demonstrated to fully eradicate a heterogenous melanoma tumor, both in a lung-metastasized melanoma model as well as in an orthotopic skin melanoma model. See FIGS. 12B-12D.

### Example 5

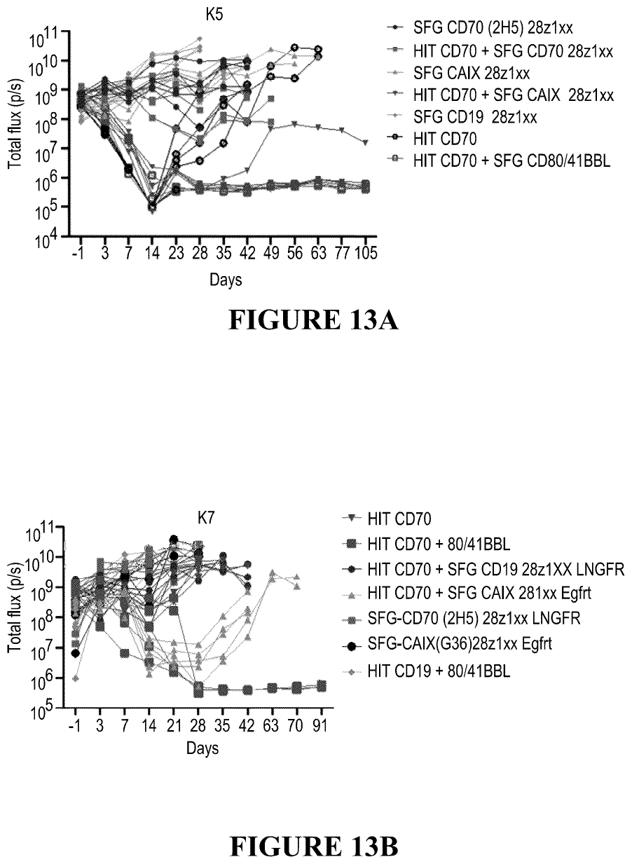

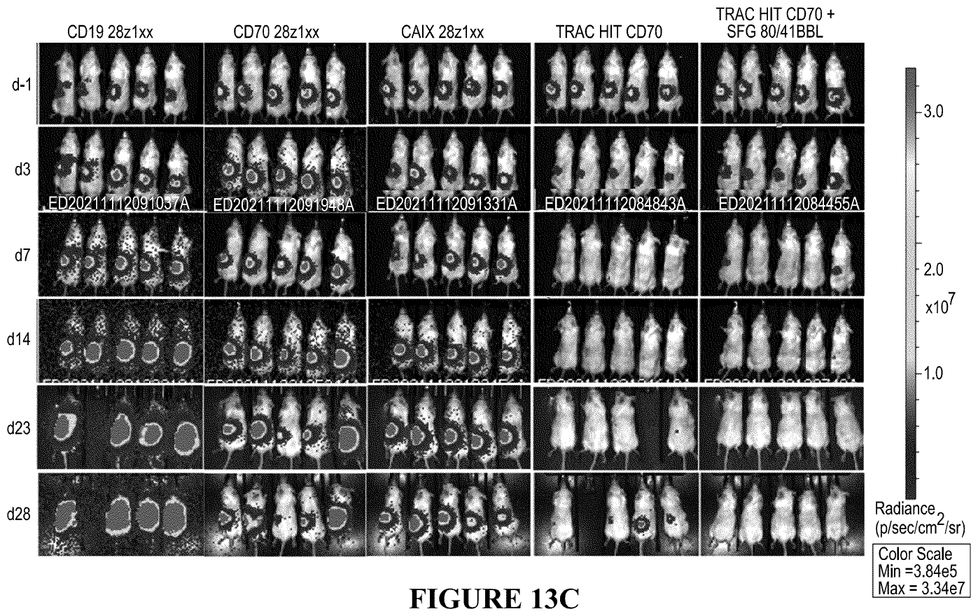

The OR-gated strategy disclosed herein was tested in a renal cell carcinoma tumor model. As shown in FIGS. 13A-13C, T cells including a HIT+CAR-28z1XX targeting CD70 and CAIX (e.g., HITCD70+SFG CAIX-28z1XX) was demonstrated to eradicate a orthotopic kidney tumor. Notably, T cells expressing both HITCD70 and a costimulatory ligand 80/41BBL cleared the tested orthotopic renal cell carcinoma model.

### Example 6

The present example shows that the HIT+CAR strategy disclosed herein is effective in B-ALL settings. To establish tumor heterogeneity for B-ALL, different populations of tumor cells were prepared. Briefly, wild-type Nalm6 cells (WT) were engineered to lack expression of CD19 or CD22 in order to obtain three initial population, as follows:
“WT only” having only wild-type Nalm6 cells;
“1:1” having Nalm6 cells knockout for CD19 (19-KO) and Nalm6 cells knockout for CD22 (22-KO) in a 1:1 ratio; or
“1:1:1” having wild-type Nalm6 cells, Nalm6 cells knockout for CD19 (19-KO), and Nalm6 cells knockout for CD22 (22-KO) in a 1:1:1 ratio.

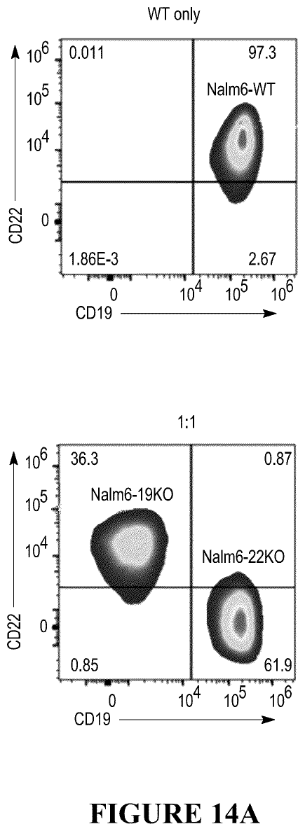

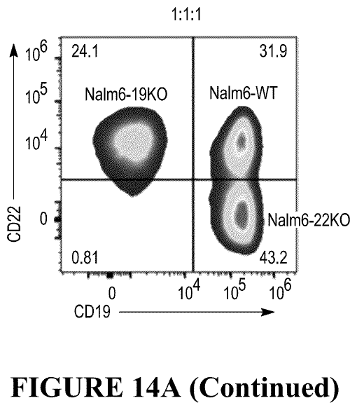

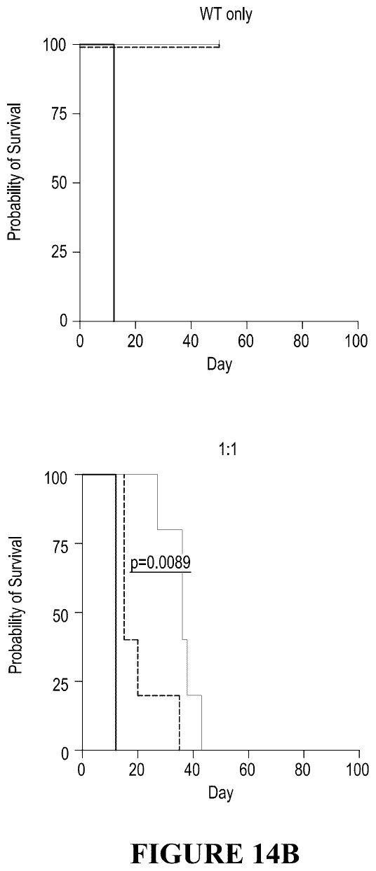

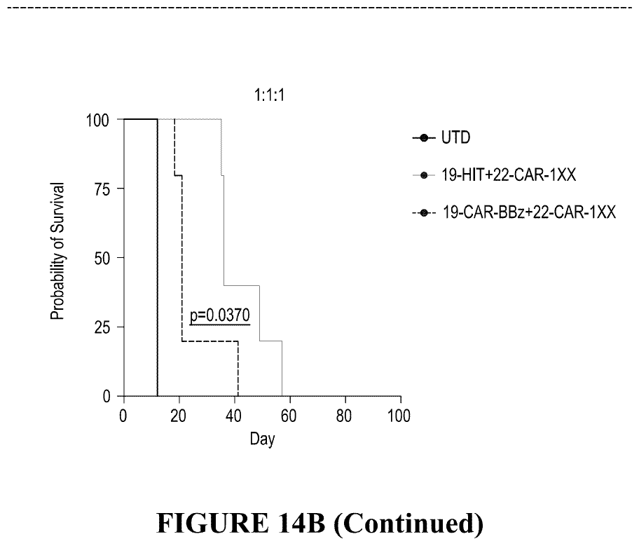

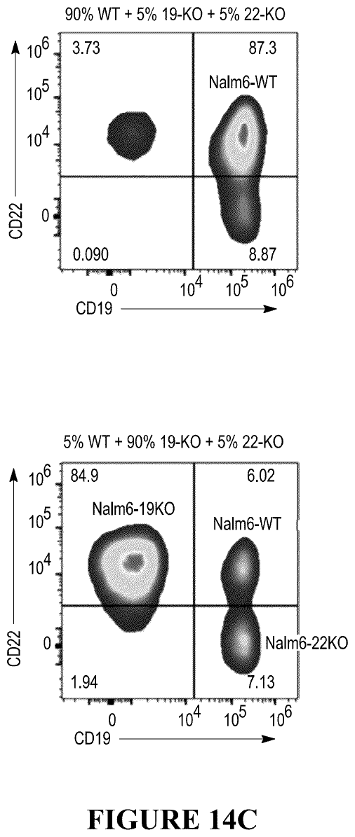

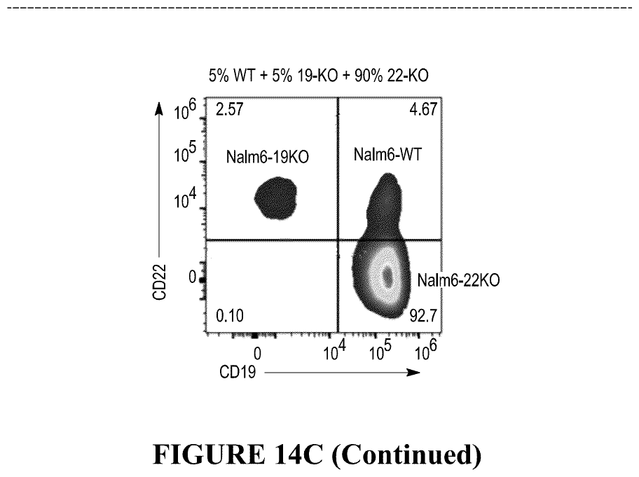

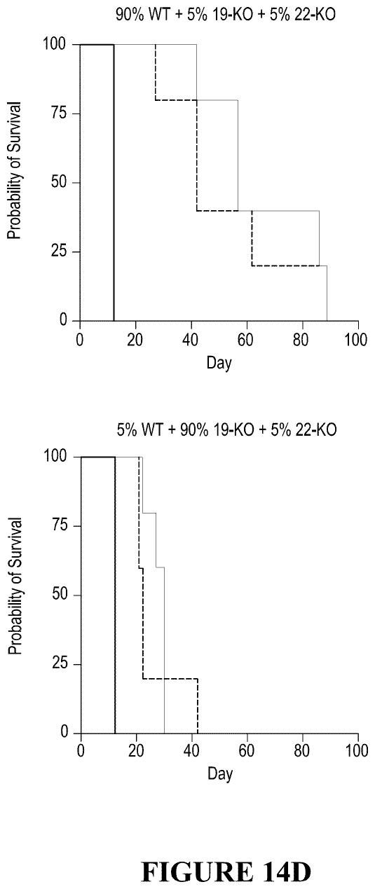

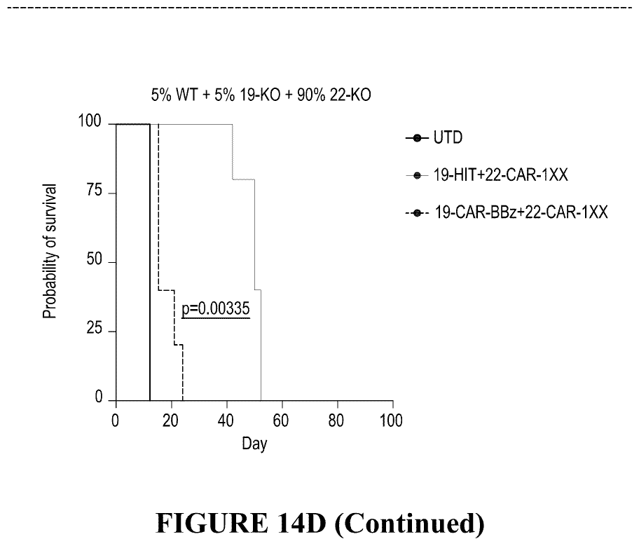

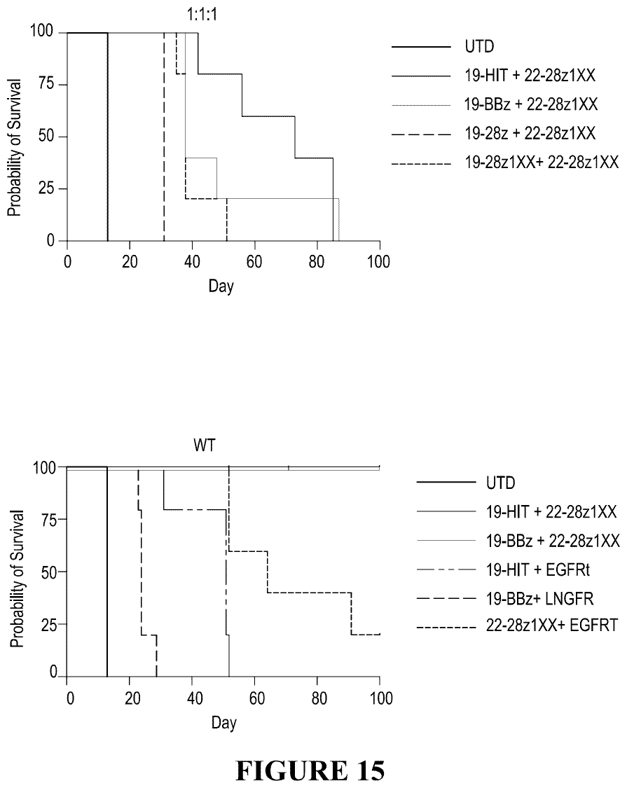

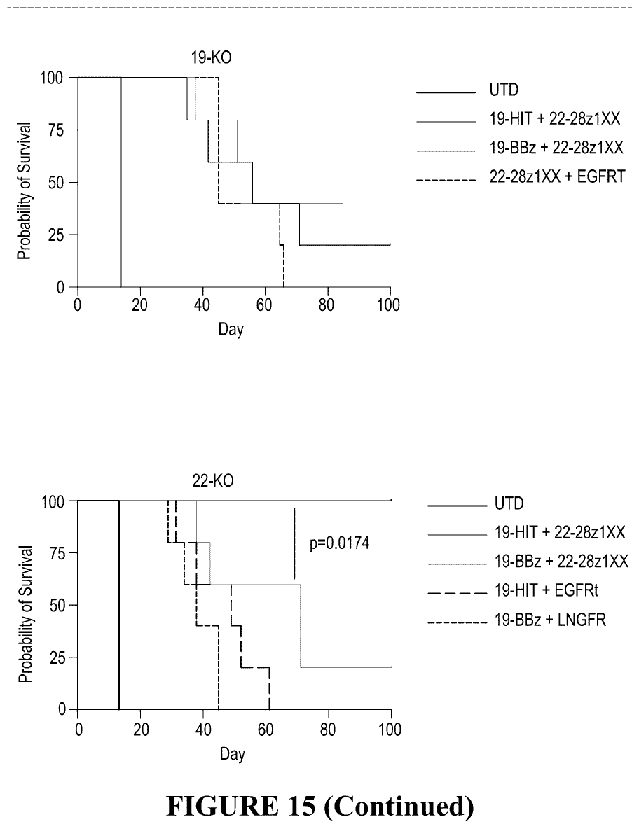

Mice were challenged with the tumor cell populations described herein and received approx. 5E5 T cells expressing i) a HIT targeting CD19 and a CAR targeting CD22 and including a 1XX domain (19-HIT+22-CAR-1XX), or ii) a first CAR targeting CD19 including a 4-1BB/CD3zeta intracellular and a second CAR targeting CD22 including a 1XX domain (19-CAR-BBz+22-CAR-1XX). As shown in FIG. 14B, the 19-HIT+22-CAR-1XX outperformed the 19-CAR-BBz+22-CAR-1XX and significantly increased survival of the animals. Importantly, similar results were obtained when mice were challenged with different tumor populations (FIGS. 14C and 14D). Notably, survival was further increased when mice received higher doses of T cells (FIG. 15).

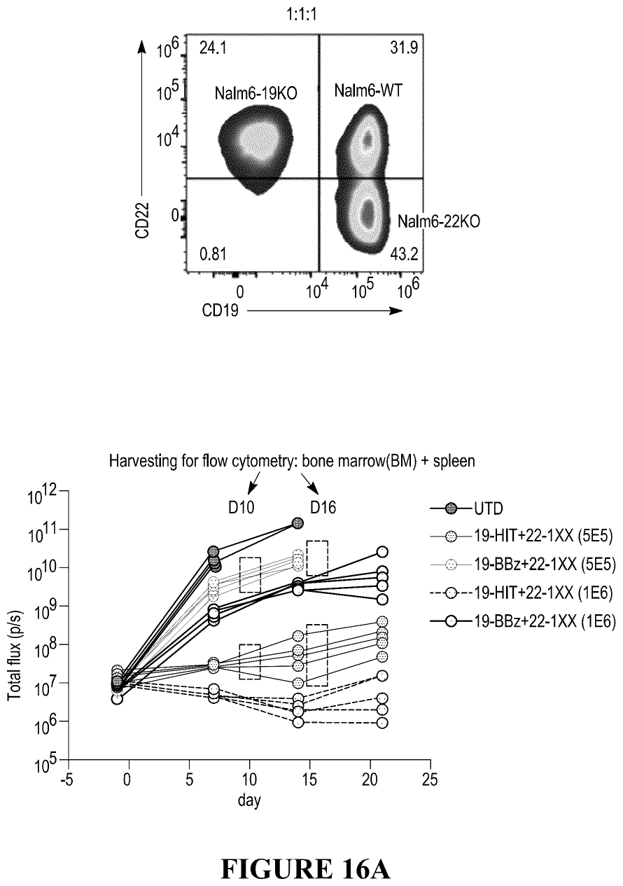

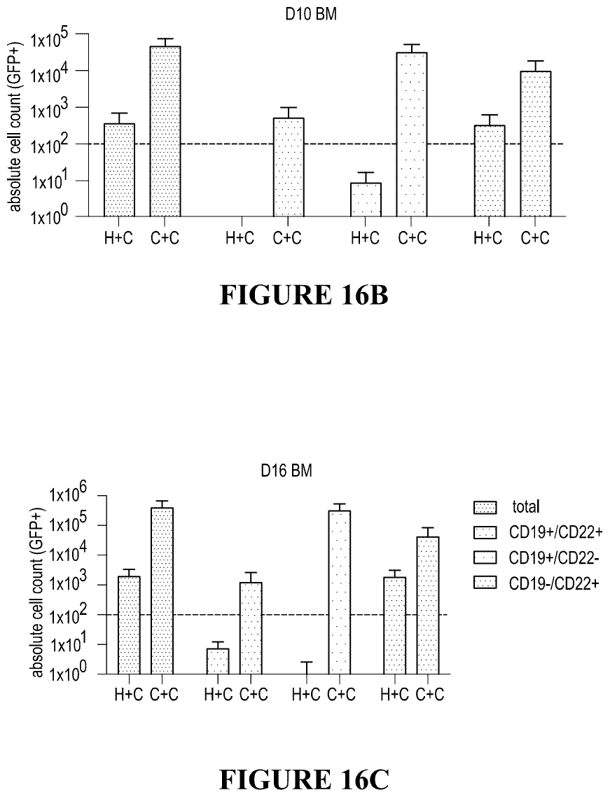

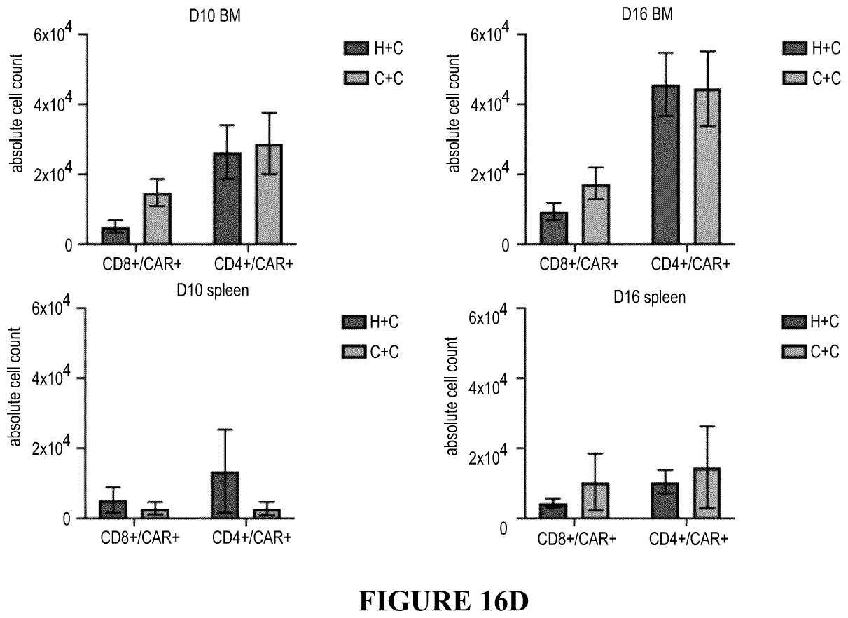

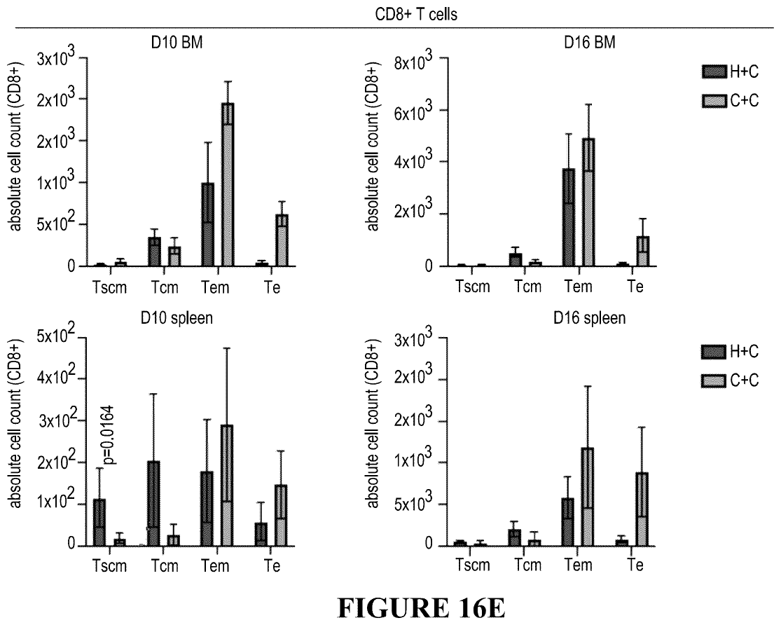

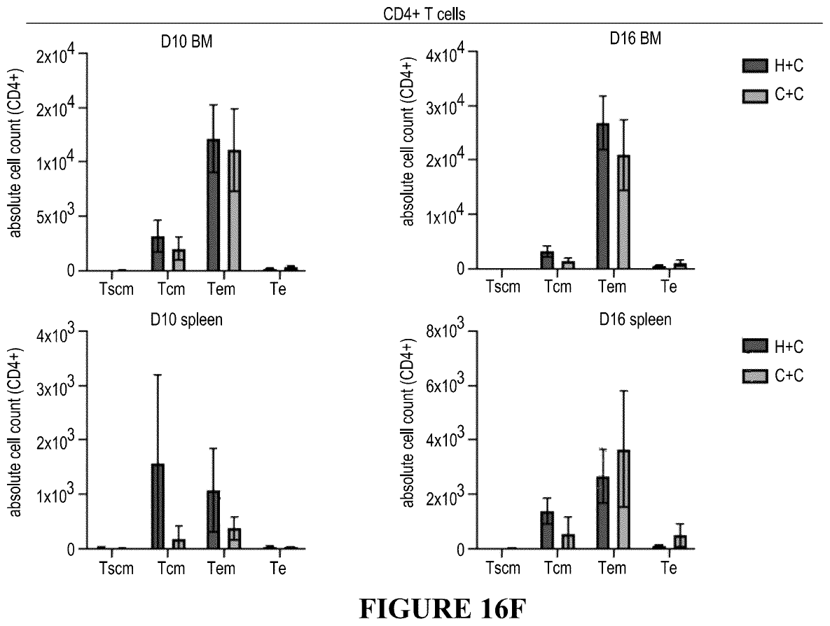

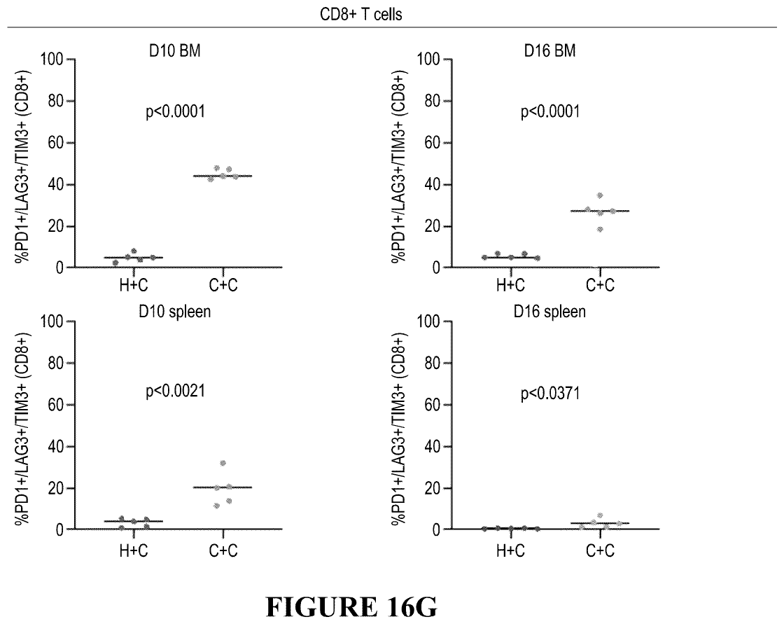

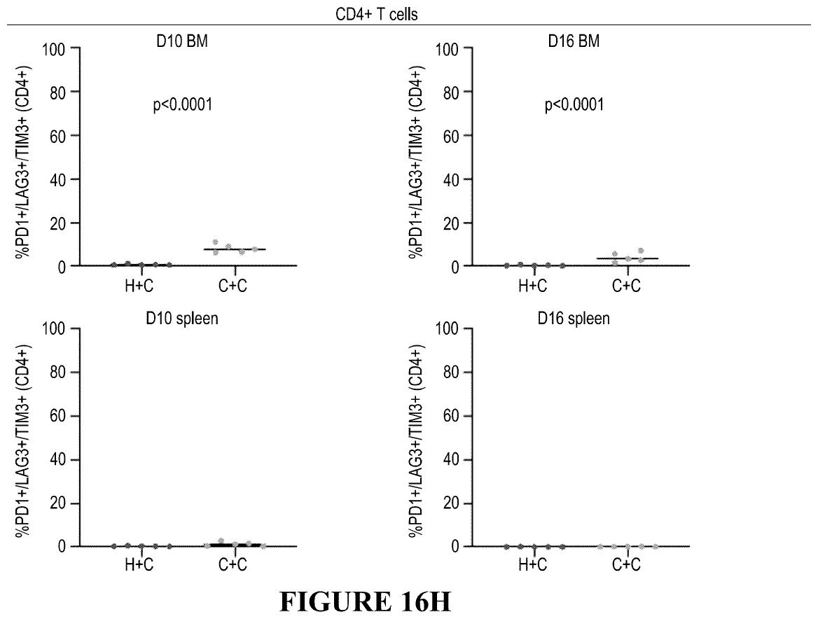

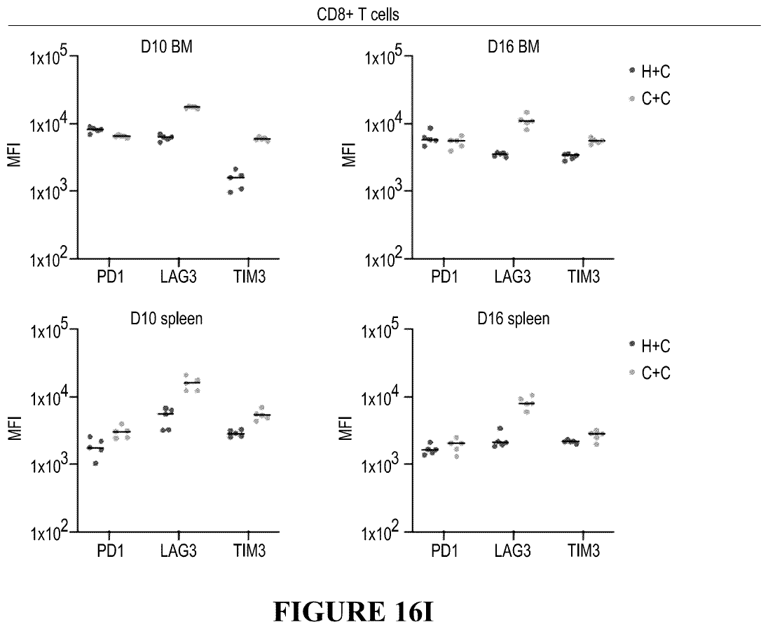

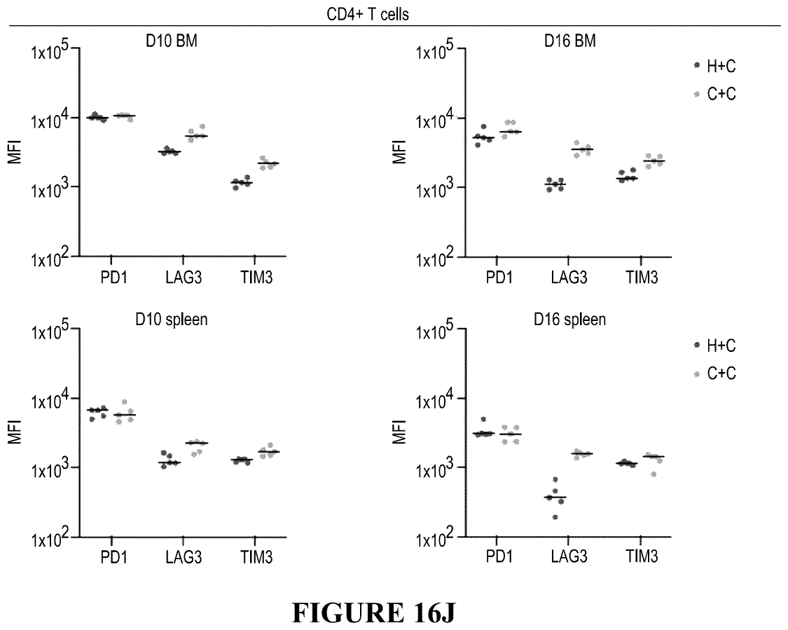

Next, it was investigated whether the 19-HIT+22-CAR-1XX could have an effect on the phenotype of the T cells within the animals. Thus, cells from bone marrow (BM) and spleen of mice challenged with 1:1:1 tumors were collected at days 10 and 16 after administration of different doses of either 19-HIT+22-CAR-1XX or 19-CAR-BBz+22-CAR-1XX. Since 19-HIT+22-CAR-1XX reduced tumor burden (FIG. 16A), the amount of tumor cells in bone marrow were analyzed. As seen in FIGS. 16B and 16C, the amounts of CD19+ and CD22+ cells were significantly reduced after administration of 19-HIT+22-CAR-1XX T cells as compared to 19-CAR-BBz+22-CAR-1XX T cells. Thus, these data indicate that the HIT+CAR strategy reduces the total number of tumor cells as compared to alternative strategies. It was then hypothesized that these differences could be related to functional features of the T cells. The analysis of the amount of T cells in the bone marrow or spleen did not reveal any significant difference (FIG. 16D). However, as seen in FIGS. 16E and 16F, it was observed a significant increase in CD8⁺ T memory stem cells (T_{SCM}), which are a subset of memory lymphocytes endowed with the stem cell-like ability to self-renew and the multipotent capacity to reconstitute the entire spectrum of memory and effector T cell subsets.

T cell exhaustion is a phenomenon widely observed in humans. T_{SCM} or CAR-modified T_{SCM} expresses high levels of PD-1, TIM-3 or CTLA-4 after infiltrating the tumor, indicating that they have become exhausted T cells (Li et al., Frontiers in oncology 11 (2021): 723888). In order to verify whether the T_{SCM} were exhausted, the markers PD1, LAG3, and TIM3 were analyzed in T cells. Surprisingly, the number of CD8 and CD4 T cells that expressed these markers was significantly reduced after administration of 19-HIT+22-CAR-1XX T cells (FIGS. 16G and 16H). Furthermore, the expression levels of these markers were also reduced after administration of 19-HIT+22-CAR-1XX T cells (FIGS. 161 and 16J).

Overall, given the improved survival data associated with less differentiated T cell phenotype and lower expression of exhaustion-associated inhibitory receptors, HIT+CAR is an important strategy for treatment of B-ALL.

### Example 7

It was recently found that CD70 is highly expressed on a substantial fraction of T cells, which may lead to T cell fratricide/suicide and impair efficacy of cells expressing an antigen recognizing receptor targeting CD70. Thus, it is desirable to reduce the expression of CD70 in T cells. In addition, in order to avoid unspecific coupling of between endogenous TCR chain with a recombinant TCR chain (e.g., a first or a second chain of a TCR-like fusion molecule), reduced expression (e.g., knockdown) of a TRAC and/or TRBC gene is also desired.

The present example describes novel strategies for achieving a highly efficient knockdown of CD70, TRAC, and TRBC in T cells. siRNA sequences and corresponding shRNA 97mer sequences were designed using the SplashRNA algorithm (Pelossof et al., Nature Biotechnology. 2017 Apr; 35(4):350-353) and are provided in Table 9 below.

The knockdown results were determined 48h after siRNA transfection (electroporation), via flow cytometric staining of CD3 (for TRAC/TRBC knockdown) and CD70 (for CD70 knockdown). The numeric knockdown values are included in Table 10. Based on these results, shRNA constructs designated as “CD70_2,” “TRAC_5,” and “TRBC_1” (97mer miR-E based shRNA) were identified as candidates for expression using SFG-gammaretroviral vector. Overall, the present example shows that multiplex editing of the T cells allows minimal residual CD70-fratricide killing, and reduced risk of unwanted TCR coupling. [[please confirm]]

TABLE 9
siRNA sequences (validated for knockdown in primary human T cells)

| siRNA/shRNA | | SEQ | | SEQ |
|---|---|---|---|---|
| label | sense strand (5′→3′) | ID NO: | antisense strand (5′→3′) | ID NO: |
| CD70_1 | AUGACAGGUUGAAGCAAGUAGAUA | 161 | UCUACUUGCUUCAACCUGUCAGUG | 166 |
| CD70_2 | CAAGUUCAAGAGAAAAAGUGUAUA | 162 | UACACUUUUUCUCUUGAACUUAUG | 167 |
| CD70_3 | CGCUGCUGAUUAGGGUUUUUUAUA | 163 | UAAAAAACCCUAAUCAGCAGCAUG | 168 |
| CD70_4 | CCGUGAUGGCAUCUACAUGGUAUA | 164 | UACCAUGUAGAUGCCAUCACGAUG | 169 |
| CD70_5 | AAAGAGAAAAAGUGUACACACAUA | 165 | UGUGUGUACACUUUUUCUCUUGUG | 170 |

shRNA sequences (incorporating siRNA sequences (left) and miR-E backbone)

| siRNA/shRNA | | SEQ |
|---|---|---|
| label | 97 mer miR-E shRNA | ID NO |
| CD70_1 | TGCTGTTGACAGTGAGCGATGACAGGTTGAAGCAAGTAGATAGTGAAGCCACAGATGT | 171 |
| | ATCTACTTGCTTCAACCTGTCAGTGCCTACTGCCTCGGA | |
| CD70_2 | TGCTGTTGACAGTGAGCGCAAGTTCAAGAGAAAAAGTGTATAGTGAAGCCACAGATGT | 172 |
| | ATACACTTTTTCTCTTGAACTTATGCCTACTGCCTCGGA | |
| CD70_3 | TGCTGTTGACAGTGAGCGCGCTGCTGATTAGGGTTTTTTATAGTGAAGCCACAGATGT | 173 |
| | ATAAAAAACCCTAATCAGCAGCATGCCTACTGCCTCGGA | |
| CD70_4 | TGCTGTTGACAGTGAGCGCCGTGATGGCATCTACATGGTATAGTGAAGCCACAGATGT | 174 |
| | ATACCATGTAGATGCCATCACGATGCCTACTGCCTCGGA | |
| CD70_5 | TGCTGTTGACAGTGAGCGAAAGAGAAAAAGTGTACACACATAGTGAAGCCACAGATGT | 175 |
| | ATGTGTGTACACTTTTTCTCTTGTGCCTACTGCCTCGGA | |

siRNA sequences (valiated for knockdown in primary human T cells)

| siRNA/shRNA | | SEQ | | SEQ |
|---|---|---|---|---|
| label | sense strand (5′→3′) | ID NO: | ANTISENSE STRAND (5′→3′) | ID NO: |
| TRAC_1 | CUCACCGAUUUUGAUUCUCAAAUA | 176 | UUUGAGAAUCAAAAUCGGUGAAUG | 181 |
| TRAC_2 | AUGGUCGAGAAAAGCUUUGAAAUA | 177 | UUUCAAAGCUUUUCUCGACCAGUG | 182 |
| TRAC_3 | ACGAUUUUGAUUCUCAAACAAAUA | 178 | UUUGUUUGAGAAUCAAAAUCGGUG | 183 |
| TRAC_4 | CUUCACCGAUUUUGAUUCUCAAUA | 179 | UUGAGAAUCAAAAUCGGUGAAUUG | 184 |
| TRAC_5 | AAGAUACGAACCUAAACUUUCAUA | 180 | UGAAAGUUUAGGUUCGUAUCUGUG | 185 |

shRNA sequences (incorporating siRNA sequences (left) and miR-E backbone)

| siRNA/shRNA | | SEQ |
|---|---|---|
| label | 97 mer miR-E shRNA | ID NO |
| TRAC_1 | TGCTGTTGACAGTGAGCGCTCACCGATTTTGATTCTCAAATAGTGAAGCCACAGATGT | 186 |
| | ATTTGAGAATCAAAATCGGTGAATGCCTACTGCCTCGGA | |
| TRAC_2 | TGCTGTTGACAGTGAGCGATGGTCGAGAAAAGCTTTGAAATAGTGAAGCCACAGATGT | 187 |
| | ATTTCAAAGCTTTTCTCGACCAGTGCCTACTGCCTCGGA | |
| TRAC_3 | TGCTGTTGACAGTGAGCGACGATTTTGATTCTCAAACAAATAGTGAAGCCACAGATGT | 188 |
| | ATTTGTTTGAGAATCAAAATCGGTGCCTACTGCCTCGGA | |
| TRAC_4 | TGCTGTTGACAGTGAGCGCTTCACCGATTTTGATTCTCAATAGTGAAGCCACAGATGT | 189 |
| | ATTGAGAATCAAAATCGGTGAATTGCCTACTGCCTCGGA | |
| TRAC_5 | TGCTGTTGACAGTGAGCGAAGATACGAACCTAAACTTTCATAGTGAAGCCACAGATGT | 190 |
| | ATGAAAGTTTAGGTTCGTATCTGTGCCTACTGCCTCGGA | |

siRNA sequences (validated for knockdown in primary human T cells)

| siRNA/shRNA | | SEQ | | SEQ |
|---|---|---|---|---|
| label | sense strand (5′→3′) | ID NO: | antisense strand (5′→3′) | ID NO: |
| TRBC_1 | CCCGCUGUCAAGUCCAGUUCUAUA | 191 | UAGAACUGGACUUGACAGCGGAUG | 197 |
| TRBC_2 | ACGCCCUCAAUGACUCCAGAUAUA | 192 | UAUCUGGAGUCAUUGAGGGCGGUG | 198 |
| TRBC_3 | AACCCGAGGUCGCUGUGUUUGAUA | 193 | UCAAACACAGCGACCUCGGGUGUG | 199 |
| TRBC_4 | ACGCAACCACUUCCGCUGUCAAUA | 194 | UUGACAGCGGAAGUGGUUGCGGUG | 200 |
| TRBC_5 | CGCAGAGAUCUCCCACACCCAAUA | 195 | UUGGGUGUGGGAGAUCUCUGCUUG | 201 |
| TRBC_6 | CCGCUGUGUUUGAGCCAUCAGAUA | 196 | UCUGAUGGCUCAAACACAGCGAUG | 202 |

shRNA sequences (incorporating siRNA sequences (left) and miR-E backbone)

| siRNA/shRNA | | SEQ |
|---|---|---|
| label | 97 mer miR-E shRNA | ID NO |
| TRBC_1 | TGCTGTTGACAGTGAGCGCCCGCTGTCAAGTCCAGTTCTATAGTGAAGCCACAGATGT | 203 |
| | ATAGAACTGGACTTGACAGCGGATGCCTACTGCCTCGGA | |
| TRBC_2 | TGCTGTTGACAGTGAGCGACGCCCTCAATGACTCCAGATATAGTGAAGCCACAGATGT | 204 |
| | ATATCTGGAGTCATTGAGGGCGGTGCCTACTGCCTCGGA | |
| TRBC_3 | TGCTGTTGACAGTGAGCGAACCCGAGGTCGCTGTGTTTGATAGTGAAGCCACAGATGT | 205 |
| | ATCAAACACAGCGACCTCGGGTGTGCCTACTGCCTCGGA | |
| TRBC_4 | TGCTGTTGACAGTGAGCGACGCAACCACTTCCGCTGTCAATAGTGAAGCCACAGATGT | 206 |
| | ATTGACAGCGGAAGTGGTTGCGGTGCCTACTGCCTCGGA | |
| TRBC_5 | TGCTGTTGACAGTGAGCGCGCAGAGATCTCCCACACCCAATAGTGAAGCCACAGATGT | 207 |
| | ATTGGGTGTGGGAGATCTCTGCTTGCCTACTGCCTCGGA | |
| TRBC_6 | TGCTGTTGACAGTGAGCGCCGCTGTGTTTGAGCCATCAGATAGTGAAGCCACAGATGT | 208 |
| | ATCTGATGGCTCAAACACAGCGATGCCTACTGCCTCGGA | |

TABLE 10
(lead candidates bolded and italicized)

| siRNA/shRNA label | % knockdown |
|---|---|
| CD70_1 | −0.49751244 |
| CD70_{—}2 | 67.11442786 |
| CD70_3 | 23.38308458 |
| CD70_4 | 64.47761194 |
| CD70_5 | 55.42288557 |
| TRAC_1 | 4.62962963 |
| TRAC_2 | 8.12757202 |
| TRAC_3 | 2.36625514 |
| TRAC_4 | 23.25102881 |
| TRAC_{—}5 | 24.89711934 |
| TRBC_{—}1 | 58.68886576 |
| TRBC_2 | 7.38813736 |
| TRBC_3 | 9.26118626 |
| TRBC_4 | 29.44849116 |
| TRBC_5 | 26.95109261 |
| TRBC_6 | 25.18210198 |

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A cell comprising:
a) a chimeric antigen receptor (CAR) that targets a first antigen; and
b) a TCR-like fusion molecule that targets a second antigen.

2. The cell of claim 1, wherein the CAR comprises an extracellular antigen-binding domain that binds to the first antigen, and an intracellular signaling domain that is capable of delivering an activation signal to the cell.

3. The cell of claim 2, wherein the intracellular signaling domain of the CAR comprises a CD3ζ polypeptide.

4. The cell of claim 3, wherein the CD3ζ polypeptide is a native CD3ζ polypeptide or a modified CD3ζ polypeptide.

5. The cell of claim 4, wherein the modified CD3ζ polypeptide comprises a native ITAM1, an ITAM2 variant consisting of two loss-of-function mutations, and an ITAM3 variant consisting of two loss-of-function mutations.

6. The cell of claim 4, wherein the modified CD3ζ polypeptide comprises the amino acid sequence set forth in SEQ ID NO: 22.

7. The cell of claim 6, wherein the intracellular signaling domain of the CAR further comprises at least one costimulatory signaling region.

8. The cell of claim 7, wherein the at least one costimulatory signaling region comprises at least an intracellular domain of a co-stimulatory molecule or a portion thereof.

9. The cell of claim 8, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

10. The cell of claim 9, wherein the costimulatory molecule comprises amino acids 180 to 220 of SEQ ID NO: 7.

11. The cell of claim 6, wherein the CAR comprises a transmembrane domain.

12. The cell of claim 6, wherein the TCR-like fusion molecule comprises i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the second antigen, wherein the TCR-like fusion molecule binds to the second antigen in an HLA-independent manner.

13. The cell of claim 12, wherein at least one of the TRAC polypeptide and the TRBC polypeptide is endogenous.

14. The cell of claim 12, wherein the first and the second antigen-binding chains bind to the second antigen with a dissociation constant (KD) of about 1×10⁻⁸ M or less.

15. The cell of claim 14, wherein the first and the second antigen-binding chains bind to the second antigen with a dissociation constant (KD) of about 5×10⁻⁹ M or less.

16. The cell of claim 12, wherein the first antigen-binding chain comprises an antigen-binding fragment of a VH of an antibody and a TRBC polypeptide, and the second antigen-binding chain comprises an antigen-binding fragment of a VL of the antibody and a TRAC polypeptide.

17. The cell of claim 12, wherein the first antigen-binding chain comprises an antigen-binding fragment of a VH of an antibody and a TRAC polypeptide, and the second antigen-binding chain comprises an antigen-binding fragment of a VL of the antibody and a TRBC polypeptide.

18. The cell of claim 12, wherein the first and second antigen binding chains are capable of associating with a CD3ζ polypeptide.

19. The cell of claim 18, wherein the first and second antigen binding chains, upon binding to the second antigen, are capable of activating the CD3ζ polypeptide.

20. The cell of claim 19, wherein the activation of the CD3ζ polypeptide is capable of activating the cell.

21. The cell of claim 12, wherein the cell further comprises a gene disruption of a TRAC locus and/or a TRBC locus.

22. The cell of claim 12, wherein the cell further comprises a gene disruption of a CD70 locus.

23. The cell of claim 12, wherein the cell further comprises a gene disruption of a TRAC locus, a TRBC locus, and/or a CD70 locus.

24. The cell of claim 12, wherein the cell further comprises a gene modification of a TRAC gene and/or a TRBC gene.

25. The cell of claim 12, wherein the cell further comprises a gene modification of a CD70 gene.

26. The cell of claim 12, wherein the cell further comprises a gene modification of a TRAC gene, a TRBC gene, and/or a CD70 gene.

27. The cell of claim 12, wherein the cell is a cell of the lymphoid lineage or a cell of the myeloid lineage.

28. The cell of claim 27, wherein the cell of the lymphoid lineage is selected from the group consisting of a T cell, a B cell, a Natural Killer (NK) cell, and a dendritic cell.

29. The cell of claim 28, wherein the cell is a T cell.

30. The cell of claim 29, wherein the T cell is derived from an induced pluripotent stem cell.

31. The cell of claim 29, wherein the T cell is a CD8⁺ T cell.

32. The cell of claim 31, wherein the CD8⁺ T cell is CD4 independent.

33. The cell of claim 29, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

34. The cell of claim 29, wherein the T cell is CD62L⁺.

35. The cell of claim 29, wherein the T cell is CD45RA⁺.

36. The cell of claim 29, wherein the T cell is CD45RA⁺ and CD62L⁺.

37. The cell of claim 12, wherein the CAR and/or the TCR-like fusion molecule is integrated at a locus within the genome of the T cell.

38. The cell of claim 37, wherein the locus is selected from the group consisting of a TRAC locus, a TRBC locus, a TRDC locus, and a TRGC locus.

39. The cell of claim 37, wherein the locus is a TRAC locus or a TRBC locus.

40. The cell of claim 39, wherein the locus is a TRAC locus.

41. The cell of claim 12, wherein the first antigen and/or the second antigen is a tumor antigen or a pathogen antigen.

42. The cell of claim 41, wherein the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1,
oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

43. The cell of claim 41, wherein the first antigen is selected from the group consisting of CD312, CD19, CD20, CD22, CD276, and CAIX.

44. The cell of claim 43, wherein the second antigen is selected from the group consisting of CD70, CD19, CD20, and CD22.

45. The cell of claim 41, wherein the first antigen and the second antigen are CD312 and CD70.

46. The cell of claim 45, wherein the extracellular antigen-binding domain of the CAR targeting CD312 comprises a V_{H} comprising a CDR1 comprising amino acids having the sequence set forth in SEQ ID NO: 73, a CDR2 comprising the amino acid sequence set forth in SEQ ID NO: 74, and a CDR3 comprising the amino acid sequence set forth in SEQ ID NO: 75; and a V_{L} comprising a CDR1 comprising the amino acid sequence set forth in SEQ ID NO: 76, a CDR2 comprising the amino acid sequence set forth in SEQ ID NO: 77, and a CDR3 comprising the amino acid sequence set forth in SEQ ID NO: 78.

47. The cell of claim 46, wherein the TCR-like fusion molecule targeting CD70 comprises a first antigen binding chain comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and a second antigen binding chain comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138.

48. The cell of claim 38, wherein the first antigen and the second antigen are CD276 and CD70.

49. The cell of claim 48, wherein the extracellular antigen-binding domain of the CAR targeting CD276 comprises a V_{H} comprising a CDR1 comprising amino acids having the sequence set forth in SEQ ID NO: 85, a CDR2 comprising the amino acid sequence set forth in SEQ ID NO: 86, and a CDR3 comprising the amino acid sequence set forth in SEQ ID NO: 87; and a V_{L} comprising a CDR1 comprising the amino acid sequence set forth in SEQ ID NO: 88, a CDR2 comprising the amino acid sequence set forth in SEQ ID NO: 89, and a CDR3 comprising the amino acid sequence set forth in SEQ ID NO: 90.

50. The cell of claim 49, wherein the TCR-like fusion molecule targeting CD70 comprises a first antigen binding chain comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135, and a second antigen binding chain comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138.

51. The cell of claim 41, wherein the first antigen and the second antigen are CAIX and CD70.

52. The cell of claim 41, wherein the first antigen and the second antigen are CD19 and CD22.

53. The cell of claim 41, wherein the first antigen and the second antigen are CD19 and CD20.

54. The cell of claim 41, wherein the first antigen and the second antigen are CD20 and CD22.

55. The cell of claim 41, wherein the first antigen and the second antigen are CD20 and CD19.

56. The cell of claim 41, wherein the first antigen and the second antigen are CD22 and CD20.

57. The cell of claim 41, wherein the first antigen and the second antigen are CD22 and CD19.

58. The cell of claim 57, wherein the extracellular antigen-binding domain of the CAR targeting CD22 comprises:
a) a V_{H} comprising a CDR1 comprising amino acids having the sequence set forth in SEQ ID NO: 97, a CDR2 comprising the amino acid sequence set forth in SEQ ID NO: 98, and a CDR3 comprising the amino acid sequence set forth in SEQ ID NO: 99; and a V_{L} Comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102;
b) a V_{H} comprising a CDR1 comprising amino acids having the sequence set forth in SEQ ID NO: 97, a CDR2 comprising the amino acid sequence set forth in SEQ ID NO: 153, and a CDR3 comprising the amino acid sequence set forth in SEQ ID NO: 99; and a V_{L} Comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102;
c) a V_{H} comprising a CDR1 comprising amino acids having the sequence set forth in SEQ ID NO: 119, a CDR2 comprising the amino acid sequence set forth in SEQ ID NO: 120, and a CDR3 comprising the amino acid sequence set forth in SEQ ID NO: 121; and a V_{L} Comprising a CDR1 comprising the amino acid sequence set forth in SEQ ID NO: 122, a CDR2 comprising the amino acid sequence set forth in SEQ ID NO: 123, and a CDR3 comprising the amino acid sequence set forth in SEQ ID NO: 124; or
d) a V_{H} comprising a CDR1 comprising amino acids having the sequence set forth in SEQ ID NO: 97, a CDR2 comprising the amino acid sequence set forth in SEQ ID NO: 154, and a CDR3 comprising the amino acid sequence set forth in SEQ ID NO: 99; and a V_{L} Comprising a CDR1 comprising the amino acid sequence set forth in SEQ ID NO: 100, a CDR2 comprising the amino acid sequence set forth in SEQ ID NO: 101, and a CDR3 comprising the amino acid sequence set forth in SEQ ID NO: 102.

59. The cell of claim 58, wherein the TCR-like fusion molecule targeting CD19 comprises a first antigen binding chain comprising a CDR1 comprising the amino acid sequence set forth in SEQ ID NO: 143, a CDR2 comprising the amino acid sequence set forth in SEQ ID NO: 144, and a CDR3 comprising the amino acid sequence set forth in SEQ ID NO: 145, and a second antigen binding chain comprising a CDR1 comprising the amino acid sequence set forth in SEQ ID NO: 146, a CDR2 comprising the amino acid sequence set forth in SEQ ID NO: 147, and a CDR3 comprising the amino acid sequence set forth in SEQ ID NO: 148.

60. The cell of claim 41, wherein the first antigen and the second antigen are selected from Table 8.

61. The cell of claim 12, further comprising a chimeric co-stimulating receptor (CCR).

62. The cell of claim 61, wherein the CCR comprises an extracellular antigen-binding domain that binds to the third antigen and an intracellular domain that is capable of delivering a costimulatory signal to the cell but does not alone deliver an activation signal to the cell.

63. The cell of claim 62, wherein the intracellular domain of the CCR comprises at least an intracellular domain of a co-stimulatory molecule or a portion thereof.

64. The cell of claim 63, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

65. The cell of claim 12, wherein the cell further comprises at least one exogenous costimulatory ligand.

66. The cell of claim 65, wherein the at least one exogenous co-stimulatory ligand is selected from the group consisting of a tumor necrosis factor (TNF) family member, an immunoglobulin (Ig) superfamily member, and combinations thereof.

67. The cell of claim 66, wherein the TNF family member is selected from the group consisting of 4-1BBL, OX40L, CD70, FasL, GITRL, TNF-related apoptosis-inducing ligand (TRAIL), CD30L, LIGHT (TNFSF14), CD40L.

68. The cell of claim 66, wherein the Ig superfamily member is selected from the group consisting of CD80, CD86, ICOSLG, and combinations thereof.

69. The cell of claim 65, wherein the at least one exogenous costimulatory ligand comprises CD80.

70. The cell of claim 65, wherein the at least one exogenous a costimulatory ligand comprises 4-1BBL.

71. The cell of claim 65, wherein the cell comprises two exogenous costimulatory ligands.

72. The cell of claim 71, wherein the at least two exogenous costimulatory ligands comprise CD80 and 4-1BBL.

73. The cell of claim 72, wherein the at least two exogenous costimulatory ligands comprise the amino acid sequence set forth in SEQ ID NO: 67 and/or the amino acid sequence set forth in SEQ ID NO: 69.

74. The cell of claim 12, wherein the cell further comprises a fusion polypeptide comprising:
a) an extracellular domain and a transmembrane domain of a co-stimulatory ligand, and b) an intracellular domain of a first co-stimulatory molecule.

75. The cell of claim 74, wherein the co-stimulatory ligand is selected from the group consisting of a tumor necrosis factor (TNF) family member, an immunoglobulin (Ig) superfamily member, and combinations thereof.

76. The cell of claim 75, wherein the TNF family member is selected from the group consisting of 4-1BBL, OX40L, CD70, GITRL, CD40L, and combinations thereof.

77. The cell of claim 75, wherein the Ig superfamily member is selected from the group consisting of CD80, CD86, ICOSLG, and combinations thereof.

78. The cell of claim 74, wherein the co-stimulatory ligand is CD80.

79. The cell of claim 74, wherein the first co-stimulatory molecule is selected from the group consisting of CD28, 4-1BB, OX40, ICOS, DAP-10, CD27, CD40, NKG2D, CD2, and combinations thereof.

80. The cell of claim 79, wherein the first co-stimulatory molecule is 4-1BB.

81. The cell of claim 74, wherein the co-stimulatory ligand is CD80 and the first co-stimulatory molecule is 4-1BB.

82. The cell of claim 81, wherein the fusion polypeptide comprises the amino acid sequence set forth in SEQ ID NO: 71.

83. The cell of claim 74, wherein the fusion polypeptide further comprises an intracellular domain of a second co-stimulatory molecule.

84. The cell of claim 83, wherein the second co-stimulatory molecule is selected from the group consisting of CD28, 4-1BB, OX40, ICOS, DAP-10, CD27, CD40, NKG2D, CD2, and combinations thereof.

85. The cell of claim 83, wherein the second co-stimulatory molecule is CD28.

86. The cell of claim 74, wherein the co-stimulatory ligand is CD80, the first co-stimulatory molecule is 4-1BB, and the second co-stimulatory molecule is CD28.

87. The cell of claim 86, wherein the fusion polypeptide comprises the amino acid sequence set forth in SEQ ID NO: 72.

88. The cell of any one of claims 1-87, wherein the cell is autologous.

89. The cell of any one of claims 1-87, wherein the cell is allogeneic.

90. A composition comprising the cell of any one of claims 1-89.

91. The composition of claim 90, which is a pharmaceutical composition further comprising a pharmaceutically acceptable excipient.

92. A nucleic acid composition comprising a first polynucleotide encoding a chimeric antigen receptor (CAR) that targets a first antigen and a second polynucleotide encoding a TCR-like fusion molecule that targets a second antigen.

93. A vector comprising a first polynucleotide encoding a chimeric antigen receptor (CAR) that targets a first antigen and a second polynucleotide encoding a TCR-like fusion molecule that targets a second antigen.

94. The vector of claim 93, wherein the vector is a lentiviral vector.

95. The vector of claim 93 or 94, wherein the vector is a y-retroviral vector.

96. A lipid nanoparticle comprising a first polynucleotide encoding a chimeric antigen receptor (CAR) that targets a first antigen and a second polynucleotide encoding a TCR-like fusion molecule that targets a second antigen.

97. A polynucleotide encoding a chimeric antigen receptor (CAR) that targets a first antigen and a TCR-like fusion molecule that targets a second antigen.

98. A vector comprising the polynucleotide of claim 97.

99. The vector of claim 98, wherein the vector is a lentiviral vector.

100. The vector of claim 98 or 99, wherein the vector is a y-retroviral vector.

101. A lipid nanoparticle comprising the polynucleotide of claim 97.

102. A composition comprising the polynucleotide of claim 97, the vector of any one of claims 93-95 or 98-100, or the lipid nanoparticle of claim 96 or 101.

103. The composition of claim 102, which is a pharmaceutical composition further comprising a pharmaceutically acceptable excipient.

104. A method for producing a cell of any one of claims 1-89, the method comprising introducing into the cell the nucleic acid composition of claim 92, a vector of any one of claims 93-95 or 98-100, a polynucleotide of claim 97, a lipid nanoparticle of claim 96 or 101, or a composition of claim 102 or 103.

105. The method of claim 104, further comprising generating a gene disruption of a CD70 locus.

106. The method of claim 104 or 105, further comprising generating a gene disruption of a TRAC locus.

107. The method of claim 105 or 106, wherein the gene disruption comprises a substitution, a deletion, an insertion, a mutation, or a combination thereof.

108. The method of claim 107, wherein the mutation comprises a missense mutation, a nonsense mutation, or a combination thereof.

109. The method of claim 107, wherein the deletion comprises a non-frameshift deletion, a frameshift deletion, or a combination thereof.

110. The method of claim 107, wherein the insertion comprises a non-frameshift insertion, a frameshift insertion, or a combination thereof.

111. The method of any one of claims 104-110, wherein the gene disruption of the CD70 locus results in a non-functional CD70 protein or in knockout of the CD70 gene expression.

112. The method of any one of claims 104-111, wherein the gene disruption of the TRAC locus results in a non-functional TRAC protein or in knockout of the TRAC gene expression.

113. The method of any one of claims 104-112, wherein generating the gene disruption of comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

114. The method any one of claims 104-113, wherein the gene disruption of a CD70 locus or a TRAC locus is generated before activation of a cell.

115. The method any one of claims 104-114, wherein the gene disruption of a CD70 locus or a TRAC locus is generated after activation of a cell.

116. The method any one of claims 104-114, wherein a) the gene disruption of a CD70 locus is generated before activation of a cell, and b) the gene disruption of a TRAC locus is generated after activation of a cell.

117. The method any one of claims 104-114, wherein a) the gene disruption of a TRAC locus is generated before activation of a cell, and b) the gene disruption of a CD70 locus is generated after activation of a cell.

118. The method of claim 104, further comprising generating a gene modification of a CD70 gene.

119. The method of claim 104 or 118, further comprising generating a gene modification of a TRAC gene and/or a TRBC gene.

120. The method of claim 118 or 119, wherein the gene modification of the CD70 gene results in a non-functional CD70 protein or in knockdown of the CD70 gene expression.

121. The method of any one of claims 118-120, wherein the gene modification of the TRAC locus results in a non-functional TRAC protein or in knockdown of the TRAC gene expression.

122. The method of any one of claims 118-121, wherein the gene modification of the TRBC locus results in a non-functional TRBC protein or in knockdown of the TRBC gene expression.

123. The method of any one of claims 104-122, further comprising introducing a chimeric co-simulating receptor (CCR).

124. The method of any one of claims 104-123, further comprising introducing at least one exogenous costimulatory ligand.

125. The method of any one of claims 104-124, further comprising introducing a fusion polypeptide comprising: a) an extracellular domain and a transmembrane domain of a co-stimulatory ligand, and b) an intracellular domain of a first co-stimulatory molecule.

126. A cell produced by the method of any one of claims 104-125.

127. A method of reducing tumor burden in a subject, the method comprising administering to the subject an effective amount of the cells of any one of claims 1-89 or 126, or the composition of claim 90 or 91.

128. The method of claim 127, wherein the method reduces the number of tumor cells, reduces tumor size, and/or eradicates the tumor in the subject.

129. A method of preventing and/or treating a neoplasm or a tumor in the subject, administering to the subject an effective amount of the cells of any one of claims 1-89 or 126, or the composition of claim 90 or 91.

130. The method of any one of claims 127-129, wherein the neoplasm or tumor is cancer.

131. The method of any one of claims 127-130, wherein the neoplasm or tumor comprises antigen heterogeneity of the first antigen and the second antigen.

132. The method of claim 131, wherein the second antigen has a low antigen density.

133. The method of claim 131 or 132, wherein the second antigen is expressed on tumor cells having a low tumor cell frequency.

134. The method of any one of claims 131-133, wherein the first antigen and the second antigen are independently selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

135. The method of any one of claims 131-134, wherein the first antigen is selected from the group consisting of CD312, CD19, CD20, CD22, CD276, and CAIX.

136. The method of any one of claims 131-134, wherein the second antigen is selected from the group consisting of CD70, CD19, CD20, and CD22.

137. The method of any one of claims 131-134, wherein the first antigen and the second antigen are selected from a) CD312 and CD70; b) CD276 and CD70; c) CAIX and CD70; d) CD19 and CD22; e) CD19 and CD20; f) CD20 and CD22; g) CD20 and CD19; h) CD22 and CD20; or i) CD22 and CD19.

138. The method of any one of claims 131-134, wherein the first antigen and the second antigen are selected from Table 8.

139. The method of any one of claims 127-138, wherein the neoplasm or tumor is a solid tumor.

140. The method of claim 139, wherein the solid tumor is selected from the group consisting of melanoma, renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

141. The method of claim 140, wherein the solid tumor is melanoma.

142. The method of any one of claims 127-138, wherein the neoplasm or tumor is a blood cancer.

143. The method of any one of claims 127-138 or 142, wherein the neoplasm or tumor is a myeloid disorder.

144. The method of claim 143, wherein the myeloid disorder is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

145. The method of claim 144, wherein the myeloid disorder is acute myeloid leukemia (AML).

146. The method of any one of claims 127-138 or 142, wherein the neoplasm or tumor is a B-cell malignancy.

147. The method of claim 146, wherein the B-cell malignancy is selected from the group consisting of B cell non-Hodgkin lymphoma (NHL), B cell Hodgkin's lymphoma, B cell acute lymphocytic leukemia (ALL), B cell chronic lymphocytic leukemia (CLL), multiple myeloma (MM), CLL with Richter's transformation, and CNS lymphoma.

148. The method of claim 147, wherein the B-cell malignancy is B cell acute lymphocytic leukemia.

149. The method of any one of claims 127-138 or 142, wherein the neoplasm or tumor is a leukemia.

150. The method of claim 149, wherein the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, B-cell precursor acute lymphoblastic leukemia, and T-cell precursor acute lymphoblastic leukemia.

151. The method of any one of claims 127-138 or 142, wherein the neoplasm or tumor is a lymphoma.

152. The method of claim 151, wherein the lymphoma is selected from the group consisting of Hodgkin's lymphoma, non-Hodgkin's lymphoma, B-cell non-Hodgkin's lymphoma, T-cell non-Hodgkin's lymphoma, and T-cell precursor acute lymphoblastic lymphoma.

153. The method of any one of claims 127-152, wherein the subject has a relapse of the neoplasm or tumor.

154. The method of any one of claims 127-153, wherein the subject received treatment which leads to residual tumor cells.

155. A method of preventing and/or treating a pathogen infection in a subject, the method comprising administering to the subject an effective amount of the cells of any one of claims 1-89 or 126, or the composition of claim 90 or 91.

156. A method of preventing and/or treating an autoimmune disease in a subject, the method comprising administering to the subject an effective amount of the cells of any one of claims 1-89 or 126, or the composition of claim 90 or 91.

157. A method of preventing and/or treating an infectious disease in a subject, the method comprising administering to the subject an effective amount of the cells of any one of claims 1-89 or 126, or the composition of claim 90 or 91.

158. The cells of any one of claims 1-89 or 126, or the composition of claim 90 or 91 for use in reducing tumor burden, treating and/or preventing a neoplasm or a tumor, preventing and/or treating a pathogen infection, preventing and/or treating an autoimmune disease, and/or preventing and/or treating an infectious disease, in a subject.

159. A kit comprising the cell of any one of claims 1-89 or 126, or the composition of claim 90 or 91.

160. The kit of claim 159, wherein the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.


SEQUENCE LISTING (208 sequences)

SEQ ID NO: 1 - protein, 15 aa, synthetic construct
GGGGSGGGGSGGGGS

SEQ ID NO: 2 - protein, 19 aa, synthetic construct
GGGGSGGGGSGGGSGGGGS

SEQ ID NO: 3 - protein, 24 aa, synthetic construct
GGGGSGGGGSGGGGSGGGSGGGGS

SEQ ID NO: 4 - protein, 29 aa, synthetic construct
GGGGSGGGGSGGGGSGGGGSGGGSGGGGS

SEQ ID NO: 5 - protein, 5 aa, synthetic construct
GGGGS

SEQ ID NO: 6 - protein, 10 aa, synthetic construct
GGGGSGGGGS

SEQ ID NO: 7 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 22 aa, synthetic construct
QNQLYNELNLGRREEYDVLDKR

SEQ ID NO: 11 - DNA, 66 bp, synthetic construct
  source 1..66: mol_type = other DNA
cagaaccagctctataacgagctcaatctaggacgaagagaggagtacgatgttttggacaagaga

SEQ ID NO: 12 - protein, 22 aa, synthetic construct
QNQLFNELNLGRREEFDVLDKR

SEQ ID NO: 13 - DNA, 66 bp, synthetic construct
  source 1..66: mol_type = other DNA
cagaaccagctctttaacgagctcaatctaggacgaagagaggagttcgatgttttggacaagaga

SEQ ID NO: 14 - protein, 23 aa, synthetic construct
QEGLYNELQKDKMAEAYSEIGMK

SEQ ID NO: 15 - DNA, 69 bp, synthetic construct
  source 1..69: mol_type = other DNA
caggaaggcctgtacaatgaactgcagaaagataagatggcggaggcctacagtgagattgggatgaaa

SEQ ID NO: 16 - protein, 23 aa, synthetic construct
QEGLFNELQKDKMAEAFSEIGMK

SEQ ID NO: 17 - DNA, 69 bp, synthetic construct
  source 1..69: mol_type = other DNA
caggaaggcctgttcaatgaactgcagaaagataagatggcggaggccttcagtgagattgggatgaaa

SEQ ID NO: 18 - protein, 22 aa, synthetic construct
HDGLYQGLSTATKDTYDALHMQ

SEQ ID NO: 19 - DNA, 66 bp, synthetic construct
  source 1..66: mol_type = other DNA
cacgatggcctttaccagggtctcagtacagccaccaaggacacctacgacgcccttcacatgcag

SEQ ID NO: 20 - protein, 22 aa, synthetic construct
HDGLFQGLSTATKDTFDALHMQ

SEQ ID NO: 21 - DNA, 66 bp, synthetic construct
  source 1..66: mol_type = other DNA
cacgatggccttttccaggggctcagtacagccaccaaggacaccttcgacgcccttcacatgcag

SEQ ID NO: 22 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 23 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 24 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 25 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 26 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 27 - DNA, 657 bp, synthetic construct
  source 1..657: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 28 - DNA, 657 bp, synthetic construct
  source 1..657: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 29 - DNA, 657 bp, synthetic construct; identical to SEQ ID NO: 28
  source 1..657: mol_type = other DNA

SEQ ID NO: 30 - DNA, 675 bp, synthetic construct
  source 1..675: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 31 - DNA, 675 bp, synthetic construct
  source 1..675: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagtttagccggagtgccgatgctccagcctatcagcaaggacaaaaccaactttacaacgaacttaatctcggtaggcgcgaggaatacgatgtgcttgataaacgccggggtcgcgatcccgaaatgggcgggaaacctcggcgcaagaatcctcaagaaggactctataatgagttgcagaaggacaagatggctgaggcatatagtgagatcggtatgaagggagaacggagaagggggaaagggcatgatggattgtaccaaggacttagcacagctactaaagatacatatgacgccctgcacatgcaagcattgcctccacgc

SEQ ID NO: 32 - protein, 20 aa, Homo sapiens
MYRMQLLSCIALSLALVTNS

SEQ ID NO: 33 - protein, 20 aa, Mus sp.
MYSMQLASCVTLTLVLLVNS

SEQ ID NO: 34 - protein, 20 aa, Homo sapiens
METPAQLLFLLLLWLPDTTG

SEQ ID NO: 35 - protein, 20 aa, Mus sp.
METDTLLLWVLLLWVPGSTG

SEQ ID NO: 36 - protein, 21 aa, Homo sapiens
MALPVTALLLPLALLLHAARP

SEQ ID NO: 37 - protein, 18 aa, Homo sapiens
MALPVTALLLPLALLLHA

SEQ ID NO: 38 - protein, 16 aa, Homo sapiens
MKWVTFISLLFSSAYS

SEQ ID NO: 39 - protein, 30 aa, Homo sapiens
MDSKGSSQKGSRLLLLLVVSNLLLCQGVVS

SEQ ID NO: 40 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 41 - DNA, 420 bp, Homo sapiens
  source 1..420: mol_type = genomic DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 42 - protein, 143 aa, Homo sapiens
IPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 43 - DNA, 429 bp, Homo sapiens
  source 1..429: mol_type = genomic DNA
attcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 44 - DNA, 4627 bp, Homo sapiens
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 45 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 46 - DNA, 534 bp, Homo sapiens
  source 1..534: mol_type = genomic DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 47 - protein, 180 aa, Homo sapiens
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 48 - DNA, 540 bp, Homo sapiens
  source 1..540: mol_type = genomic DNA
ctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 49 - protein, 175 aa, Homo sapiens
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 50 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 51 - DNA, 528 bp, Homo sapiens
  source 1..528: mol_type = genomic DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 52 - DNA, 1448 bp, Homo sapiens
  source 1..1448: mol_type = genomic DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 53 - DNA, 1489 bp, Homo sapiens
  source 1..1489: mol_type = genomic DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 54 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 55 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 56 - DNA, 5591 bp, Homo sapiens
  source 1..5591: mol_type = genomic DNA
ataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacaggtatgtgtttacgcatatcatctgtcagaacacttctttgaaagtgaatgctgcattttttcctttcagtattaatgaaaaacaaacataaatctttcttaaatattgttacatttaatggtagcataaatgccctgctacttttctatagaattaaaatggtataggttttggagaaaacaaaattgaaaaagttactgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaatgatgtttatctgttgttgacagaatgatgtttatctaaaaacagttccaattttctatttcctttgctgagacacaaaggggaggcaaatgtgcaaagcttgagggtagtcttaccactgtgcttaagtgttctgatttttctagtgatcagggcaaaataaaaagtatagtaagttccaaggcagtgaatattatacaggagagaagttacagttttataatgtgttttcctttacactaaattctaaaagtaaaaagtctttttttttttttgacagagtttcactcttgttgcccaagcaggtgtgctatggtatgatctcagctcactgcaacctccacctcccgggttcaagtgattctcttacttcagcctcccgacaggctgggattgcaggcgcctgccaccacacctggctaatttttgtgtttttagtagagatggggtttcaccatgttggccaggctggtctcaaattcctgacctcaagtgatccatccacctcggcctccaagtgctgggattatgggcgtcagccactgtgcccagcctaaaagtaaaatgtctttcatgagcttcccaaggcagctacgttaaggaggacacttctcttaatgtcattctacagtagatttctaatgctctttcttggaagtttgtttttctgagaaaagctaaaaatataacatggaagtgatcatattatataatcaatgaagtgcttttcaaggagataaaactaatctggtccacacttgcaaccaaccttgattgagagagagagagaactcaggatacacttgaagattttattatggggaacagttactttattctttttacctcaatcaatgcatggaaataagtgatagtcattttcatttatcttttaataaatgaagtcaccatgaggaaaataaaaagacattgaaaacccattaaagtcagcccttaaagatatttggacatgcagacttgataactaacgtttgcattcttgagacttacccaaaacccatacctcaagtccaagtttttagaattcatgaaataaagatctcagtgagtgcataaaattgcgcaccagaatcatatccgtatagacaagaacacatctactagaaaaataataaaccaacacaccaatgcaactgtgttttcttctgttttaaagtatgttgtctttgtatgcatgtttgcttcttcctttttttttttaacatcacagataaattcaactctcacctcaggttttattgagagaactgtcaatgtgacttggcctctgtctttctagtcccagaaagaattgcactgaaatctgagctcctgtaataaaaacaaccatttgctgagagtaattaacatactgaaagagattttcttagagtacacaatggtgacattatattgcctctttataaataactttctatctatttctgtggattattcctacaaagtacttttcatatgtccaatttcttttcttcccctacaactactgtctgaatactggctctgctatttgctgatatgattctcggcaagttgcctgcactttttaaactttatttcctcattcagaacatggggccatacataatacaactcacttcagtgttattggggaattaaacaaaaaatgcatgggaagcatttaacatagtgcctgacacaataatgagtactcagtagatgttagcttttattaatattgttgttgttatgtccagaaacactatacctccagaaaatcatgggtacttgctggggacattggggatatgcatgatttggaaaagaatgactgctttttttgcttagatgagaaatttttctaagccagactccttcaaatatgtaagattctgttgtggattcaaggactgaaagaattcttggccgagtgtggtggcttatccctgtaatcccagcattttgtgaggacaaggcaggaagattgcttgagtccaggagtttgaaaccagcctgcgcaacatggcgaaaccctgtctctacaaaaaatacaaacattagctcggagtgagtgctgacatgtgcctgtactcccagctactcagaaggctgagatgggaggatctcatgagcctggggagtttgaggcttcagtgagccgtgatgacaccgtactatactccactccagcctgggtgacagtgagaccctgcctcaaaaaacaaacaaacaaacaaacaaaacaaaattaatctttttgctgatgtcatgtcagcagtgtgtgttgaaggctgtaaagcagccatttgttcagtttatttttccattgaacaagtatttatcaaaaacatactttgtggcagtcactatgctaggagctatgaatacagaaggaaaagtaaatgctcttggatactacactccagttgtgataaaaaagaaaaaatgtattcttcaccaacttcaacatcttgatgtgcaaaaacataatacatgaattagatctacctaattacacagaattagaccaattgtttctggaattgtgggctcatatttttaataactgtcctcctgcctctctgtcgacaggttttataaatattcatttaattacacacacacacacgaacaattgactagtacttgctctcattcttctagatgtcatcacaatggatcccaaagacaattgttcaaaagatgcaaatggtaagcttttgtgtttttcccttcctcctgatcattttgttttgaacttctctggcttgaaaaatcagggaatggattttgctaggttggatgctgcagaatggacctagtgatattttaaattagtccctcattttctaggagttgtattaacaaacctaactactgctttggggtatgagatgactgtaaattagagagggtacagtggtatagtgatatgcttttaattatttcaaaaaaaagattttattcattcatgtgtcttttttctttttcttttcttttttttttttttttggacagagtcttgctctgtcacccaggctggagtgcggtggcagtatctcagctcaccacaacctccgcctcccggcttcaagtgattctcctgcctcagcttctcgagtagctgggactacaggcgcgtgccaccatgcccggctaatttttgtatttttagtagagttggggtttcaccatgttggccaggatggcctcgaatttgtgacctcgtgatctgccccctcgccctcccgaactgttgggattacaggcgtgagtcactgtgcccggcctcctgtcctgtcttttgtttaatgactgggaaaaacatgataccatgttgcttctcgagttgttttgttttagtctttggtctttgctagtagctaataacacgaactagtgtttatcaagtgctttttacacagaagggcttgggctgtgttctgcattttcttgtttaaccctcttaaaactcctataaaatggtacatatttttctcccaatttacagtccctttaaagcaaataattataaaaatccctatacatgtcacacagctagatctgggatttcaaatcaggccatcaaacaaagagtttatgtacttagtaagttttctgttctttttctacaatagagtcagatagcaagaaattaccaagccaggaacctgaaacaaaacggacatcatgtggggctgggtgggtgcatgggctttgcagactggactttcactccagctcttttaatgattaggtgtaagtgacctacattttgtgagcaacagttttctcatcagccaacaaagaataattacaccagattcacagttattgaagagataaaggcatgaatgtgagatgtctggcatagggcatctcatttagcagacacagaatgagtacttgtttctggctttttctctctacatatgcacaaagaatgcgactagaagcatgggctctagccctgctcaactttcctctatttccaataccaaggggctctgacttaggctgccacaccaggcaaggagggcagtaccacctcacttgaccaagggcagggagtcacggacacatcacttcttgagatccttttccacaccaaggactgatgtttctggaattctcactttatgaagacaaaacatataaatggaaattttctcaggtagagactcactcttgtagctcattgagtaggcactagtggtccacccccactgtctttacttattccttgacatcacatatctcttgcaaaacctcaaataatattaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatgtatcttattctgtaaaaagaacatgtgcaatttctcttagatacactactgctgcagctcacaaacacctctgcatattacatgtacctcctcctgctcctcaagagtgtggtctattttgccatcatcacctgctgtctgcttagaagaacggctttctgctgcaatggagagaaatcataa

SEQ ID NO: 57 - DNA, 9549 bp, Homo sapiens
  source 1..9549: mol_type = genomic DNA
ataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacaggtatgtgtttacacatatcatctgtcagaacacttctttgaaagtgaatgctgcattttttcctttcagtattaatgaaaaacataaatctttcttaaaaattgttacatttaatggtagcgtaaatgccctgctacttttctatagaattaaaatggtataggttttggagaaaacaaaattgaaaaagttgctgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaataatgtttatctgaagataactgtagatcatatttatctgtagataatgtttatctgtggagtgtggctctacaaaacatagaatagtcttggtcactgcagttttatagaggccttgggtttttcagagtttcattttatatatcaccataaagtaacatttcataattacaggttggtaaggcttacatgtacaaacattcttccattttccataataaatgcatttcctgccattggtgaatgcagctcaataaacatttattgtacaattatgacacgccaggcttagtggaaatgtggatgaacagacaaggatgagttactgtcctaaggatgatgcatgacagtgcagagaatatactctcttcctgatcactcagggtcactcatgattcatgcgcgaggtcccaaaacagtgcctttgatgcagattctgtacatctctagacgattggtccaagggctgaatgtgctctggcccagtggtccagtctgtcactatatgtcaacatcctgaatatgaacataacagtccaacatctcaagagtgggcatgaaaaggactcattttgtgctttttcctgtggttaacaagtcctttttagcctgggggaacaagcattaacaaaatgtttgaagatctttgccacgtaccattccaaatttctagggtaagtctttagcttttcagatcctgagtttctgcaatgatcaaatgtgatttggacagttgcgttgactttctcctggggctataatggagtgcaaaggaaacaatggcagggaaaatgcttgctttcaaaatggtagcatggatgtgttcattcgtgtagttactgtattaggtatagcctttcctgaaactaactgaagtggggttataaaaacagtcccaattttctatttcctttgctgagacacaaagaggagacaaaagagcaaagcttgagggtagttttaccactgtgcttaagtgttctgatttttccagtgatcagggtgaaataaaaagcatagtaagttccagggcagtgaataccatacaggagacaagttacagttttataatgtgttttactttacactaaattctaaaagtaaaatgtctttttttttttccgagacagagtttcactcttgtagcccaggcaggagtgctatggtgtgatctcggctcacagcaacctccacctcccagtttcaagcgattcttctgcctcagcctcccgagaagttgaaattacaggtgcctggcaccatatctcgctaattattctatttttagtagagatcgggttttaccatgttggccaggctggtctcgaactcctgacttcaagtgatccacccgcctcagcctcccaaagtgctgggattacaggtgtgagtcactgtgccggacctaacagtaaaatgtctttcatgtgcttctcaaggcaactacattaaggaggacacatctcttaatgtcattctacagtagatttctaatgctctttcttggaagtttgtttttctgagaagagctaaaaatataataacatggaagtgatcatattatataatcaatgaagtgctttcaaaggagataaaactaacctggtctgcatttgcaaccagccttgattgagagagagagaactcaggatacacttagagattttattatggggaatagttactttattcattttacctcaatcaatgcatggaaataagtgacagtcattttcatttatcttttaataaataaagtcaccatgaggaaaatgaaaacccattaaagtcagtccttaaagatatttggacatgcagacatgataactaacatttccattcgtgagacttacccaaaacctatacctcaagtccatttcttagaatacatgaaataaagatctcagtgagtgtataaaactgcacaccagaatcatatccgtatagacaagaatacatctactagaaaaatataaaccaaaacaccaaggtgactctgtttttttctgttttaaaatatgttgtctttgtatgcatgtttgcttcttcctttttttttttaaacatcgcagataaattcaactctcacctcagttgagagagaactgtcaatgtgacttggcctctctctttctagtcccagaaagaattgcactgaaatgctgagctcctgtaataaaaatgaccatttgctgagagtaattaacatactgaaagagattttcttagaatagtgcacaatggcccaatggtgacattatattgtctctttataaattattttctatctatttctgtggattatttctacaaagcacttttcatatgtccaattccttttattcccctacaagtactgactgactactggctctgctgttcactgatatgactttcggcaagttgcctgcactttttaaacgttatttcctcattcagaacatggggccatacaaaatacaactcacttcagtgttattggggaattaaacaaataaatgcatgggaagcatttaacatagtgcctgacacaataatgagcactcagtagatgttagcttttattaatattgttgttgctatgtccagaaacactatacctccagaaaatcatgggtacttgctggggacgttggggatatgcatgattttgaaaggagtgactgctctttactgctcagatgagaaatttttctaagccagactccttcaaacatgtaagattctgttgtggattctaggactgaaagaattcttggccgagtgtggtggcttatcctggtaatctcatcatttgggaggacaaggcaggaagattgcttgagcccaggagttggaaacaagcctggacaacatggcgaaaccctgtctctacaaaaaatacaaacattagctggtcatgggagtgagtgcctgtactcccagctactcaggaggctaagataggaggatcacctgagcctgggcagtttgaggtttcagtgagccgtgatgacaccatactatactccactccagcctgggtgacagtgacatcctgcctcaaaaaaacccccaaaattattctttttgctgatttcatgtcagcagtgtgtgctgaaggctgtaaagtagccacttgttctgtttatttttccattgaacaagtatttatcaaaaacgtactttgtggaaggcactgtgctaggaactatgcatacagaaggaaaaccaaatgttcttggatactacactccagttgtgataaaaaagaaaaaagtattcttcacaaacttcaacattttgatgtgcaaaaacataatatatgaattagatctacctaactacacagaattagaccaattatttctgggattatgggctcatatttttaataactgtcctcctacctctctgttgacaggttttataaatattcatttaattacacacagtcacagacacactcagacacacacacatacacacacacacacaccttgacaaataatgggcatgaacaattgactggtacttgctctcattcttctagatgtcaccacagtggatcccaaatacaattattcaaaggatgcaaatggtaagtttttgtgttttttatttcctcctgatcattttaagttttgaacttctctggcttgaaaaatcagggaatggattttgctaggttggatgctgcagaatggacctaatcatattttaaattagtccctctttttctaggagttgtattaacaaacctaactactgcttcatgtaagagatgactgtaaattgaagggtacagtgatatgctttcagttatttcaaaaaacagactttactcatccatgtgtcttttttcttttcttttttttcttttttgagacggagtctcgctctgttgaacaggctggattgcagtgacgcgatctcacctcactacaacctccgcctctggagttcaagcgattctccagcctcagcttctcaagtagctgggactacaggcacatgccaccatgtccgggtcatctttgtatttttagcagagaccgggtttcactatgttggccaggctggtctagaattcctgacttcgtgatctgccccctcagccctccgaagtgctgggattacagacgtgagtcactgtgcccggcctaacagtaaaatgtctttcatgcgcttctcaaggcaactacgttaaggaggacacttctcttaatgtcattctacagtagatttctaatgctctttcttggaagtttgtttttctgagaaaagctaaaaatataacatggaagtgatcatattgtataatcaatgaagtgcttttcaaggagataaaactaatctggtccacgtttgcaaccaaccttgattgagagagagagagaactcaggatacacttggagattttattatggggaatagttactttattcttttttcctcaatcaattcatggaaataagtgatagtcatattcatttatcttttaataaatgaagtcaccatgaggaaaataaaaagacattgaaaacccattaaagttagcccttaaagatatttggacatgcagacttgataactaacgtttgcattcttgagacttacccaaaacccatacctcaagtccatgtttttagaattcatgaaataaagatctcagtgagtgcataaaattgcgcaccagaatcatatccgtatagacaagaacacatctactagaaaaataataaaccaacacaccaatgcaactgtgttttcttctgttttaaaatatgttgtctttgtatgcatgtttgcttcttccttttttttttttaacatcacagataaattcaactctcacctcaggttttattgagagaactgtcaatgtgacttggcctctgtctttctagtcccagaaagaatcgcactgaaatgctgagctcctgtaataaaaatgaccatttgctgagagtaattaacatactgaaagagattttcttagagtacacaatggtgacattatattgtctctttataaataactttctatctatttctgtggattattcctacaaagtacttttcatatgtccagtttcttttcttcccctacaactaccgtctgaatactggctctgctatttgctgatatgattctcggcaagttgcctgcactttttaaactttatttcctcattcagaacatggggccatgtaatactcatgtacgtgagtattacgtaataatgctcacttaagtgttactggggaattaaacaaaaaaatgcatggcaagcatttaacatagtgcctgacacaataatgagcactcagtagatgttagattttattaatattgttgttgttatgtccggaaacactatacctccagaaaatcatgggtacttgcttgggatgttggggatatgcatgatttggaaaggtatgactgcttttttctgcttagatgagaaatttttctaagccagactccttcaaatatgtaagattctgttgtggattctaggacggaaagaattcttggtcaggtgtggtttcttatccctgtaatcccagaattttgggaggacaaggcaggaagattgcttgagcccaggagtttgaaaccagcctgggcaacaagacgaaaccctgtctctacaaaagtacataaattagcttggcttggtggtgtgtgcctgtattaccagctattcgggagactgagatgggaggatctcctgaacctgtgaagtttgaggcttcagtgagccgtgatgacaccatactatactcgactccagcctgtgcgacagtgagactctgcgtcaaaaaaaaaaccccaaaattattgtttttgctgatttcaggtcagcagtgtgtgctgaagggtgtaaagtagccacttgatcagtttatttttccactgaacaagtatttatcaaaaacatactttgtggtctgtttttgataaataaaaaggcactgtgctaggagccatgaatacagaaggaaaaccaaatgttcttggatactacactccagttgtgataaaaaagaaaaatgtattcttcacgaacttcaacattttgatatgcaaaaacatagtatataaattagatctacctgattacgtagaatcagaccaattatttctggaattgagggctcatatttttaataactgtcctcctgcctctctgttgacaggttttataaatattcatttaattacacacacacacacacacaccttgacaaataatggacatgaacaattgactagtacttgctctcattcttctagatgtcatcacaatggatcccaaagacaattggtcaaaagatgcaaatggtaagcttttgtgtttttcctttcctcctgatcattttaagttttgaacttctctggcttgaaaaatcagggaatgggccgggtgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcttagtggcgggcgcctgtagtcccagctacttgggaggctgaggcaggagaatggcgtgaacccgggaggcggagcttgcagtgagccgagattgcgccactgcactccactccagcctgggcgacagagcgagactccgtctcaaaaaaaaaaaaaaaaaaaaaaaaagaaaaatcagggaatggattttgctaggttggatgctgcagaatggacctagtgatattttaaattagtccctctttttctaggagttgtattaacaaacctaactactgcttcgggtatgagatgactgtaaattagagggtacagtgatatgctttcagttatttcaaaaaacagactttattcatccgtctgtcttttttttttttttttttttttttttttgagacggaggagtctcactctatcacccaggctggagtgcagtggcgcgatctcggctcaccataacctccgccttactggttcaagcgattctccagcctcagcttctcaagtagctgggactacaggtgcacaccaccatacctggctaatttttgtatttttaatagagatggggtttcaccacgctggccaggatggtcttgaattcttgacctcgtgatctgccccctcgggctcccaaacttctgggattataggcgtgagccactgtgcccggccttctgtcttttgttataatgactggggaaaacatgataccatgttgcttcttgagttgttttgttttagtctttggtctttgctagtagctaataacacgaactagtgtttatcaagtgctttttacacagaagggcttgttctgcattttctagtttaatcatcttaatactcctataaagtagtacaatatattttctcccattttacagtccctttaaagtaaataactataaaaatcccttatacatgtcacacagctaggtctggcatttcaaatcaggacatcaaacaaagaattcgtgcagttactaagtcctctattttttctacaatagaaaaaatagcaagaattacagatagcaagacattacaaggcaggaatctgaaacgaaagggacataatgtggggctgggtgggtgcatgagctttgcagactagactttcattccagctcttttaatgattaggtgtaagtgacctacattttgtgagtaacagttttctcatcagccaactaagaataattacaccagattcacagttattgaagagataagggcatgaatgtgagatgtctggcgtagggtatctcatttagcagacacagaatgaatacttgtttctggctttttctctctacatatgcacaaagaatgtgactagaagcattggctctagccctgctcaactttcctctatttccaataccaaggggctctgacttaggctgccacaccaggcaaggaggggcagtaccacctcacttgaccaagggcagggagtcacggacacatcacttcctgagatccttttccacaccaaggactgatgtttctggaattctcactttatgaagacaaaacatataaatggaaatttctgcaggaagagactcactcttgtagctcattgagtaggcactagtggtccacccccactgtctttacttattccttgacatcacatatctcttgtaaaacctcaaataatgttaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatatatcttattctgtaaaaagaacatgtgcaatttctcttagatacactactgctgcagctcacaaacacctctgcatattacacgtacctcctcctgctcctcaagagtgtggtctattttgccatcatcacctgctgtctgcttagaagaacggctttctgctgcaatggagagaaatcataa

SEQ ID NO: 58 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 59 - protein, 10 aa, synthetic construct
IPNIQNPDPA

SEQ ID NO: 60 - DNA, 30 bp, synthetic construct
  source 1..30: mol_type = other DNA
attcccaatatccagaaccctgaccctgcc

SEQ ID NO: 61 - protein, 11 aa, synthetic construct
LEDLKNVFPPE

SEQ ID NO: 62 - DNA, 33 bp, synthetic construct
  source 1..33: mol_type = other DNA
ctggaggatctgaaaaacgtgttccctcctgaa

SEQ ID NO: 63 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 64 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 65 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 66 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 67 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 68 - DNA, 762 bp, Homo sapiens
  source 1..762: mol_type = genomic DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 69 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 70 - DNA, 864 bp, Homo sapiens
  source 1..864: mol_type = genomic DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 71 - protein, 309 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 72 - protein, 349 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFRSKRSRLLHSDYMNMTPRRPGPTRKHYQPYAPPRDFAAYRKRGRKKLLYIFKQPFMRPVQTTQEEDGCSCRFPEEEEGGCEL

SEQ ID NO: 73 - protein, 5 aa, synthetic construct
NYWMQ

SEQ ID NO: 74 - protein, 17 aa, synthetic construct
AVYPGDGDTRHTQKFKG

SEQ ID NO: 75 - protein, 9 aa, synthetic construct
GFTAYGMDY

SEQ ID NO: 76 - protein, 10 aa, synthetic construct
SASSSVSYMH

SEQ ID NO: 77 - protein, 7 aa, synthetic construct
DTSKLAS

SEQ ID NO: 78 - protein, 9 aa, synthetic construct
QQWSSNPLT

SEQ ID NO: 79 - protein, 118 aa, synthetic construct
QVQLQQSGAELARPGASVKLSCKASGYTFTNYWMQWIKQRPGQGLEWIGAVYPGDGDTRHTQKFKGKATLTADKSSSTAYMQVSSLASEDSAVYYCARGFTAYGMDYWGQGTSVTVSS

SEQ ID NO: 80 - DNA, 354 bp, synthetic construct
  source 1..354: mol_type = other DNA
caagttcagctgcaacagtctggcgccgagcttgctagacctggcgcttctgtgaagctgagctgtaaagccagcggctacaccttcaccaactactggatgcagtggatcaagcagcggcctggacagggactcgagtggattggagctgtgtatcctggcgacggcgacaccagacacacccagaagttcaagggcaaagccacactgaccgccgacaagagcagcagcacagcctacatgcaggtttccagcctggccagcgaagatagcgccgtgtactattgcgccagaggctttaccgcctacggcatggattattggggccagggcacaagcgtgaccgtgtctagc

SEQ ID NO: 81 - protein, 107 aa, synthetic construct
QIVLTQSPAVMSASPGEKVTMTCSASSSVSYMHWYQQKSGTSPKRWIYDTSKLASGVPNRFSGSGSGTSYSFTIRSMEAEDAATYYCQQWSSNPLTFGAGTKLELKR

SEQ ID NO: 82 - DNA, 321 bp, synthetic construct
  source 1..321: mol_type = other DNA
caaattgtgctgacacagagccctgccgtgatgtctgctagccctggcgagaaagtgaccatgacctgtagcgccagcagcagcgtgtcctacatgcactggtatcagcagaagtccggcacaagccccaagcggtggatctacgatacaagcaagctggcctccggcgtgcccaatagattttctggcagcggctctggcaccagctacagcttcaccatcagatccatggaagccgaggatgccgccacctactactgccagcagtggtccagcaatcccctgacatttggagccggcaccaagctggaactgaagaga

SEQ ID NO: 83 - protein, 258 aa, synthetic construct
MALPVTALLLPLALLLHAQVQLQQSGAELARPGASVKLSCKASGYTFTNYWMQWIKQRPGQGLEWIGAVYPGDGDTRHTQKFKGKATLTADKSSSTAYMQVSSLASEDSAVYYCARGFTAYGMDYWGQGTSVTVSSGGGGSGGGGSGGGGSQIVLTQSPAVMSASPGEKVTMTCSASSSVSYMHWYQQKSGTSPKRWIYDTSKLASGVPNRFSGSGSGTSYSFTIRSMEAEDAATYYCQQWSSNPLTFGAGTKLELKR

SEQ ID NO: 84 - DNA, 774 bp, synthetic construct
  source 1..774: mol_type = other DNA
atggctctcccagtgactgccctactgcttcccctagcgcttctcctgcatgcacaagttcagctgcaacagtctggcgccgagcttgctagacctggcgcttctgtgaagctgagctgtaaagccagcggctacaccttcaccaactactggatgcagtggatcaagcagcggcctggacagggactcgagtggattggagctgtgtatcctggcgacggcgacaccagacacacccagaagttcaagggcaaagccacactgaccgccgacaagagcagcagcacagcctacatgcaggtttccagcctggccagcgaagatagcgccgtgtactattgcgccagaggctttaccgcctacggcatggattattggggccagggcacaagcgtgaccgtgtctagcggtggaggtggatcaggtggaggtggatctggtggaggtggatctcaaattgtgctgacacagagccctgccgtgatgtctgctagccctggcgagaaagtgaccatgacctgtagcgccagcagcagcgtgtcctacatgcactggtatcagcagaagtccggcacaagccccaagcggtggatctacgatacaagcaagctggcctccggcgtgcccaatagattttctggcagcggctctggcaccagctacagcttcaccatcagatccatggaagccgaggatgccgccacctactactgccagcagtggtccagcaatcccctgacatttggagccggcaccaagctggaactgaagaga

SEQ ID NO: 85 - protein, 11 aa, synthetic construct
KASQDVRTAVA

SEQ ID NO: 86 - protein, 7 aa, synthetic construct
SASYRYT

SEQ ID NO: 87 - protein, 10 aa, synthetic construct
QQHYGTPPWT

SEQ ID NO: 88 - protein, 5 aa, synthetic construct
SYAMS

SEQ ID NO: 89 - protein, 17 aa, synthetic construct
AISGGGRYTYYPDSMKG

SEQ ID NO: 90 - protein, 8 aa, synthetic construct
HYDGYLDY

SEQ ID NO: 91 - protein, 108 aa, synthetic construct
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIK

SEQ ID NO: 92 - DNA, 324 bp, synthetic construct
  source 1..324: mol_type = other DNA
gacattgtgatgacccagtctcacaaattcatgtccacatcaattggagccagggtcagcatcacctgcaaggccagtcaggatgtgagaactgctgtagcctggtatcaacagaaaccaggccagtctcctaaactactaatttactcggcatcctaccggtacactggagtccctgatcgcttcactggcagtggatctgggacggatttcactttcaccatcagcagtgtgcaggctgaagacctggcagtttattactgtcagcaacattatggtactcctccgtggacgttcggtggaggcaccaagctggaaatcaaa

SEQ ID NO: 93 - protein, 117 aa, synthetic construct
EVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 94 - DNA, 351 bp, synthetic construct
  source 1..351: mol_type = other DNA
gaagtgcagctggtggagtctgggggaggcttagtgaagcctggagggtccctgaaactctcctgtgaagcctctagattcactttcagtagctatgccatgtcttgggttcgccagactccggagaagaggctggagtgggtcgcagccattagtggaggtggtaggtacacctactatccagacagtatgaagggtcgattcaccatctccagagacaatgccaagaatttcctgtacctgcaaatgagcagtctgaggtctgaggacacggccatgtattactgtgcaagacactatgatggttatcttgactactggggccaaggcaccactctcacagtctcctca

SEQ ID NO: 95 - protein, 259 aa, synthetic construct
MEFGLSWLFLVAILKGVQCDIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIKGGGGSGGGGSGGGGSEVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 96 - DNA, 777 bp, synthetic construct
  source 1..777: mol_type = other DNA
atggaattcggcctgagctggctgttcctggtggccatcctgaagggcgtgcagtgcgacattgtgatgacccagtctcacaaattcatgtccacatcaattggagccagggtcagcatcacctgcaaggccagtcaggatgtgagaactgctgtagcctggtatcaacagaaaccaggccagtctcctaaactactaatttactcggcatcctaccggtacactggagtccctgatcgcttcactggcagtggatctgggacggatttcactttcaccatcagcagtgtgcaggctgaagacctggcagtttattactgtcagcaacattatggtactcctccgtggacgttcggtggaggcaccaagctggaaatcaaaggcggcggaggatctggcggaggcggaagtggcggagggggctctgaagtgcagctggtggagtctgggggaggcttagtgaagcctggagggtccctgaaactctcctgtgaagcctctagattcactttcagtagctatgccatgtcttgggttcgccagactccggagaagaggctggagtgggtcgcagccattagtggaggtggtaggtacacctactatccagacagtatgaagggtcgattcaccatctccagagacaatgccaagaatttcctgtacctgcaaatgagcagtctgaggtctgaggacacggccatgtattactgtgcaagacactatgatggttatcttgactactggggccaaggcaccactctcacagtctcctca

SEQ ID NO: 97 - protein, 7 aa, synthetic construct
SNSAAWN

SEQ ID NO: 98 - protein, 18 aa, synthetic construct
RTYYRSKWYNDYAVSVKS

SEQ ID NO: 99 - protein, 12 aa, synthetic construct
EVTGDLEDAFDI

SEQ ID NO: 100 - protein, 11 aa, synthetic construct
RASQTIWSYLN

SEQ ID NO: 101 - protein, 7 aa, synthetic construct
AASSLQS

SEQ ID NO: 102 - protein, 9 aa, synthetic construct
QQSYSIPQT

SEQ ID NO: 103 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 104 - DNA, 378 bp, synthetic construct
  source 1..378: mol_type = other DNA
cagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctcc

SEQ ID NO: 105 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 106 - DNA, 321 bp, synthetic construct
  source 1..321: mol_type = other DNA
gacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaa

SEQ ID NO: 107 - protein, 265 aa, synthetic construct
MELGLSWIFLLAILKGVQCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 108 - DNA, 795 bp, synthetic construct
  source 1..795: mol_type = other DNA
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggtggaggtggatcaggtggaggtggatctggtggaggtggatctgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaa

SEQ ID NO: 109 - protein, 255 aa, synthetic construct
MELGLSWIFLLAILKGVQCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 110 - DNA, 765 bp, synthetic construct
  source 1..765: mol_type = other DNA
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggaggaggaggcagcgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaa

SEQ ID NO: 111 - protein, 124 aa, synthetic construct; identical to SEQ ID NO: 103

SEQ ID NO: 112 - DNA, 372 bp, synthetic construct
  source 1..372: mol_type = other DNA
caagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctcc

SEQ ID NO: 113 - protein, 107 aa, synthetic construct; identical to SEQ ID NO: 105

SEQ ID NO: 114 - DNA, 321 bp, synthetic construct; identical to SEQ ID NO: 106
  source 1..321: mol_type = other DNA

SEQ ID NO: 115 - protein, 240 aa, synthetic construct
MELGLSWIFLLAILKGVQCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAED

SEQ ID NO: 116 - DNA, 720 bp, synthetic construct
  source 1..720: mol_type = other DNA
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggtggaggtggatcaggtggaggtggatctggtggaggtggatctgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagat

SEQ ID NO: 117 - protein, 255 aa, synthetic construct; identical to SEQ ID NO: 109

SEQ ID NO: 118 - DNA, 765 bp, synthetic construct; identical to SEQ ID NO: 110
  source 1..765: mol_type = other DNA

SEQ ID NO: 119 - protein, 5 aa, synthetic construct
IYDMS

SEQ ID NO: 120 - protein, 17 aa, synthetic construct
YISSGGGTTYYPDTVKG

SEQ ID NO: 121 - protein, 14 aa, synthetic construct
HSGYGTHWGVLFAY

SEQ ID NO: 122 - protein, 11 aa, synthetic construct
RASQDIGRYLN

SEQ ID NO: 123 - protein, 7 aa, synthetic construct
YTSILHS

SEQ ID NO: 124 - protein, 9 aa, synthetic construct
QQGNTLPWT

SEQ ID NO: 125 - protein, 123 aa, synthetic construct
EVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSA

SEQ ID NO: 126 - DNA, 369 bp, synthetic construct
  source 1..369: mol_type = other DNA
gaagtgcagctggtggagtctgggggaggcttagtgaagcctggagggtccctgaaactctcctgtgcagcctctggattcgctttcagtatctatgacatgtcttgggttcgccagactccggagaagaggctggagtgggtcgcatacattagtagtggtggtggtaccacctactatccagacactgtgaagggccgattcaccatctccagagacaatgccaagaacaccctgtacctgcaaatgagcagtctgaagtctgaggacacagccatgtattactgtgcaagacatagtggctacggtacccactggggggttttgtttgcttactggggccaagggactctggtcactgtctctgca

SEQ ID NO: 127 - protein, 107 aa, synthetic construct
DIQMTQTTSSLSASLGDRVTISCRASQDIGRYLNWYQQKPDGTVKLLIYYTSILHSGVPSRFSGSGSGTDYSLTISNLEQEDFATYFCQQGNTLPWTFGGGTKLEIK

SEQ ID NO: 128 - DNA, 321 bp, synthetic construct
  source 1..321: mol_type = other DNA
gatatccagatgacccagactacatcctccctgtctgcctctctgggagacagagtcaccattagttgcagggcaagtcaggacattggccgctatttaaactggtatcagcagaaaccagatggaactgttaaactcctgatctactacacatcaatattacactcaggagtcccatcaaggttcagtggcagtgggtctggaacagattattctctcaccattagcaacctggagcaagaagattttgccacttacttttgccaacagggtaatacgcttccgtggacgttcggtggaggcaccaagctggaaatcaaa

SEQ ID NO: 129 - protein, 264 aa, synthetic construct
MEFGLSWIFLAAILKGVQCEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDIGRYLNWYQQKPDGTVKLLIYYTSILHSGVPSRFSGSGSGTDYSLTISNLEQEDFATYFCQQGNTLPWTFGGGTKLEIK

SEQ ID NO: 130 - DNA, 792 bp, synthetic construct
  source 1..792: mol_type = other DNA
atggagtttgggctgagctggattttccttgctgctattttaaaaggtgtccagtgtgaagtgcagctggtggagtctgggggaggcttagtgaagcctggagggtccctgaaactctcctgtgcagcctctggattcgctttcagtatctatgacatgtcttgggttcgccagactccggagaagaggctggagtgggtcgcatacattagtagtggtggtggtaccacctactatccagacactgtgaagggccgattcaccatctccagagacaatgccaagaacaccctgtacctgcaaatgagcagtctgaagtctgaggacacagccatgtattactgtgcaagacatagtggctacggtacccactggggggttttgtttgcttactggggccaagggactctggtcactgtctctgcaggtggaggtggatcaggtggaggtggatctggtggaggtggatctgatatccagatgacccagactacatcctccctgtctgcctctctgggagacagagtcaccattagttgcagggcaagtcaggacattggccgctatttaaactggtatcagcagaaaccagatggaactgttaaactcctgatctactacacatcaatattacactcaggagtcccatcaaggttcagtggcagtgggtctggaacagattattctctcaccattagcaacctggagcaagaagattttgccacttacttttgccaacagggtaatacgcttccgtggacgttcggtggaggcaccaagctggaaatcaaa

SEQ ID NO: 131 - protein, 254 aa, synthetic construct
MEFGLSWIFLAAILKGVQCEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSAGGGGSDIQMTQTTSSLSASLGDRVTISCRASQDIGRYLNWYQQKPDGTVKLLIYYTSILHSGVPSRFSGSGSGTDYSLTISNLEQEDFATYFCQQGNTLPWTFGGGTKLEIK

SEQ ID NO: 132 - DNA, 762 bp, synthetic construct
  source 1..762: mol_type = other DNA
atggagtttgggctgagctggattttccttgctgctattttaaaaggtgtccagtgtgaagtgcagctggtggagtctgggggaggcttagtgaagcctggagggtccctgaaactctcctgtgcagcctctggattcgctttcagtatctatgacatgtcttgggttcgccagactccggagaagaggctggagtgggtcgcatacattagtagtggtggtggtaccacctactatccagacactgtgaagggccgattcaccatctccagagacaatgccaagaacaccctgtacctgcaaatgagcagtctgaagtctgaggacacagccatgtattactgtgcaagacatagtggctacggtacccactggggggttttgtttgcttactggggccaagggactctggtcactgtctctgcaggaggaggaggcagcgatatccagatgacccagactacatcctccctgtctgcctctctgggagacagagtcaccattagttgcagggcaagtcaggacattggccgctatttaaactggtatcagcagaaaccagatggaactgttaaactcctgatctactacacatcaatattacactcaggagtcccatcaaggttcagtggcagtgggtctggaacagattattctctcaccattagcaacctggagcaagaagattttgccacttacttttgccaacagggtaatacgcttccgtggacgttcggtggaggcaccaagctggaaatcaaa

SEQ ID NO: 133 - protein, 5 aa, synthetic construct
SYIMH

SEQ ID NO: 134 - protein, 17 aa, synthetic construct
VISYDGRNKYYADSVKG

SEQ ID NO: 135 - protein, 9 aa, synthetic construct
DTDGYDFDY

SEQ ID NO: 136 - protein, 11 aa, synthetic construct
RASQSVSSYLA

SEQ ID NO: 137 - protein, 7 aa, synthetic construct
DASNRAT

SEQ ID NO: 138 - protein, 9 aa, synthetic construct
QQRTNWPLT

SEQ ID NO: 139 - protein, 135 aa, synthetic construct
MELGLSWIFLLAILKGVQCQVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTV

SEQ ID NO: 140 - DNA, 405 bp, synthetic construct
  source 1..405: mol_type = other DNA
atggaactcggtctgtcctggatttttctgcttgcgatcttgaaaggagtgcagtgccaggtgcagctggtggagtctgggggaggcgtggtccagcctgggaggtccctgagactctcctgtgcagcctctggatttaccttcagtagctatattatgcactgggtccgccaggctccaggcaaggggctggagtgggtggcagttatatcatatgatggaagaaacaaatactacgcagactccgtgaagggccgattcaccatctccagagacaattccaagaacacgctgtatctgcaaatgaacagcctgagagctgaggacacggctgtgtattactgtgcgagagatacggatggctacgattttgactactggggccagggaaccctggtcaccgtc

SEQ ID NO: 141 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 142 - DNA, 378 bp, synthetic construct
  source 1..378: mol_type = other DNA
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaccaactggccgctcactttcggcggagggaccaaggtggagatc

SEQ ID NO: 143 - protein, 5 aa, synthetic construct
SYWMN

SEQ ID NO: 144 - protein, 17 aa, synthetic construct
QIYPGDGDTNYNGKFKG

SEQ ID NO: 145 - protein, 13 aa, synthetic construct
KTISSVVDFYFDY

SEQ ID NO: 146 - protein, 11 aa, synthetic construct
KASQNVGTNVA

SEQ ID NO: 147 - protein, 7 aa, synthetic construct
SATYRNS

SEQ ID NO: 148 - protein, 9 aa, synthetic construct
QQYNRYPYT

SEQ ID NO: 149 - protein, 138 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTV

SEQ ID NO: 150 - DNA, 414 bp, synthetic construct
  source 1..414: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtc

SEQ ID NO: 151 - protein, 124 aa, synthetic construct
MALPVTALLLPLALLLHADIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 152 - DNA, 372 bp, synthetic construct
  source 1..372: mol_type = other DNA
atggcactgcccgtgaccgctctgcttctcccactagctctgcttctccacgcagacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatc

SEQ ID NO: 153 - protein, 18 aa, synthetic construct
RTYYRSKWYNDYAVSVKS

SEQ ID NO: 154 - protein, 18 aa, synthetic construct
RTYRRSKWYNDYAVSVKS

SEQ ID NO: 155 - protein, 124 aa, synthetic construct
QVQLQQSGPGLVKPSQTLSLTCAISGDSVSSNSAAWNWIRQSPSRGLEWLGRTYRRSKWYNDYAVSVKSRITINPDTSKNQFSLQLNSVTPEDTAVYYCAREVTGDLEDAFDIWGQGTMVTVSS

SEQ ID NO: 156 - DNA, 372 bp, synthetic construct
  source 1..372: mol_type = other DNA
caagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctatcgcaggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctcc

SEQ ID NO: 157 - protein, 246 aa, synthetic construct
QVQLQQSGPGLVKPSQTLSLTCAISGDSVSSNSAAWNWIRQSPSRGLEWLGRTYRRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 158 - protein, 265 aa, synthetic construct
MELGLSWIFLLAILKGVQCQVQLQQSGPGLVKPSQTLSLTCAISGDSVSSNSAAWNWIRQSPSRGLEWLGRTYRRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 159 - DNA, 738 bp, synthetic construct
  source 1..738: mol_type = other DNA
caagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctatcgcaggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggtggaggtggatcaggtggaggtggatctggtggaggtggatctgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaa

SEQ ID NO: 160 - DNA, 795 bp, synthetic construct
  source 1..795: mol_type = other DNA
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctatcgcaggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggtggaggtggatcaggtggaggtggatctggtggaggtggatctgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaa

SEQ ID NO: 161 - RNA, 24 nt, synthetic construct
  source 1..24: mol_type = other RNA
atgacaggttgaagcaagtagata

SEQ ID NO: 162 - RNA, 24 nt, synthetic construct
  source 1..24: mol_type = other RNA
caagttcaagagaaaaagtgtata

SEQ ID NO: 163 - RNA, 24 nt, synthetic construct
  source 1..24: mol_type = other RNA
cgctgctgattagggttttttata

SEQ ID NO: 164 - RNA, 24 nt, synthetic construct
  source 1..24: mol_type = other RNA
ccgtgatggcatctacatggtata

SEQ ID NO: 165 - RNA, 24 nt, synthetic construct
  source 1..24: mol_type = other RNA
aaagagaaaaagtgtacacacata

SEQ ID NO: 166 - RNA, 24 nt, synthetic construct
  source 1..24: mol_type = other RNA
tctacttgcttcaacctgtcagtg

SEQ ID NO: 167 - RNA, 24 nt, synthetic construct
  source 1..24: mol_type = other RNA
tacactttttctcttgaacttatg

SEQ ID NO: 168 - RNA, 24 nt, synthetic construct
  source 1..24: mol_type = other RNA
taaaaaaccctaatcagcagcatg

SEQ ID NO: 169 - RNA, 24 nt, synthetic construct
  source 1..24: mol_type = other RNA
taccatgtagatgccatcacgatg

SEQ ID NO: 170 - RNA, 24 nt, synthetic construct
  source 1..24: mol_type = other RNA
tgtgtgtacactttttctcttgtg

SEQ ID NO: 171 - DNA, 97 bp, synthetic construct
  source 1..97: mol_type = other DNA
tgctgttgacagtgagcgatgacaggttgaagcaagtagatagtgaagccacagatgtatctacttgcttcaacctgtcagtgcctactgcctcgga

SEQ ID NO: 172 - DNA, 97 bp, synthetic construct
  source 1..97: mol_type = other DNA
tgctgttgacagtgagcgcaagttcaagagaaaaagtgtatagtgaagccacagatgtatacactttttctcttgaacttatgcctactgcctcgga

SEQ ID NO: 173 - DNA, 97 bp, synthetic construct
  source 1..97: mol_type = other DNA
tgctgttgacagtgagcgcgctgctgattagggttttttatagtgaagccacagatgtataaaaaaccctaatcagcagcatgcctactgcctcgga

SEQ ID NO: 174 - DNA, 97 bp, synthetic construct
  source 1..97: mol_type = other DNA
tgctgttgacagtgagcgccgtgatggcatctacatggtatagtgaagccacagatgtataccatgtagatgccatcacgatgcctactgcctcgga

SEQ ID NO: 175 - DNA, 97 bp, synthetic construct
  source 1..97: mol_type = other DNA
tgctgttgacagtgagcgaaagagaaaaagtgtacacacatagtgaagccacagatgtatgtgtgtacactttttctcttgtgcctactgcctcgga

SEQ ID NO: 176 - RNA, 24 nt, synthetic construct
  source 1..24: mol_type = other RNA
ctcaccgattttgattctcaaata

SEQ ID NO: 177 - RNA, 24 nt, synthetic construct
  source 1..24: mol_type = other RNA
atggtcgagaaaagctttgaaata

SEQ ID NO: 178 - RNA, 24 nt, synthetic construct
  source 1..24: mol_type = other RNA
acgattttgattctcaaacaaata

SEQ ID NO: 179 - RNA, 24 nt, synthetic construct
  source 1..24: mol_type = other RNA
cttcaccgattttgattctcaata

SEQ ID NO: 180 - RNA, 24 nt, synthetic construct
  source 1..24: mol_type = other RNA
aagatacgaacctaaactttcata

SEQ ID NO: 181 - RNA, 24 nt, synthetic construct
  source 1..24: mol_type = other RNA
tttgagaatcaaaatcggtgaatg

SEQ ID NO: 182 - RNA, 24 nt, synthetic construct
  source 1..24: mol_type = other RNA
tttcaaagcttttctcgaccagtg

SEQ ID NO: 183 - RNA, 24 nt, synthetic construct
  source 1..24: mol_type = other RNA
tttgtttgagaatcaaaatcggtg

SEQ ID NO: 184 - RNA, 24 nt, synthetic construct
  source 1..24: mol_type = other RNA
ttgagaatcaaaatcggtgaattg

SEQ ID NO: 185 - RNA, 24 nt, synthetic construct
  source 1..24: mol_type = other RNA
tgaaagtttaggttcgtatctgtg

SEQ ID NO: 186 - DNA, 97 bp, synthetic construct
  source 1..97: mol_type = other DNA
tgctgttgacagtgagcgctcaccgattttgattctcaaatagtgaagccacagatgtatttgagaatcaaaatcggtgaatgcctactgcctcgga

SEQ ID NO: 187 - DNA, 97 bp, synthetic construct
  source 1..97: mol_type = other DNA
tgctgttgacagtgagcgatggtcgagaaaagctttgaaatagtgaagccacagatgtatttcaaagcttttctcgaccagtgcctactgcctcgga

SEQ ID NO: 188 - DNA, 97 bp, synthetic construct
  source 1..97: mol_type = other DNA
tgctgttgacagtgagcgacgattttgattctcaaacaaatagtgaagccacagatgtatttgtttgagaatcaaaatcggtgcctactgcctcgga

SEQ ID NO: 189 - DNA, 97 bp, synthetic construct
  source 1..97: mol_type = other DNA
tgctgttgacagtgagcgcttcaccgattttgattctcaatagtgaagccacagatgtattgagaatcaaaatcggtgaattgcctactgcctcgga

SEQ ID NO: 190 - DNA, 97 bp, synthetic construct
  source 1..97: mol_type = other DNA
tgctgttgacagtgagcgaagatacgaacctaaactttcatagtgaagccacagatgtatgaaagtttaggttcgtatctgtgcctactgcctcgga

SEQ ID NO: 191 - RNA, 24 nt, synthetic construct
  source 1..24: mol_type = other RNA
cccgctgtcaagtccagttctata

SEQ ID NO: 192 - RNA, 24 nt, synthetic construct
  source 1..24: mol_type = other RNA
acgccctcaatgactccagatata

SEQ ID NO: 193 - RNA, 24 nt, synthetic construct
  source 1..24: mol_type = other RNA
aacccgaggtcgctgtgtttgata

SEQ ID NO: 194 - RNA, 24 nt, synthetic construct
  source 1..24: mol_type = other RNA
acgcaaccacttccgctgtcaata

SEQ ID NO: 195 - RNA, 24 nt, synthetic construct
  source 1..24: mol_type = other RNA
cgcagagatctcccacacccaata

SEQ ID NO: 196 - RNA, 24 nt, synthetic construct
  source 1..24: mol_type = other RNA
ccgctgtgtttgagccatcagata

SEQ ID NO: 197 - RNA, 24 nt, synthetic construct
  source 1..24: mol_type = other RNA
tagaactggacttgacagcggatg

SEQ ID NO: 198 - RNA, 24 nt, synthetic construct
  source 1..24: mol_type = other RNA
tatctggagtcattgagggcggtg

SEQ ID NO: 199 - RNA, 24 nt, synthetic construct
  source 1..24: mol_type = other RNA
tcaaacacagcgacctcgggtgtg

SEQ ID NO: 200 - RNA, 24 nt, synthetic construct
  source 1..24: mol_type = other RNA
ttgacagcggaagtggttgcggtg

SEQ ID NO: 201 - RNA, 24 nt, synthetic construct
  source 1..24: mol_type = other RNA
ttgggtgtgggagatctctgcttg

SEQ ID NO: 202 - RNA, 24 nt, synthetic construct
  source 1..24: mol_type = other RNA
tctgatggctcaaacacagcgatg

SEQ ID NO: 203 - DNA, 97 bp, synthetic construct
  source 1..97: mol_type = other DNA
tgctgttgacagtgagcgcccgctgtcaagtccagttctatagtgaagccacagatgtatagaactggacttgacagcggatgcctactgcctcgga

SEQ ID NO: 204 - DNA, 97 bp, synthetic construct
  source 1..97: mol_type = other DNA
tgctgttgacagtgagcgacgccctcaatgactccagatatagtgaagccacagatgtatatctggagtcattgagggcggtgcctactgcctcgga

SEQ ID NO: 205 - DNA, 97 bp, synthetic construct
  source 1..97: mol_type = other DNA
tgctgttgacagtgagcgaacccgaggtcgctgtgtttgatagtgaagccacagatgtatcaaacacagcgacctcgggtgtgcctactgcctcgga

SEQ ID NO: 206 - DNA, 97 bp, synthetic construct
  source 1..97: mol_type = other DNA
tgctgttgacagtgagcgacgcaaccacttccgctgtcaatagtgaagccacagatgtattgacagcggaagtggttgcggtgcctactgcctcgga

SEQ ID NO: 207 - DNA, 97 bp, synthetic construct
  source 1..97: mol_type = other DNA
tgctgttgacagtgagcgcgcagagatctcccacacccaatagtgaagccacagatgtattgggtgtgggagatctctgcttgcctactgcctcgga

SEQ ID NO: 208 - DNA, 97 bp, synthetic construct
  source 1..97: mol_type = other DNA
tgctgttgacagtgagcgccgctgtgtttgagccatcagatagtgaagccacagatgtatctgatggctcaaacacagcgatgcctactgcctcgga